(19) United States
(12) Patent Application Publication
(10) Pub. No.: US 2008/0318943 A1
(43) Pub. Date: Dec. 25, 2008

(54) Compounds and Uses Thereof - 848

(75) Inventors: Hui-Fang Chang, Wilmington, DE (US); Marc Chapdelaine, Wilmington, DE (US); Bruce Thomas Dembofsky, Wilmington, DE (US); Keith John Herzog, Wilmington, DE (US); Carey Horchler, Wilmington, DE (US); Richard Jon Schmiesing, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/141,190

(22) Filed: Jun. 18, 2008

Related U.S. Application Data
(60) Provisional application No. 60/944,879, filed on Jun. 19, 2007.

(51) Int. Cl. A61K 31/5377; C07D 471/04; C07K 14/705; A61P 25/28; A61P 25/18; A61K 31/437

(52) U.S. Cl. 514/232.8; 546/84; 514/292; 544/126; 530/409

(57) ABSTRACT

This invention relates to novel compounds having the structural formula I below:

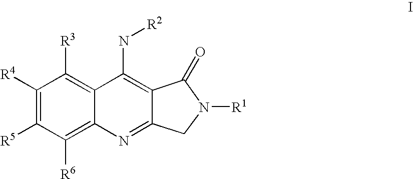

and their pharmaceutically acceptable salts, tautomers or in vivo-hydrolysable precursors, compositions and methods of use thereof, wherein R¹, R², R³, R⁴, R⁵, and R⁶ are defined in the specification. These novel compounds provide a treatment or prophylaxis of anxiety disorders, schizophrenia, cognitive disorders, and/or mood disorders.

The present application claims the benefit of U.S. Provisional Application 60/944,879, filed Jun. 19, 2007 under 35 U.S.C. §119(e), the entirety of which is incorporated herein by reference.

### FIELD OF THE INVENTION

The present invention relates to novel quinoline compounds, their pharmaceutical compositions, methods of use and processes to make such compounds. In addition, the present invention relates to therapeutic methods for the treatment and/or prevention of anxiety disorders, schizophrenia, cognitive disorders, and/or mood disorders.

### BACKGROUND OF THE INVENTION

gamma-Aminobutyric acid (GABA) is a common inhibitory neurotransmitter in the mammalian brain and is estimated to be present at about one third of all synapses. When GABA binds to a GABA receptor, it affects the ability of neurons expressing the receptors to conduct neural impulses. In the adult mammalian nervous system, GABA typically inhibits neuron firing (depolarization). Neurons in the brain express three main types of GABA receptors: GABA type A receptors (GABAA), GABA type B receptors (GABAB), and GABA type C receptors (GABAC). GABAA receptors function as ligand-gated ion channels to mediate fast inhibitory synaptic transmissions that regulate neuronal excitability involved in such responses as seizure threshold, skeletal muscle tone, and emotional status. GABAA receptors are targets of many sedating drugs, such as benzodiazepines, barbiturates and neurosteroids.

The intrinsic inhibitory signal of GABA is transduced principally by GABAA receptors. GABAA receptors are pentameric, ligand-gated chloride ion (Cl⁻) channels belonging to a superfamily of ligand-gated ionotropic receptors that includes the nicotinic acetylcholine receptor. GABAA receptors are very heterogeneous, with at least 16 different subunits producing potentially thousands of different receptor types.

GABAA receptor subunits aggregate into complexes that form chloride ion selective channels and contain sites that bind GABA along with a variety of pharmacologically active substances. When GABA binds to this receptor, the anion channel is activated, causing it to open and allowing chloride ions (Cl⁻) to enter the neuron. This influx of Cl⁻ ions hyperpolarizes the neuron, making it less excitable. The resultant decrease in neuronal activity following activation of the GABAA receptor complex can rapidly alter brain function, to such an extent that consciousness and motor control may be impaired.

The numerous possible combinations of GABAA receptor subunits and the widespread distribution of these receptors in the nervous system likely contributes to the diverse and variable physiological functions of GABAA receptors, which have been implicated in many neurological and psychiatric disorders, and related conditions, including: stroke, head trauma, epilepsy, pain, migraine, mood disorders, anxiety, post traumatic stress disorder, obsessive compulsive disorders, schizophrenia, seizures, convulsions, tinnitus, neurodegenerative disorders including Alzheimer's disease, amyotrophic lateral sclerosis, Huntington's Chorea, Parkinson's disease, depression, bipolar disorders, mania, trigeminal and other neuralgia, neuropathic pain, hypertension, cerebral ischemia, cardiac arrhythmia, myotonia, substance abuse, myoclonus, essential tremor, dyskinesia and other movement disorders, neonatal cerebral hemorrhage, and spasticity. GABAA receptors are also believed to play a role in cognition, consciousness, and sleep.

Currently available drugs for modulating GABAA receptor activity include barbiturates, such as pentobarbital and secobarbital, and benzodiazepines such as diazepam, chlordiazepoxide and midazolam. Barbiturates can directly activate GABAA receptors, significantly increasing Cl⁻ currents in the absence of further intervention by GABA itself and can also indirectly augment GABAergic neural transmission. In contrast, benzodiazepines act as indirect allosteric modulators, and are largely incapable of increasing Cl⁻ currents in the absence of GABA, but enhance GABA-activated increases in Cl⁻ conductance. This latter property is thought to be responsible for the usefulness of benzodiazepines for treating a number of disorders, including generalized anxiety disorder, panic disorder, seizures, movement disorders, epilepsy, psychosis, mood disorders, and muscle spasms, as well as the relative safety of benzodiazepines compared to barbiturates.

Both barbiturates and benzodiazepines are addictive and can cause drowsiness, poor concentration, ataxia, dysarthria, motor incoordination, diplopia, muscle weakness, vertigo and mental confusion. These side effects can interfere with an individual's ability to perform daily routines such as driving, operating heavy machinery or performing other complex motor tasks while under therapy, making barbiturates and benzodiazepines less than optimal for treating chronic disorders involving GABA and GABAA receptors.

GABAA receptors and GABAergic neural transmissions are implicated as targets for therapeutic intervention in a myriad of neurological and psychiatric disorders. Adverse side effects, including addictive properties exhibited by currently available GABA and GABAA receptor modulating drugs, make these drugs unsuitable in many therapeutic contexts. Accordingly, there remains an important, unmet need in the art for alternative compositions, methods and tools that will be useful in broad clinical applications to modulate the function and activity of GABA and GABA receptors in mammalian subjects, including humans, and/or to target GABAergic neural transmission. Certain embodiments of the present invention are, inter alia, directed toward this end.

Some quinoline compounds are disclosed in U.S. Pat. No. 4,975,435. However, there is still a need for new quinolines that have improved pharmacological properties, improved efficacy and additional therapeutic effects.

### DESCRIPTION OF EMBODIMENTS

Provided herein are novel compounds of formula I:

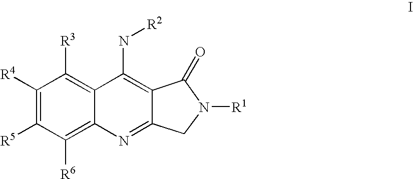

or a pharmaceutically acceptable salt, tautomer, atropisomer, or in vivo-hydrolysable precursor thereof, wherein:

R¹ is C₁₋₆ alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl, wherein each of the C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl is optionally substituted by 1, 2, 3, 4 or 5 R⁷;

R² is H, —C(═O)R^{b}, —C(═O)NR^{c}R_{d}, —C(═O)OR^{a}, —S(═O)₂R^{b}, C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl, wherein each of the C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl is optionally substituted by 1, 2, 3, 4 or 5 R⁸;

R³, R⁴ and R⁵ are each, independently, H, halo, —Si(C₁₋₁₀alkyl)₃, —CN, —NO₂, —OR^{a}, —SR^{a}, —OC(═O)R^{a}, —OC(═O)OR^{b}, —OC(═O)NR^{c}R^{d}, —C(═O)R^{a}, —C(═O)OR^{b}, —C(═O)NR^{c}R^{d}, —NR^{c}R^{d}, —NR^{c}C(═O)R^{a}, —NR^{c}C(═O)OR^{b}, —NR^{c}S(═O)₂R^{b}, —S(═O)R^{a}, —S(═O)NR^{c}R^{d}, —S(═O)₂R^{a}, —S(═O)₂NR^{c}R^{d}, C₁₋₆ alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl, wherein each of the C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl is optionally substituted by 1, 2 or 3 R⁹;

R⁶ is C₆₋₁₀aryl, C₆₋₁₀aryloxy, C₂₋₅heteroaryloxy, or C₂₋₅heteroaryl, each optionally substituted by 1, 2, 3, 4 or 5 A¹;

R⁷, R⁸ and R⁹ are each, independently, halo, C₁₋₄alkyl, C₁₋₄haloalkyl, C₆₋₁₀aryl, C₃₋₇cycloalkyl, C₂₋₅heteroaryl, C₂₋₅heterocycloalkyl, —CN, —NO₂, —OR^{a′}, —SR^{a′}, —C(═O)R^{b′}, —C(═O)NR^{c′}R^{d′}, —C(═O)OR^{a′}, —OC(═O)R^{b′}, —OC(═O)NR^{c′}R^{d′}, —NR^{c′}R^{d′}, —NR^{c′}C(═O)R^{b′}, —NR^{c′}C(═O)OR^{a′}, —NR^{c′}S(═O)₂R^{b′}, —S(═O)R^{b′}, —S(═O)NR^{c′}R^{d′}, —S(═O)₂R^{b′}, or —S(═O)₂NR^{c′}R^{d′};

A¹ is halo, —CN, —NO₂, —OR^{a}, —SR^{a}, —C(═O)R^{b}, —C(═O)NR^{c}R^{d}, —C(═O)OR^{a}, —OC(═O)R^{b}, —OC(═O)NR^{c}R^{d}, —NR^{c}R^{d}. —NR^{c}C(═O)R^{d}, —NR^{c}C(═O)OR^{a}, —NR^{c}S(═O)R^{b}, —NR^{c}S(═O)₂R^{b}, —S(═O)R^{b}, —S(═O)NR^{c}R^{d}, —S(═O)₂R^{b}, —S(═O)₂NR^{c}R^{d}, C₁₋₄alkoxy, C₁₋₄haloalkoxy, amino, C₁₋₄alkylamino, C₂₋₈dialkylamino, C₁₋₆alkyl, C₂₋₆alkenyl, C₂₋₆alkynyl, C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl, wherein each of the C₁₋₆alkyl, C₂₋₆alkenyl, C₂₋₆alkynyl, C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl is optionally substituted by 1, 2, 3, 4 or 5 substituents independently selected from halo, C₁₋₆alkyl, C₂₋₆alkenyl, C₂₋₆alkynyl, C₁₋₄haloalkyl, C₆₋₁₀aryl, C₃₋₇cycloalkyl, C₂₋₅heteroaryl, C₂₋₅heterocycloalkyl, —CN, —NO₂, —OR^{a′}, —SR^{a′}, —C(═O)R^{b′}, —C(═O)NR^{c′}R^{d′}, —C(═O)OR^{a′}, —OC(═O)R^{b′}, —OC(═O)NR^{c′}R^{d′}, NR^{c′}R^{d′}, —NR^{c′}C(═O)R^{b′}, —NR^{c′}C(═O)OR^{a′}, —NR^{c′}S(═O)R^{b′}, —NR^{c′}S(═O)₂R^{b′}, —S(═O)R^{b′}, —S(═O)NR^{c′}R^{d′}, —S(═O)₂R^{b′}, or —S(═O)₂NR^{c′}R^{d′};

R^{a} and R^{a′} are each, independently, H, C₁₋₆alkyl, C₁₋₆haloalkyl, C₂₋₆alkenyl, C₂₋₆alkynyl, C₁₋₆alkyl, C₆₋₁₀aryl, C₂₋₅heteroaryl, C₃₋₇cycloalkyl, C₂₋₅heterocycloalkyl-C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl;

R^{b} and R^{b′} are each, independently, H, C₁₋₆alkyl, C₁₋₆haloalkyl, C₂₋₆alkenyl, C₂₋₆alkynyl, C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl;

R^{c} and R^{d} are each, independently, H, C₁₋₆alkyl, C₁₋₆haloalkyl, C₂₋₆alkenyl, C₂₋₆alkynyl, C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl;

or R^{c} and R^{d} together with the N atom to which they are attached form a 4-, 5-, 6- or 7-membered heterocycloalkyl group; and

R^{c′} and R^{d′} are each, independently, H, C₁₋₆alkyl, C₁₋₆haloalkyl, C₂₋₆ alkenyl, C₂₋₆alkynyl, C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl;

or R^{c′} and R^{d′} together with the N atom to which they are attached form a 4-, 5-, 6- or 7-membered heterocycloalkyl group;

with the proviso that when R², R³, R⁴ and R⁵ are each H, then R⁶ is not selected from unsubstituted phenyl, 4-fluorophenyl, 4-chlorophenyl, 4-methoxyphenyl, 4-methylphenyl, 3-methoxyphenyl, 2-methoxyphenyl, and 4-N,N-dimethylaminophenyl.

In some embodiments, R¹ is selected from C₁₋₆ alkyl, C₃₋₆cycloalkyl, C₃₋₆cycloalkyl-C₁₋₃alkyl, C₆₋₁₀aryl-C₁₋₃alkyl, and C₂₋₅heteroaryl-C₁₋₃alkyl, each optionally substituted by 1, 2, 3, 4 or 5 substituents independently selected from halo, C₁₋₄alkyl, C₁₋₄haloalkyl, —CN, —NO₂, —OH, C₁₋₄alkoxy, —O—(CH₂)ₙ—O—, C₁₋₄haloalkoxy, amino, C₁₋₄alkylamino, and C₂₋₈dialkylamino, wherein n is 1, 2, or 3.

In some embodiments, R¹ is selected from C₁₋₆alkyl, C₃₋₆cycloalkyl and benzyl optionally substituted with one or more substitutents selected from halogen, methoxy, and —O—CH₂—O—.

In some embodiments, R¹ is selected from 4-methoxybenzyl, 3,4-dimethoxybenzyl, 2,5-dimethoxybenzyl, benzo[1,3]dioxol-5-ylmethyl, cyclopropyl, ethyl, cyclobutyl, methyl, 1-butyl, and 1-propyl.

In some embodiments, R² is H, —C(═O)-(C₁₋₄alkyl), —C(═O)-(aryl-C₁₋₃alkyl), —C(═O)O—(C₁₋₄ alkyl), —C(═O)O-(aryl-C₁₋₃alkyl), —C(═O)NH₂, —C(═O)NH(C₁₋₄alkyl), —C(═O)N(C₁₋₄alkyl)₂, or C₁₋₃alkyl.

In some embodiments, R² is H.

In some embodiments, R³, R⁴ and R⁵ are each, independently, —H, halo, C₁₋₃alkyl, C₁₋₃alkoxy, —CN, —NO₂, —OH, halogenated C₁₋₃alkyl, or halogenated C₁₋₃alkoxy.

In some embodiments, R³, R⁴ and R⁵ are each, independently, —H or halo.

In some embodiments, R³ and R⁴ are each —H and R⁵ is fluoro.

In some embodiments, R⁶ is phenyl or heteroaryl, each optionally substituted by 1, 2, 3, 4 or 5 substituents independently selected from halo, C₁₋₄alkoxy, C₁₋₄alkyl, halogenated C₁₋₄alkyl, —OH, amino, C₁₋₄alkylamino, C₂₋₈dialkylamino and —CN.

In some embodiments, R⁶ is phenyl, naphthyl, pyridyl, pyrimidinyl, pyrazinyl, pyrazolyl, quinolyl or indolyl, each optionally substituted by 1, 2, 3, 4 or 5 substituents independently selected from halo, C₁₋₄alkoxy, C₁₋₄alkyl, halogenated C₁₋₄alkyl, —OH, amino, C₁₋₄alkylamino, C₂₋₈dialkylamino and —CN.

In some embodiments, R⁶ is phenyl or phenoxy, each optionally substituted by 2 substituents independently selected from halo, —CN, —OH, C₁₋₄alkoxy, C₁₋₄haloalkoxy, amino, C₁₋₄ alkylamino, C₂₋₈ dialkylamino, C₁₋₆alkyl, and C₁₋₆haloalkyl.

In some embodiments, R⁶ is phenyl substituted by 2 substituents independently selected from fluoro, chloro, —CN, methyl and methoxy.

In some embodiments, R⁶ is selected from pyridyl and pyrimidinyl, wherein said pyridyl and primidinyl are optionally substituted by 1, 2, or substitutents independently selected from fluoro, chloro, —CN, methyl and methoxy.

In some embodiments, the present invention provides a compound selected from:
9-amino-5-(2-fluoro-6-methoxyphenyl)-2-(4-methoxybenzyl)-2,3-dihydropyrrolo[3,4-b]quinolin-1-one;
9-amino-5-(2,5-difluorophenyl)-2-(4-methoxybenzyl)-2,3-dihydropyrrolo[3,4-b]quinolin-1-one;
9-amino-2-(4-methoxybenzyl)-5-(2-methoxypyridin-3-yl)-2,3-dihydropyrrolo[3,4-b]quinolin-1-one;
9-amino-2-(2,5-dimethoxybenzyl)-5-(2-methoxypyridin-3-yl)-2,3-dihydropyrrolo[3,4-b]quinolin-1-one;
9-amino-5-(2-fluoro-6-methoxyphenyl)-2-propyl-2,3-dihydropyrrolo[3,4-b]quinolin-1-one;
9-amino-5-(2,3-dimethylphenyl)-2-propyl-2,3-dihydropyrrolo[3,4-b]quinolin-1-one;
9-amino-5-(3,5-dimethylphenyl)-2-propyl-2,3-dihydropyrrolo[3,4-b]quinolin-1-one;
9-amino-5-(6-chloropyridin-3-yl)-2-(3,4-dimethoxybenzyl)-2,3-dihydropyrrolo[3,4-b]quinolin-1-one;
9-amino-5-(2,6-dimethoxypyridin-3-yl)-2-propyl-2,3-dihydropyrrolo[3,4-b]quinolin-1-one;
9-amino-5-(6-methylpyridin-3-yl)-2-propyl-2,3-dihydropyrrolo[3,4-b]quinolin-1-one;
9-amino-2-(3,4-dimethoxybenzyl)-5-(2,5-dimethoxyphenyl)-2,3-dihydropyrrolo[3,4-b]quinolin-1-one;
9-amino-5-(6-methoxy-4-methylpyridin-3-yl)-2-propyl-2,3-dihydropyrrolo[3,4b]quinolin-1-one;
9-amino-5-(2-fluoropyridin-3-yl)-2-propyl-2,3-dihydropyrrolo[3,4-b]quinolin-1-one;
9-amino-2-benzo[1,3]dioxol-5-ylmethyl-5-(2-methoxy-5-methylphenyl)-2,3-dihydropyrrolo[3,4-b]quinolin-1-one;
9-amino-5-(2-chloro-6-methylpyridin-3-yl)-2-propyl-2,3-dihydropyrrolo[3,4-b]quinolin-1-one;
9-amino-2-cyclopropyl-5-(2-fluoro-6-methoxyphenyl)-2,3-dihydropyrrolo[3,4-b]quinolin-1-one;
9-amino-2-ethyl-5-(2-fluoro-6-methoxyphenyl)-2,3-dihydropyrrolo[3,4-b]quinolin-1-one;
9-amino-2-cyclobutyl-5-(2-fluoro-6-methoxyphenyl)-2,3-dihydropyrrolo[3,4-b]quinolin-1-one;
9-amino-2-ethyl-5-(2-methoxypyridin-3-yl)-2,3-dihydropyrrolo[3,4-b]quinolin-1-one;
9-amino-5-(2-fluoro-6-methoxyphenyl)-2-methyl-2,3-dihydropyrrolo[3,4-b]quinolin-1-one;
9-amino-2-cyclopropyl-5-(2,5-dimethoxyphenyl)-2,3-dihydropyrrolo[3,4-b]quinolin-1-one;
9-amino-2-cyclopropyl-5-(2-fluoro-3-methoxyphenyl)-2,3-dihydropyrrolo[3,4-b]quinolin-1-one;
9-amino-5-(2-chloro-6-methylpyridin-3-yl)-2-(3,4-dimethoxybenzyl)-2,3-dihydropyrrolo[3,4-b]quinolin-1-one;
9-amino-5-(2,6-dimethoxypyridin-3-yl)-6-fluoro-2-propyl-2,3-dihydropyrrolo[3,4-b]quinolin-1-one;
2-(9-amino-2-ethyl-1-oxo-2,3-dihydro-1h-pyrrolo[3,4-b]quinolin-5-yl)-benzonitrile;
9-amino-5-(2,6-dimethoxypyridin-3-yl)-2-ethyl-6-fluoro-2,3-dihydropyrrolo[3,4b]quinolin-1-one;
9-amino-5-(2,6-dimethoxypyridin-3-yl)-2-ethyl-2,3-dihydropyrrolo[3,4-b]quinolin-1-one;
9-amino-2-cyclopropyl-5-(2,4-dimethoxyphenyl)-6-fluoro-2,3-dihydropyrrolo[3,4-b]quinolin-1-one;
9-amino-2-ethyl-5-(3,4-dimethoxyphenyl)-2,3-dihydropyrrolo[3,4-b]quinolin-1-one;
9-amino-5-(2,5-dimethoxyphenyl)-2-ethyl-6-fluoro-2,3-dihydro-1h-pyrrolo[3,4-b]quinolin-1-one;
9-amino-5-(2,6-dimethoxypyridin-3-yl)-2-methyl-2,3-dihydropyrrolo[3,4-b]quinolin-1-one;
9-amino-2-ethyl-5-(4-fluoro-2-methoxyphenyl)-2,3-dihydropyrrolo[3,4-b]quinolin-1-one;
9-amino-2-ethyl-5-(2-fluoro-3-methoxyphenyl)-2,3-dihydropyrrolo[3,4-b]quinolin-1-one;
9-amino-5-(2,4-dimethoxypyrimidin-5-yl)-2-ethyl-6-fluoro-2,3-dihydropyrrolo[3,4-b]quinolin-1-one;
9-amino-2-cyclopropyl-6-fluoro-5-(2-fluoro-6-methoxyphenyl)-2,3-dihydropyrrolo[3,4-b]quinolin-1-one;
9-amino-2-ethyl-6-fluoro-5-(4-methoxy-pyridin-3-yl)-2,3-dihydropyrrolo[3,4-b]quinolin-1-one;
9-amino-2-cyclopropyl-5-(2,5-dimethoxy-phenyl)-6-fluoro-2,3-dihydropyrrolo[3,4-b]quinolin-1-one;
9-amino-2-cyclopropyl-6-fluoro-5-(2-methoxypyridin-3-yl)-2,3-dihydropyrrolo[3,4-b]quinolin-1-one;
9-amino-2-(2,5-dimethoxybenzyl)-5-(4-methoxypyridin-3-yl)-2,3-dihydropyrrolo[3,4-b]quinolin-1-one;
9-amino-2-propyl-5-pyridin-3-yl-2,3-dihydropyrrolo[3,4-b]quinolin-1-one;
9-amino-2-cyclopropyl-6-fluoro-5-(4-methoxypyridin-3-yl)-2,3-dihydropyrrolo[3,4-b]quinolin-1-one;
9-amino-2-cyclobutyl-5-(2,5-dimethoxyphenyl)-2,3-dihydropyrrolo[3,4-b]quinolin-1-one;
9-amino-2-butyl-5-(2,6-dimethoxypyridin-3-yl)-2,3-dihydropyrrolo[3,4-b]quinolin-1-one;
9-amino-2-cyclopropyl-5-(2,4-dimethoxypyrimidin-5-yl)-6-fluoro-2,3-dihydropyrrolo[3,4-b]quinolin-1-one;
9-amino-2-ethyl-6-fluoro-5-(2-methoxyphenyl)-2,3-dihydropyrrolo[3,4-b]quinolin-1-one;
9-amino-2-ethyl-6-fluoro-5-(5-fluoro-2-methoxyphenyl)-2,3-dihydropyrrolo[3,4-b]quinolin-1-one;
9-amino-5-(3,4-dimethoxyphenyl)-2-ethyl-6-fluoro-2,3-dihydropyrrolo[3,4-b]quinolin-1-one;
9-amino-2-cyclobutyl-5-(2-methoxypyridin-3-yl)-2,3-dihydropyrrolo[3,4-b]quinolin-1-one;
9-amino-5-(5-chloro-2-methoxyphenyl)-2-cyclobutyl-2,3-dihydropyrrolo[3,4-b]quinolin-1-one;
9-amino-2-cyclopropyl-5-(3,4-dimethoxyphenoxy)-2,3-dihydro-1h-pyrrolo[3,4-b]quinolin-1-one;
9-amino-2-cyclobutyl-5-(2,6-dimethoxypyridin-3-yl)-6-fluoro-2,3-dihydropyrrolo[3,4-b]quinolin-1-one;
9-amino-2-cyclobutyl-5-(2,4-dimethoxyphenyl)-2,3-dihydro-pyrrolo[3,4-b]quinolin-1-one;
9-amino-2-cyclopropyl-5-(2,6-dimethoxypyridin-3-yl)-6-fluoro-2,3-dihydropyrrolo[3,4-b]quinolin-1-one;
9-amino-6-fluoro-5-(2-fluoro-6-methoxyphenyl)-2-propyl-2,3-dihydropyrrolo[3,4-b]quinolin-1-one;
9-amino-5-(2,6-difluoro-phenyl)-2-propyl-2,3-dihydro-pyrrolo[3,4-b]quinolin-1-one;
9-amino-2-ethyl-5-(4-methoxypyridin-3-yl)-2,3-dihydropyrrolo[3,4-b]quinolin-1-one;
9-amino-2-ethyl-6-fluoro-5-(2-methoxypyridin-3-yl)-2,3-dihydropyrrolo[3,4-b]quinolin-1-one;
9-amino-2-cyclopropyl-5-(2,5-dichlorophenyl)-2,3-dihydropyrrolo[3,4-b]quinolin-1-one;
9-amino-2-cyclopropyl-5-(2-fluoro-5-methoxyphenyl)-2,3-dihydropyrrolo[3,4-b]quinolin-1-one;
9-amino-2-cyclopropyl-6-fluoro-5-(5fluoro-2-methoxyphenyl)-2,3-dihydropyrrolo[3,4-b]quinolin-1-one;
9-amino -2-cyclobutyl-5-(4-methoxy-pyridin-3-yl)-2,3-dihydropyrrolo[3,4-b]quinolin-1-one;
9-amino-2-cyclobutyl-5-(2-methoxyphenyl)-2,3-dihydropyrrolo[3,4-b]quinolin-1-one;
9-amino-2-cyclobutyl-5-(2,6-dimethoxypyridin-3-yl)-2,3-dihydropyrrolo[3,4-b]quinolin-1-one;
9-amino-2-(3,4-dimethoxybenzyl)-5-(2-fluoro-6-methoxyphenyl)-2,3-dihydro-pyrrolo[3,4-b]quinolin-1-one;
9-amino-2-cyclopropyl-5-(2-methoxypyridin-3-yl)-2,3-dihydropyrrolo[3,4-b]quinolin-1-one;
9-Amino-2-ethyl-6-fluoro-5-(2-fluoro-6-methoxyphenyl)-2,3-dihydropyrrolo[3,4-b]quinolin-1-one;
9-Amino-5-(2,4-dimethoxy-phenyl)-2-ethyl-6-fluoro-2,3-dihydropyrrolo[3,4-b]quinolin-1-one;
9-Amino-5-(2-fluoro-6-methoxyphenyl)-2-isopropyl-2,3-dihydropyrrolo[3,4-b]quinolin-1-one;
9-Amino-6-fluoro-5-(2-fluoro-6-methoxyphenyl)-2-methyl-2,3-dihydropyrrolo[3,4-b]quinolin-1-one;
9-Amino-5-(2-fluoro-3-methoxyphenyl)-2-methyl-2,3-dihydropyrrolo[3,4-b]quinolin-1-one;
9-Amino-2-ethyl-5-(2-fluoro-5-methoxyphenyl)-2,3-dihydropyrrolo[3,4b]quinolin-1-one;
9-Amino-2-ethyl-5-(5-fluoro-2-methoxyphenyl)-2,3-dihydropyrrolo[3,4-b]quinolin-1-one;
9-Amino-2-ethyl-5-(4-fluoro-3-methoxyphenyl)-2,3-dihydropyrrolo[3,4-b]quinolin-1-one;
9-Amino-2-ethyl-5-(4-methylpyridin-3-yl)-2,3-dihydropyrrolo[3,4-b]quinolin-1-one;
2-(9-Amino-2-cyclobutyl-1-oxo-2,3-dihydro-1H-pyrrolo[3,4-b]quinolin-5-yl)benzonitrile;
9-Amino-2-cyclobutyl-5-(4-fluoro-2-methoxyphenyl)-2,3-dihydropyrrolo[3,4-b]quinolin-1-one;
9-Amino-2-cyclobutyl-5-(2-fluoro-3-methoxyphenyl)-2,3-dihydropyrrolo[3,4-b]quinolin-1-one;
9-Amino-2-cyclobutyl-5-(2-fluoro-5-methoxyphenyl)-2,3-dihydropyrrolo[3,4-b]quinolin-1-one;
9-Amino-2-cyclobutyl-5-pyrazin-2-yl-2,3-dihydropyrrolo[3,4-b]quinolin-1-one;
9-Amino-2-cyclobutyl-5-(3-methoxypyridin-4-yl)-2,3-dihydropyrrolo[3,4-b]quinolin-1-one;
9-Amino-2-cyclobutyl-5-pyridin-4-yl-2,3-dihydropyrrolo[3,4-b]quinolin-1-one;
9-Amino-2-cyclobutyl-5-pyridin-2-yl-2,3-dihydropyrrolo[3,4-b]quinolin-1-one;
9-Amino-2-cyclobutyl-5-(3,6-dimethoxypyridazin-4-yl)-2,3-dihydropyrrolo[3,4-b]quinolin-1-one;
9-Amino-2-cyclobutyl-5-(6-methoxypyridin-2-yl)-2,3-dihydropyrrolo[3,4-b]quinolin-1-one;
9-Amino-2-cyclobutyl-3-hydroxy-5-(6-methylpyridin-2-yl)-2,3-dihydropyrrolo[3,4-b]quinolin-1-one;
9-Amino-2-cyclobutyl-5-(5-methylpyridin-2-yl)-2,3-dihydropyrrolo[3,4-b]quinolin-1-one;
9-Amino-2-cyclobutyl-5-pyrimidin-2-yl-2,3-dihydropyrrolo[3,4-b]quinolin-1-one;
6-(9-Amino-2-cyclobutyl-1-oxo-2,3-dihydro-1H-pyrrolo[3,4-b]quinolin-5-yl)-nicotinonitrile;
5-(9-Amino-2-cyclobutyl-1-oxo-2,3-dihydro-1H-pyrrolo[3,4-b]quinolin-5-yl)-nicotinonitrile;
9-Amino-2-cyclobutyl-5-(3-methoxypyridazin-4-yl)-2,3-dihydropyrrolo[3,4-b]quinolin-1-one;
9-Amino-2-cyclobutyl-5-(4-methoxy-pyrimidin-5-yl)-2,3-dihydropyrrolo[3,4-b]quinolin-1-one;
9-Amino-2-cyclobutyl-5-(3-fluoropyridin-2-yl)-2,3-dihydropyrrolo[3,4-b]quinolin-1-one;
2-(9-Amino-2-cyclobutyl-1-oxo-2,3-dihydro-1H-pyrrolo[3,4-b]quinolin-5-yl)-5-fluorobenzonitrile;
2-(9-Amino-2-cyclobutyl-1-oxo-2,3-dihydro-1H-pyrrolo[3,4-b]quinolin-5-yl)-4-fluorobenzonitrile;
4-(9-Amino-2-cyclobutyl-1-oxo-2,3-dihydro-1H-pyrrolo[3,4-b]quinolin-5-yl)-6-methoxynicotinonitrile;
9-Amino-5-(1,3-dimethyl-1H-pyrazol-4-yl)-6-fluoro-2-(R)-tetrahydro-furan-3-yl-2,3-dihydropyrrolo[3,4-b]quinolin-1-one;
9-Amino-2-cyclobutyl-5-(5-fluoro-2-methoxypyridin-4-yl)-2,3-dihydropyrrolo[3,4-b]quinolin-1-one;
9-Amino-2-cyclobutyl-5-(5-fluoro-2-methoxyphenyl)-2,3-dihydropyrrolo[3,4-b]quinolin-1-one;
9-Amino-2-cyclobutyl-5-(2,4-dimethoxy-phenyl)-6-fluoro-2,3-dihydropyrrolo[3,4-b]quinolin-1-one;
9-Amino-2-cyclobutyl-6-fluoro-5-(6-methylpyridin-3-yl)-2,3-dihydropyrrolo[3,4-b]quinolin-1-one;
9-Amino-2-cyclobutyl-6-fluoro-5-(2-fluoropyridin-3-yl)-2,3-dihydropyrrolo[3,4-b]quinolin-1-one;
9-Amino-2-cyclobutyl-6-fluoro-5-(4-fluoro-2-methoxyphenyl)-2,3-dihydropyrrolo[3,4-b]quinolin-1-one;
9-Amino-2-cyclobutyl-6-fluoro-5-pyrimidin-5-yl-2,3-dihydropyrrolo[3,4-b]quinolin-1-one;
9-Amino-2-cyclobutyl-6-fluoro-5-(3-methoxypyridin-4-yl)-2,3-dihydropyrrolo[3,4-b]quinolin-1-one;
9-Amino-2-cyclobutyl-6-fluoro-5-(2-fluoro-3-methoxyphenyl)-2,3-dihydropyrrolo[3,4-b]quinolin-1-one;
9-Amino-2-cyclobutyl-6-fluoro-5-(6-methoxypyridin-3-yl)-2,3-dihydropyrrolo[3,4-b]quinolin-1-one;
9-Amino-2-cyclobutyl-6-fluoro-5-(2-vinylphenyl)-2,3-dihydropyrrolo[3,4-b]quinolin-1-one;
9-Amino-2-(3-chloro-4-methoxybenzyl)-5-(2-fluoro-6-methoxyphenyl)-2,3-dihydropyrrolo[3,4-b]quinolin-1-one;
2-(9-Amino-2-cyclopropyl-1-oxo-2,3-dihydro-1H-pyrrolo[3,4-b]quinolin-5-yl)-benzonitrile;
9-Amino-2-cyclopropyl-5-(6-methyl-pyridin-3-yl)-2,3-dihydropyrrolo[3,4-b]quinolin-1-one;
9-Amino-2-cyclopropyl-5-(2,5-difluoro-phenyl)-2,3-dihydropyrrolo[3,4-b]quinolin-1-one;
9-Amino-2-cyclopropyl-5-(2-fluorophenyl)-2,3-dihydropyrrolo[3,4-b]quinolin-1-one;
9-Amino-2-cyclopropyl-5-(2,6-difluoro-phenyl)-2,3-dihydropyrrolo[3,4-b]quinolin-1-one;
9-Amino-2-cyclopropyl-5-(2-fluoro-4-methoxy-phenyl)-2,3-dihydropyrrolo[3,4-b]quinolin-1-one;
9-Amino-5-(2-chloro-5-methoxyphenyl)-2-cyclopropyl-2,3-dihydropyrrolo[3,4-b]quinolin-1-one;
9-Amino-2-cyclopropyl-5-(2,6-difluoro-4-methoxyphenyl)-2,3-dihydropyrrolo[3,4-b]quinolin-1-one;
9-Amino-2-cyclopropyl-5-(2,3-difluorophenyl)-2,3-dihydropyrrolo[3,4-b]quinolin-1-one;
9-Amino-2-cyclopropyl-5-(2,4-difluorophenyl)-2,3-dihydropyrrolo[3,4-b]quinolin-1-one;
9-Amino-2-cyclopropyl-5-(2-fluoro-6-methylpyridin-3-yl)-2,3-dihydropyrrolo[3,4-b]quinolin-1-one;
9-Amino-2-cyclobutyl-5-(6-methylpyridin-3-yl)-2,3-dihydropyrrolo[3,4-b]quinolin-1-one;
9-Amino-2-cyclobutyl-5-(2,6-difluoro-4-methoxyphenyl)-2,3-dihydropyrrolo[3,4-b]quinolin-1-one;
9-Amino-2-cyclobutyl-5-(2,4-dimethoxy-pyrimidin-5-yl)-2,3-dihydropyrrolo[3,4-b]quinolin-1-one;
9-Amino-2-cyclobutyl-5-(2-fluorophenyl)-2,3-dihydro-pyrrolo[3,4-b]quinolin-1-one;
5-(9-Amino-2-cyclobutyl-1-oxo-2,3-dihydro-1H-pyrrolo[3,4-b]quinolin-5-yl)-pyridine-2-carbonitrile;
9-Amino-2-cyclobutyl-5-(6-fluoro-2-methylpyridin-3-yl)-2,3-dihydropyrrolo[3,4-b]quinolin-1-one;
9-Amino-2-cyclobutyl-5-(2-fluoropyridin-3-yl)-2,3-dihydropyrrolo[3,4-b]quinolin-1-one;
9-Amino-2-cyclobutyl-5-(6-methoxy-5-methylpyridin-3-yl)-2,3-dihydropyrrolo[3,4-b]quinolin-1-one;
9-Amino-2-cyclobutyl-5-((P)-2-fluoro-6-methoxyphenyl)-2,3-dihydro-pyrrolo[3,4-b]quinolin-1-one;
9-Amino-2-cyclobutyl-5-((M)-2-fluoro-6-methoxyphenyl)-2,3-dihydro-pyrrolo[3,4-b]quinolin-1-one;
2-(9-Amino-2-cyclobutyl-1-oxo-2,3-dihydro-1H-pyrrolo[3,4-b]quinolin-5-yl)-6-methoxybenzonitrile;
2-(9-Amino-2-cyclobutyl-1-oxo-2,3-dihydro-1H-pyrrolo[3,4-b]quinolin-5-yl)-3-methoxybenzonitrile;
9-Amino-2-cyclobutyl-5-(2,6-difluoropyridin-3-yl)-2,3-dihydropyrrolo[3,4-b]quinolin-1-one;
9-Amino-5-(2-fluoro-6-methoxyphenyl)-2-(3-methylcyclobutyl)-2,3-dihydropyrrolo[3,4-b]quinolin-1-one;
9-Amino-2-cyclobutyl-5-(1-methyl-1H-pyrazol-4-yl)-2,3-dihydropyrrolo[3,4-b]quinolin-1-one;
9-Amino-5-(6-methoxy-2-methylpyridin-3-yl)-2-((1s,3s)-3-methylcyclobutyl)-2,3-dihydro-1H-pyrrolo[3,4-b]quinolin-1-one;
9-Amino-5-(2-fluoro-6-methoxyphenyl)-2-((1s,3s)-3-methylcyclobutyl)-2,3-dihydro-1H-pyrrolo[3,4-b]quinolin-1-one;
9-Amino-5-(2-methoxypyridin-3-yl)-2-((1s,3s)-3-methylcyclobutyl)-2,3-dihydro-1H-pyrrolo[3,4-b]quinolin-1-one;
2-(9-Amino-2-cyclopropyl-1-oxo-2,3-dihydro-1H-pyrrolo[3,4-b]quinolin-5-yl)-3-methoxybenzonitrile;
9-Amino-2-cyclopropyl-5-((P)2-fluoro-6-methoxyphenyl)-2,3-dihydropyrrolo[3,4-b]quinolin-1-one;
9-Amino-2-cyclobutyl-5-(2-fluoro-6-methylpyridin-3-yl)-2,3-dihydropyrrolo[3,4-b]quinolin-1-one;
9-Amino-2-cyclobutyl-5-(6-methoxy-2-methylpyridin-3-yl)-2,3-dihydropyrrolo[3,4-b]quinolin-1-one;
9-Amino-2-cyclobutyl-5-(1,3-dimethyl-1H-pyrazol-4-yl)-2,3-dihydropyrrolo[3,4-b]quinolin-1-one;
9-Amino-2-cyclobutyl-5-(6-fluoro-5-methylpyridin-3-yl)-2,3-dihydropyrrolo[3,4-b]quinolin-1-one;
9-Amino-2-cyclopentyl-5-(2-fluoro-6-methoxyphenyl)-2,3-dihydropyrrolo[3,4-b]quinolin-1-one;
2-(9-Amino-2-cyclopentyl-1-oxo-2,3-dihydro-1H-pyrrolo[3,4-b]quinolin-5-yl)-benzonitrile;
9-Amino-2-cyclopentyl-5-(6-methoxy-pyridin-3-yl)-2,3-dihydro-pyrrolo[3,4-b]quinolin-1-one;
9-Amino-2-cyclobutyl-5-(6-morpholin-4-yl-pyridin-3-yl)-2,3-dihydropyrrolo[3,4-b]quinolin-1-one;
9-Amino-2-cyclobutyl-5-(6-methoxypyridin-3-yl)-2,3-dihydropyrrolo[3,4-b]quinolin-1-one;
9-Amino-2-cyclobutyl-5-(4-methylpyridin-3-yl)-2,3-dihydropyrrolo[3,4-b]quinolin-1-one;
9-Amino-2-cyclobutyl-5-(3-fluoropyrazin-2-yl)-2,3-dihydropyrrolo[3,4-b]quinolin-1-one;
9-Amino-2-cyclobutyl-5-(5-methoxy-pyridin-3-yl)-2,3-dihydropyrrolo[3,4-b]quinolin-1-one;
9-Amino-2-cyclopropyl-6-fluoro-5-(2-fluoro-3-methoxyphenyl)-2,3-dihydropyrrolo[3,4-b]quinolin-1-one;
9-Amino-2-cyclopropyl-5-(2,6-difluoro-3-methoxyphenyl)-6-fluoro-2,3-dihydropyrrolo[3,4-b]quinolin-1-one;
9-Amino-2-cyclopropyl-6-fluoro-5-(2-fluoro-5-methoxyphenyl)-2,3-dihydropyrrolo[3,4-b]quinolin-1-one;
9-Amino-2-ethyl-6-fluoro-5-(4-methylpyridin-3-yl)-2,3-dihydropyrrolo[3,4-b]quinolin-1-one;
9-Amino-2-ethyl-6-fluoro-5-(2-fluoro-5-methoxyphenyl)-2,3-dihydropyrrolo[3,4-b]quinolin-1-one;
9-Amino-2-cyclobutyl-5-(2,4-dimethoxypyrimidin-5-yl)-6-fluoro-2,3-dihydropyrrolo[3,4-b]quinolin-1-one;
9-Amino-2-cyclobutyl-5-(2,5-dimethoxyphenyl)-6-fluoro-2,3-dihydropyrrolo[3,4-b]quinolin-1-one;
9-Amino-2-cyclobutyl-6-fluoro-5-(2-methoxypyridin-3-yl)-2,3-dihydropyrrolo[3,4-b]quinolin-1-one;
9-Amino-2-cyclopropyl-6-fluoro-5-(2-methoxyphenyl)-2,3-dihydropyrrolo[3,4-b]quinolin-1-one;
9-Amino-2-cyclobutyl-6-fluoro-5-(2-methoxyphenyl)-2,3-dihydropyrrolo[3,4-b]quinolin-1-one;
9-Amino-5-(5-chloro-2-methoxyphenyl)-2-ethyl-6-fluoro-2,3-dihydropyrrolo[3,4-b]quinolin-1-one;
9-Amino-2-cyclobutyl-6-fluoro-5-(2-fluoro-5-methoxyphenyl)-2,3-dihydropyrrolo[3,4-b]quinolin-1-one;
9-Amino-2-cyclobutyl-6-fluoro-5-(5-fluoro-2-methoxyphenyl)-2,3-dihydropyrrolo[3,4-b]quinolin-1-one;
9-Amino-2-cyclobutyl-6-fluoro-5-(6-methoxy-4-methylpyridin-3-yl)-2,3-dihydropyrrolo[3,4-b]quinolin-1-one;
9-Amino-2-cyclobutyl-6-fluoro-5-(6-methoxy-2-methylpyridin-3-yl)-2,3-dihydropyrrolo[3,4-b]quinolin-1-one;
9-Amino-2-cyclobutyl-5-(2,5-dimethoxypyridin-3-yl)-2,3-dihydropyrrolo[3,4-b]quinolin-1-one;
9-Amino-6-fluoro-5-(2-methoxypyridin-3-yl)-2-(R)-tetrahydrofuran-3-yl-2,3-dihydropyrrolo[3,4-b]quinolin-1-one;
9-Amino-6-fluoro-5-(2-methoxypyridin-3-yl)-2-(S)-tetrahydrofuran-3-yl-2,3-dihydro-pyrrolo[3,4-b]quinolin-1-one;
9-Amino-2-cyclobutyl-5-(3,4-dimethoxyphenyl)-6-fluoro-2,3-dihydropyrrolo[3,4-b]quinolin-1-one;
   and pharmaceutically acceptable salts thereof.

In some embodiments, the the present invention provides a compound selected from:
9-amino-2-cyclopropyl-5-(2-fluoro-6-methoxyphenyl)-2,3-dihydro-1H-pyrrolo[3,4-b]quinolin-1-one;
9-amino-2-cyclobutyl-5-(2,5-dimethoxyphenyl)-6-fluoro-2,3-dihydro-1H-pyrrolo[3,4-b]quinolin-1-one;
9-amino-2-cyclobutyl-6-fluoro-5-(2-methoxypyridin-3-yl)-2,3-dihydro-1H-pyrrolo[3,4-b]quinolin-1-one;
9-amino-2-cyclobutyl-5-(2-fluorophenyl)-2,3-dihydro-1H-pyrrolo[3,4-b]quinolin-1-one;
9-amino-2-cyclobutyl-5-(2,4-dimethoxypyrimidin-5-yl)-6-fluoro-2,3-dihydro-1H-pyrrolo[3,4-b]quinolin-1-one;
2-(9-amino-2-cyclobutyl-1-oxo-2,3-dihydro-1H-pyrrolo[3,4-b]quinolin-5-yl)benzonitrile;
9-amino-2-ethyl-6-fluoro-5-(2-fluoro-5-methoxyphenyl)-2,3-dihydro-1H-pyrrolo[3,4-b]quinolin-1-one;
9-amino-2-cyclobutyl-5-(2,6-difluoro-4-methoxyphenyl)-2,3-dihydro-1H-pyrrolo[3,4-b]quinolin-1-one;
9-amino-2-ethyl-6-fluoro-5-(2-fluoro-4-methoxyphenyl)-2,3-dihydro-1H-pyrrolo[3,4-b]quinolin-1-one;
9-amino-2-(3,4-dimethoxybenzyl)-5-(4-methoxypyridin-3-yl)-2,3-dihydro-1H-pyrrolo[3,4-b]quinolin-1-one;
9-amino-2-ethyl-6-fluoro-5-(2-fluorophenyl)-2,3-dihydro-1H-pyrrolo[3,4-b]quinolin-1-one;
9-amino-2-ethyl-6-fluoro-5-(4-methylpyridin-3-yl)-2,3-dihydro-1H-pyrrolo[3,4-b]quinolin-1-one;
9-amino-2-(benzo[d][1,3]dioxol-5-ylmethyl)-5-(4-methoxypyridin-3-yl)-2,3-dihydro-1H-pyrrolo[3,4-b]quinolin-1-one;
9-amino-5-(2-chloro-6-methoxyphenyl)-2-cyclobutyl-2,3-dihydro-1H-pyrrolo[3,4-b]quinolin-1-one;
9-amino-2-(3,4-dimethoxybenzyl)-5-(2-methoxypyridin-3-yl)-2,3-dihydro-1H-pyrrolo[3,4-b]quinolin-1-one;
9-amino-5-(2,6-dimethylpyridin-3-yl)-2-propyl-2,3-dihydro-1H-pyrrolo[3,4-b]quinolin-1-one;
9-amino-2-(3,4-dimethoxybenzyl)-5-(2-fluoro-6-methoxyphenyl)-2,3-dihydro-1H-pyrrolo[3,4-b]quinolin-1-one;
9-amino-2-cyclopropyl-6-fluoro-5-(2-fluoro-3-methoxyphenyl)-2,3-dihydro-1H-pyrrolo[3,4-b]quinolin-1-one;
9-amino-2-(3,4-dimethoxybenzyl)-5-(2,6-dimethylpyridin-3-yl)-2,3-dihydro-1H-pyrrolo[3,4-b]quinolin-1-one;
9-amino-2-cyclopentyl-5-(2-fluoro-6-methoxyphenyl)-2,3-dihydro-1H-pyrrolo[3,4-b]quinolin-1-one;
9-amino-2-cyclobutyl-5-(2,5-dichlorophenyl)-2,3-dihydro-1H-pyrrolo[3,4-b]quinolin-1-one;
9-amino-2-(4-methoxybenzyl)-5-(4-methoxypyridin-3-yl)-2,3-dihydro-1H-pyrrolo[3,4-b]quinolin-1-one;
9-amino-2-ethyl-6-fluoro-5-(2-fluoro-6-methoxyphenyl)-2,3-dihydro-1H-pyrrolo[3,4-b]quinolin-1-one;
9-amino-2-cyclopropyl-5-(2,6-difluorophenyl)-2,3-dihydro-1H-pyrrolo[3,4-b]quinolin-1-one;
9-amino-5-(2-methoxy-5-methylpyridin-3-yl)-2-propyl-2,3-dihydro-1H-pyrrolo[3,4-b]quinolin-1-one;
9-amino-5-(2,4-difluorophenyl)-2-ethyl-6-fluoro-2,3-dihydro-1H-pyrrolo[3,4-b]quinolin-1-one;
9-amino-2-cyclobutyl-5-(2-methoxy-5-methylphenyl)-2,3-dihydro-1H-pyrrolo[3,4-b]quinolin-1-one;
9-amino-2-cyclopropyl-5-(2,6-difluoro-3-methoxyphenyl)-6-fluoro-2,3-dihydro-1H-pyrrolo[3,4-b]quinolin-1-one;
9-amino-2-cyclobutyl-5-(2,4-dimethoxypyrimidin-5-yl)-2,3-dihydro-1H-pyrrolo[3,4-b]quinolin-1-one;
9-amino-2-cyclobutyl-5-(6-methylpyridin-3-yl)-2,3-dihydro-1H-pyrrolo[3,4-b]quinolin-1-one;
9-amino-5-(2,6-difluoro-4-methoxyphenyl)-2-ethyl-6-fluoro-2,3-dihydro-1H-pyrrolo[3,4-b]quinolin-1-one;
9-amino-2-butyl-5-(2-fluoro-6-methoxyphenyl)-2,3-dihydro-1H-pyrrolo[3,4-b]quinolin-1-one;
9-amino-2-(3,4-dimethoxybenzyl)-5-(6-methoxy-2-methylpyridin-3-yl)-2,3-dihydro-1H-pyrrolo[3,4-b]quinolin-1-one;
9-amino-2-cyclopropyl-5-(2-methoxy-5-methylphenyl)-2,3-dihydro-1H-pyrrolo[3,4-b]quinolin-1-one;
9-amino-5-(2-chlorophenyl)-2-propyl-2,3-dihydro-1H-pyrrolo[3,4-b]quinolin-1-one;
9-amino-5-(2,4-dimethoxyphenyl)-2-ethyl-6-fluoro-2,3-dihydro-1H-pyrrolo[3,4-b]quinolin-1-one;
9-amino-5-(2-chloro-6-methoxyphenyl)-2-cyclopropyl-6-fluoro-2,3-dihydro-1H-pyrrolo[3,4-b]quinolin-1-one;
9-amino-2-ethyl-6-fluoro-5-(2-fluoro-3-methoxyphenyl)-2,3-dihydro-1H-pyrrolo[3,4-b]quinolin-1-one;
9-amino-2-propyl-5-(2-methylphenyl)-2,3-dihydro-1H-pyrrolo[3,4-b]quinolin-1-one;
9-amino-2-(benzo[d][1,3]dioxol-5-ylmethyl)-5-(2-methoxypyridin-3-yl)-2,3-dihydro-pyrrolo[3,4-b]quinolin-1-one;
9-amino-2-(4-methoxybenzyl)-5-(5-methoxypyridin-3-yl)-2,3-dihydro-1H-pyrrolo[3,4-b]quinolin-1-one;
9-amino-5-(5-fluoro-2-methylphenyl)-2-propyl-2,3-dihydro-1H-pyrrolo[3,4-b]quinolin-1-one;
9-amino-5-(2-chloro-5-methylpyridin-3-yl)-2-propyl-2,3-dihydro-1H-pyrrolo[3,4-b]quinolin-1-one;
9-amino-5-(2-fluoro-5-methylphenyl)-2-propyl-2,3-dihydro-1H-pyrrolo[3,4-b]quinolin-1-one;
9-amino-5-(2-fluoropyridin-3-yl)-2-(4-methoxybenzyl)-2,3-dihydro-1H-pyrrolo[3,4-b]quinolin-1-one;
9-amino-2-cyclopropyl-5-(2,5-difluorophenyl)-2,3-dihydro-1H-pyrrolo[3,4-b]quinolin-1-one;
9-amino-2-ethyl-5-(2-fluoro-4-methoxyphenyl)-2,3-dihydro-1H-pyrrolo[3,4-b]quinolin-1-one;
9-amino-2-cyclopropyl-6-fluoro-5-(2-fluoro-4-methoxyphenyl)-2,3-dihydro-1H-pyrrolo[3,4-b]quinolin-1-one;
9-amino-2-cyclopropyl-6-fluoro-5-(2-fluoro-5-methoxyphenyl)-2,3-dihydro-1H-pyrrolo[3,4-b]quinolin-1-one;
9-amino-5-(2-fluoro-5-methylpyridin-3-yl)-2-propyl-2,3-dihydro-1H-pyrrolo[3,4-b]quinolin-1-one;
9-amino-2-cyclopropyl-6-fluoro-5-(2-methoxy-5-methylphenyl)-2,3-dihydro-1H-pyrrolo[3,4-b]quinolin-1-one;
9-amino-5-(2-chloro-6-methylpyridin-3-yl)-2-cyclopropyl-2,3-dihydro-1H-pyrrolo[3,4-b]quinolin-1-one;
9-amino-5-(2-fluoro-3-methoxyphenyl)-2-(4-methoxybenzyl)-2,3-dihydro-1H-pyrrolo[3,4-b]quinolin-1-one;
9-amino-5-(2-fluoro-5-methoxypyridin-4-yl)-2-propyl-2,3-dihydro-1H-pyrrolo[3,4-b]quinolin-1-one;
9-amino-2-cyclopropyl-5-(4-methoxypyridin-3-yl)-2,3-dihydro-1H-pyrrolo[3,4-b]quinolin-1-one;
9-amino-6-fluoro-5-(2-fluoro-6-methoxyphenyl)-2-methyl-2,3-dihydro-1H-pyrrolo[3,4-b]quinolin-1-one;
9-amino-2-cyclopropyl-5-(2,6-difluoro-4-methoxyphenyl)-2,3-dihydro-1H-pyrrolo[3,4-b]quinolin-1-one;
9-amino-5-(2,4-dimethoxypyrimidin-5-yl)-6-fluoro-2-propyl-2,3-dihydro-1H-pyrrolo[3,4-b]quinolin-1-one;
9-amino-5-(5-chloro-2-methoxyphenyl)-2-cyclopropyl-2,3-dihydro-1H-pyrrolo[3,4-b]quinolin-1-one;
9-amino-2-(3,4-dimethoxybenzyl)-5-(2-methoxy-5-methylphenyl)-2,3-dihydro-1H-pyrrolo[3,4-b]quinolin-1-one;
9-amino-5-(2-chloro-5-methoxypyridin-3-yl)-2-propyl-2,3-dihydro-1H-pyrrolo[3,4-b]quinolin-1-one;
9-amino-2-(3-chloro-4-methoxybenzyl)-5-(2-methoxypyridin-3-yl)-2,3-dihydro-1H-pyrrolo[3,4-b]quinolin-1-one;
9-amino-5-(1,3-dimethyl-1H-pyrazol-5-yl)-2-(4-methoxybenzyl)-2,3-dihydro-1H-pyrrolo[3,4-b]quinolin-1-one;
9-amino-2-(3-chloro-4-methoxybenzyl)-5-(2-fluoro-6-methoxyphenyl)-2,3-dihydro-1H-pyrrolo[3,4-b]quinolin-1-one;
9-amino-5-(4-methoxy-2-(trifluoromethyl)phenyl)-2-propyl-2,3-dihydro-1H-pyrrolo[3,4-b]quinolin-1-one;
9-amino-5-(5-bromo-2-methoxypyridin-3-yl)-2-propyl-2,3-dihydro-1H-pyrrolo[3,4-b]quinolin-1-one;
9-amino-5-(2-methylpyridin-3-yl)-2-propyl-2,3-dihydro-1H-pyrrolo[3,4-b]quinolin-1-one;
9-amino-5-(2-fluoro-3-methoxyphenyl)-2-propyl-2,3-dihydro-1H-pyrrolo[3,4-b]quinolin-1-one;
9-amino-5-(2,5-dichlorophenyl)-2-propyl-2,3-dihydro-1H-pyrrolo[3,4-b]quinolin-1-one;
9-amino-5-(2,6-difluoro-4-methoxyphenyl)-2-propyl-2,3-dihydro-1H-pyrrolo[3,4-b]quinolin-1-one;
9-amino-5-(3,4-dimethoxyphenyl)-2-propyl-2,3-dihydro-1H-pyrrolo[3,4-b]quinolin-1-one;
9-amino-5-(2-fluoro-5-methoxyphenyl)-2-propyl-2,3-dihydro-1H-pyrrolo[3,4-b]quinolin-1-one;
9-amino-5-(2-fluoro-4-methoxyphenyl)-2-propyl-2,3-dihydro-1H-pyrrolo[3,4-b]quinolin-1-one;
9-amino-2-cyclopentyl-5-(2,5-dimethoxyphenyl)-2,3-dihydro-1H-pyrrolo[3,4-b]quinolin-1-one;
9-amino-2-ethyl-5-(2-fluoro-5-methoxyphenyl)-2,3-dihydro-1H-pyrrolo[3,4-b]quinolin-1-one;
9-amino-2-(3,4-dimethoxybenzyl)-5-(2,4-dimethoxypyrimidin-5-yl)-2,3-dihydro-1H-pyrrolo[3,4-b]quinolin-1-one;
9-amino-5-(2,5-dimethoxyphenyl)-2-ethyl-6-fluoro-2,3-dihydro-1H-pyrrolo[3,4-b]quinolin-1-one;
9-amino-5-(2,4-dimethoxyphenyl)-6-fluoro-2-propyl-2,3-dihydro-1H-pyrrolo[3,4-b]quinolin-1-one;
9-amino-5-(3,5-dimethylphenyl)-2-ethyl-2,3-dihydro-1H-pyrrolo[3,4-b]quinolin-1-one;
9-amino-2-cyclopropyl-5-(2-fluoro-4-methoxyphenyl)-2,3-dihydro-1H-pyrrolo[3,4-b]quinolin-1-one;
9-amino-5-(2,4-difluorophenyl)-2-propyl-2,3-dihydro-1H-pyrrolo[3,4-b]quinolin-1-one;
9-amino-5-(2-chloro-6-methoxyphenyl)-2-cyclopropyl-2,3-dihydro-1H-pyrrolo[3,4-b]quinolin-1-one;
9-amino-5-(2-ethoxypyridin-3-yl)-2-propyl-2,3-dihydro-1H-pyrrolo[3,4-b]quinolin-1-one;
9-amino-5-(3,5-difluorophenyl)-2-(3,4-dimethoxybenzyl)-2,3-dihydro-1H-pyrrolo[3,4-b]quinolin-1-one;
2-(9-amino-2-cyclopropyl-1-oxo-2,3-dihydro-1H-pyrrolo[3,4-b]quinolin-5-yl)-3-methoxybenzonitrile;
9-amino-5-(2-chloro-5-fluoropyridin-3-yl)-2-propyl-2,3-dihydro-1H-pyrrolo[3,4-b]quinolin-1-one;
2-(9-amino-2-cyclopropyl-1-oxo-2,3-dihydro-1H-pyrrolo[3,4-b]quinolin-5-yl)benzonitrile;
9-amino-5-(6-methoxy-2-methylpyridin-3-yl)-2-propyl-2,3-dihydro-1H-pyrrolo[3,4-b]quinolin-1-one;
9-amino-2-propyl-5-(2-(trifluoromethyl)phenyl)-2,3-dihydro-1H-pyrrolo[3,4-b]quinolin-1-one;
9-amino-2-cyclopropyl-5-(2,3-difluorophenyl)-2,3-dihydro-1H-pyrrolo[3,4-b]quinolin-1-one;
9-amino-2-cyclopropyl-5-(2-fluorophenyl)-2,3-dihydro-1H-pyrrolo[3,4-b]quinolin-1-one;
9-amino-2-propyl-5-(quinolin-6-yl)-2,3-dihydro-1H-pyrrolo[3,4-b]quinolin-1-one;
9-amino-5-(2-fluoro-6-methylpyridin-3-yl)-2-propyl-2,3-dihydro-1H-pyrrolo[3,4-b]quinolin-1-one;
9-amino-2-butyl-5-(2-methoxypyridin-3-yl)-2,3-dihydro-1H-pyrrolo[3,4-b]quinolin-1-one;
9-amino-2-cyclopropyl-5-(2,4-difluorophenyl)-2,3-dihydro-1H-pyrrolo[3,4-b]quinolin-1-one;
9-amino-5-(6′-chloro-2,3′-bipyridin-5-yl)-2-(3,4-dimethoxybenzyl)-2,3-dihydro-1H-pyrrolo[3,4-b]quinolin-1-one;
9-amino-5-(2-fluoro-6-methoxyphenyl)-2-((R)-1-(4-methoxyphenyl)ethyl)-2,3-dihydro-1H-pyrrolo[3,4-b]quinolin-1-one;
3-(9-amino-1-oxo-2-propyl-2,3-dihydro-1H-pyrrolo[3,4-b]quinolin-5-yl)benzonitrile;
9-amino-2-butyl-5-(2-methoxy-5-methylphenyl)-2,3-dihydro-1H-pyrrolo[3,4-b]quinolin-1-one;
9-amino-5-(1,3-dimethyl-1H-pyrazol-5-yl)-2-propyl-2,3-dihydro-1H-pyrrolo[3,4-b]quinolin-1-one;
9-amino-5-(5-chloro-2-methoxypyridin-3-yl)-2-propyl-2,3-dihydro-1H-pyrrolo[3,4-b]quinolin-1-one;
9-amino-5-(2,6-dichloropyridin-3-yl)-2-propyl-2,3-dihydro-1H-pyrrolo[3,4-b]quinolin-1-one;
9-amino-5-(6-fluoro-2-methylpyridin-3-yl)-2-propyl-2,3-dihydro-1H-pyrrolo[3,4-b]quinolin-1-one;
9-amino-2-ethyl-5-(3-fluoro-2-methoxyphenyl)-2,3-dihydro-1H-pyrrolo[3,4-b]quinolin-1-one;
9-amino-2-cyclopropyl-5-(2-(trifluoromethyl)phenyl)-2,3-dihydro-1H-pyrrolo[3,4-b]quinolin-1-one;
9-amino-5-(6-chloro-2-methylpyridin-3-yl)-2-propyl-2,3-dihydro-1H-pyrrolo[3,4-b]quinolin-1-one;
9-amino-2-ethyl-5-(4-methylpyridin-3-yl)-2,3-dihydro-1H-pyrrolo[3,4-b]quinolin-1-one;
9-amino-2-cyclopropyl-5-(2,6-dimethylphenyl)-2,3-dihydro-1H-pyrrolo[3,4-b]quinolin-1-one;
9-amino-5-(2-methoxy-6-methylpyridin-3-yl)-2-propyl-2,3-dihydro-1H-pyrrolo[3,4-b]quinolin-1-one;
9-amino-5-(2-fluoro-5-(trifluoromethyl)phenyl)-2-propyl-2,3-dihydro-1H-pyrrolo[3,4-b]quinolin-1-one;
9-amino-5-(2,3-dimethoxyphenyl)-2-propyl-2,3-dihydro-1H-pyrrolo[3,4-b]quinolin-1-one;
9-amino-2-propyl-5-(pyridin-4-yl)-2,3-dihydro-1H-pyrrolo[3,4-b]quinolin-1-one;
9-amino-2-ethyl-5-(4-fluoro-3-methoxyphenyl)-2,3-dihydro-1H-pyrrolo[3,4-b]quinolin-1-one;
9-amino-2-ethyl-5-(2-methoxy-5-methylphenyl)-2,3-dihydro-1H-pyrrolo[3,4-b]quinolin-1-one;
9-amino-5-(5-fluoro-2-methylphenyl)-2-(4-methoxybenzyl)-2,3-dihydro-1H-pyrrolo[3,4-b]quinolin-1-one;
9-amino-5-(1H-indol-5-yl)-2-propyl-2,3-dihydro-1H-pyrrolo[3,4-b]quinolin-1-one;
9-amino-5-(2-fluoro-6-methoxyphenyl)-2-isopropyl-2,3-dihydro-1H-pyrrolo[3,4-b]quinolin-1-one;
9-amino-5-(2,5-dimethoxyphenyl)-2-(3-methoxybenzyl)-2,3-dihydro-1H-pyrrolo[3,4-b]quinolin-1-one;
9-amino-5-(3,5-dimethoxyphenyl)-2-ethyl-2,3-dihydro-1H-pyrrolo[3,4-b]quinolin-1-one;
9-amino-2-ethyl-5-(5-fluoro-2-methoxyphenyl)-2,3-dihydro-1H-pyrrolo[3,4-b]quinolin-1-one;
9-amino-2-(3-chlorobenzyl)-5-(2-fluoro-6-methoxyphenyl)-2,3-dihydro-1H-pyrrolo[3,4-b]quinolin-1-one;
9-amino-5-(6-chloro-2-methylpyridin-3-yl)-2-cyclopropyl-2,3-dihydro-1H-pyrrolo[3,4-b]quinolin-1-one;
9-amino-2-cyclopropyl-5-(6-methylpyridin-3-yl)-2,3-dihydro-1H-pyrrolo[3,4-b]quinolin-1-one;
9-amino-2-(benzo[d][1,3]dioxol-5-ylmethyl)-5-(2,4-dimethoxypyrimidin-5-yl)-2,3-dihydro-1H-pyrrolo[3,4-b]quinolin-1-one;
9-amino-5-(6-fluoro-5-methylpyridin-3-yl)-2-propyl-2,3-dihydro-1H-pyrrolo[3,4-b]quinolin-1-one;
2-(9-amino-1-oxo-2-propyl-2,3-dihydro-1H-pyrrolo[3,4-b]quinolin-5-yl)benzonitrile;
9-amino-2-propyl-5-(thiophen-3-yl)-2,3-dihydro-1H-pyrrolo[3,4-b]quinolin-1-one;
9-amino-5-(4-fluoro-2-methoxyphenyl)-2-(4-methoxybenzyl)-2,3-dihydro-1H-pyrrolo[3,4-b]quinolin-1-one;
9-amino-2-cyclopropyl-5-(4-methoxy-2-(trifluoromethyl)phenyl)-2,3-dihydro-1H-pyrrolo[3,4-b]quinolin-1-one;
9-amino-5-(2,4-dimethoxyphenyl)-2-(4-methoxybenzyl)-2,3-dihydro-1H-pyrrolo[3,4-b]quinolin-1-one;
9-amino-5-(2-fluoro-3-methoxyphenyl)-2-methyl-2,3-dihydro-1H-pyrrolo[3,4-b]quinolin-1-one;
9-amino-5-(2-chloro-5-methoxyphenyl)-2-cyclopropyl-2,3-dihydro-1H-pyrrolo[3,4-b]quinolin-1-one;
9-amino-2-(4-methoxybenzyl)-5-(2-(trifluoromethyl)phenyl)-2,3-dihydro-1H-pyrrolo[3,4-b]quinolin-1-one;
9-amino-2-ethyl-5-(3-fluoro-5-methoxyphenyl)-2,3-dihydro-1H-pyrrolo[3,4-b]quinolin-1-one;
9-amino-5-(2,5-difluorophenyl)-2-propyl-2,3-dihydro-1H-pyrrolo[3,4-b]quinolin-1-one;
9-amino-2-cyclopropyl-5-(2-methoxypyridin-3-yl)-2,3-dihydro-1H-pyrrolo[3,4-b]quinolin-1-one;
9-amino-5-(6-methoxy-5-methylpyridin-3-yl)-2-propyl-2,3-dihydro-1H-pyrrolo[3,4-b]quinolin-1-one;
9-amino-2-cyclopropyl-5-(2,5-dimethoxyphenyl)-6-(methylthio)-2,3-dihydro-1H-pyrrolo[3,4-b]quinolin-1-one;
9-amino-2-cyclopropyl-5-(2,5-difluoro-4-methoxyphenyl)-2,3-dihydro-1H-pyrrolo[3,4-b]quinolin-1-one;
9-amino-5-(3-(dimethylamino)phenyl)-2-propyl-2,3-dihydro-1H-pyrrolo[3,4-b]quinolin-1-one;
9-amino-5-(furan-3-yl)-2-propyl-2,3-dihydro-1H-pyrrolo[3,4-b]quinolin-1-one;
9-amino-5-(4-methoxypyridin-3-yl)-2-propyl-2,3-dihydro-1H-pyrrolo[3,4-b]quinolin-1-one;
9-amino-2-cyclobutyl-5-(3,5-difluorophenyl)-2,3-dihydro-1H-pyrrolo[3,4-b]quinolin-1-one;
9-amino-5-(5-chloro-2-methoxyphenyl)-2-propyl-2,3-dihydro-1H-pyrrolo[3,4-b]quinolin-1-one;
9-amino-5-(2-methoxypyridin-3-yl)-2-propyl-2,3-dihydro-1H-pyrrolo[3,4-b]quinolin-1-one;
9-amino-2-ethyl-5-(3-fluoro-4-methoxyphenyl)-2,3-dihydro-1H-pyrrolo[3,4-b]quinolin-1-one;
9-amino-2-cyclopropyl-5-(2-fluoro-6-methylpyridin-3-yl)-2,3-dihydro-1H-pyrrolo[3,4-b]quinolin-1-one;
9-amino-5-phenyl-2-propyl-2,3-dihydro-1H-pyrrolo[3,4-b]quinolin-1-one;
9-amino-2-propyl-5-(3-methylphenyl)-2,3-dihydro-1H-pyrrolo[3,4-b]quinolin-1-one;
9-amino-2-(2-butyl)-5-(2-fluoro-6-methoxyphenyl)-2,3-dihydro-1H-pyrrolo[3,4-b]quinolin-1-one;
9-amino-5-(5-fluoro-2-methoxyphenyl)-2-(4-methoxybenzyl)-2,3-dihydro-1H-pyrrolo[3,4-b]quinolin-1-one;
9-amino-5-(3,5-dimethoxyphenyl)-2-propyl-2,3-dihydro-1H-pyrrolo[3,4-b]quinolin-1-one;
9-amino-5-(2-methoxy-5-methylphenyl)-2-propyl-2,3-dihydro-1H-pyrrolo[3,4-b]quinolin-1-one;
9-amino-5-(3,4-dimethoxyphenyl)-2-(4-methoxybenzyl)-2,3-dihydro-1H-pyrrolo[3,4-b]quinolin-1-one;
9-amino-5-(2,5-difluoro-4-methoxyphenyl)-2-(4-methoxybenzyl)-2,3-dihydro-1H-pyrrolo[3,4-b]quinolin-1-one;
9-amino-5-(2,4-dimethoxyphenyl)-2-propyl-2,3-dihydro-1H-pyrrolo[3,4-b]quinolin-1-one;
9-amino-2-propyl-5-(2,3,4-trimethoxyphenyl)-2,3-dihydro-1H-pyrrolo[3,4-b]quinolin-1-one;
9-amino-5-(2-methoxy-5-(pyridin-4-yl)phenyl)-2-propyl-2,3-dihydro-1H-pyrrolo[3,4-b]quinolin-1-one;
4-(9-amino-1-oxo-2-propyl-2,3-dihydro-1H-pyrrolo[3,4-b]quinolin-5-yl)benzonitrile;
9-amino-2-cyclopropyl-5-(2-methoxyphenyl)-2,3-dihydro-1H-pyrrolo[3,4-b]quinolin-1-one;
9-amino-5-(2-methoxy-5-methylphenyl)-2-(4-methoxybenzyl)-2,3-dihydro-1H-pyrrolo[3,4-b]quinolin-1-one;
9-amino-2-cyclopropyl-5-(6-methoxy-5-methylpyridin-3-yl)-2,3-dihydro-1H-pyrrolo[3,4-b]quinolin-1-one;
9-amino-5-(4-methylpyridin-3-yl)-2-propyl-2,3-dihydro-1H-pyrrolo[3,4-b]quinolin-1-one;
9-amino-5-(2-fluoro-5-methoxyphenyl)-2-methyl-2,3-dihydro-1H-pyrrolo[3,4-b]quinolin-1-one;
9-amino-5-(2,5-dimethylphenyl)-2-propyl-2,3-dihydro-1H-pyrrolo[3,4-b]quinolin-1-one;
9-amino-5-(3-fluoro-2-methoxyphenyl)-2-propyl-2,3-dihydro-1H-pyrrolo[3,4-b]quinolin-1-one;
9-amino-2-cyclopropyl-5-(2,4-dimethoxyphenyl)-2,3-dihydro-1H-pyrrolo[3,4-b]quinolin-1-one;
9-amino-5-(2,5-dimethoxyphenyl)-2-ethyl-2,3-dihydro-1H-pyrrolo[3,4-b]quinolin-1-one;
9-amino-2-(4-methoxybenzyl)-5-(6-methylpyridin-3-yl)-2,3-dihydro-1H-pyrrolo[3,4-b]quinolin-1-one;
9-amino-2-(benzo[d][1,3]dioxol-5-ylmethyl)-5-(2,5-dimethoxyphenyl)-2,3-dihydro-1H-pyrrolo[3,4-b]quinolin-1-one;
9-amino-5-(4-fluoro-2-methoxyphenyl)-2-propyl-2,3-dihydro-1H-pyrrolo[3,4-b]quinolin-1-one;
9-amino-5-(3-methoxyphenyl)-2-propyl-2,3-dihydro-1H-pyrrolo[3,4-b]quinolin-1-one;
9-amino-5-(5-methoxypyridin-3-yl)-2-propyl-2,3-dihydro-1H-pyrrolo[3,4-b]quinolin-one;
2-(9-amino-2-methyl-1-oxo-2,3-dihydro-1H-pyrrolo[3,4-b]quinolin-5-yl)benzonitrile;
9-amino-2-cyclopropyl-5-(pyridin-3-yl)-2,3-dihydro-1H-pyrrolo[3,4-b]quinolin-1-one;
5-(9-amino-1-oxo-2-propyl-2,3-dihydro-1H-pyrrolo[3,4-b]quinolin-5-yl)nicotinonitrile;
9-amino-2-methyl-5-(4-methylpyridin-3-yl)-2,3-dihydro-1H-pyrrolo[3,4-b]quinolin-1-one;
9-amino-5-(3,5-dimethylphenyl)-2-methyl-2,3-dihydro-1H-pyrrolo[3,4-b]quinolin-1-one;
9-amino-5-(2,5-dimethoxyphenyl)-2-(4-methoxybenzyl)-2,3-dihydro-1H-pyrrolo[3,4-b]quinolin-1-one;
9-amino-5-(2-methoxy-5-methylphenyl)-2-methyl-2,3-dihydro-1H-pyrrolo[3,4-b]quinolin-1-one;
9-amino-2-(4-methoxybenzyl)-5-(1-methyl-1H-pyrazol-4-yl)-2,3-dihydro-1H-pyrrolo[3,4-b]quinolin-1-one;
9-amino-2-cyclopropyl-5-(5-fluoro-2-methoxyphenyl)-2,3-dihydro-1H-pyrrolo[3,4-b]quinolin-1-one;
9-amino-5-(3,5-dimethoxyphenyl)-2-methyl-2,3-dihydro-1H-pyrrolo[3,4-b]quinolin-1-one;
5-(9-amino-1-oxo-2-propyl-2,3-dihydro-1H-pyrrolo[3,4-b]quinolin-5-yl)-2-fluorobenzonitrile;
9-amino-5-(2,6-difluoropyridin-3-yl)-2-propyl-2,3-dihydro-1H-pyrrolo[3,4-b]quinolin-1-one;
2-(9-amino-1-oxo-2-propyl-2,3-dihydro-1H-pyrrolo[3,4-b]quinolin-5-yl)-3-methoxybenzonitrile;
9-amino-5-(2-methoxypyrimidin-5-yl)-2-propyl-2,3-dihydro-1H-pyrrolo[3,4-b]quinolin-1-one;
9-amino-5-(3-chlorophenyl)-2-propyl-2,3-dihydro-1H-pyrrolo[3,4-b]quinolin-1-one;
9-amino-2-ethyl-5-(6-methylpyridin-3-yl)-2,3-dihydro-1H-pyrrolo[3,4-b]quinolin-1-one;
9-amino-5-(2-fluoro-4-methoxyphenyl)-2-methyl-2,3-dihydro-1H-pyrrolo[3,4-b]quinolin-1-one;
9-amino-2-cyclopropyl-5-(2,4-dimethoxypyrimidin-5-yl)-2,3-dihydro-1H-pyrrolo[3,4-b]quinolin-1-one;
9-amino-5-(4-fluorophenyl)-2-propyl-2,3-dihydro-1H-pyrrolo[3,4-b]quinolin-1-one;
9-amino-5-(2,4-dimethoxyphenyl)-2-methyl-2,3-dihydro-1H-pyrrolo[3,4-b]quinolin-1-one;
9-amino-5-(5-fluoro-2-methoxyphenyl)-2-propyl-2,3-dihydro-1H-pyrrolo[3,4-b]quinolin-1-one;
9-amino-5-(3-fluoro-5-methoxyphenyl)-2-methyl-2,3-dihydro-1H-pyrrolo[3,4-b]quinolin-1-one;
9-amino-2-propyl-5-(4-methylphenyl)-2,3-dihydro-1H-pyrrolo[3,4-b]quinolin-1-one;
9-amino-5-(5-fluoro-6-methoxypyridin-3-yl)-2-propyl-2,3-dihydro-1H-pyrrolo[3,4-b]quinolin-1-one;
9-amino-5-(2,3-dimethoxyphenyl)-2-ethyl-2,3-dihydro-1H-pyrrolo[3,4-b]quinolin-1-one;
9-amino-5-(2,6-dimethoxyphenyl)-2-ethyl-2,3-dihydro-1H-pyrrolo[3,4-b]quinolin-1-one;
9-amino-2-cyclopropyl-5-(4-fluoro-2-methoxyphenyl)-2,3-dihydro-1H-pyrrolo[3,4-b]quinolin-1-one;
9-amino-5-(6-chloro-5-methylpyridin-3-yl)-2-propyl-2,3-dihydro-1H-pyrrolo[3,4-b]quinolin-1-one;
9-amino-5-(3,4-dimethoxyphenyl)-2-methyl-2,3-dihydro-1H-pyrrolo[3,4-b]quinolin-1-one;
9-amino-2-propyl-5-(3,4,5-trimethoxyphenyl)-2,3-dihydro-1H-pyrrolo[3,4-b]quinolin-1-one;
9-amino-2-butyl-5-(6-methylpyridin-3-yl)-2,3-dihydro-1H-pyrrolo[3,4-b]quinolin-1-one;
9-amino-5-(2,3-difluorophenyl)-2-(4-methoxybenzyl)-2,3-dihydro-1H-pyrrolo[3,4-b]quinolin-1-one;
9-amino-5-(2,4-dimethoxyphenyl)-2-ethyl-2,3-dihydro-1H-pyrrolo[3,4-b]quinolin-1-one;
9-amino-5-(1-methyl-1H-pyrazol-4-yl)-2-propyl-2,3-dihydro-1H-pyrrolo[3,4-b]quinolin-1-one;
9-amino-2-(3-methoxybenzyl)-5-(4-methoxypyridin-3-yl)-2,3-dihydro-1H-pyrrolo[3,4-b]quinolin-1-one;
9-amino-5-(2,5-difluoro-4-methoxyphenyl)-2-propyl-2,3-dihydro-1H-pyrrolo[3,4-b]quinolin-1-one;
9-amino-2-cyclopropyl-5-(3,4-dimethoxyphenyl)-6-fluoro-2,3-dihydro-1H-pyrrolo[3,4-b]quinolin-1-one;
9-amino-2-cyclobutyl-5-(2-fluoro-6-methylpyridin-3-yl)-2,3-dihydro-1H-pyrrolo[3,4-b]quinolin-1-one;
9-amino-5-(3,4-dimethoxyphenyl)-6-fluoro-2-propyl-2,3-dihydro-1H-pyrrolo[3,4-b]quinolin-1-one;
9-amino-2-cyclobutyl-5-(3,4-dimethoxyphenyl)-6-fluoro-2,3-dihydro-1H-pyrrolo[3,4-b]quinolin-1-one;
9-amino-2-cyclopentyl-5-(2,4-dimethoxypyrimidin-5-yl)-2,3-dihydro-1H-pyrrolo[3,4-b]quinolin-1-one;
9-amino-2-cyclopentyl-5-(2-methoxypyridin-3-yl)-2,3-dihydro-1H-pyrrolo[3,4-b]quinolin-1-one;
9-amino-5-(2-chloro-6-methylpyridin-3-yl)-2-cyclobutyl-2,3-dihydro-1H-pyrrolo[3,4-b]quinolin-1-one;
5-(9-amino-2-cyclobutyl-1-oxo-2,3-dihydro-1H-pyrrolo[3,4-b]quinolin-5-yl)picolinonitrile;
9-amino-5-(3,4-dimethoxyphenyl)-2-ethyl-6-(methylthio)-2,3-dihydro-1H-pyrrolo[3,4-b]quinolin-1-one;
9-amino-2-cyclobutyl-5-(pyridazin-4-yl)-2,3-dihydro-1H-pyrrolo[3,4-b]quinolin-1-one;
2-(9-amino-2-cyclopentyl-1-oxo-2,3-dihydro-1H-pyrrolo[3,4-b]quinolin-5-yl)benzonitrile;
9-amino-2-cyclopentyl-5-(2,5-difluoro-4-methoxyphenyl)-2,3-dihydro-1H-pyrrolo[3,4-b]quinolin-1-one;
9-amino-2-cyclopentyl-5-(5-methoxypyridin-3-yl)-2,3-dihydro-1H-pyrrolo[3,4-b]quinolin-1-one;
9-amino-2-cyclopentyl-5-(6-methoxypyridin-3-yl)-2,3-dihydro-1H-pyrrolo[3,4-b]quinolin-1-one;
9-amino-2-cyclopentyl-5-(2,6-dimethoxypyridin-3-yl)-2,3-dihydro-1H-pyrrolo[3,4-b]quinolin-1-one;
9-amino-2-cyclopentyl-5-(2-ethoxypyridin-3-yl)-2,3-dihydro-1H-pyrrolo[3,4-b]quinolin-1-one; and
   pharmaceutically acceptable salts thereof.

The definitions set forth in this application are intended to clarify terms used throughout this application. The term “herein” means the entire application.

As used in this application, the term “optionally substituted,” as used herein, means that substitution is optional and therefore it is possible for the designated atom or moiety to be unsubstituted. In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example, if a methyl group (i.e., CH₃) is optionally substituted, then 3 hydrogens on the carbon atom can be replaced. Examples of suitable substituents include, but are not limited to: halogen, CN, NH₂, OH, SO, SO₂, COOH, OC₁₋₆alkyl, CH₂OH, SO₂H, C₁₋₆alkyl, OC₁₋₆alkyl, C(═O)C₁₋₆alkyl, C(═O)OC₁₋₆alkyl, C(═O)NH₂, C(═O)NHC₁₋₆alkyl, C(═O)N(C₁₋₆alkyl)2, SO₂C₁₋₆alkyl, SO₂NHC₁₋₆alkyl, SO₂N(C₁₋₆alkyl)2, NH(C₁₋₆alkyl), N(C₁₋₆alkyl)2, NHC(═O)C₁₋₆alkyl, NC(═O)(C₁₋₆alkyl)₂, C₅₋₆aryl, OC₅₋₆aryl, C(═O)C₅₋₆aryl, C(═O)OC₅₋₆aryl, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NHC₅₋₆heterocyclyl, C(═O)N(C₅₋₆heterocyclyl)2, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(═O)C₅₋₆heterocyclyl, NC(═O)(C₅₋₆heterocyclyl)₂.

A variety of compounds in the present invention may exist in particular stereoisomeric forms. The present invention takes into account all such compounds, including cis- and trans isomers, R— and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. Many stereoisomers of olefins, C═N double bonds, and the like can also be present in the compounds described herein, and all such stable isomers are contemplated in the present invention. Cis and trans isomers of the compounds of the present invention are described and may be isolated as a mixture of isomers or as separated isomeric forms. All chiral, diastereomeric, racemic forms and all stereoisomeric forms of a structure are intended, unless the specific stereochemistry or isomeric form is specifically indicated. Where the compounds contain chiral centres, all individual optical forms such as enantiomers, epimers, atropisomers and diastereoisomers, as well as racemic mixtures of the compounds are within the scope of the invention.

Compounds may exist in a number of tautomeric forms and references to compounds include all such forms. For the avoidance of doubt, where a compound can exist in one of several tautomeric forms and only one is specifically described or shown, all others are nevertheless embraced by the scope of this invention.

The compounds of the invention may form isolable atropisomers in certain solvents (e.g. supercritical CO₂ containing 25-35% methanol) at room temperature. The atropisomers of the compounds may be isolated using chiral LC. All atropisomers of a structure are intended, unless the specific atropisomer is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

The term “Cₘ₋ₙ” or “Cₘ₋ₙ group” used alone or as a prefix, refers to any group having m to n carbon atoms.

The term “alkyl” used alone or as a suffix or prefix, refers to a saturated monovalent straight or branched chain hydrocarbon radical comprising 1 to about 12 carbon atoms. Illustrative examples of alkyls include, but are not limited to, C₁₋₆alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl.

The term “alkylene” used alone or as suffix or prefix, refers to divalent straight or branched chain hydrocarbon radicals comprising 1 to about 12 carbon atoms, which serves to link two structures together.

As used herein, “alkenyl” refers to an alkyl group having one or more double carbon-carbon bonds. Example alkenyl groups include ethenyl, propenyl, cyclohexenyl, and the like. The term “alkenylenyl” refers to a divalent linking alkenyl group.

As used herein, “alkynyl” refers to an alkyl group having one or more triple carbon-carbon bonds. Example alkynyl groups include ethynyl, propynyl, and the like. The term “alkynylenyl” refers to a divalent linking alkynyl group.

As used herein, “aromatic” refers to hydrocarbyl groups having one or more polyunsaturated carbon rings having aromatic characters, (e.g., 4n+2 delocalized electrons) and comprising up to about 14 carbon atoms.

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be a polycyclic moiety in which at least one carbon is common to any two adjoining rings therein (for example, the rings are “fused rings”), for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls or cycloalkynyls. The terms ortho, meta and para apply to 1,2-, 1,3- and 1,4-disubstituted benzenes, respectively. For example, the names 1,2-dimethylbenzene and ortho-dimethylbenzene are synonymous.

The term “cycloalkyl,” used alone or as suffix or prefix, refers to a saturated monovalent ring-containing hydrocarbon radical comprising at least 3 up to about 12 carbon atoms. Examples of cycloalkyls include, but are not limited to, C₃₋₇cycloalkyl groups, such as cyclopropyl, cyclobutyl, cyclopentyl, cyclohexyl, and cycloheptyl, and saturated cyclic and bicyclic terpenes. A cycloalkyl can be unsubstituted or substituted by one or two suitable substituents. Preferably, the cycloalkyl is a monocyclic ring or bicyclic ring.

As used herein, “cycloalkenyl” refers to ring-containing hydrocarbyl groups having at least one carbon-carbon double bond in the ring, and having from 3 to 12 carbons atoms.

As used herein, “halo” or “halogen” refers to fluoro, chloro, bromo, and iodo.

“Counterion” is used to represent a small, negatively or positively charged species such as chloride (Cl⁻), bromide (Br⁻), hydroxide (OH⁻), acetate (CH₃COO⁻), sulfate (SO₄²⁻), tosylate (CH₃-phenyl-SO₃⁻), benezensulfonate (phenyl-SO₃⁻), sodium ion (Na⁺), potassium (K⁺), ammonium (NH₄⁺), and the like.

The term “heterocycle” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroaromatic” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen therefrom.

The term “heterocyclylene” used alone or as a suffix or prefix, refers to a divalent radical derived from a heterocycle by removing two hydrogens therefrom, which serves to link two structures together.

The term “heteroaryl” used alone or as a suffix or prefix, refers to a heterocyclyl having aromatic character.

The term “heterocylcoalkyl” used alone or as a suffix or prefix, refers to a monocyclic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as C₃₋₆heterocycloalkyl.

The term “heteroarylene” used alone or as a suffix or prefix, refers to a heterocyclylene having aromatic character.

The term “heterocycloalkylene” used alone or as a suffix or prefix, refers to a heterocyclylene that does not have aromatic character.

The term “six-membered” used as prefix refers to a group having a ring that contains six ring atoms.

The term “five-membered” used as prefix refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

Examples of heterocyclyls include, but are not limited to, 1H-indazole, 2-pyrrolidonyl, 2H, 6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azabicyclo, azetidine, azepane, aziridine, azocinyl, benzimidazolyl, benzodioxol, benzofuranyl, benzothiofuranyl, benzothiophenyl, benzoxazolyl, benzthiazolyl, benzotriazolyl, benzotetrazolyl, benzisoxazolyl, benzisothiazolyl, benzimidazalonyl, carbazolyl, 4aH-carbazolyl, b-carbolinyl, chromanyl, chromenyl, cinnolinyl, diazepane, decahydroquinolinyl, 2H,6H-1,5,2-dithiazinyl, dioxolane, furyl, 2,3-dihydrofuran, 2,5-dihydrofuran, dihydrofuro[2,3-b]tetrahydrofuran, furanyl, furazanyl, homopiperidinyl, imidazolidine, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxirane, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, purinyl, pyranyl, pyrrolidinyl, pyrroline, pyrrolidine, pyrazinyl, pyrazolidinyl, pyrazolinyl, pyrazolyl, pyridazinyl, pyridooxazole, pyridoimidazole, pyridothiazole, pyridinyl, N-oxide-pyridinyl, pyridyl, pyrimidinyl, pyrrolidinyl, pyrrolidinyl dione, pyrrolinyl, pyrrolyl, pyridine, quinazolinyl, quinolinyl, 4H-quinolizinyl, quinoxalinyl, quinuclidinyl, carbolinyl, tetrahydrofuranyl, tetramethylpiperidinyl, tetrahydroquinoline, tetrahydroisoquinolinyl, thiophane, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiopheneyl, thiirane, triazinyl, 1,2,3-triazolyl, 1,2,4-triazolyl, 1,2,5-triazolyl, 1,3,4-triazolyl, xanthenyl.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, isopropoxy, n-butoxy, isobutoxy, t-butoxy, n-pentoxy, isopentoxy, cyclopropylmethoxy, allyloxy and propargyloxy. Similarly, “alkylthio” or “thioalkoxy” represent an alkyl group as defined above with the indicated number of carbon atoms attached through a sulphur bridge.

“Halogenated,” used as a prefix of a group, means one or more hydrogens on the group is replaced with one or more halogens.

As used herein, the term “carbonyl” is art recognized and includes the —C(═O) groups of such moieties as can be represented by the general formula:

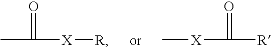

wherein X is a bond or represents an oxygen or sulfur, and R represents a hydrogen, an alkyl, an alkenyl, —(CH₂)ₘ—R″ or a pharmaceutically acceptable salt, R′ represents a hydrogen, an alkyl, an alkenyl or —(CH₂)ₘ—R″, where m is an integer less than or equal to ten, and R″ is alkyl, cycloalkyl, alkenyl, aryl, or heteroaryl. Where X is an oxygen and R and R′ is not hydrogen, the formula represents an “ester”. Where X is an oxygen, and R is as defined above, the moiety is referred to herein as a carboxyl group, and particularly when R′ is a hydrogen, the formula represents a “carboxylic acid.” Where X is oxygen, and R′ is a hydrogen, the formula represents a “formate.” In general, where the oxygen atom of the above formula is replaced by sulfur, the formula represents a “thiolcarbonyl” group. Where X is a sulfur and R and R′ is not hydrogen, the formula represents a “thiolester.” Where X is sulfur and R is hydrogen, the formula represents a “thiolcarboxylic acid.” Where X is sulfur and R′ is hydrogen, the formula represents a “thiolformate.” On the other hand, where X is a bond, and R is not a hydrogen, the above formula represents a “ketone” group. Where X is a bond, and R is hydrogen, the above formula is represents an “aldehyde” group.

As used herein, the term “sulfonyl” refers to the —S(═O)₂— of a moiety that can be represented by the general formula:

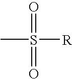

wherein R is represented by but not limited to hydrogen, alkyl, cycloalkyl, alkenyl, aryl, heteroaryl, aralkyl, or heteroaralkyl.

As used herein, some substituents are described in a combination of two or more groups. For example, the expression of “C(═O)C₃₋₉cycloalkylR^{d}” is meant to refer to a structure:

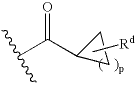

wherein p is 1, 2, 3, 4, 5, 6 or 7 (i.e., C₃₋₉cycloalkyl); the C₃₋₉cycloalkyl is substituted by R^{d}; and the point of attachment of the “C(═O)C₃₋₉cycloalkylR^{d}” is through the carbon atom of the carbonyl group, which is on the left of the expression.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof (i.e., also include counterions). Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, phosphoric, and the like; and the salts prepared from organic acids such as lactic, maleic, citric, benzoic, methanesulfonic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; nonaqueous media like ether, ethyl acetate, ethanol, isopropanol, or acetonitrile can be used.

As used herein, “in vivo hydrolysable precursors” means an in vivo hydrolysable (or cleavable) ester of a compound of any of the formulas described herein that contains a carboxy or a hydroxy group. For example amino acid esters, C₁₋₆ alkoxymethyl esters like methoxymethyl; C₁₋₆alkanoyloxymethyl esters like pivaloyloxymethyl; C₃₋₈cycloalkoxycarbonyloxy C₁₋₆alkyl esters like 1-cyclohexylcarbonyloxyethyl, acetoxymethoxy, or phosphoramidic cyclic esters.

As used herein, “tautomer” means other structural isomers that exist in equilibrium resulting from the migration of a hydrogen atom. For example, keto-enol tautomerism where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and formulation into an efficacious therapeutic agent.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S and ⁸²Br.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), esters such as in vivo hydrolysable esters, free acids or bases, polymorphic forms of the compounds, solvates (e.g. hydrates), prodrugs or lipids, coupling partners and protecting groups. By “prodrugs” is meant for example any compound that is converted in vivo into a biologically active compound.

Salts of the compounds of the invention are preferably physiologically well tolerated and non-toxic. Many examples of salts are known to those skilled in the art. All such salts are within the scope of this invention, and references to compounds include the salt forms of the compounds.

Compounds having acidic groups, such as carboxylate, phosphates or sulfates, can form salts with alkaline or alkaline earth metals such as Na, K, Mg and Ca, and with organic amines such as triethylamine and Tris (2-hydroxyethyl)amine. Salts can be formed between compounds with basic groups, e.g. amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Acid addition salts may be formed with a wide variety of acids, both inorganic and organic. Examples of acid addition salts include salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

If the compound is anionic, or has a functional group which may be anionic (e.g., COOH may be COO), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Esters can be formed between hydroxyl or carboxylic acid groups present in the compound and an appropriate carboxylic acid or alcohol reaction partner, using techniques well known in the art. Examples of esters are compounds containing the group C(═O)OR, wherein R is an ester substituent, for example, a C₁₋₇alkyl group, a C₃₋₂₀ heterocyclyl group, or a C₅₋₂₀aryl group, preferably a C₁₋₇alkyl group. Particular examples of ester groups include, but are not limited to, C(═O)OCH₃, C(═O)OCH₂CH₃, C(═O)OC(CH₃)₃, and —C(═O)OPh. Examples of acyloxy (reverse ester) groups are represented by OC(═O)R, wherein R is an acyloxy substituent, for example, a C₁₋₇alkyl group, a C₃₋₂₀heterocyclyl group, or a C₅₋₂₀aryl group, preferably a C₁₋₇alkyl group. Particular examples of acyloxy groups include, but are not limited to, OC(═O)CH₃ (acetoxy), OC(═O)CH₂CH₃, OC(═O)C(CH₃)₃, OC(═O)Ph, and OC(═O)CH₂Ph.

Derivatives which are prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it. Some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is: C₁₇alkyl (e.g., Me, Et, -nPr, -iPr, -nBu, -sBu, -iBu, tBu); C₁₇aminoalkyl (e.g., aminoethyl; 2-(N,N-diethylamino)ethyl; 2(4morpholino)ethyl); and acyloxy-C₁₇alkyl (e.g., acyloxymethyl; acyloxyethyl; pivaloyloxymethyl; acetoxymethyl; 1acetoxyethyl; 1-(1-methoxy-1-methyl)ethyl-carbonyloxyethyl; 1-(benzoyloxy)ethyl; isopropoxy-carbonyloxymethyl; 1isopropoxy-carbonyloxyethyl; cyclohexyl-carbonyloxymethyl; 1cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1(4tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group. Other derivatives include formulating the compounds with liposomes.

The present invention further provides compositions comprising a compound of any of the formulas described herein, or a pharmaceutically acceptable salt, tautomer, atropisomer, or in vivo-hydrolysable precursor thereof, and at least one pharmaceutically acceptable carrier, diluent or excipient.

The present invention further provides methods of treating or preventing an anxiety disorder in a patient, comprising administering to the patient a therapeutically effective amount of a compound of any of the formulas described herein, or a pharmaceutically acceptable salt, tautomer, atropisomer, or in vivo-hydrolysable precursor thereof.

The present invention further provides methods of treating or preventing a cognitive disorder in a patient, comprising administering to the patient a therapeutically effective amount of a compound of any of the formulas described herein, or a pharmaceutically acceptable salt, tautomer, atropisomer, or in vivo-hydrolysable precursor thereof.

The present invention further provides methods of treating or preventing a mood disorder in a patient, comprising administering to the patient a therapeutically effective amount of a compound of any of the formulas described herein, or a pharmaceutically acceptable salt, tautomer, atropisomer, or in vivo-hydrolysable precursor thereof.

The present invention further provides a compound of any of the formulas described herein, or a pharmaceutically acceptable salt, tautomer, atropisomer, or in vivo-hydrolysable precursor thereof, described herein for use as a medicament.

The present invention further provides a compound of any of the formulas described herein, or a pharmaceutically acceptable salt, tautomer, atropisomer, or in vivo-hydrolysable precursor thereof, described herein for the manufacture of a medicament.

The present invention further provides methods of modulating activity of GABAA receptor comprising contacting the GABAA receptor with a compound of any of the formulas described herein, or a pharmaceutically acceptable salt, tautomer, atropisomer, or in vivo-hydrolysable precursor thereof.

The present invention further provides synthetic methods of making a compound of any of the formulas described herein, or a pharmaceutically acceptable salt, tautomer, atropisomer, or in vivo-hydrolysable precursor thereof.

Compounds of the present invention also include pharmaceutically acceptable salts, tautomers and in vivo-hydrolysable precursors of the compounds of any of the formulas described herein. Compounds of the invention further include hydrates and solvates.

Compounds of the invention can be used as medicaments. In some embodiments, the present invention provides compounds of any of the formulas described herein, or pharmaceutically acceptable salts, tautomers or in vivo-hydrolysable precursors thereof, for use as medicaments. In some embodiments, the present invention provides compounds described herein for use as medicaments for treating or preventing an anxiety disorder, cognitive disorder, or mood disorder.

In some embodiments, the present invention provides compounds of any of the formulas described herein, or pharmaceutically acceptable salts, tautomers or in vivo-hydrolysable precursors thereof, in the manufacture of a medicament for the treatment or prophylaxis of an anxiety disorder, cognitive disorder, or mood disorder.

In some embodiments, the present invention provides a method for the treatment or prophylaxis of an anxiety disorder comprising administering to a mammal (including a human) a therapeutically effective amount of a compound of any of the formulas described herein, or a pharmaceutically acceptable salt, tautomer or in vivo-hydrolysable precursor thereof. As used herein, the phrase “anxiety disorder” includes, but is not limited to, one or more of the following: panic disorder, panic disorder without agoraphobia, panic disorder with agoraphobia, agoraphobia without history of panic disorder, specific phobia, social phobia, social anxiety disorder, obsessive-compulsive disorder, posttraumatic stress disorder, acute stress disorder, generalized anxiety disorder, generalized anxiety disorder due to a general medical condition, and the like.

In some embodiments, the present invention provides a method for the treatment or prophylaxis of a cognitive disorder comprising administering to a mammal (including a human) a therapeutically effective amount of a compound of any of the formulas described herein, or a pharmaceutically acceptable salt, tautomer or in vivo-hydrolysable precursor thereof As used herein, the phrase “cognitive disorder” includes, but is not limited to, one or more of the following: Alzheimer's disease, dementia, dementia due to Alzheimer's disease, dementia due to Parkinson's disease, and the like.

In some embodiments, the present invention provides a method for the treatment or prophylaxis of a mood disorder comprising administering to a mammal (including a human) a therapeutically effective amount of a compound of any of the formulas described herein, or a pharmaceutically acceptable salt, tautomer or in vivo-hydrolysable precursor thereof As used herein, the phrase “mood disorder” is a depressive disorder including, but is not limited to, one or more of the following: major depressive disorder, dysthymic disorder, bipolar depression and/or bipolar mania, bipolar I with or without manic, depressive or mixed episodes, bipolar II, cyclothymic disorder, mood disorder due to a general medical condition, manic episodes associated with bipolar disorder, mixed episodes associated with bipolar disorder, and the like.

Anxiety disorders, cognitive disorders, and mood disorders are defined, for example, in the American Psychiatric Association: Diagnostic and Statistical Manual of Mental Disorders, Fourth Edition, Text Revision, Washington, D.C., American Psychiatric Association, 2000.

In some embodiments, the present invention provides a method of treating or preventing an anxiety disorder, cognitive disorder, or mood disorder (such as any of those described herein), by administering to a mammal (including a human) a compound of any of the formulas described herein or a pharmaceutically acceptable salt, tautomer or in vivo-hydrolysable precursors and a cognitive and/or memory enhancing agent.

In some embodiments, the present invention provides a method of treating or preventing an anxiety disorder, cognitive disorder, or mood disorder (such as any of those described herein), by administering to a mammal (including a human) a compound of any of the formulas described herein or a pharmaceutically acceptable salt, tautomer or in vivo-hydrolysable precursors thereof wherein constituent members are provided herein, and a choline esterase inhibitor or anti-inflammatory agent.

In some embodiments, the present invention provides a method of treating or preventing an anxiety disorder, cognitive disorder, or mood disorder (such as any of those described herein), by administering to a mammal (including human) a compound of the present invention, and an atypical antipsychotic agent. Atypical antipsychotic agents include, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodon) and Olanzapine/Fluoxetine (marketed as Symbyax).

In some embodiments, the mammal or human being treated with a compound of the present invention, has been diagnosed with a particular disease or disorder, such as those described herein. In these cases, the mammal or human being treated is in need of such treatment. Diagnosis, however, need not be previously performed.

The present invention also includes pharmaceutical compositions which contain, as the active ingredient, one or more of the compounds of the invention herein together with at least one pharmaceutically acceptable carrier, diluent or excipent.

When used for pharmaceutical compositions, medicaments, manufacture of a medicament, or treating or preventing an anxiety disorder, cognitive disorder, or mood disorder (such as any of those described herein), compounds of the present invention include the compounds of any of the formulas described herein, and pharmaceutically acceptable salts, tautomers and in vivo-hydrolysable precursors thereof Compounds of the present invention further include hydrates and solvates.

The antidementia treatment defined herein may be applied as a sole therapy or may involve, in addition to the compound of the invention, conventional chemotherapy.

Such conjoint treatment may be achieved by way of the simultaneous, sequential or separate dosing of the individual components of the treatment. Such combination products employ the compounds of this invention.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in patients with symptoms of dementia the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. For example, such conventional non-toxic salts include those derived from inorganic acids such as hydrochloric, phosphoric, and the like; and the salts prepared from organic acids such as lactic, maleic, citric, benzoic, methanesulfonic, trifluoroacetate and the like.

In some embodiments, the present invention provides a compound of any of the formulas described herein or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The quantity of the compound to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

In some embodiments, the compounds described herein are central nervous system depressants and may be used as tranquilizers or ataractic agents for the relief of anxiety and tension states, for example, in mice, cats, rats, dogs and other mammalian species such as humans, in the same manner as chlordiazepoxide. For this purpose a compound or mixture of compounds of any of the formulas described herein, or non-toxic physiologically acceptable salts, such as acid addition salts thereof, may be administered orally or parenterally in a conventional dosage form such as tablet, pill, capsule, injectable or the like. The dosage in mg/kg of body weight of compounds of the present invention in mammals will vary according to the size of the animal and particularly with respect to the brain/body weight ratio. In general, a higher mg/kg dosage for a small animal such as a dog will have the same effect as a lower mg/kg dosage in an adult human. A minimum effective dosage for a compound of formula (I) will be at least about 0.1 mg/kg of body weight per day for mammals with a maximum dosage for a small mammal such as a dog, of about 100 mg/kg per day. For humans, a dosage of about 0.1 to 12 mg/kg per day will be effective, for example, about 5 to 600 mg/day for an average man. The dosage can be given once daily or in divided doses, for example, 2 to 4 doses daily, and such dosage will depend on the duration and maximum level of activity of a particular compound. The dose may be conventionally formulated in an oral or parenteral dosage form by compounding about 5 to 250 mg per unit of dosage of conventional vehicle, excipient, binder, preservative, stabilizer, flavor or the like as called for by accepted pharmaceutical practice, for example, as described in U.S. Pat. No. 3,755,340. The compounds of this invention may be used in pharmaceutical compositions comprising a compound of any of the formulas described herein or can be contained in the same formulation with or co-administered with one or more known drugs.

Some example tests that can be conducted to demonstrate the anxiolytic activity of the present compounds include binding tests of GABAA receptors. In some embodiments, the binding test is directed to a subtype of GABAA receptors, such as GABAA1 receptors (i.e., those containing the α₁ subunit), GABAA2 receptors (i.e., those containing the α₂ subunit), GABAA₃ receptors (i.e., those containing the α₃ subunit) and GABAA5 receptors (i.e., those containing the α₅ subunit).

Presently available GABAA modulator anxiolytics work via interactions at the classical benzodiazepine binding site. To a large degree these anxiolytics lack GABAA receptor subtype-selectivity. The subtype-selective GABAA receptor modulators may offer more advantages. For example, a growing body of work suggests that desirable anxiolytic activity is driven primarily by interactions with GABAA receptors containing the α₂ subunit. Sedation, a side-effect common to all marketed benzodiazepines, is believed to be mediated by interactions at GABAA receptors containing the α₁ subunit. To develop anxiolytics with minimal liabilities due to interactions with other subunits, an electrophysiological assay is developed to screen modulatory effects of various compounds on different GABA subunit combinations heterologously expressed in Xenopus oocytes.

GABAA receptors were heterologously expressed in Xenopus oocytes by injecting cRNA corresponding to human α₁, α₂, α₃, α₅, β₂, P₃ and γ₂ subunits of the GABAA receptor genes. The specific subunit combinations (subtypes) were as follows: α₁β₂γ₂, α₂β₃γ₂, α₃β₃γ₂, and α₅β₃γ₂. The EC10 of GABA is approximated for each cell. Stability of GABA-mediated (EC10) current is established. Modulatory effect of test compound is determined and compared across subtypes. The assay developed has reproducibility which allows discrimination of modulatory activity down to minimal effect of about 25% potentiation (prior to normalization to standard) for all four subtypes. Thus, the assay can characterize modulatory effects and determine subtype selectivity of test compounds on major subtypes of GABAA receptors. In some embodiments, a compound can selectively bind to one subtype of GABAA receptor (by showing about 25% or more of binding comparing to another subtype of GABAA receptor).

Anxiolytic activity is indicated in the GABAA binding test by a displacement of the flunitrazepam such as is exhibited by benzodiazepines or by enhancement of the binding such as is shown by cartazolate and tracazolate.

In some embodiments, the compounds of the invention can bind to GABAA receptors. In some embodiments, the compounds of the invention can bind to GABAA receptors by displacement of benzodiazepines. Accordingly, the compounds of the invention can be used to modulate activities of GABAA receptors. In some embodiments, the compounds of the invention can selectively bind to a subtype of GABAA receptors, such as such as GABAA1 receptors (i.e., those containing the α₁ subunit), GABAA2 receptors (i.e., those containing the α₂ subunit), GABAA3 receptors (i.e., those containing the α₃ subunit) or GABAA5 receptors (i.e., those containing the α₅ subunit). In some embodiments, the compounds of the invention can selectively bind to a subtype of GABAA receptors by displacement of benzodiazepines. Accordingly, the compounds of the invention can be used to selectively modulate activities of a subtype of GABAA receptors, such as GABAA 1 receptors, GABAA2 receptors, GABAA3 receptors or GABAA5 receptors.

In some embodiments, certain compounds of the invention are GABAA1 receptor antagonists and GABAA2 receptor agonists.

Because the compounds of the invention can be used to modulate activities of GABAA receptors, or to selectively modulate activities of a subtype of GABAA receptors, the compounds of the invention are envisioned to be useful for treating or preventing diseases mediated by GABAA receptors or a subtype of GABAA receptors. Such disease, include, but is not limited to, stroke, head trauma, epilepsy, pain, migraine, mood disorders, anxiety, post traumatic stress disorder, obsessive compulsive disorders, schizophrenia, seizures, convulsions, tinnitus, neurodegenerative disorders including Alzheimer's disease, amyotrophic lateral sclerosis, Huntington's Chorea, Parkinson's disease, depression, bipolar disorders, mania, trigeminal and other neuralgia, neuropathic pain, hypertension, cerebral ischemia, cardiac arrhythmia, myotonia, substance abuse, myoclonus, essential tremor, dyskinesia and other movement disorders, neonatal cerebral hemorrhage, spasticity, cognitive disorder, and sleeping disorder.

It is known that melatonin receptor agonists are effective in treating depression. Certain compounds of the invention may selectively modulate activities of a subtype of melatonin receptors, melatonin receptor 1 (MT-1). In certain embodiments, certain compounds of the invention are MT1 agonists. As a results, these compounds of the invention may be effective in treating depression disorders such as major depressive disorder, dysthymic disorder, bipolar depression and/or bipolar mania, bipolar I with or without manic, depressive or mixed episodes, bipolar II, cyclothymic disorder, mood disorder due to a general medical condition, manic episodes associated with bipolar disorder, or mixed episodes associated with bipolar disorder. To treat depression disorders, an effective amount of one or more compounds of the invention is administered to a patient with such a need.

In another embodiment, certain compounds of the invention may be useful in treating schizophrenia. In a particular embodiment, certain compounds of the invention may be useful in treating cognitive disorders associated with schizophrenia. The existing non-selective GABAergic agents are generally not suitable for treating information/cognitive processing deficits in schizophrenia due to the unacceptable competing side effects, such as overt sedation and memory impairment. Certain compounds of the invention are capable of selective modification of function at the specific GABAergic synapses affected by the schizophrenic disease state. Therefore, these certain compounds of the invention acting selectively at GABAA α2 subunits may be used for treating cognitive deficits in schizophrenia. The therapeutic effects of certain compound of the invention in treating cognitive deficits associated with schizophrenia may be demonstrated by testing one or more these compounds using Method JJ, which involves altering the power spectrum of frequencies comprising the spontaneous electroencephalogram (EEG) in behaving rats.

The EEG protocol (Method JJ) may show that spontaneous EEG from behaving animals in the presence of certain compounds of the invention with selective α2/α3 pharmacologies exhibits dose dependent increases in high frequency oscillations in both the high beta and gamma ranges with no significant increases at lower frequencies. In contrast, the α1-selective compound, zolpidem, exhibits no significant increase at gamma frequencies, and the non-selective GABA compound, Lorazepam, leads to broad changes in spontaneous EEG across a range of oscillation frequencies. The selective nature of α2/α3 on high frequency EEG in vivo suggests that these compounds may be useful in attenuating the high frequency EEG deficits seen in schizophrenic patients, and, to the extent that these EEG deficits reflect impaired cognitive function, demonstrates that certain GABAA α2/α3 selective compounds of the invention may be used to treat cognitive deficits in schizophrenia.

In another embodiment, certain compounds of the present invention may be effective in treating insomnia.

In a further embodiment, a compound of formula I or a pharmaceutically acceptable salt, solvate or in vivo hydrolysable ester thereof, or a pharmaceutical composition or formulation comprising a compound of formula I may be administered concurrently, simultaneously, sequentially or separately with one or more pharmaceutically active compound(s) selected from the following:

(i) antidepressants such as amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, reboxetine, robalzotan, sertraline, sibutramine, thionisoxetine, tranylcypromaine, trazodone, trimipramine, venlafaxine and equivalents and pharmaceutically active isomer(s) and metabolite(s) thereof;

(ii) atypical antipsychotics including for example quetiapine and pharmaceutically active isomer(s) and metabolite(s) thereof; amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, lithium, loxapine, mesoridazine, olanzapine, paliperidone, perlapine, perphenazine, phenothiazine, phenylbutlypiperidine, pimozide, prochlorperazine, risperidone, quetiapine, sertindole, sulpiride, suproclone, suriclone, thioridazine, trifluoperazine, trimetozine, valproate, valproic acid, zopiclone, zotepine, ziprasidone and equivalents thereof;

(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutlypiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof;

(iv) anxiolytics including for example alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof;

(v) anticonvulsants including, for example, carbamazepine, valproate, lamotrogine, gabapentin and equivalents and pharmaceutically active isomer(s) and metabolite(s) thereof;

(vi) Alzheimer's therapies including, for example, donepezil, memantine, tacrine and equivalents and pharmaceutically active isomer(s) and metabolite(s) thereof;

(vii) Parkinson's therapies including, for example, deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof;

(viii) migraine therapies including, for example, almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof;

(ix) stroke therapies including, for example, abciximab, activase, NXY-059, citicoline, crobenetine, desmoteplase, repinotan, traxoprodil and equivalents and pharmaceutically active isomer(s) and metabolite(s) thereof;

(x) over active bladder urinary incontinence therapies including, for example, darafenacin, falvoxate, oxybutynin, propiverine, robalzotan, solifenacin, tolterodine and and equivalents and pharmaceutically active isomer(s) and metabolite(s) thereof;

(xi) neuropathic pain therapies including, for example, gabapentin, lidoderm, pregablin and equivalents and pharmaceutically active isomer(s) and metabolite(s) thereof;

(xii) nociceptive pain therapies such as celecoxib, etoricoxib, lumiracoxib, rofecoxib, valdecoxib, diclofenac, loxoprofen, naproxen, paracetamol and equivalents and pharmaceutically active isomer(s) and metabolite(s) thereof;

(xiii) insomnia therapies including, for example, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, roletamide, triclofos, secobarbital, zaleplon, zolpidem and equivalents and pharmaceutically active isomer(s) and metabolite(s) thereof; and

(xiv) mood stabilizers including, for example, carbamazepine, divalproex, gabapentin, lamotrigine, lithium, olanzapine, quetiapine, valproate, valproic acid, verapamil, and equivalents and pharmaceutically active isomer(s) and metabolite(s) thereof.

Such combinations employ the compounds of this invention within the dosage range described herein and the other pharmaceutically active compound or compounds within approved dosage ranges and/or the dosage described in the publication reference.

### Synthesis

Certain compounds of the present invention can be synthesized using the methods described below, together with synthetic methods known in the art of synthetic organic chemistry, or variations thereon as appreciated by those skilled in the art. The starting materials and precursors used in the processes described herein are either commercially available or readily prepared by established organic synthesis methods. It is understood by one skilled in the art of organic synthesis that the functionality present on various portions of the molecule must be compatible with the reagents and reactions proposed.

In one embodiment, the invention provides a synthetic method of making a compound of formula I:

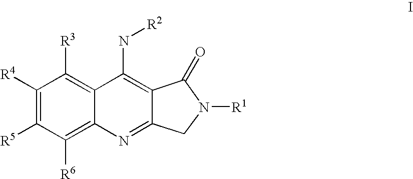

or a pharmaceutically acceptable salt, tautomer, or in vivo-hydrolysable precursor thereof, wherein:

R¹ is C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl, wherein each of the C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl is optionally substituted by 1, 2, 3, 4 or 5 R⁷;

R² is H, C(═O)R^{b}, C(═O)NR^{c}R^{d}, C(═O)OR^{a}, S(═O)₂R^{b}, C₁₋₆ alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl, wherein each of the C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl is optionally substituted by 1, 2, 3, 4 or 5 R⁸;

R³, R⁴ and R⁵ are each, independently, H, halo, Si(C₁₋₁₀alkyl)₃, CN, NO₂, OR^{a}, SR^{a}, OC(═O)R^{a}, OC(═O)OR^{b}, OC(═O)NR^{c}R^{d}, C(═O)R^{a}, C(═O)OR^{b}, C(═O)NR^{c}R^{d}, NR^{c}R^{d}, NR^{c}C(═O)R^{a}, NR^{c}C(═O)OR^{b}, NR^{c}S(═O)₂R^{b}, S(═O)R^{a}, S(═O)NR^{c}R^{d}, S(═O)₂R^{a}, S(═O)₂NR^{c}R^{d}, C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl, wherein each of the C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl is optionally substituted by 1, 2 or 3 R⁹;

R⁶ is C₆₋₁₀aryl or C₂₋₅heteroaryl, each optionally substituted by 1, 2, 3, 4 or 5 A¹;

R⁷, R⁸ and R⁹ are each, independently, halo, C₁₋₄alkyl, C₁₋₄haloalkyl, C₆₋₁₀aryl, C₃₋₇cycloalkyl, C₂₋₅heteroaryl, C₂₋₅heterocycloalkyl, —CN, —NO₂, —OR^{a′}, —SR^{a′}, —C(═O)R^{b′}, —C(═O)NR^{c′}R^{d′}, —C(═O)OR^{a′}, —OC(═O)R^{b′}, —OC(═O)NR^{c′}R^{d′}, —NR^{c′}R^{d′}, —NR^{c′}C(═O)R^{b′}, —NR^{c′}C(═O)OR^{a′}, —NR^{c′}S(═O)₂R^{b′}, —S(═O)R^{b′}, —S(═O)NR^{c′}R^{d′}, —S(═O)₂R^{b′}, or —S(═O)₂NR^{c′}R^{d′};

A¹ is halo, —CN, —NO₂, —OR^{a}, —SR^{a}, —C(═O)R^{b}, —C(═O)NR^{c}R^{d}, —C(═O)OR^{a}, —OC(═O)R^{b}, —OC(═O)NR^{c}R^{d}, —NR^{c}R^{d}, —NR^{c}C(═O)R^{d}, —NR^{c}C(═O)OR^{a}, —NR^{c}S(═O)R^{b}, —NR^{c}S(═O)₂R^{b}, —S(═O)R^{b}, —S(═O)NR^{c}R^{d}, —S(═O)₂R^{b}, —S(═O)₂NR^{c}R^{d}, C₁₋₄alkoxy, C₁₋₄haloalkoxy, amino, C₁₋₄alkylamino, C₂₋₈dialkylamino, C₁₋₆alkyl, C₂₋₆alkenyl, C₂₋₆alkynyl, C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl, wherein each of the C₁₋₆alkyl, C₂₋₆alkenyl, C₂₋₆alkynyl, C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl is optionally substituted by 1, 2, 3, 4 or 5 substituents independently selected from halo, C₁₋₆alkyl, C₂₋₆alkenyl, C₂₋₆alkynyl, C₁₋₄haloalkyl, C₆₋₁₀aryl, C₃₋₇cycloalkyl, C₂₋₅heteroaryl, C₂₋₅heterocycloalkyl, —CN, —NO₂, —OR^{a′}, —SR^{a′}, —C(═O)R^{b′}, —C(═O)NR^{c′}R^{d′}, —C(═O)OR^{a′}, —OC(═O)R^{b′}, —OC(═O)NR^{c′}R^{d′}, —NR^{c′}R^{d′}, —NR^{c′}C(═O)R^{b′}, —NR^{c′}C(═O)OR^{a′}, —NR^{c′}S(═O)R^{b′}, —NR^{c′}S(═O)₂R^{b′}, —S(═O)R^{b′}, —S(═O)NR^{c′}R^{d′}, —S(═O)₂R^{b′}, or —S(═O)₂NR^{c′}R^{d′};

R^{a} and R^{a′} are each, independently, H, C₁₋₆alkyl, C₁₋₆haloalkyl, C₂₋₆ alkenyl, C₂₋₆alkynyl, C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl;

R^{b} and R^{b′} are each, independently, H, C₁₋₆alkyl, C₁₋₆haloalkyl, C₂₋₆alkenyl, C₂₋₆alkynyl, C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl;

R^{c} and R^{d} are each, independently, H, C₁₋₆alkyl, C₁₋₆haloalkyl, C₂₋₆alkenyl, C₂₋₆alkynyl, C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl;

or R^{c} and R^{d} together with the N atom to which they are attached form a 4-, 5-, 6- or 7-membered heterocycloalkyl group; and

R^{c′} and R^{d′} are each, independently, H, C₁₋₆alkyl, C₁₋₆haloalkyl, C₂₋₆alkenyl, C₂₋₆alkynyl, C₁₋₆alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl;

or R^{c′} and R^{d′} together with the N atom to which they are attached form a 4-, 5-, 6- or 7-membered heterocycloalkyl group;

with the proviso that when R², R³, R⁴ and R⁵ are each H, then R⁶ is not selected from unsubstituted phenyl, 4-fluorophenyl, 4-chlorophenyl, 4-methoxyphenyl, 4-methylphenyl, 3-methoxyphenyl, 2-methoxyphenyl, and 4-N,N-dimethylaminophenyl, comprising reacting a compound of Formula II:

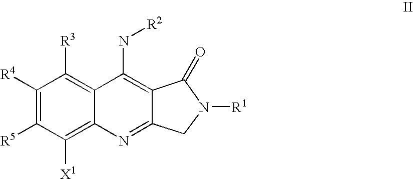

wherein X¹ is bromo or iodo,
with a compound of formula III:

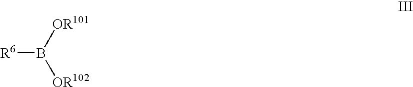

wherein:

R¹⁰¹ and R¹⁰² are each, independently, hydrogen or C₁₋₆alkyl;

or R¹⁰¹ and R¹⁰², together with the two oxygen atoms to which they are attached and the boron atom to which the two oxygen atoms are attached, form a 4-7 membered heterocyclic ring whose ring-forming atoms comprises B, O and C atoms and which is optionally substituted by 1, 2, 3, or 4 C₁₋₆alkyl,

in the presence of a catalyst and a base for a time and under conditions sufficient to form the compound of Formula I.

In another embodiment, the R¹⁰¹ and R¹⁰² are each, independently, hydrogen.

In another embodiment, the compound of formula III has formula IV:

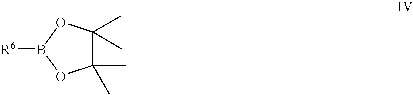

In another embodiment, the catalyst is a palladium catalyst.

In another embodiment, the palladium catalyst is bis(triphenylphosphine)palladium(II)dichloride.

In another embodiment, the palladium catalyst is tetrakis(triphenylphosphine)palladium(0).

In another embodiment, the base is cesium carbonate, sodium carbonate or potassium phosphate.

In another embodiment, the reacting is carried out in a solvent which comprises an organic solvent.

In another embodiment, the organic solvent is selected from 1,2-dimethoxyethane, tetrahydrofuran and ethanol.

In another embodiment, the solvent further comprises water.

In a further embodiment, some example compounds of the invention in Table 1 may be made according to the methods described herein below.

TABLE 1

| | Synthesis | | |
|---|---|---|---|
| Example | Method | Compound Name | Structure |
| 1 | A | 9-Amino-5-(2-fluoro-6-methoxyphenyl)-2-(4-methoxybenzyl)-2,3-dihydropyrrolo[3,4-b]quinolin-1-one | 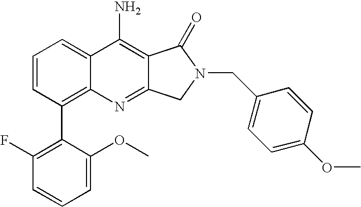 |
| 2 | A | 9-Amino-5-(2,5-difluorophenyl)-2-(4-methoxyphenyl)-2,3-dihydropyrrolo[3,4-b]quinolin-1-one | 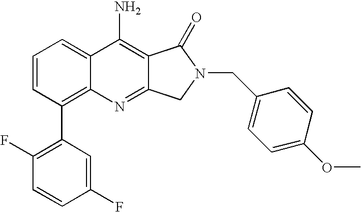 |
| 3 | A | 9-Amino-2-(4-methoxybenzyl)-5-(2-methoxypyridin-3-yl)-2,3-dihydropyrrolo[3,4-b]quinolin-1-one | 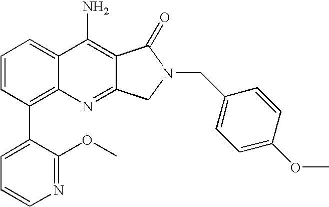 |
| 4 | A | 9-Amino-2-(2,5-dimethoxybenzyl)-5-(2-methoxypyridin-3-yl)-2,3-dihydropyrrolo[3,4-b]quinolin-1-one | 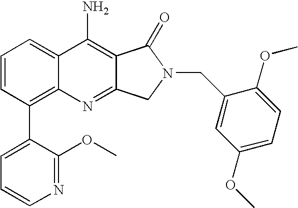 |
| 5 | B | 9-Amino-5-(2-fluoro-6-methoxyphenyl)-2-propyl-2,3-dihydropyrrolo[3,4-b]quinolin-1-one | 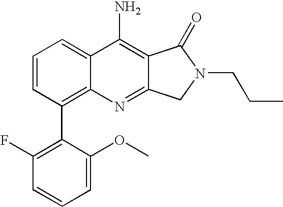 |
| 6 | A | 9-Amino-5-(2,3-dimethylphenyl)-2-propyl-2,3-dihydropyrrolo[3,4-b]quinolin-1-one | 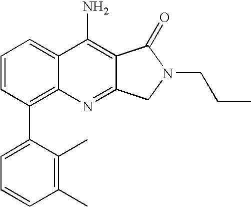 |
| 7 | C | 9-Amino-5-(3,5-dimethylphenyl)-2-propyl-2,3-dihydropyrrolo[3,4-b]quinolin-1-one | 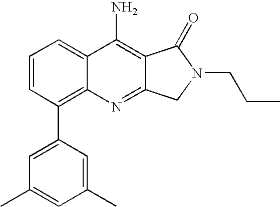 |
| 8 | A | 9-Amino-5-(6-chloropyridin-3-yl)-2-(3,4-dimethoxybenzyl)-2,3-dihydropyrrolo[3,4-b]quinolin-1-one | 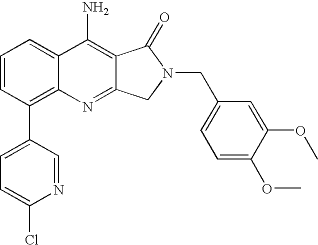 |
| 9 | D | 9-Amino-5-(2,6-dimethoxypyridin-3-yl)-2-propyl-2,3-dihydropyrrolo[3,4-b]quinolin-1-one | 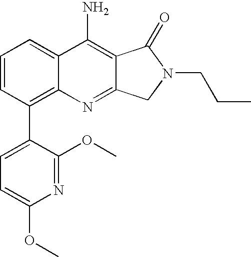 |
| 10 | D | 9-Amino-5-(6-methylpyridin-3-yl)-2-propyl-2,3-dihydropyrrolo[3,4-b]quinolin-1-one | 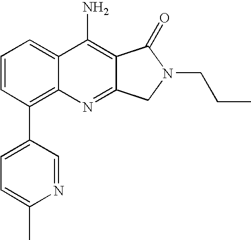 |
| 11 | A | 9-Amino-2-(3,4-dimethoxybenzyl)-5-(2,5-dimethoxyphenyl)-2,3-dihydropyrrolo[3,4-b]quinolin-1-one | 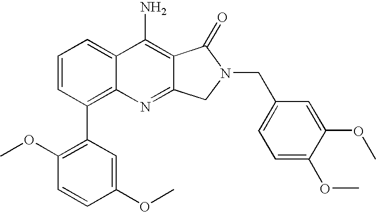 |
| 12 | D | 9-Amino-5-(6-methoxy-4-methylpyridin-3-yl)-2-propyl-2,3-dihydropyrrolo[3,4-b]quinolin-1-one | 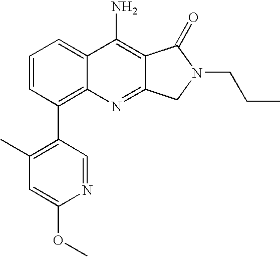 |
| 13 | D | 9-Amino-5-(2-fluoropyridin-3-yl)-2-propyl-2,3-dihydropyrrolo[3,4-b]quinolin-1-one | 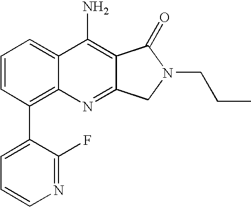 |
| 14 | A | 9-Amino-2-benzo[1,3]dioxol-5-ylmethyl-5-(2-methoxy-5-methylphenyl)-2,3-dihydropyrrolo[3,4-b]quinolin-1-one | 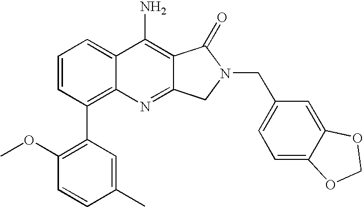 |
| 15 | D | 9-Amino-5-(2-chloro-6-methylpyridin-3-yl)-2-propyl-2,3-dihydropyrrolo[3,4-b]quinolin-1-one | 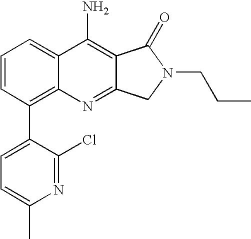 |
| 16 | A | 9-Amino-2-cyclopropyl-5-(2-fluoro-6-methoxyphenyl)-2,3-dihydropyrrolo[3,4-b]quinolin-1-one | 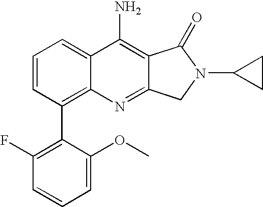 |
| 17 | A | 9-Amino-2-ethyl-5-(2-fluoro-6-methoxyphenyl)-2,3-dihydropyrrolo[3,4-b]quinolin-1-one | 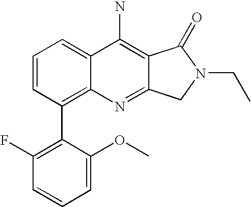 |
| 18 | A | 9-Amino-2-cyclobutyl-5-(2-fluoro-6-methoxyphenyl)-2,3-dihydropyrrolo[3,4-b]quinolin-1-one | 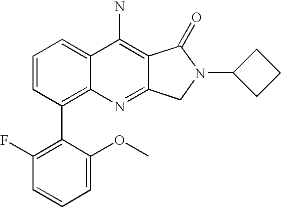 |
| 19 | A | 9-Amino-2-ethyl-5-(2-methoxypyridin-3-yl)-2,3-dihydropyrrolo[3,4-b]quinolin-1-one | 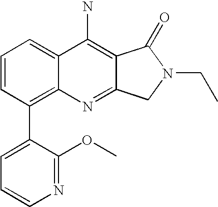 |
| 20 | A | 9-Amino-5-(2-fluoro-6-methoxyphenyl)-2-methyl-2,3-dihydropyrrolo[3,4-b]quinolin-1-one | 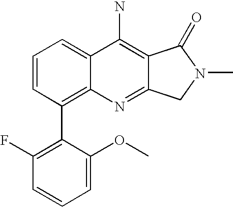 |
| 21 | A | 9-Amino-2-cyclopropyl-5-(2,5-dimethoxyphenyl)-2,3-dihydropyrrolo[3,4-b]quinolin-1-one | 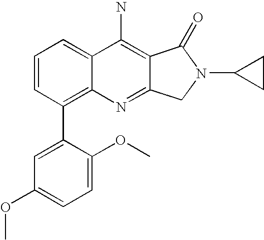 |
| 22 | A | 9-Amino-2-cyclopropyl-5-(2-fluoro-3-methoxyphenyl)-2,3-dihydropyrrolo[3,4-b]quinolin-1-one | 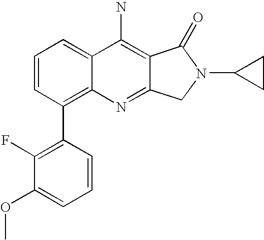 |
| 23 | A | 9-Amino-5-(2-chloro-6-methylpyridin-3-yl)-2-(3,4-dimethoxybenzyl)-2,3-dihydropyrrolo[3,4-b]quinolin-1-one | 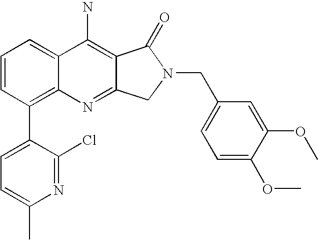 |
| 24 | D | 9-Amino-5-(2,6-dimethoxypyridin-3-yl)-6-fluoro-2-propyl-2,3-dihydropyrrolo[3,4-b]quinolin-1-one | 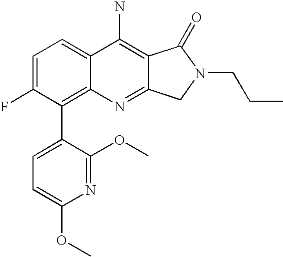 |
| 25 | A | 2-(9-Amino-2-ethyl-1-oxo-2,3-dihydro-1H-pyrrolo[3,4-b]quinolin-5-yl)-benzonitrile | 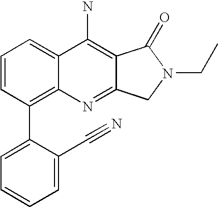 |
| 26 | A | 9-Amino-5-(2,6-dimethoxypyridin-3-yl)-2-ethyl-6-fluoro-2,3-dihydropyrrolo[3,4-b]quinolin-1-one | 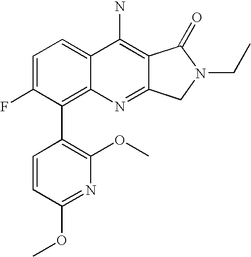 |
| 27 | A | 9-Amino-5-(2,6-dimethoxypyridin-3-yl)-2-ethyl-2,3-dihydropyrrolo[3,4-b]quinolin-1-one | 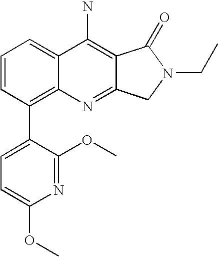 |
| 28 | D | 9-Amino-2-cyclopropyl-5-(2,4-dimethoxyphenyl)-6-fluoro-2,3-dihydropyrrolo[3,4-b]quinolin-1-one | 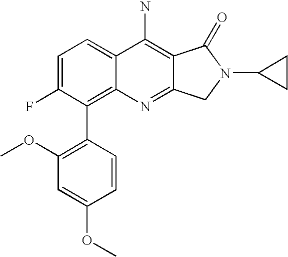 |
| 29 | A | 9-Amino-2-ethyl-5-(3,4-dimethoxyphenyl)-2,3-dihydropyrrolo[3,4-b]quinolin-1-one | 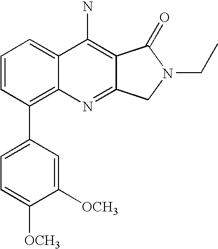 |
| 30 | D | 9-Amino-5-(2,5-dimethoxyphenyl)-2-ethyl-6-fluoro-2,3-dihydro-1H-pyrrolo[3,4-]quinolin-1-one | 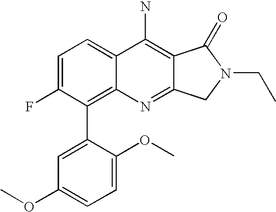 |
| 31 | A | 9-Amino-5-(2,6-dimethoxypyridin-3-yl)-2-methyl-2,3-dihydropyrrolo[3,4-b]quinolin-1-one | 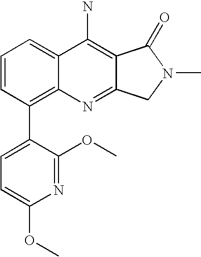 |
| 32 | A | 9-Amino-2-ethyl-5-(4-fluoro-2-methoxyphenyl)-2,3-dihydropyrrolo[3,4-b]quinolin-1-one | 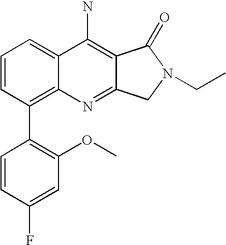 |
| 33 | A | 9-Amino-2-ethyl-5-(2-fluoro-3-methoxyphenyl)-2,3-dihydropyrrolo[3,4-b]quinolin-1-one | 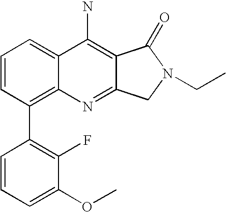 |
| 34 | A | 9-Amino-5-(2,4-dimethoxypyrimidin-5-yl)-2-ethyl-6-fluoro-2,3-dihydropyrrolo[3,4-b]quinolin-1-one | 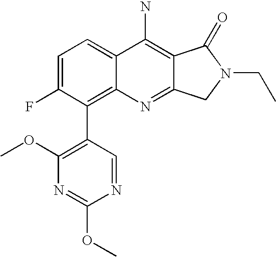 |
| 35 | D | 9-Amino-2-cyclopropyl-6-fluoro-5-(2-fluoro-6-methoxyphenyl)-2,3-dihydropyrrolo[3,4-b]quinolin-1-one | 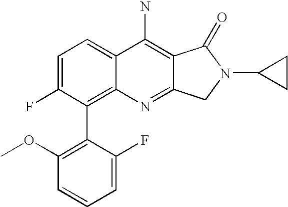 |
| 36 | D | 9-Amino-2-ethyl-6-fluoro-5-(4-methoxypyridin-3-yl)-2,3-dihydropyrrolo[3,4-b]quinolin-1-one | 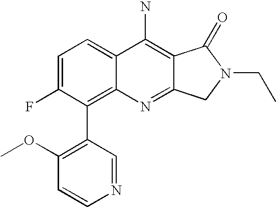 |
| 37 | D | 9-Amino-2-cyclopropyl-5-(2,5-dimethoxyphenyl)-6-fluoro-2,3-dihydropyrrolo[3,4-b]quinolin-1-one | 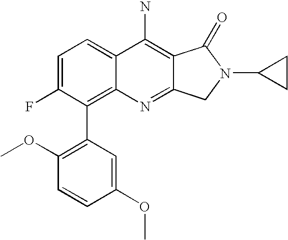 |
| 38 | D | 9-Amino-2-cyclopropyl-6-fluoro-5-(2-methoxypyridin-3-yl)-2,3-dihydropyrrolo[3,4-b]quinolin-1-one | 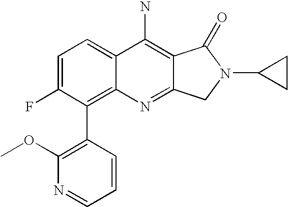 |
| 39 | A | 9-Amino-2-(2,5-dimethoxybenzyl)-5-(4-methoxypyridin-3-yl)-2,3-dihydropyrrolo[3,4-b]quinolin-1-one | 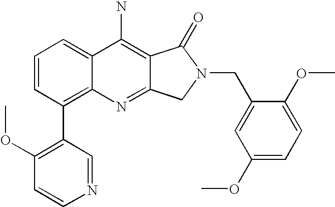 |
| 40 | A | 9-Amino-2-propyl-5-pyridin-3-yl-2,3-dihydropyrrolo[3,4-b]quinolin-1-one | 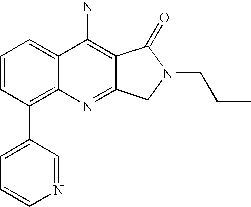 |
| 41 | D | 9-Amino-2-cyclopropyl-6-fluoro-5-(4-methoxypyridin-3-yl)-2,3-dihydropyrrolo[3,4-b]quinolin-1-one | 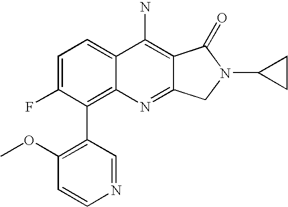 |
| 42 | A | 9-Amino-2-cyclobutyl-5-(2,5-dimethoxyphenyl)-2,3-dihydropyrrolo[3,4-b]quinolin-1-one | 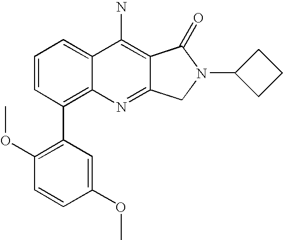 |
| 43 | A | 9-Amino-2-butyl-5-(2,6-dimethoxypyridin-3-yl)-2,3-dihydropyrrolo[3,4-b]quinolin-1-one | 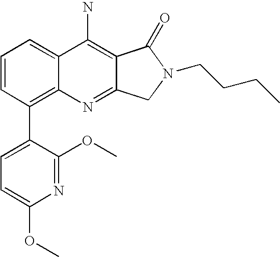 |
| 44 | D | 9-Amino-2-cyclopropyl-5-(2,4-dimethoxypyrimidin-5-yl)-6-fluoro-2,3-dihydropyrrolo[3,4-b]quinolin-1-one | 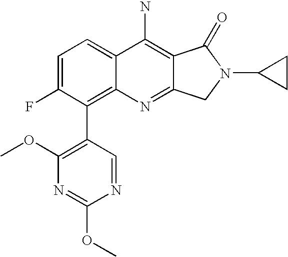 |
| 45 | D | 9-Amino-2-ethyl-6-fluoro-5-(2-methoxyphenyl)-2,3-dihydropyrrolo[3,4-b]quinolin-1-one | 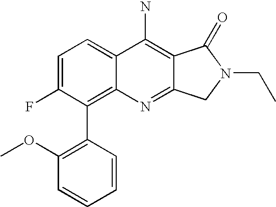 |
| 46 | D | 9-Amino-2-ethyl-6-fluoro-5-(5-fluoro-2-methoxyphenyl)-2,3-dihydropyrrolo[3,4-b]quinolin-1-one | 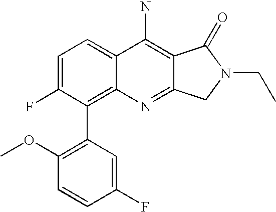 |
| 47 | D | 9-Amino-5-(3,4-dimethoxyphenyl)-2-ethyl-6-fluoro-2,3-dihydropyrrolo[3,4-b]quinolin-1-one | 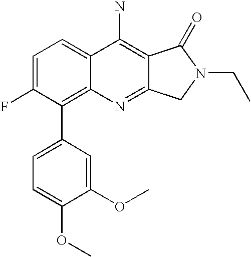 |
| 48 | A | 9-Amino-2-cyclobutyl-5-(2-methoxypyridin-3-yl)-2,3-dihydropyrrolo[3,4-b]quinolin-1-one | 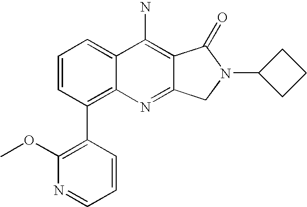 |
| 49 | A | 9-Amino-5-(5-chloro-2-methoxyphenyl)-2-cyclobutyl-2,3-dihydropyrrolo[3,4-b]quinolin-1-one | 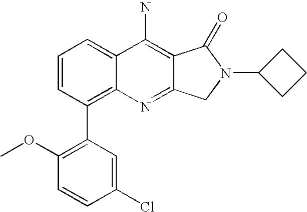 |
| 51 | | 9-Amino-2-cyclopropyl-5-(3,4-dimethoxyphenoxy)-2,3-dihydro-1H-pyrrolo[3,4-b]quinolin-1-one | 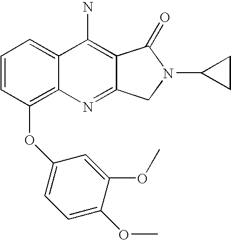 |
| 52 | D | 9-Amino-2-cyclobutyl-5-(2,6-dimethoxypyridin-3-yl)-6-fluoro-2,3-dihydropyrrolo[3,4-b]quinolin-1-one | 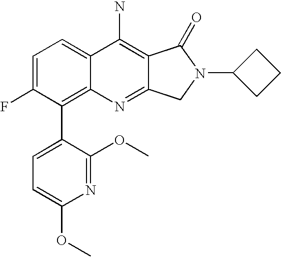 |
| 53 | A | 9-Amino-2-cyclobutyl-5-(2,4-dimethoxyphenyl)-2,3-dihydropyrrolo[3,4-b]quinolin-1-one | 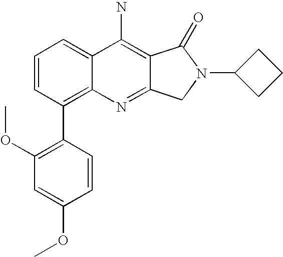 |
| 54 | D | 9-Amino-2-cyclopropyl-5-(2,6-dimethoxypyridin-3-yl)-6-fluoro-2,3-dihydropyrrolo[3,4-b]quinolin-1-one | 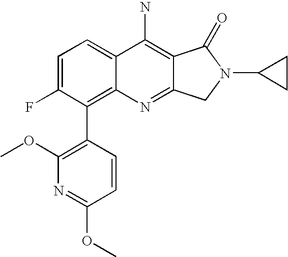 |
| 55 | D | 9-Amino-6-fluoro-5-(2-fluoro-6-methoxyphenyl)-2-propyl-2,3-dihydropyrrolo[3,4-b]quinolin-1-one | 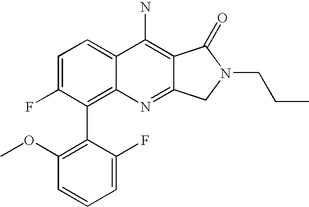 |
| 56 | D | 9-Amino-5-(2,6-difluorophenyl)-2-propyl-2,3-dihydro-pyrrolo[3,4-b]quinolin-1-one | 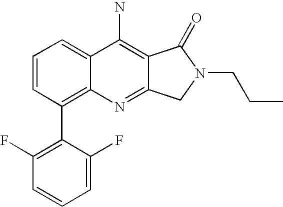 |
| 57 | A | 9-Amino-2-ethyl-5-(4-methoxypyridin-3-yl)-2,3-dihydropyrrolo[3,4-b]quinolin-1-one | 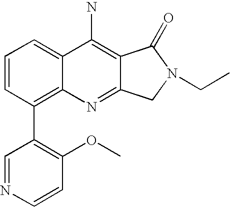 |
| 58 | D | 9-Amino-2-ethyl-6-fluoro-5-(2-methoxypyridin-3-yl)-2,3-dihydropyrrolo[3,4-b]quinolin-1-one | 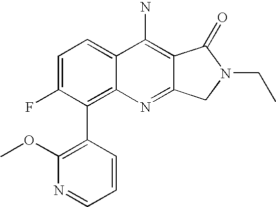 |
| 59 | A | 9-Amino-2-cyclopropyl-5-(2,5-dichlorophenyl)-2,3-dihydropyrrolo[3,4-b]quinolin-1-one | 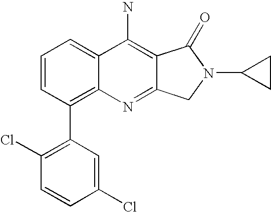 |
| 60 | A | 9-Amino-2-cyclopropyl-5-(2-fluoro-5-methoxyphenyl)-2,3-dihydropyrrolo[3,4-b]quinolin-1-one | 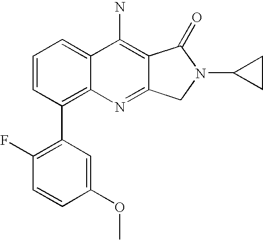 |
| 61 | D | 9-Amino-2-cyclopropyl-6-fluoro-5-(5-fluoro-2-methoxyphenyl)-2,3-dihydropyrrolo[3,4-b]quinolin-1-one | 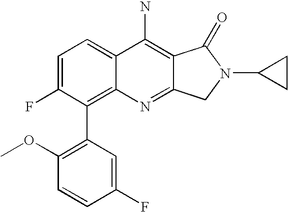 |
| 62 | A | 9-Amino-2-cyclobutyl-5-(4-methoxypyridin-3-yl)-2,3-dihydropyrrolo[3,4-b]quinolin-1-one | 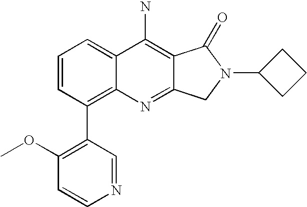 |
| 63 | A | 9-Amino-2-cyclobutyl-5-(2-methoxyphenyl)-2,3-dihydropyrrolo[3,4-b]quinolin-1-one | 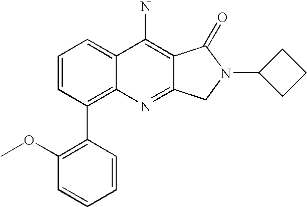 |
| 64 | A | 9-Amino-2-cyclobutyl-5-(2,6-dimethoxypyridin-3-yl)-2,3-dihydropyrrolo[3,4-b]quinolin-1-one | 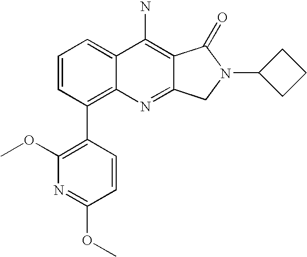 |
| 65 | A | 9-Amino-2-(3,4-dimethoxybenzyl)-5-(2-fluoro-6-methoxyphenyl)-2,3-dihydropyrrolo[3,4-b]quinolin-1-one | 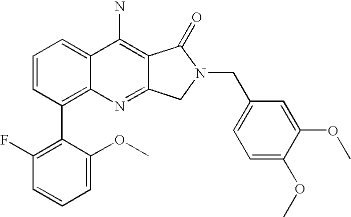 |
| 66 | A | 9-Amino-2-cyclopropyl-5-(2-methoxypyridin-3-yl)-2,3-dihydropyrrolo[3,4-b]quinolin-1-one | 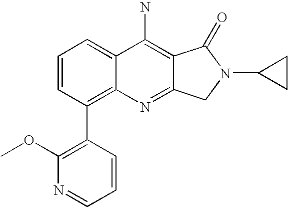 |
| 67 | A | 9-Amino-2-ethyl-6-fluoro-5-(2-fluoro-6-methoxyphenyl)-2,3-dihydropyrrolo[3,4-b]quinolin-1-one | 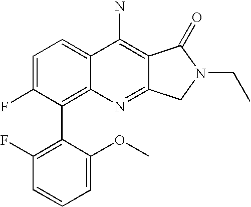 |
| 68 | A | 9-Amino-5-(2,4-dimethoxy-phenyl)-2-ethyl-6-fluoro-2,3-dihydropyrrolo[3,4-b]quinolin-1-one | 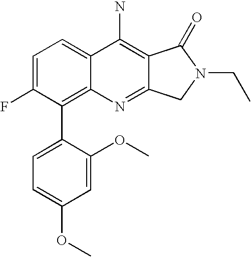 |
| 69 | A | 9-Amino-5-(2-fluoro-6-methoxyphenyl)-2-isopropyl-2,3-dihydropyrrolo[3,4-b]quinolin-1-one | 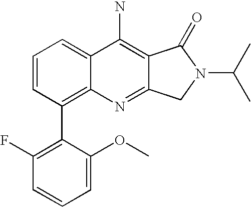 |
| 70 | A | 9-Amino-6-fluoro-5-(2-fluoro-6-methoxyphenyl)-2-methyl-2,3-dihydropyrrolo[3,4-b]quinolin-1-one | 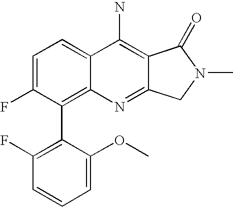 |
| 71 | A | 9-Amino-5-(2-fluoro-3-methoxyphenyl)-2-methyl-2,3-dihydropyrrolo[3,4-b]quinolin-1-one | 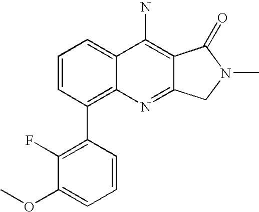 |
| 72 | A | 9-Amino-2-ethyl-5-(2-fluoro-5-methoxyphenyl)-2,3-dihydropyrrolo[3,4-b]quinolin-1-one | 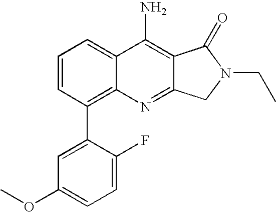 |
| 73 | A | 9-Amino-2-ethyl-5-(5-fluoro-2-methoxyphenyl)-2,3-dihydropyrrolo[3,4-b]quinolin-1-one | 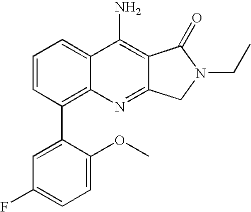 |
| 74 | A | 9-Amino-2-ethyl-5-(4-fluoro-3-methoxyphenyl)-2,3-dihydropyrrolo[3,4-b]quinolin-1-one | 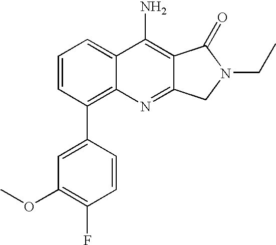 |
| 75 | A | 9-Amino-2-ethyl-5-(4-methylpyridin-3-yl)-2,3-dihydropyrrolo[3,4-b]quinolin-1-one | 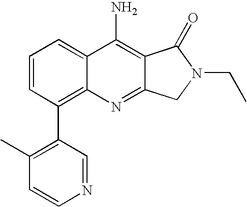 |
| 76 | A | 2-(9-Amino-2-cyclobutyl-1-oxo-2,3-dihydro-1H-pyrrolo[3,4-b]quinolin-5-yl)benzonitrile | 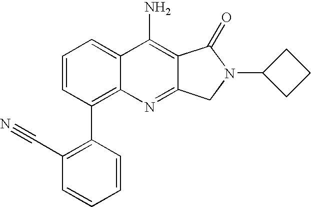 |
| 77 | A | 9-Amino-2-cyclobutyl-5-(4-fluoro-2-methoxyphenyl)-2,3-dihydropyrrolo[3,4-b]quinolin-1-one | 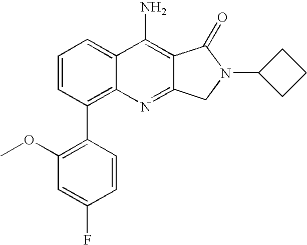 |
| 78 | A | 9-Amino-2-cyclobutyl-5-(2-fluoro-3-methoxyphenyl)-2,3-dihydropyrrolo[3,4-b]quinolin-1-one | 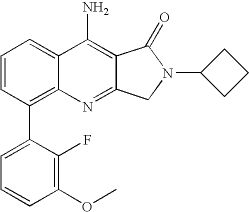 |
| 79 | A | 9-Amino-2-cyclobutyl-5-(2-fluoro-5-methoxyphenyl)-2,3-dihydropyrrolo[3,4-b]quinolin-1-one | 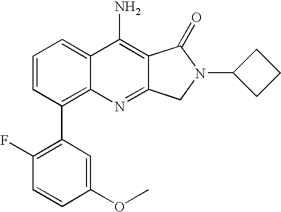 |
| 80 | E | 9-Amino-2-cyclobutyl-5-pyrazin-2-yl-2,3-dihydropyrrolo[3,4-b]quinolin-1-one | 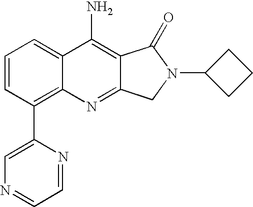 |
| 81 | A | 9-Amino-2-cyclobutyl-5-(3-methoxypyridin-4-yl)-2,3-dihydropyrrolo[3,4-b]quinolin-1-one | 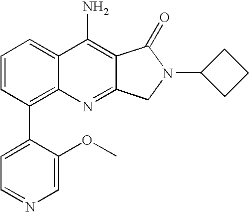 |
| 82 | E | 9-Amino-2-cyclobutyl-5-pyridin-4-yl-2,3-dihydropyrrolo[3,4-b]quinolin-1-one | 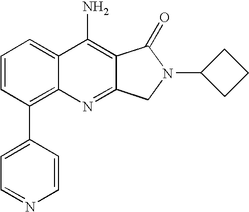 |
| 83 | E | 9-Amino-2-cyclobutyl-5-pyridin-2-yl-2,3-dihydropyrrolo[3,4-b]quinolin-1-one | 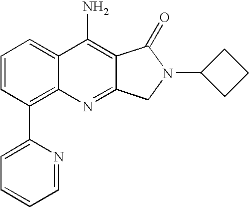 |
| 84 | E | 9-Amino-2-cyclobutyl-5-(3,6-dimethoxypyradazin-4-yl)-2,3-dihydropyrrolo[3,4-b]quinolin-1-one | 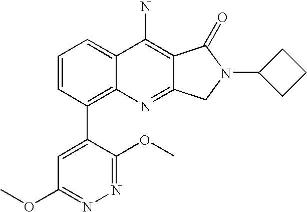 |
| 85 | E | 9-Amino-2-cyclobutyl-5-(6-methoxypyridin-2-yl)-2,3-dihydropyrrolo[3,4-b]quinolin-1-one | 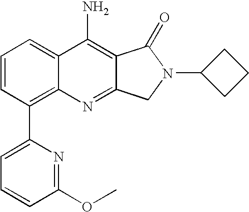 |
| 86 | E | 9-Amino-2-cyclobutyl-3-hydroxy-5-(6-methylpyridin-2-yl)-2,3-dihydropyrrolo[3,4-b]quinolin-1-one | 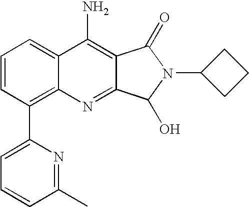 |
| 87 | E | 9-Amino-2-cyclobutyl-5-(5-methylpyridin-2-yl)-2,3-dihydropyrrolo[3,4-b]quinolin-1-one | 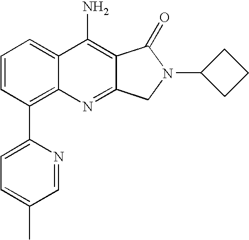 |
| 88 | — | 9-Amino-2-cyclobutyl-5-pyrimidin-2-yl-2,3-dihydropyrrolo[3,4-b]quinolin-1-one | 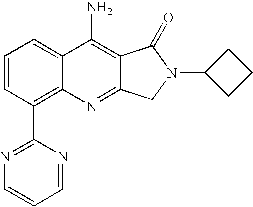 |
| 89 | E | 6-(9-Amino-2-cyclobutyl-1-oxo-2,3-dihydro-1H-pyrrolo[3,4-b]quinolin-5-yl)-nicotinonitrile | 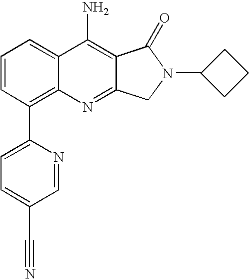 |
| 90 | E | 5-(9-Amino-2-cyclobutyl-1-oxo-2,3-dihydro-1H-pyrrolo[3,4-b]quinolin-5-yl)-nicotinonitrile | 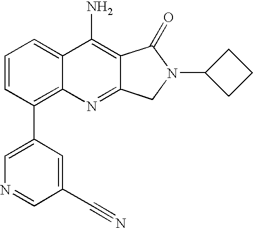 |
| 91 | E | 9-Amino-2-cyclobutyl-5-(3-methoxypyridazin-4-yl)-2,3-dihydropyrrolo[3,4-b]quinolin-1-one | 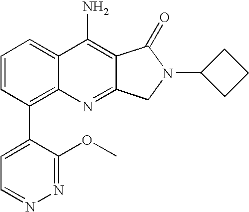 |
| 92 | E | 9-Amino-2-cyclobutyl-5-(4-methoxy-pyrimidin-5-yl)-2,3-dihydropyrrolo[3,4-b]quinolin-1-one | 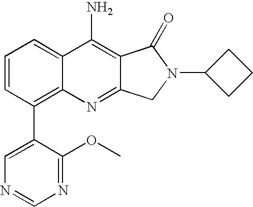 |
| 93 | E | 9-Amino-2-cyclobutyl-5-(3-fluoropyridin-2-yl)-2,3-dihydropyrrolo[3,4-b]quinolin-1-one | 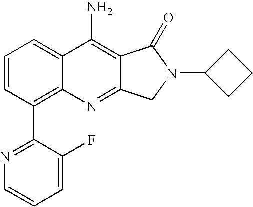 |
| 94 | E | 2-(9-Amino-2-cyclobutyl-1-oxo-2,3-dihydro-1H-pyrrolo[3,4-b]quinolin-5-yl)-5-fluorobenzonitrile | 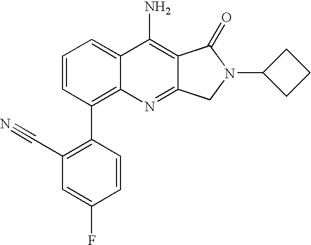 |
| 95 | E | 2-(9-Amino-2-cyclobutyl-1-oxo-2,3-dihydro-1H-pyrrolo[3,4-b]quinolin-5-yl)-4-fluorobenzonitrile | 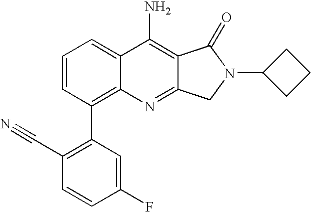 |
| 96 | E | 4-(9-Amino-2-cyclobutyl-1-oxo-2,3-dihydro-1H-pyrrolo[3,4-b]quinolin-5-yl)-6-methoxyniconitrile | 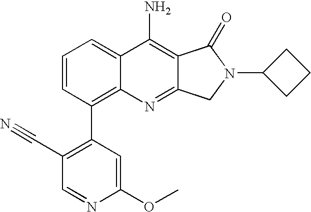 |
| 97 | A | 9-Amino-5-(1,3-dimethyl-1H-pyrazol-4-yl)-6-fluoro-2-(R)-tetrahydrofuran-3-yl-2,3-dihydropyrrolo[3,4-b]quinolin-1-one | 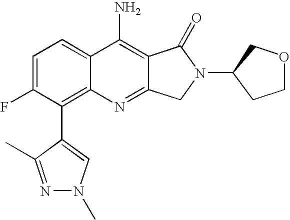 |
| 98 | E | 9-Amino-2-cyclobutyl-5-(5-fluoro-2-methoxypyridin-4-yl)-2,3-dihydropyrrolo[3,4-b]quinolin-1-one | 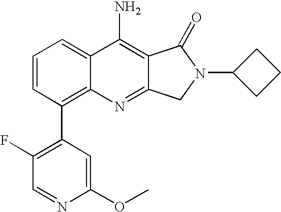 |
| 99 | A | 9-Amino-2-cyclobutyl-5-(5-fluoro-2-methoxyphenyl)-2,3-dihydropyrrolo[3,4-b]quinolin-1-one | 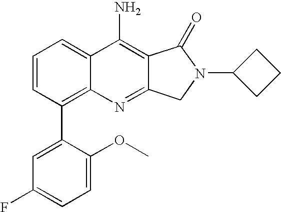 |
| 100 | F | 9-Amino-2-cyclobutyl-5-(2,4-dimethoxyphenyl)-6-fluoro-2,3-dihydropyrrolo[3,4-b]quinolin-1-one | 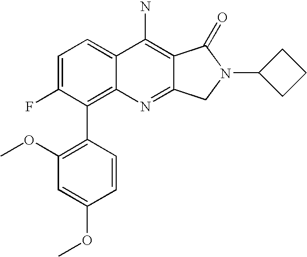 |
| 101 | F | 9-Amino-2-cyclobutyl-6-fluoro-5-(6-methylpyridin-3-yl)-2,3-dihydropyrrolo[3,4-b]quinolin-1-one | 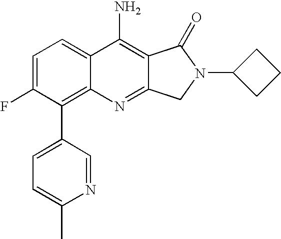 |
| 102 | | 9-Amino-2-cyclobutyl-6-fluoro-5-(2-fluoropyridin-3-yl)-2,3-dihydropyrrolo[3,4-b]quinolin-1-one | 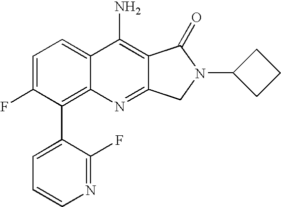 |
| 103 | F | 9-Amino-2-cyclobutyl-6-fluoro-5-(4-fluoro-2-methoxyphenyl)-2,3-dihydropyrrolo[3,4-b]quinolin-1-one | 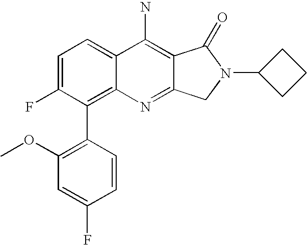 |
| 104 | F | 9-Amino-2-cyclobutyl-6-fluoro-5-pyrimidin-5-yl-2,3-dihydropyrrolo[3,4-b]quinolin-1-one | 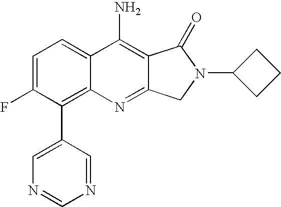 |
| 105 | F | 9-Amino-2-cyclobutyl-6-fluoro-5-(3-methoxypyridin-4-yl)-2,3-dihydropyrrolo[3,4-b]quinolin-1-one | 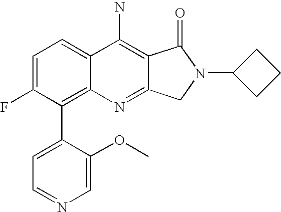 |
| 106 | | 9-Amino-2-cyclobutyl-6-fluoro-5-(2-fluoro-3-methoxyphenyl)-2,3-dihydropyrrolo[3,4-b]quinolin-1-one | 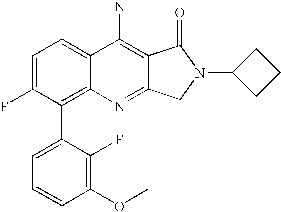 |
| 107 | F | 9-Amino-2-cyclobutyl-6-fluoro-5-(6-methoxypyridin-3-yl)-2,3-dihydropyrrolo[3,4-b]quinolin-1-one | 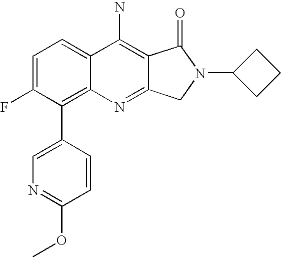 |
| 108 | F | 9-Amino-2-cyclobutyl-6-fluoro-5-(2-vinylphenyl)-2,3-dihydropyrrolo[3,4-b]quinolin-1-one | 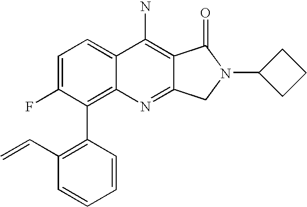 |
| 109 | A | 9-Amino-2-(3-chloro-4-methoxybenzyl)-5-(2-fluoro-6-methoxyphenyl)-2,3-dihydropyrrolo[3,4-b]quinolin-1-one | 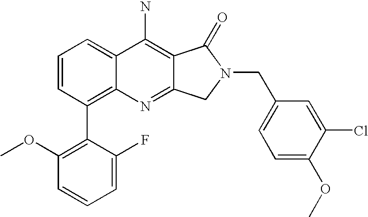 |
| 110 | A | 2-(9-Amino-2-cyclopropyl-1-oxo-2,3-dihydro-1H-pyrrolo[3,4-b]quinolin-5-yl)-benzonitrile | 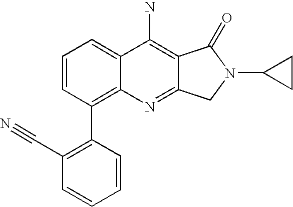 |
| 111 | A | 9-Amino-2-cyclopropyl-5-(6-methyl-pyridin-3-yl)-2,3-dihydropyrrolo[3,4-b]quinolin-1-one | 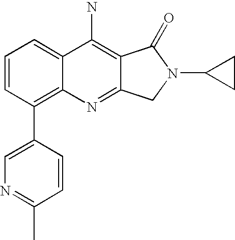 |
| 112 | A | 9-Amino-2-cyclopropyl-5-(2,5-difluorophenyl)-2,3-dihydropyrrolo[3,4-b]quinolin-1-one | 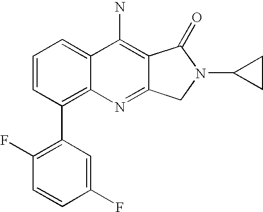 |
| 113 | A | 9-Amino-2-cyclopropyl-5-(2-fluorophenyl)-2,3-dihydropyrrolo[3,4-b]quinolin-1-one | 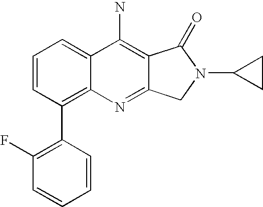 |
| 114 | F | 9-Amino-2-cyclopropyl-5-(2,6-difluorophenyl)-2,3-dihydropyrrolo[3,4-b]quinolin-1-one | 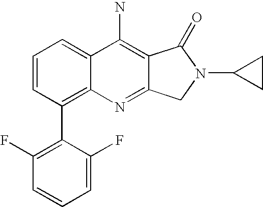 |
| 115 | A | 9-Amino-2-cyclopropyl-5-(2-fluoro-4-methoxyphenyl)-2,3-dihydropyrrolo[3,4-b]quinolin-1-one | 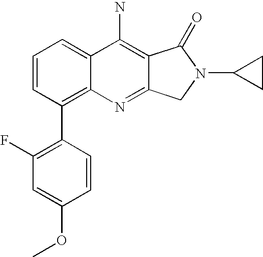 |
| 116 | A | 9-Amino-5-(2-chloro-5-methoxyphenyl)-2-cyclopropyl-2,3-dihydropyrrolo[3,4-b]quinolin-1-one | 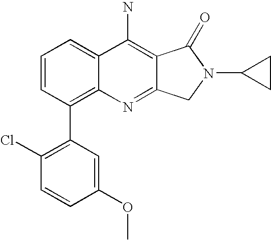 |
| 117 | G | 9-Amino-2-cyclopropyl-5-(2,6-difluoro-4-methoxyphenyl)-2,3-dihydropyrrolo[3,4-b]quinolin-1-one | 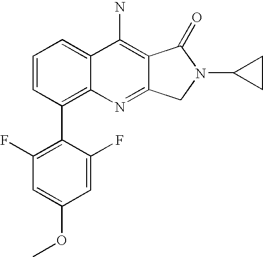 |
| 118 | A | 9-Amino-2-cyclopropyl-5-(2,3-difluorophenyl)-2,3-dihydropyrrolo[3,4-b]quinolin-1-one | 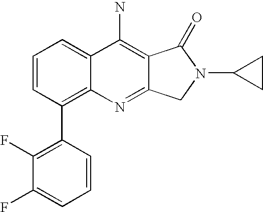 |
| 119 | A | 9-Amino-2-cyclopropyl-5-(2,4-difluorophenyl)-2,3-dihydropyrrolo[3,4-b]quinolin-1-one | 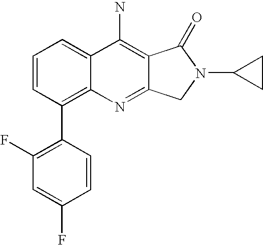 |
| 120 | G | 9-Amino-2-cyclopropyl-5-(2-fluoro-6-methylpyridin-3-yl)-2,3-dihydropyrrolo[3,4-b]quinolin-1-one | 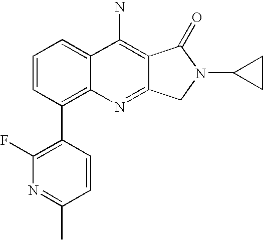 |
| 121 | A | 9-Amino-2-cyclobutyl-5-(6-methylpyridin-3-yl)-2,3-dihydropyrrolo[3,4-b]quinolin-1-one | 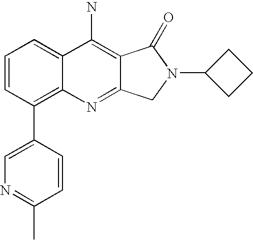 |
| 122 | G | 9-Amino-2-cyclobutyl-5-(2,6-difluoro-4-methoxyphenyl)-2,3-dihydropyrrolo[3,4-b]quinolin-1-one | 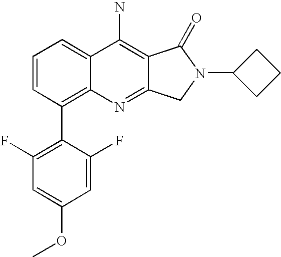 |
| 123 | A | 9-Amino-2-cyclobutyl-5-(2,4-dimethoxypyrimidin-5-yl)-2,3-dihydropyrrolo[3,4-b]quinolin-1-one | 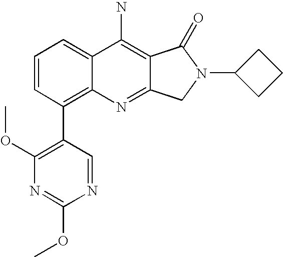 |
| 124 | A | 9-Amino-2-cyclobutyl-5-(2-fluorophenyl)-2,3-dihydropyrrolo[3,4-b]quinolin-1-one | 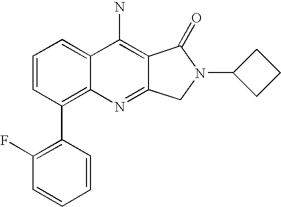 |
| 125 | A | 5-(9-Amino-2-cyclobutyl-1-oxo-2,3-dihydro-1H-pyrrolo[3,4-b]quinolin-5-yl)-pyridine-2-carbonitrile | 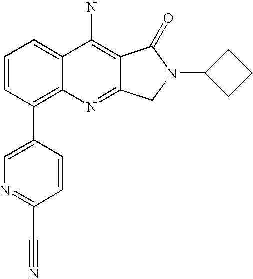 |
| 126 | A | 9-Amino-2-cyclobutyl-5-(6-fluoro-2-methylpyridin-3-yl)-2,3-dihydropyrrolo[3,4-b]quinolin-1-one | 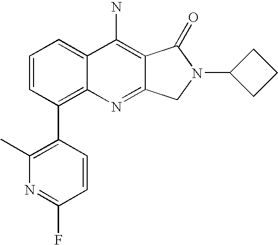 |
| 127 | A | 9-Amino-2-cyclobutyl-5-(2-fluoropyridin-3-yl)-2,3-dihydropyrrolo[3,4-b]quinolin-1-one | 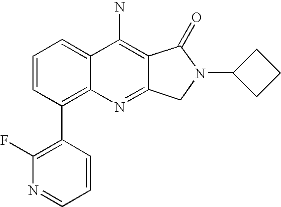 |
| 128 | H | 9-Amino-2-cyclobutyl-5-(6-methoxy-5-methylpyridin-3-yl)-2,3-dihydropyrrolo[3,4-b]quinolin-1-one | 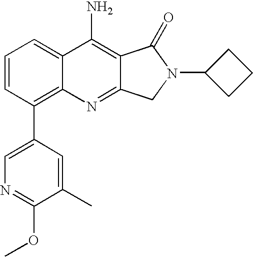 |
| 129 | A | 9-Amino-2-cyclobutyl-5-((P)-2-fluoro-6-methoxyphenyl)-2,3-dihydropyrrolo[3,4-b]quinolin-1-one | 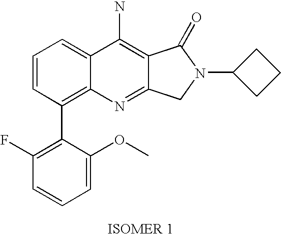 |
| 130 | A | 9-Amino-2-cyclobutyl-5-((M)-2-fluoro-6-methoxyphenyl)-2,3-dihydropyrrolo[3,4-b]quinolin-1-one | 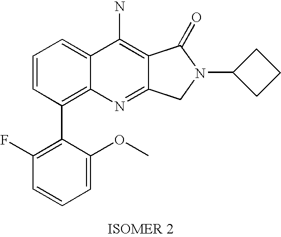 |
| 131 | I | 2-(9-Amino-2-cyclobutyl-1-oxo-2,3-dihydro-1H-pyrrolo[3,4-b]quinolin-5-yl)-6-methoxybenzonitrile | 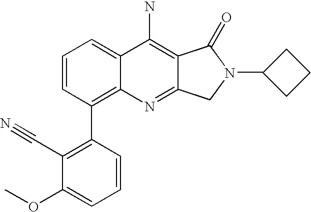 |
| 132 | I | 2-(9-Amino-2-cyclobutyl-1-oxo-2,3-dihydro-1H-pyrrolo[3,4-b]quinolin-5-yl)-3-methoxybenzonitrile | 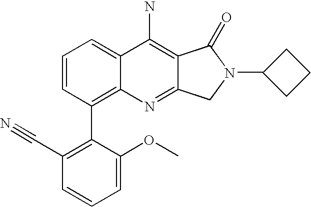 |
| 133 | A | 9-Amino-2-cyclobutyl-5-(2,6-difluoropyridin-3-yl)-2,3-dihydropyrrolo[3,4-b]quinolin-1-one | 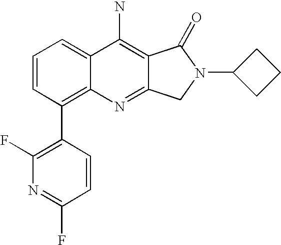 |
| 134 | A | 9-Amino-5-(2-fluoro-6-methoxyphenyl)-2-(3-methylcyclobutyl)-2,3-dihydropyrrolo[3,4-b]quinolin-1-one | 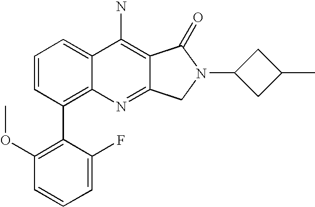 |
| 135 | A | 9-Amino-2-cyclobutyl-5-(1-methyl-1H-pyrazol-4-yl)-2,3-dihydropyrrolo[3,4-b]quinolin-1-one | 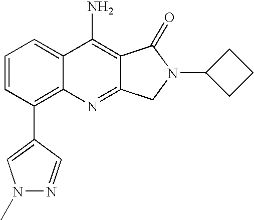 |
| 136 | A | 9-Amino-5-(6-methoxy-2-methylpyridin-3-yl)-2-((1s,3s)-3-methylcyclobutyl)-2,3-dihydro-1H-pyrrolo[3,4-b]quinolin-1-one | 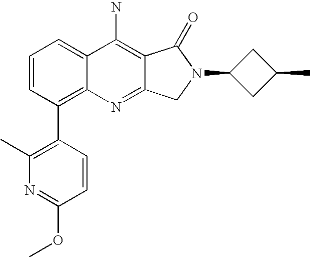 |
| 137 | A | 9-Amino-5-(2-fluoro-6-methoxyphenyl)-2-((1s,3s)-3-methylcyclobutyl)-2,3-dihydro-1H-pyrrolo[3,4-b]quinolin-1-one | 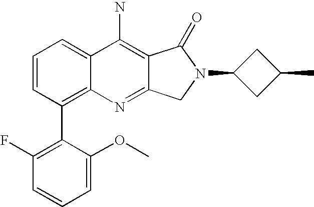 |
| 138 | A | 9-Amino-5-(2-methoxypyridin-3-yl)-2-((1s,3s)-3-methylcyclobutyl)-2,3-dihydro-1H-pyrrolo[3,4-b]quinolin-1-one | 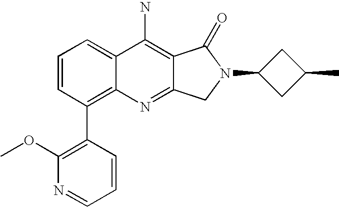 |
| 139 | I | 2-(9-Amino-2-cyclopropyl-1-oxo-2,3-dihydro-1H-pyrrolo[3,4-b]quinolin-5-yl)-3-methoxybenzonitrile | 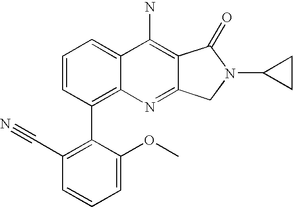 |
| 140 | A | 9-Amino-2-cyclopropyl-5-((P)2-fluoro-6-methoxyphenyl)-2,3-dihydropyrrolo[3,4-b]quinolin-1-one | 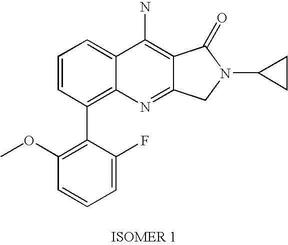 |
| 141 | G | 9-Amino-2-cyclobutyl-5-(2-fluoro-6-methylpyridin-3-yl)-2,3-dihydropyrrolo[3,4-b]quinolin-1-one | 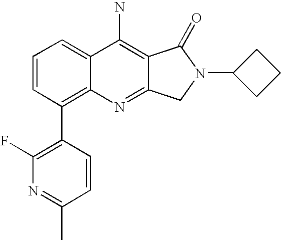 |
| 142 | A | 9-Amino-2-cyclobutyl-5-(6-methoxy-2-methylpyridin-3-yl)-2,3-dihydropyrrolo[3,4-b]quinolin-1-one | 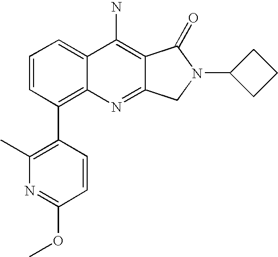 |
| 143 | A | 9-Amino-2-cyclobutyl-5-(1,3-dimethyl-1H-pyrazol-4-yl)-2,3-dihydropyrrolo[3,4-b]quinolin-1-one | 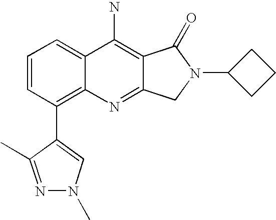 |
| 144 | A | 9-Amino-2-cyclobutyl-5-(6-fluoro-5-methylpyridin-3-yl)-2,3-dihydropyrrolo[3,4-b]quinolin-1-one | 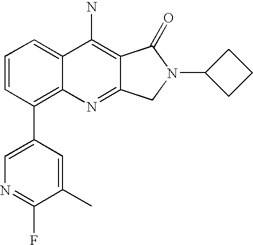 |
| 145 | A | 9-Amino-2-cyclopentyl-5-(2-fluoro-6-methoxyphenyl)-2,3-dihydropyrrolo[3,4-b]quinolin-1-one | 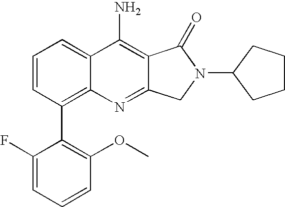 |
| 146 | A | 2-(9-Amino-2-cyclopentyl-1-oxo-2,3-dihydro-1H-pyrrolo[3,4-b]quinolin-5-yl)-benzonitrile | 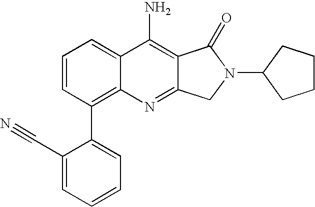 |
| 147 | A | 9-Amino-2-cyclopentyl-5-(6-methoxypyridin-3-yl)-2,3-dihydropyrrolo[3,4-b]quinolin-1-one | 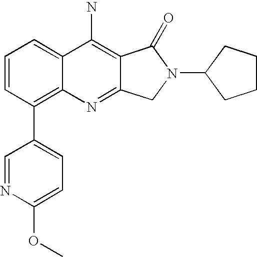 |
| 148 | A | 9-Amino-2-cyclobutyl-5-(6-morpholin-4-yl-pyridin-3-yl)-2,3-dihydropyrrolo[3,4-b]quinolin-1-one | 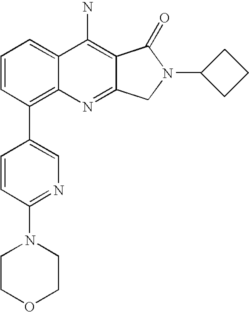 |
| 149 | A | 9-Amino-2-cyclobutyl-5-(6-methoxypyridin-3-yl)-2,3-dihydropyrrolo[3,4-b]quinolin-1-one | 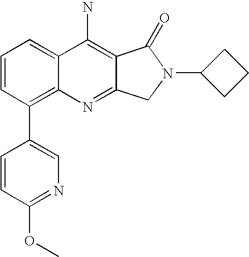 |
| 150 | A | 9-Amino-2-cyclobutyl-5-(4-methylpyridin-3-yl)-2,3-dihydropyrrolo[3,4-b]quinolin-1-one | 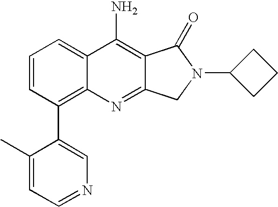 |
| 151 | A | 9-Amino-2-cyclobutyl-5-(3-fluoropyrazin-2-yl)-2,3-dihydropyrrolo[3,4-b]quinolin-1-one | 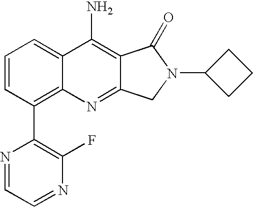 |
| 152 | A | 9-Amino-2-cyclobutyl-5-(5-methoxy-pyridin-3-yl)-2,3-dihydropyrrolo[3,4-b]quinolin-1-one | 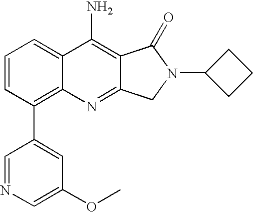 |
| 153 | D | 9-Amino-2-cyclopropyl-6-fluoro-5-(2-fluoro-3-methoxyphenyl)-2,3-dihydropyrrolo[3,4-b]quinolin-1-one | 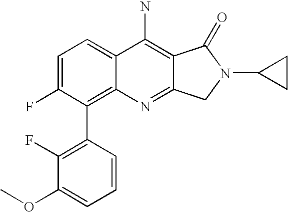 |
| 154 | D | 9-Amino-2-cyclopropyl-5-(2,6-difluoro-3-methoxyphenyl)-6-fluoro-2,3-dihydropyrrolo[3,4-b]quinolin-1-one | 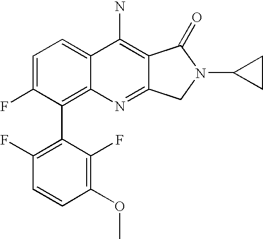 |
| 155 | D | 9-Amino-2-cyclopropyl-6-fluoro-5-(2-fluoro-5-methoxyphenyl)-2,3-dihydropyrrolo[3,4-b]quinolin-1-one | 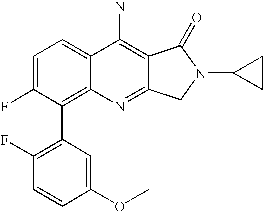 |
| 156 | D | 9-Amino-2-ethyl-6-fluoro-5-(4-methylpyridin-3-yl)-2,3-dihydropyrrolo[3,4-b]quinolin-1-one | 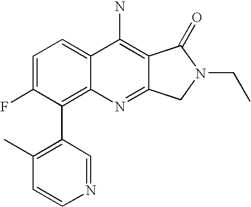 |
| 157 | D | 9-Amino-2-ethyl-6-fluoro-5-(2-fluoro-5-methoxyphenyl)-2,3-dihydropyrrolo[3,4-b]quinolin-1-one | 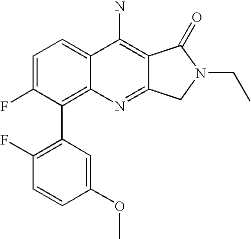 |
| 158 | D | 9-Amino-2-cyclobutyl-5-(2,4-dimethoxypyrimidin-5-yl)-6-fluoro-2,3-dihydropyrrolo[3,4-b]quinolin-1-one | 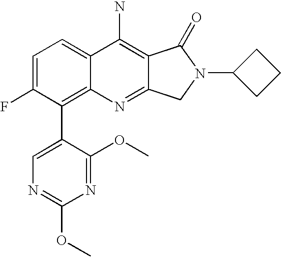 |
| 159 | D | 9-Amino-2-cyclobutyl-5-(2,5-dimethoxyphenyl)-6-fluoro-2,3-dihydropyrrolo[3,4-b]quinolin-1-one | 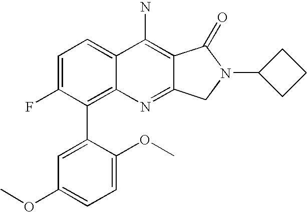 |
| 160 | D | 9-Amino-2-cyclobutyl-6-fluoro-5-(2-methoxypyridin-3-yl)-2,3-dihydropyrrolo[3,4-b]quinolin-1-one | 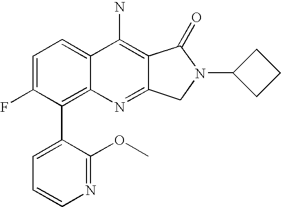 |
| 161 | D | 9-Amino-2-cyclopropyl-6-fluoro-5-(2-methoxyphenyl)-2,3-dihydropyrrolo[3,4-b]quinolin-1-one | 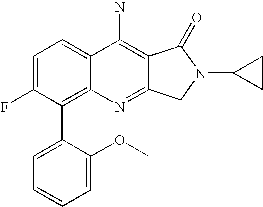 |
| 162 | D | 9-Amino-2-cyclobutyl-6-fluoro-5-(2-methoxyphenyl)-2,3-dihydropyrrolo[3,4-b]quinolin-1-one | 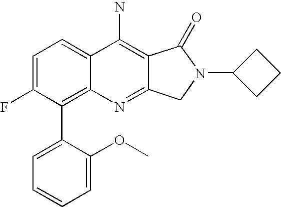 |
| 163 | D | 9-Amino-5-(5-chloro-2-methoxyphenyl)-2-ethyl-6-fluoro-2,3-dihydropyrrolo[3,4-b]quinolin-1-one | 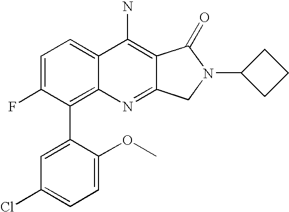 |
| 164 | D | 9-Amino-2-cyclobutyl-6-fluoro-5-(2-fluoro-5-methoxyphenyl)-2,3-dihydropyrrolo[3,4-b]quinolin-1-one | 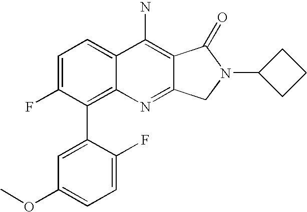 |
| 165 | D | 9-Amino-2-cyclobutyl-6-fluoro-5-(5-fluoro-2-methoxyphenyl)-2,3-dihydropyrrolo[3,4-b]quinolin-1-one | 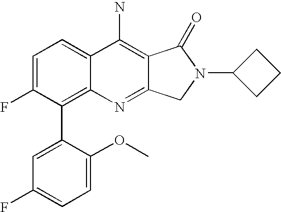 |
| 166 | D | 9-Amino-2-cyclobutyl-6-fluoro-5-(6-methoxy-4-methylpyridin-3-yl)-2,3-dihydropyrrolo[3,4-b]quinolin-1-one | 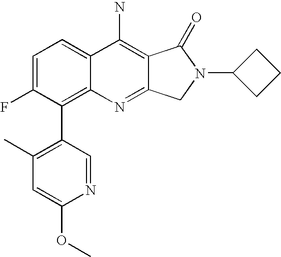 |
| 167 | D | 9-Amino-2-cyclobutyl-6-fluoro-5-(6-methoxy-2-methylpyridin-3-yl)-2,3-dihydropyrrolo[3,4-b]quinolin-1-one | 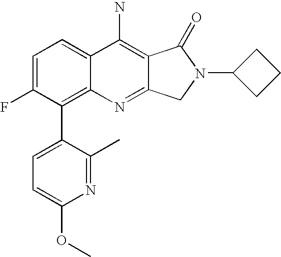 |
| 168 | E | 9-Amino-2-cyclobutyl-5-(2,5-dimethoxypyridin-3-yl)-2,3-dihydropyrrolo[3,4-b]quinolin-1-one | 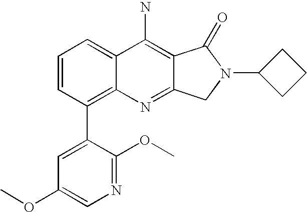 |
| 169 | D | 9-Amino-6-fluoro-5-(2-methoxypyridin-3-yl)-2-(R)-tetrahydrofuran-3-yl-2,3-dihydropyrrolo[3,4-b]quinolin-1-one | 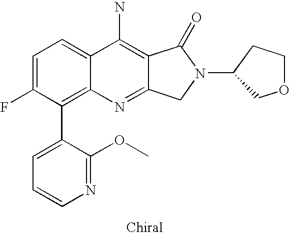 |
| 170 | D | 9-Amino-6-fluoro-5-(2-methoxypyridin-3-yl)-2-(S)-tetrahydrofuran-3-yl-2,3-dihydropyrrolo[3,4-b]quinolin-1-one | 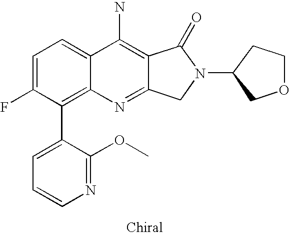 |
| 171 | D | 9-Amino-2-cyclobutyl-5-(3,4-dimethoxyphenyl)-6-fluoro-2,3-dihydropyrrolo[3,4-b]quinolin-1-one | 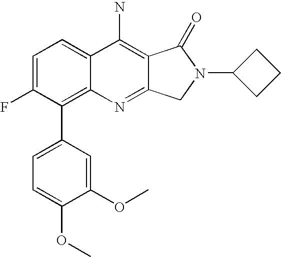 |

In addition, the following compounds may be made using one or more methods described below or similar methods thereof. These compounds may include:
9-amino-2-cyclopropyl-5-(2-fluoro-6-methoxyphenyl)-2,3-dihydro-1H-pyrrolo[3,4-b]quinolin-1-one;
9-amino-2-cyclobutyl-5-(2,5-dimethoxyphenyl)-6-fluoro-2,3-dihydro-1H-pyrrolo[3,4-b]quinolin-1-one;
9-amino-2-cyclobutyl-6-fluoro-5-(2-methoxypyridin-3-yl)-2,3-dihydro-1H-pyrrolo[3,4-b]quinolin-1-one;
9-amino-2-cyclobutyl-5-(2-fluorophenyl)-2,3-dihydro-1H-pyrrolo[3,4-b]quinolin-1-one;
9-amino-2-cyclobutyl-5-(2,4-dimethoxypyrimidin-5-yl)-6-fluoro-2,3-dihydro-1H-pyrrolo[3,4-b]quinolin-1-one;
2-(9-amino-2-cyclobutyl-1-oxo-2,3-dihydro-1H-pyrrolo[3,4-b]quinolin-5-yl)benzonitrile;
9-amino-2-ethyl-6-fluoro-5-(2-fluoro-5-methoxyphenyl)-2,3-dihydro-1H-pyrrolo[3,4-b]quinolin-1-one;
9-amino-2-cyclobutyl-5-(2,6-difluoro-4-methoxyphenyl)-2,3-dihydro-1H-pyrrolo[3,4-b]quinolin-1-one;
9-amino-2-ethyl-6-fluoro-5-(2-fluoro-4-methoxyphenyl)-2,3-dihydro-1H-pyrrolo[3,4-b]quinolin-1-one;
9-amino-2-(3,4-dimethoxybenzyl)-5-(4-methoxypyridin-3-yl)-2,3-dihydro-1H-pyrrolo[3,4-b]quinolin-1-one;
9-amino-2-ethyl-6-fluoro-5-(2-fluorophenyl)-2,3-dihydro-1H-pyrrolo[3,4-b]quinolin-1-one;
9-amino-2-ethyl-6-fluoro-5-(4-methylpyridin-3-yl)-2,3-dihydro-1H-pyrrolo[3,4-b]quinolin-1-one;
9-amino-2-(benzo[d][1,3]dioxol-5-ylmethyl)-5-(4-methoxypyridin-3-yl)-2,3-dihydro-1H-pyrrolo[3,4-b]quinolin-1-one;
9-amino-5-(2-chloro-6-methoxyphenyl)-2-cyclobutyl-2,3-dihydro-1H-pyrrolo[3,4-b]quinolin-1-one;
9-amino-2-(3,4-dimethoxybenzyl)-5-(2-methoxypyridin-3-yl)-2,3-dihydro-1H-pyrrolo[3,4-b]quinolin-1-one;
9-amino-5-(2,6-dimethylpyridin-3-yl)-2-propyl-2,3-dihydro-1H-pyrrolo[3,4-b]quinolin-1-one;
9-amino-2-(3,4-dimethoxybenzyl)-5-(2-fluoro-6-methoxyphenyl)-2,3-dihydro-1H-pyrrolo[3,4-b]quinolin-1-one;
9-amino-2-cyclopropyl-6-fluoro-5-(2-fluoro-3-methoxyphenyl)-2,3-dihydro-1H-pyrrolo[3,4-b]quinolin-1-one;
9-amino-2-(3,4-dimethoxybenzyl)-5-(2,6-dimethylpyridin-3-yl)-2,3-dihydro-1H-pyrrolo[3,4-b]quinolin-1-one;
9-amino-2-cyclopentyl-5-(2-fluoro-6-methoxyphenyl)-2,3-dihydro-1H-pyrrolo[3,4-b]quinolin-1-one;
9-amino-2-cyclobutyl-5-(2,5-dichlorophenyl)-2,3-dihydro-1H-pyrrolo[3,4-b]quinolin-1-one;
9-amino-2-(4-methoxybenzyl)-5-(4-methoxypyridin-3-yl)-2,3-dihydro-1H-pyrrolo[3,4-b]quinolin-1-one;
9-amino-2-ethyl-6-fluoro-5-(2-fluoro-6-methoxyphenyl)-2,3-dihydro-1H-pyrrolo[3,4-b]quinolin-1-one;
9-amino-2-cyclopropyl-5-(2,6-difluorophenyl)-2,3-dihydro-1H-pyrrolo[3,4-b]quinolin-1-one;
9-amino-5-(2-methoxy-5-methylpyridin-3-yl)-2-propyl-2,3-dihydro-1H-pyrrolo[3,4-b]quinolin-1-one;
9-amino-5-(2,4-difluorophenyl)-2-ethyl-6-fluoro-2,3-dihydro-1H-pyrrolo[3,4-b]quinolin-1-one;
9-amino-2-cyclobutyl-5-(2-methoxy-5-methylphenyl)-2,3-dihydro-1H-pyrrolo[3,4-b]quinolin-1-one;
9-amino-2-cyclopropyl-5-(2,6-difluoro-3-methoxyphenyl)-6-fluoro-2,3-dihydro-1H-pyrrolo[3,4-b]quinolin-1-one;
9-amino-2-cyclobutyl-5-(2,4-dimethoxypyrimidin-5-yl)-2,3-dihydro-1H-pyrrolo[3,4-b]quinolin-1-one;
9-amino-2-cyclobutyl-5-(6-methylpyridin-3-yl)-2,3-dihydro-1H-pyrrolo[3,4-b]quinolin-1-one;
9-amino-5-(2,6-difluoro-4-methoxyphenyl)-2-ethyl-6-fluoro-2,3-dihydro-1H-pyrrolo[3,4-b]quinolin-1-one;
9-amino-2-butyl-5-(2-fluoro-6-methoxyphenyl)-2,3-dihydro-1H-pyrrolo[3,4-b]quinolin-1-one;
9-amino-2-(3,4-dimethoxybenzyl)-5-(6-methoxy-2-methylpyridin-3-yl)-2,3-dihydro-1H-pyrrolo[3,4-b]quinolin-1-one;
9-amino-2-cyclopropyl-5-(2-methoxy-5-methylphenyl)-2,3-dihydro-1H-pyrrolo[3,4-b]quinolin-1-one;
9-amino-5-(2-chlorophenyl)-2-propyl-2,3-dihydro-1H-pyrrolo[3,4-b]quinolin-1-one;
9-amino-5-(2,4-dimethoxyphenyl)-2-ethyl-6-fluoro-2,3-dihydro-1H-pyrrolo[3,4-b]quinolin-1-one;
9-amino-5-(2-chloro-6-methoxyphenyl)-2-cyclopropyl-6-fluoro-2,3-dihydro-1H-pyrrolo[3,4-b]quinolin-1-one;
9-amino-2-ethyl-6-fluoro-5-(2-fluoro-3-methoxyphenyl)-2,3-dihydro-1H-pyrrolo[3,4-b]quinolin-1-one;
9-amino-2-propyl-5-(2-methylphenyl)-2,3-dihydro-1H-pyrrolo[3,4-b]quinolin-1-one;
9-amino-2-(benzo[d][1,3]dioxol-5-ylmethyl)-5-(2-methoxypyridin-3-yl)-2,3-dihydro-1H-pyrrolo[3,4-b]quinolin-1-one;
9-amino-2-(4-methoxybenzyl)-5-(5-methoxypyridin-3-yl)-2,3-dihydro-1H-pyrrolo[3,4-b]quinolin-1-one;
9-amino-5-(5-fluoro-2-methylphenyl)-2-propyl-2,3-dihydro-1H-pyrrolo[3,4-b]quinolin-1-one;
9-amino-5-(2-chloro-5-methylpyridin-3-yl)-2-propyl-2,3-dihydro-1H-pyrrolo[3,4-b]quinolin-1-one;
9-amino-5-(2-fluoro-5-methylphenyl)-2-propyl-2,3-dihydro-1H-pyrrolo[3,4-b]quinolin-1-one;
9-amino-5-(2-fluoropyridin-3-yl)-2-(4-methoxybenzyl)-2,3-dihydro-1H-pyrrolo[3,4-b]quinolin-1-one;
9-amino-2-cyclopropyl-5-(2,5-difluorophenyl)-2,3-dihydro-1H-pyrrolo[3,4-b]quinolin-1-one;
9-amino-2-ethyl-5-(2-fluoro-4-methoxyphenyl)-2,3-dihydro-1H-pyrrolo[3,4-b]quinolin-1-one;
9-amino-2-cyclopropyl-6-fluoro-5-(2-fluoro-4-methoxyphenyl)-2,3-dihydro-1H-pyrrolo[3,4-b]quinolin-1-one;
9-amino-2-cyclopropyl-6-fluoro-5-(2-fluoro-5-methoxyphenyl)-2,3-dihydro-1H-pyrrolo[3,4-b]quinolin-1-one;
9-amino-5-(2-fluoro-5-methylpyridin-3-yl)-2-propyl-2,3-dihydro-1H-pyrrolo[3,4-b]quinolin-1-one;
9-amino-2-cyclopropyl-6-fluoro-5-(2-methoxy-5-methylphenyl)-2,3-dihydro-1H-pyrrolo[3,4-b]quinolin-1-one;
9-amino-5-(2-chloro-6-methylpyridin-3-yl)-2-cyclopropyl-2,3-dihydro-1H-pyrrolo[3,4-b]quinolin-1-one;
9-amino-5-(2-fluoro-3-methoxyphenyl)-2-(4-methoxybenzyl)-2,3-dihydro-1H-pyrrolo[3,4-b]quinolin-1-one;
9-amino-5-(2-fluoro-5-methoxypyridin-4-yl)-2-propyl-2,3-dihydro-1H-pyrrolo[3,4-b]quinolin-1-one;
9-amino-2-cyclopropyl-5-(4-methoxypyridin-3-yl)-2,3-dihydro-1H-pyrrolo[3,4-b]quinolin-1-one;
9-amino-6-fluoro-5-(2-fluoro-6-methoxyphenyl)-2-methyl-2,3-dihydro-1H-pyrrolo[3,4-b]quinolin-1-one;
9-amino-2-cyclopropyl-5-(2,6-difluoro-4-methoxyphenyl)-2,3-dihydro-1H-pyrrolo[3,4-b]quinolin-1-one;
9-amino-5-(2,4-dimethoxypyrimidin-5-yl)-6-fluoro-2-propyl-2,3-dihydro-1H-pyrrolo[3,4-b]quinolin-1-one;
9-amino-5-(5-chloro-2-methoxyphenyl)-2-cyclopropyl-2,3-dihydro-1H-pyrrolo[3,4-b]quinolin-1-one;
9-amino-2-(3,4-dimethoxybenzyl)-5-(2-methoxy-5-methylphenyl)-2,3-dihydro-1H-pyrrolo[3,4-b]quinolin-1-one;
9-amino-5-(2-chloro-5-methoxypyridin-3-yl)-2-propyl-2,3-dihydro-1H-pyrrolo[3,4-b]quinolin-1-one;
9-amino-2-(3-chloro-4-methoxybenzyl)-5-(2-methoxypyridin-3-yl)-2,3-dihydro-1H-pyrrolo[3,4-b]quinolin-1-one;
9-amino-5-(1,3-dimethyl-1H-pyrazol-5-yl)-2-(4-methoxybenzyl)-2,3-dihydro-1H-pyrrolo[3,4-b]quinolin-1-one;
9-amino-2-(3-chloro-4-methoxybenzyl)-5-(2-fluoro-6-methoxyphenyl)-2,3-dihydro-1H-pyrrolo[3,4-b]quinolin-1-one;
9-amino-5-(4-methoxy-2-(trifluoromethyl)phenyl)-2-propyl-2,3-dihydro-1H-pyrrolo[3,4-b]quinolin-1-one;
9-amino-5-(5-bromo-2-methoxypyridin-3-yl)-2-propyl-2,3-dihydro-1H-pyrrolo[3,4-b]quinolin-1-one;
9-amino-5-(2-methylpyridin-3-yl)-2-propyl-2,3-dihydro-1H-pyrrolo[3,4-b]quinolin-1-one;
9-amino-5-(2-fluoro-3-methoxyphenyl)-2-propyl-2,3-dihydro-1H-pyrrolo[3,4-b]quinolin-1-one;
9-amino-5-(2,5-dichlorophenyl)-2-propyl-2,3-dihydro-1H-pyrrolo[3,4-b]quinolin-1-one;
9-amino-5-(2,6-difluoro-4-methoxyphenyl)-2-propyl-2,3-dihydro-1H-pyrrolo[3,4-b]quinolin-1-one;
9-amino-5-(3,4-dimethoxyphenyl)-2-propyl-2,3-dihydro-1H-pyrrolo[3,4-b]quinolin-1-one;
9-amino-5-(2-fluoro-5-methoxyphenyl)-2-propyl-2,3-dihydro-1H-pyrrolo[3,4-b]quinolin-1-one;
9-amino-5-(2-fluoro-4-methoxyphenyl)-2-propyl-2,3-dihydro-1H-pyrrolo[3,4-b]quinolin-1-one;
9-amino-2-cyclopentyl-5-(2,5-dimethoxyphenyl)-2,3-dihydro-1H-pyrrolo[3,4-b]quinolin-1-one;
9-amino-2-ethyl-5-(2-fluoro-5-methoxyphenyl)-2,3-dihydro-1H-pyrrolo[3,4-b]quinolin-1-one;
9-amino-2-(3,4-dimethoxybenzyl)-5-(2,4-dimethoxypyrimidin-5-yl)-2,3-dihydro-1H-pyrrolo[3,4-b]quinolin-1-one;
9-amino-5-(2,5-dimethoxyphenyl)-2-ethyl-6-fluoro-2,3-dihydro-1H-pyrrolo[3,4-b]quinolin-1-one;
9-amino-5-(2,4-dimethoxyphenyl)-6-fluoro-2-propyl-2,3-dihydro-1H-pyrrolo[3,4-b]quinolin-1-one;
9-amino-5-(3,5-dimethylphenyl)-2-ethyl-2,3-dihydro-1H-pyrrolo[3,4-b]quinolin-1-one;
9-amino-2-cyclopropyl-5-(2-fluoro-4-methoxyphenyl)-2,3-dihydro-1H-pyrrolo[3,4-b]quinolin-1-one;
9-amino-5-(2,4-difluorophenyl)-2-propyl-2,3-dihydro-1H-pyrrolo[3,4-b]quinolin-1-one;
9-amino-5-(2-chloro-6-methoxyphenyl)-2-cyclopropyl-2,3-dihydro-1H-pyrrolo[3,4-b]quinolin-1-one;
9-amino-5-(2-ethoxypyridin-3-yl)-2-propyl-2,3-dihydro-1H-pyrrolo[3,4-b]quinolin-1-one;
9-amino-5-(3,5-difluorophenyl)-2-(3,4-dimethoxybenzyl)-2,3-dihydro-1H-pyrrolo[3,4-b]quinolin-1-one;
2-(9-amino-2-cyclopropyl-1-oxo-2,3-dihydro-1H-pyrrolo[3,4-b]quinolin-5-yl)-3-methoxybenzonitrile;
9-amino-5-(2-chloro-5-fluoropyridin-3-yl)-2-propyl-2,3-dihydro-1H-pyrrolo[3,4-b]quinolin-1-one;
2-(9-amino-2-cyclopropyl-1-oxo-2,3-dihydro-1H-pyrrolo[3,4-b]quinolin-5-yl)benzonitrile;
9-amino-5-(6-methoxy-2-methylpyridin-3-yl)-2-propyl-2,3-dihydro-1H-pyrrolo[3,4-b]quinolin-1-one;
9-amino-2-propyl-5-(2-(trifluoromethyl)phenyl)-2,3-dihydro-1H-pyrrolo[3,4-b]quinolin-1-one;
9-amino-2-cyclopropyl-5-(2,3-difluorophenyl)-2,3-dihydro-1H-pyrrolo[3,4-b]quinolin-1-one;
9-amino-2-cyclopropyl-5-(2-fluorophenyl)-2,3-dihydro-1H-pyrrolo[3,4-b]quinolin-1-one;
9-amino-2-propyl-5-(quinolin-6-yl)-2,3-dihydro-1H-pyrrolo[3,4-b]quinolin-1-one;
9-amino-5-(2-fluoro-6-methylpyridin-3-yl)-2-propyl-2,3-dihydro-1H-pyrrolo[3,4-b]quinolin-1-one;
9-amino-2-butyl-5-(2-methoxypyridin-3-yl)-2,3-dihydro-1H-pyrrolo[3,4-b]quinolin-1-one;
9-amino-2-cyclopropyl-5-(2,4-difluorophenyl)-2,3-dihydro-1H-pyrrolo[3,4-b]quinolin-1-one;
9-amino-5-(6′-chloro-2,3′-bipyridin-5-yl)-2-(3,4-dimethoxybenzyl)-2,3-dihydro-1H-pyrrolo[3,4-b]quinolin-1-one;
9-amino-5-(2-fluoro-6-methoxyphenyl)-2-((R)-1-(4-methoxyphenyl)ethyl)-2,3-dihydro-1H-pyrrolo[3,4-b]quinolin-1-one;
3-(9-amino-1-oxo-2-propyl-2,3-dihydro-1H-pyrrolo[3,4-b]quinolin-5-yl)benzonitrile;
9-amino-2-butyl-5-(2-methoxy-5-methylphenyl)-2,3-dihydro-1H-pyrrolo[3,4-b]quinolin-1-one;
9-amino-5-(1,3-dimethyl-1H-pyrazol-5-yl)-2-propyl-2,3-dihydro-1H-pyrrolo[3,4-b]quinolin-1-one;
9-amino-5-(5-chloro-2-methoxypyridin-3-yl)-2-propyl-2,3-dihydro-1H-pyrrolo[3,4-b]quinolin-1-one;
9-amino-5-(2,6-dichloropyridin-3-yl)-2-propyl-2,3-dihydro-1H-pyrrolo[3,4-b]quinolin-1-one;
9-amino-5-(6-fluoro-2-methylpyridin-3-yl)-2-propyl-2,3-dihydro-1H-pyrrolo[3,4-b]quinolin-1-one;
9-amino-2-ethyl-5-(3-fluoro-2-methoxyphenyl)-2,3-dihydro-1H-pyrrolo[3,4-b]quinolin-1-one;
9-amino-2-cyclopropyl-5-(2-(trifluoromethyl)phenyl)-2,3-dihydro-1H-pyrrolo[3,4-b]quinolin-1-one;
9-amino-5-(6-chloro-2-methylpyridin-3-yl)-2-propyl-2,3-dihydro-1H-pyrrolo[3,4-b]quinolin-1-one;
9-amino-2-ethyl-5-(4-methylpyridin-3-yl)-2,3-dihydro-1H-pyrrolo[3,4-b]quinolin-1-one;
9-amino-2-cyclopropyl-5-(2,6-dimethylphenyl)-2,3-dihydro-1H-pyrrolo[3,4-b]quinolin-1-one;
9-amino-5-(2-methoxy-6-methylpyridin-3-yl)-2-propyl-2,3-dihydro-1H-pyrrolo[3,4-b]quinolin-1-one;
9-amino-5-(2-fluoro-5-(trifluoromethyl)phenyl)-2-propyl-2,3-dihydro-1H-pyrrolo[3,4-b]quinolin-1-one;
9-amino-5-(2,3-dimethoxyphenyl)-2-propyl-2,3-dihydro-1H-pyrrolo[3,4-b]quinolin-1-one;
9-amino-2-propyl-5-(pyridin-4-yl)-2,3-dihydro-1H-pyrrolo[3,4-b]quinolin-1-one;
9-amino-2-ethyl-5-(4-fluoro-3-methoxyphenyl)-2,3-dihydro-1H-pyrrolo[3,4-b]quinolin-1-one;
9-amino-2-ethyl-5-(2-methoxy-5-methylphenyl)-2,3-dihydro-1H-pyrrolo[3,4-b]quinolin-1-one;
9-amino-5-(5-fluoro-2-methylphenyl)-2-(4-methoxybenzyl)-2,3-dihydro-1H-pyrrolo[3,4-b]quinolin-1-one;
9-amino-5-(1H-indol-5-yl)-2-propyl-2,3-dihydro-1H-pyrrolo[3,4-b]quinolin-1-one;
9-amino-5-(2-fluoro-6-methoxyphenyl)-2-isopropyl-2,3-dihydro-1H-pyrrolo[3,4-b]quinolin-1-one;
9-amino-5-(2,5-dimethoxyphenyl)-2-(3-methoxybenzyl)-2,3-dihydro-1H-pyrrolo[3,4-b]quinolin-1-one;
9-amino-5-(3,5-dimethoxyphenyl)-2-ethyl-2,3-dihydro-1H-pyrrolo[3,4-b]quinolin-1-one;
9-amino-2-ethyl-5-(5-fluoro-2-methoxyphenyl)-2,3-dihydro-1H-pyrrolo[3,4-b]quinolin-1-one;
9-amino-2-(3-chlorobenzyl)-5-(2-fluoro-6-methoxyphenyl)-2,3-dihydro-1H-pyrrolo[3,4-b]quinolin-1-one;
9-amino-5-(6-chloro-2-methylpyridin-3-yl)-2-cyclopropyl-2,3-dihydro-1H-pyrrolo[3,4-b]quinolin-1-one;
9-amino-2-cyclopropyl-5-(6-methylpyridin-3-yl)-2,3-dihydro-1H-pyrrolo[3,4-b]quinolin-1-one;
9-amino-2-(benzo[d][1,3]dioxol-5-ylmethyl)-5-(2,4-dimethoxypyrimidin-5-yl)-2,3-dihydro-1H-pyrrolo[3,4-b]quinolin-1-one;
9-amino-5-(6-fluoro-5-methylpyridin-3-yl)-2-propyl-2,3-dihydro-1H-pyrrolo[3,4-b]quinolin-1-one;
2-(9-amino-1-oxo-2-propyl-2,3-dihydro-1H-pyrrolo[3,4-b]quinolin-5-yl)benzonitrile;
9-amino-2-propyl-5-(thiophen-3-yl)-2,3-dihydro-1H-pyrrolo[3,4-b]quinolin-1-one;
9-amino-5-(4-fluoro-2-methoxyphenyl)-2-(4-methoxybenzyl)-2,3-dihydro-1H-pyrrolo[3,4-b]quinolin-1-one;
9-amino-2-cyclopropyl-5-(4-methoxy-2-(trifluoromethyl)phenyl)-2,3-dihydro-1H-pyrrolo[3,4-b]quinolin-1-one;
9-amino-5-(2,4-dimethoxyphenyl)-2-(4-methoxybenzyl)-2,3-dihydro-1H-pyrrolo[3,4-b]quinolin-1-one;
9-amino-5-(2-fluoro-3-methoxyphenyl)-2-methyl-2,3-dihydro-1H-pyrrolo[3,4-b]quinolin-1-one;
9-amino-5-(2-chloro-5-methoxyphenyl)-2-cyclopropyl-2,3-dihydro-1H-pyrrolo[3,4-b]quinolin-1-one;
9-amino-2-(4-methoxybenzyl)-5-(2-(trifluoromethyl)phenyl)-2,3-dihydro-1H-pyrrolo[3,4-b]quinolin-1-one;
9-amino-2-ethyl-5-(3-fluoro-5-methoxyphenyl)-2,3-dihydro-1H-pyrrolo[3,4-b]quinolin-1-one;
9-amino-5-(2,5-difluorophenyl)-2-propyl-2,3-dihydro-1H-pyrrolo[3,4-b]quinolin-1-one;
9-amino-2-cyclopropyl-5-(2-methoxypyridin-3-yl)-2,3-dihydro-1H-pyrrolo[3,4-b]quinolin-1-one;
9-amino-5-(6-methoxy-5-methylpyridin-3-yl)-2-propyl-2,3-dihydro-1H-pyrrolo[3,4-b]quinolin-1-one;
9-amino-2-cyclopropyl-5-(2,5-dimethoxyphenyl)-6-(methylthio)-2,3-dihydro-1H-pyrrolo[3,4-b]quinolin-1-one;
9-amino-2-cyclopropyl-5-(2,5-difluoro-4-methoxyphenyl)-2,3-dihydro-1H-pyrrolo[3,4-b]quinolin-1-one;
9-amino-5-(3-(dimethylamino)phenyl)-2-propyl-2,3-dihydro-1H-pyrrolo[3,4-b]quinolin-1-one;
9-amino-5-(furan-3-yl)-2-propyl-2,3-dihydro-1H-pyrrolo[3,4-b]quinolin-1-one;
9-amino-5-(4-methoxypyridin-3-yl)-2-propyl-2,3-dihydro-1H-pyrrolo[3,4-b]quinolin-1-one;
9-amino-2-cyclobutyl-5-(3,5-difluorophenyl)-2,3-dihydro-1H-pyrrolo[3,4-b]quinolin-1-one;
9-amino-5-(5-chloro-2-methoxyphenyl)-2-propyl-2,3-dihydro-1H-pyrrolo[3,4-b]quinolin-1-one;
9-amino-5-(2-methoxypyridin-3-yl)-2-propyl-2,3-dihydro-1H-pyrrolo[3,4-b]quinolin-1-one;
9-amino-2-ethyl-5-(3-fluoro-4-methoxyphenyl)-2,3-dihydro-1H-pyrrolo[3,4-b]quinolin-1-one;
9-amino-2-cyclopropyl-5-(2-fluoro-6-methylpyridin-3-yl)-2,3-dihydro-1H-pyrrolo[3,4-b]quinolin-1-one;
9-amino-5-phenyl-2-propyl-2,3-dihydro-1H-pyrrolo[3,4-b]quinolin-1-one;
9-amino-2-propyl-5-(3-methylphenyl)-2,3-dihydro-1H-pyrrolo[3,4-b]quinolin-1-one;
9-amino-2-(2-butyl)-5-(2-fluoro-6-methoxyphenyl)-2,3-dihydro-1H-pyrrolo[3,4-b]quinolin-1-one;
9-amino-5-(5-fluoro-2-methoxyphenyl)-2-(4-methoxybenzyl)-2,3-dihydro-1H-pyrrolo[3,4-b]quinolin-1-one;
9-amino-5-(3,5-dimethoxyphenyl)-2-propyl-2,3-dihydro-1H-pyrrolo[3,4-b]quinolin-1-one;
9-amino-5-(2-methoxy-5-methylphenyl)-2-propyl-2,3-dihydro-1H-pyrrolo[3,4-b]quinolin-1-one;
9-amino-5-(3,4-dimethoxyphenyl)-2-(4-methoxybenzyl)-2,3-dihydro-1H-pyrrolo[3,4-b]quinolin-1-one;
9-amino-5-(2,5-difluoro-4-methoxyphenyl)-2-(4-methoxybenzyl)-2,3-dihydro-1H-pyrrolo[3,4-b]quinolin-1-one;
9-amino-5-(2,4-dimethoxyphenyl)-2-propyl-2,3-dihydro-1H-pyrrolo[3,4-b]quinolin-1-one;
9-amino-2-propyl-5-(2,3,4-trimethoxyphenyl)-2,3-dihydro-1H-pyrrolo[3,4-b]quinolin-1-one;
9-amino-5-(2-methoxy-5-(pyridin-4-yl)phenyl)-2-propyl-2,3-dihydro-1H-pyrrolo[3,4-b]quinolin-1-one;
4-(9-amino-1-oxo-2-propyl-2,3-dihydro-1H-pyrrolo[3,4-b]quinolin-5-yl)benzonitrile;
9-amino-2-cyclopropyl-5-(2-methoxyphenyl)-2,3-dihydro-1H-pyrrolo[3,4-b]quinolin-1-one;
9-amino-5-(2-methoxy-5-methylphenyl)-2-(4-methoxybenzyl)-2,3-dihydro-1H-pyrrolo[3,4-b]quinolin-1-one;
9-amino-2-cyclopropyl-5-(6-methoxy-5-methylpyridin-3-yl)-2,3-dihydro-1H-pyrrolo[3,4-b]quinolin-1-one;
9-amino-5-(4-methylpyridin-3-yl)-2-propyl-2,3-dihydro-1H-pyrrolo[3,4-b]quinolin-1-one;
9-amino-5-(2-fluoro-5-methoxyphenyl)-2-methyl-2,3-dihydro-1H-pyrrolo[3,4-b]quinolin-1-one;
9-amino-5-(2,5-dimethylphenyl)-2-propyl-2,3-dihydro-1H-pyrrolo[3,4-b]quinolin-1-one;
9-amino-5-(3-fluoro-2-methoxyphenyl)-2-propyl-2,3-dihydro-1H-pyrrolo[3,4-b]quinolin-1-one;
9-amino-2-cyclopropyl-5-(2,4-dimethoxyphenyl)-2,3-dihydro-1H-pyrrolo[3,4-b]quinolin-1-one;
9-amino-5-(2,5-dimethoxyphenyl)-2-ethyl-2,3-dihydro-1H-pyrrolo[3,4-b]quinolin-1-one;
9-amino-2-(4-methoxybenzyl)-5-(6-methylpyridin-3-yl)-2,3-dihydro-1H-pyrrolo[3,4-b]quinolin-1-one;
9-amino-2-(benzo[d][1,3]dioxol-5-ylmethyl)-5-(2,5-dimethoxyphenyl)-2,3-dihydro-1H-pyrrolo[3,4-b]quinolin-1-one;
9-amino-5-(4-fluoro-2-methoxyphenyl)-2-propyl-2,3-dihydro-1H-pyrrolo[3,4-b]quinolin-1-one;
9-amino-5-(3-methoxyphenyl)-2-propyl-2,3-dihydro-1H-pyrrolo[3,4-b]quinolin-1-one;
9-amino-5-(5-methoxypyridin-3-yl)-2-propyl-2,3-dihydro-1H-pyrrolo[3,4-b]quinolin-1-one;
2-(9-amino-2-methyl-1-oxo-2,3-dihydro-1H-pyrrolo[3,4-b]quinolin-5-yl)benzonitrile;
9-amino-2-cyclopropyl-5-(pyridin-3-yl)-2,3-dihydro-1H-pyrrolo[3,4-b]quinolin-1-one;
5-(9-amino-1-oxo-2-propyl-2,3-dihydro-1H-pyrrolo[3,4-b]quinolin-5-yl)nicotinonitrile;
9-amino-2-methyl-5-(4-methylpyridin-3-yl)-2,3-dihydro-1H-pyrrolo[3,4-b]quinolin-1-one;
9-amino-5-(3,5-dimethylphenyl)-2-methyl-2,3-dihydro-1H-pyrrolo[3,4-b]quinolin-1-one;
9-amino-5-(2,5-dimethoxyphenyl)-2-(4-methoxybenzyl)-2,3-dihydro-1H-pyrrolo[3,4-b]quinolin-1-one;
9-amino-5-(2-methoxy-5-methylphenyl)-2-methyl-2,3-dihydro-1H-pyrrolo[3,4-b]quinolin-1-one;
9-amino-2-(4-methoxybenzyl)-5-(1-methyl-1H-pyrazol-4-yl)-2,3-dihydro-1H-pyrrolo[3,4-b]quinolin-1-one;
9-amino-2-cyclopropyl-5-(5-fluoro-2-methoxyphenyl)-2,3-dihydro-1H-pyrrolo[3,4-b]quinolin-1-one;
9-amino-5-(3,5-dimethoxyphenyl)-2-methyl-2,3-dihydro-1H-pyrrolo[3,4-b]quinolin-1-one;
5-(9-amino-1-oxo-2-propyl-2,3-dihydro-1H-pyrrolo[3,4-b]quinolin-5-yl)-2-fluorobenzonitrile;
9-amino-5-(2,6-difluoropyridin-3-yl)-2-propyl-2,3-dihydro-1H-pyrrolo[3,4-b]quinolin-1-one;
2-(9-amino-1-oxo-2-propyl-2,3-dihydro-1H-pyrrolo[3,4-b]quinolin-5-yl)-3-methoxybenzonitrile;
9-amino-5-(2-methoxypyrimidin-5-yl)-2-propyl-2,3-dihydro-1H-pyrrolo[3,4-b]quinolin-1-one;
9-amino-5-(3-chlorophenyl)-2-propyl-2,3-dihydro-1H-pyrrolo[3,4-b]quinolin-1-one;
9-amino-2-ethyl-5-(6-methylpyridin-3-yl)-2,3-dihydro-1H-pyrrolo[3,4-b]quinolin-1-one;
9-amino-5-(2-fluoro-4-methoxyphenyl)-2-methyl-2,3-dihydro-1H-pyrrolo[3,4-b]quinolin-1-one;
9-amino-2-cyclopropyl-5-(2,4-dimethoxypyrimidin-5-yl)-2,3-dihydro-1H-pyrrolo[3,4-b]quinolin-1-one;
9-amino-5-(4-fluorophenyl)-2-propyl-2,3-dihydro-1H-pyrrolo[3,4-b]quinolin-1-one;
9-amino-5-(2,4-dimethoxyphenyl)-2-methyl-2,3-dihydro-1H-pyrrolo[3,4-b]quinolin-1-one;
9-amino-5-(5-fluoro-2-methoxyphenyl)-2-propyl-2,3-dihydro-1H-pyrrolo[3,4-b]quinolin-1-one;
9-amino-5-(3-fluoro-5-methoxyphenyl)-2-methyl-2,3-dihydro-1H-pyrrolo[3,4-b]quinolin-1-one;
9-amino-2-propyl-5-(4-methylphenyl)-2,3-dihydro-1H-pyrrolo[3,4-b]quinolin-1-one;
9-amino-5-(5-fluoro-6-methoxypyridin-3-yl)-2-propyl-2,3-dihydro-1H-pyrrolo[3,4-b]quinolin-1-one;
9-amino-5-(2,3-dimethoxyphenyl)-2-ethyl-2,3-dihydro-1H-pyrrolo[3,4-b]quinolin-1-one;
9-amino-5-(2,6-dimethoxyphenyl)-2-ethyl-2,3-dihydro-1H-pyrrolo[3,4-b]quinolin-1-one;
9-amino-2-cyclopropyl-5-(4-fluoro-2-methoxyphenyl)-2,3-dihydro-1H-pyrrolo[3,4-b]quinolin-1-one;
9-amino-5-(6-chloro-5-methylpyridin-3-yl)-2-propyl-2,3-dihydro-1H-pyrrolo[3,4-b]quinolin-1-one;
9-amino-5-(3,4-dimethoxyphenyl)-2-methyl-2,3-dihydro-1H-pyrrolo[3,4-b]quinolin-1-one;
9-amino-2-propyl-5-(3,4,5-trimethoxyphenyl)-2,3-dihydro-1H-pyrrolo[3,4-b]quinolin-1-one;
9-amino-2-butyl-5-(6-methylpyridin-3-yl)-2,3-dihydro-1H-pyrrolo[3,4-b]quinolin-1-one;
9-amino-5-(2,3-difluorophenyl)-2-(4-methoxybenzyl)-2,3-dihydro-1H-pyrrolo[3,4-b]quinolin-1-one;
9-amino-5-(2,4-dimethoxyphenyl)-2-ethyl-2,3-dihydro-1H-pyrrolo[3,4-b]quinolin-1-one;
9-amino-5-(1-methyl-1H-pyrazol-4-yl)-2-propyl-2,3-dihydro-1H-pyrrolo[3,4-b]quinolin-1-one;
9-amino-2-(3-methoxybenzyl)-5-(4-methoxypyridin-3-yl)-2,3-dihydro-1H-pyrrolo[3,4-b]quinolin-1-one;
9-amino-5-(2,5-difluoro-4-methoxyphenyl)-2-propyl-2,3-dihydro-1H-pyrrolo[3,4-b]quinolin-1-one;
9-amino-2-cyclopropyl-5-(3,4-dimethoxyphenyl)-6-fluoro-2,3-dihydro-1H-pyrrolo[3,4-b]quinolin-1-one;
9-amino-2-cyclobutyl-5-(2-fluoro-6-methylpyridin-3-yl)-2,3-dihydro-1H-pyrrolo[3,4-b]quinolin-1-one;
9-amino-5-(3,4-dimethoxyphenyl)-6-fluoro-2-propyl-2,3-dihydro-1H-pyrrolo[3,4-b]quinolin-1-one;
9-amino-2-cyclobutyl-5-(3,4-dimethoxyphenyl)-6-fluoro-2,3-dihydro-1H-pyrrolo[3,4-b]quinolin-1-one;
9-amino-2-cyclopentyl-5-(2,4-dimethoxypyrimidin-5-yl)-2,3-dihydro-1H-pyrrolo[3,4-b]quinolin-1-one;
9-amino-2-cyclopentyl-5-(2-methoxypyridin-3-yl)-2,3-dihydro-1H-pyrrolo[3,4-b]quinolin-1-one;
9-amino-5-(2-chloro-6-methylpyridin-3-yl)-2-cyclobutyl-2,3-dihydro-1H-pyrrolo[3,4-b]quinolin-1-one;
5-(9-amino-2-cyclobutyl-1-oxo-2,3-dihydro-1H-pyrrolo[3,4-b]quinolin-5-yl)picolinonitrile;
9-amino-5-(3,4-dimethoxyphenyl)-2-ethyl-6-(methylthio)-2,3-dihydro-1H-pyrrolo[3,4-b]quinolin-1-one;
9-amino-2-cyclobutyl-5-(pyridazin-4-yl)-2,3-dihydro-1H-pyrrolo[3,4-b]quinolin-1-one;
2-(9-amino-2-cyclopentyl-1-oxo-2,3-dihydro-1H-pyrrolo[3,4-b]quinolin-5-yl)benzonitrile;
9-amino-2-cyclopentyl-5-(2,5-difluoro-4-methoxyphenyl)-2,3-dihydro-1H-pyrrolo[3,4-b]quinolin-1-one;
9-amino-2-cyclopentyl-5-(5-methoxypyridin-3-yl)-2,3-dihydro-1H-pyrrolo[3,4-b]quinolin-1-one;
9-amino-2-cyclopentyl-5-(6-methoxypyridin-3-yl)-2,3-dihydro-1H-pyrrolo[3,4-b]quinolin-1-one;
9-amino-2-cyclopentyl-5-(2,6-dimethoxypyridin-3-yl)-2,3-dihydro-1H-pyrrolo[3,4-b]quinolin-1-one; and
9-amino-2-cyclopentyl-5-(2-ethoxypyridin-3-yl)-2,3-dihydro-1H-pyrrolo[3,4-b]quinolin-1-one.

Chemical abbreviations used in the Examples are defined as follows: “DMSO” denotes dimethylsulfoxide, “THF” denotes tetrahydrofuran, “DMF” denotes N,N-dimethylformamide. Unless otherwise stated reaction progress is monitored by HPLC, LC-MS or TLC. Oven-dried standard laboratory glassware is used and routine manipulations were done at ambient temperature under a blanket of nitrogen unless otherwise indicated. Commercially available reagents and anhydrous solvents were typically used as received. Evaporations were typically performed under reduced pressure using a rotary evaporator. Preparative chromatography is performed using ICN silica gel 60, 32-63 μ or a suitable equivalent. Products were dried under reduced pressure at 40° C. or a suitable temperature.

HPLC-Mass Spectroscopy data were collected utilizing an Agilent Zorbax 5 μ SB-C8 column 2.1 mm×5 cm. with a column temperature of 30° C. Solvents: A=98:2 Water: Acetonitrile with 0.1% formic acid added, B=98:2 Acetonitrile: Water with 0.05% formic acid added. Flow rate 1.4 mL/min, injection volume 2.0 μL, initial conditions 5% B, eluting with a linear gradient from 5 to 90% B from time zero to 3 minutes holding at 90% B until 4 minutes. Photodiode array UV detection is used averaging signal from 210 through 400 nm. Mass Spectral data were collected using Full Scan APCI (+), base peak index, 150.0 to 900.0 amu., 30 cone volts with a probe temperature of 450° C.

¹H NMR data (δ, ppm) were obtained at 30° C. with tetramethylsilane as an internal standard set at 0.00 ppm. The multiplicities of the NMR spectra absorptions may be abbreviated by: s, singlet; br, broad peak; bs, broad singlet; d, doublet; t, triplet; q, quartet; dd, doublet of doublets; dt, doublet of triplets; m, multiplet. In many cases proton resonances associated with the quinoline 4-amino group protons were not readily observable in the proton NMR spectra recorded at 30° C. in chloroform-d due to severe broadening into the baseline. These protons may be clearly observed by recording the spectrum at −20° C.

As shown in Scheme 1, a compound 1-3 can be made by coupling of a halogenated quinoline derivative 1-1 (wherein X¹ is halo such as bromo or iodo) to a boron compound 1-2 wherein R⁶ can be an optionally substituted aryl or heteroaryl (suitable substituents can be alkyl, CN etc.), R¹⁰¹ and R¹⁰² are each, independently, hydrogen or C₁₋₆ alkyl; or R¹⁰¹ and R¹⁰², together with the two oxygen atoms to which they are attached and the boron atom to which the two oxygen atoms are attached, form a 4-7 membered heterocyclic ring whose ring-forming atoms comprises B, O and C atoms and which is optionally substituted by 1, 2, 3, or 4 C₁₋₆ alkyl (i.e., a moiety shown as 1-2B-R wherein t1 is 0, 1, 2 or 3; t2 is 0, 1, 2, 3 or 4; and R⁴⁰⁰ is each, independently, C₁₋₆ alkyl). Two examples of the boron compound 1-2 are 1-2A (a boronic acid derivative) and 1-2B (a 4,4,5,5,-tetramethyl-1,3,2-dioxoborolane derivative). The coupling reaction can be carried out in the presence of a suitable catalyst, such as a metal catalyst. Some exemplary metal catalysts include palladium catalyst, such as bis(triphenylphosphine)palladium(II) dichloride and tetrakis(triphenylphosphine)palladium(0). The coupling reaction can be carried out in the presence of a suitable base such as an inorganic base. Some suitable inorganic bases include cesium carbonate, sodium carbonate, potassium carbonate, potassium fluoride, and potassium phosphate. The coupling reaction can be carried out in a suitable solvent such as an organic solvent. Some suitable organic solvent include polar organic solvents, such as an ether or an alcohol. Suitable ethers include 1,2-dimethoxyethane and tetrahydrofuran. Suitable alcohols include ethanol, propanol and isopropanol. A suitable solvent also includes a mixture of two or more individual solvents. Suitable solvents can further contain water. The coupling reaction can be carried out at a suitable temperature to afford the compound 1-3. In some embodiments, the reaction mixture is heated to an elevated temperature (i.e., above the room temperature). In some embodiments, the reaction mixture is heated to a temperature of about 40° C., about 50° C., about 60° C., about 70° C., about 80° C., about 90° C., about 100° C., about 110° C., about 120° C., about 130° C., about 140° C., about 150° C., about 160° C. The reaction progress can be monitored by conventional methods such as TLC, LCMS or NMR.

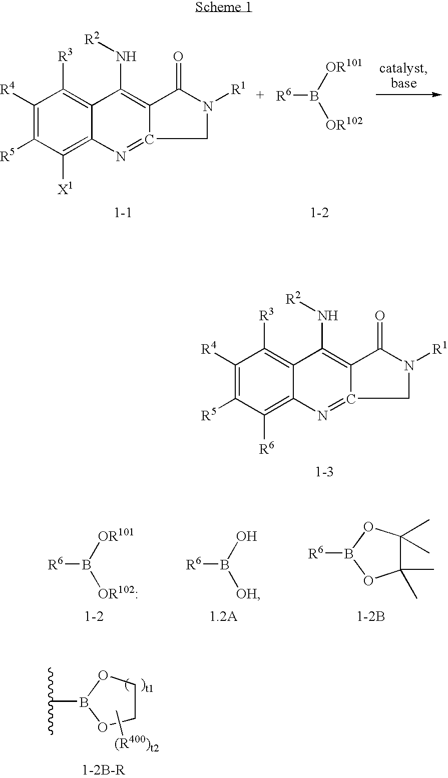

Alternatively, Compound 1-3 of Scheme 1 may be prepared, for example, by coupling Compound 1-1 with a suitable R⁶ containing precursor using the Stille reaction.

Compound 1-1 of Scheme 1 may be prepared, for example, by following the steps outlined in Scheme 2 shown below.

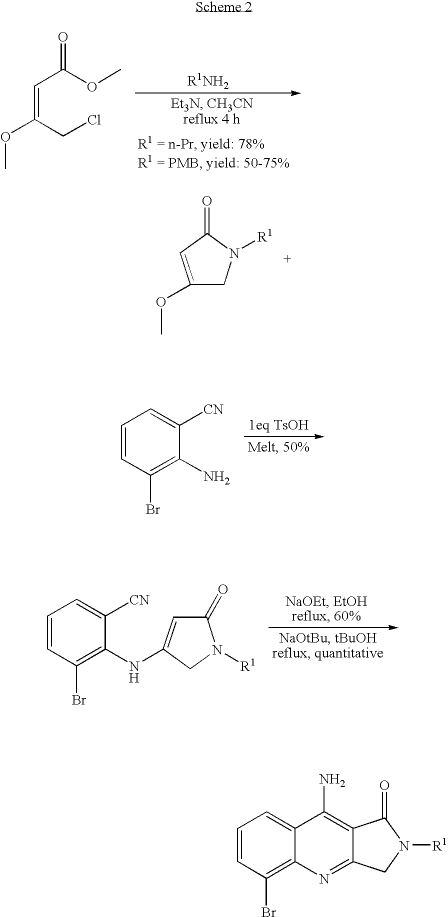

### Precursor 1

### 9-Amino-5-bromo-2-(4-methoxybenzyl)-2,3-dihydropyrrolo[3,4-b]quinolin-1-one

A solution of 3-bromo-2-[1-(4-methoxybenzyl)-5-oxo-2,5-dihydro-1H-pyrrol-3-ylamino]-benzonitrile (6.50 g, 16.3 mmol) in ethanol (110 mL) was treated with sodium ethoxide in ethanol (6.19 g of a 21% solution in 20 mL ethanol). The resulting solution was heated at 50° C. for 3 hours. The reaction was cooled to room temperature, partitioned between methylene chloride and sodium bicarbonate (saturated aqueous solution) and extracted with methylene chloride. The organic layers were dried over sodium sulfate, filtered, and concentrated under reduced pressure. The material was purified by flash chromatography on silica gel eluting with a gradient of 10 to 100% ethyl acetate in methylene chloride to afford the desired compound (3.39 g, 52%). ¹H NMR (300.132 MHz, DMSO) δ 8.40 (dd, J=8.4, 1.0 Hz, 1H), 8.08 (dd, J=7.5, 0.9 Hz, 1H), 7.82 (bs, 2H), 7.36 (t, J=7.9 Hz, 1H), 7.26 (d, J=8.6 Hz, 2H), 6.92 (dt, J=8.6, 2.6 Hz, 2H), 4.64 (s, 2H), 4.34 (s, 2H), 3.74 (s, 3H). MS APCI, m/z=398/400 (M+H). HPLC 1.62 min.

The intermediate compounds were prepared as follows:

### 3-Bromo-2-[1-(4-methoxybenzyl)-5-oxo-2,5-dihydro-1H-pyrrol-3-ylamino]-benzonitrile

The 4-methoxy-1-(4-methoxybenzyl)-1,5-dihydro-pyrrol-2-one (11.19 g, 48.0 mmol), 2-amino-3-bromobenzonitrile (11.83 g, 60.1 mmol), and p-toluene sulfonic acid (8.22 g, 43.2 mmol) were mixed together, ground to fine powder and transferred to a round-bottomed flask. The flask was placed in a preheated 130° C. oil bath and the reaction stirred for 40 minutes. The reaction mixture was removed from the bath, cooled, and dissolved in methylene chloride. The solution was washed with sodium bicarbonate (saturated aqueous solution) and the organic layer dried over sodium sulfate, filtered, and concentrated under reduced pressure to afford a brown solid (19.5 g). The crude material was purified by flash chromatography on silica gel eluting with a gradient of 20 to 40% ethyl acetate in methylene chloride to afford the desired compound (6.58 g, 34%). ¹H NMR (300.132 MHz, DMSO) δ 9.10 (s, 1H), 8.07 (dd, J=8.1, 1.3 Hz, 1H), 7.92 (dd, J=7.7, 1.2 Hz, 1H), 7.40 (t, J=8.0 Hz, 1H), 7.15 (dt, J=8.6, 2.3 Hz, 2H), 6.91 (dt, J=8.9, 2.3 Hz, 2H), 4.44-4.37 (m, 3H), 3.88 (s, 2H), 3.74 (s, 3H). MS APCI, m/z=398/400 (M+H). HPLC 1.94 min.

### 4-Methoxy-1-(4-methoxybenzyl)-1,5-dihydropyrrol-2-one

A solution of 4-methoxybenzyl amine (19.7 mL, 0.151 mol) in acetonitrile (75 mL) was heated to reflux. To this was added simultaneously a solution of (E)-4-chloro-3-methoxy-but-2-enoic acid methyl ester (20 g, 0.122 mol) in acetonitrile (85 mL) and a solution of triethylamine (15.28 mL, 0.11 mol) in acetonitrile (30 mL) over 35 minutes. After 3 hours, the reaction was cooled and allowed to stand at room temperature overnight. The resulting precipitate was removed by filtration. The mother liquor was concentrated and purified by flash chromatography on silica gel eluting with a gradient of 20 to 100% ethyl acetate in hexanes to afford the desired compound (17.04 g, 60%). ¹H NMR (300.132 MHz, DMSO) δ 7.12 (dt, J=8.9, 2.4 Hz, 2H), 6.89 (dt, J=8.8, 2.4 Hz, 2H), 5.16 (s, 1H), 4.38 (s, 2H), 3.78 (s, 2H), 3.75 (s, 3H), 3.73 (s, 3H). MS APCI, m/z=234 (M+H). HPLC 1.47 min.

### 2-Amino-3-bromobenzonitrile

The title compound was prepared as described in the literature (Campbell, J. B. Jr.; Davenport, T. W.; Syn. Comm., 19 (13&14), 2255-2263, 1989). After recrystallization from methylene chloride, the product was obtained as a shiny white solid. ¹H NMR (300.132 MHz, DMSO) δ 7.69 (dd, J=7.8, 1.4 Hz, 1H), 7.50 (dd, J=7.8, 1.4 Hz, 1H), 6.59 (t, J=7.8 Hz, 1H), 6.03 (bs, 2H). MS APCI, m/z=238/240 (M+CH₃CN). HPLC 1.81 min.

### Precursor 2

### 9-Amino-5-bromo-2-(2,5-dimethoxybenzyl)-2,3-dihydropyrrolo[3,4-b]quinolin-1-one

The title compound was prepared from 3-bromo-2-[1-(2,5-dimethoxybenzyl)-5-oxo-2,5-dihydro-1H-pyrrol-3-ylamino]-benzonitrile (3.38 g, 7.90 mmol) as described for Precursor 1 (775 mg, 23%). ¹H NMR (300.132 MHz, DMSO) δ 8.40 (dd, J=8.4, 1.0 Hz, 1H), 8.08 (dd, J=7.5, 1.0 Hz, 1H), 7.36 (t, J=7.9 Hz, 1H), 6.97 (d, J=9.0 Hz, 1H), 6.84 (dd, J=9.0, 3.0 Hz, 2H), 6.73 (d, J=3.1 Hz, 1H), 4.64 (s, 2H), 4.41 (s, 2H), 3.80 (s, 3H), 3.66 (s, 3H). MS APCI, m/z=428 (M). HPLC 1.55 min.

The intermediate compounds were prepared as follows:

### 3-Bromo-2-[1-(2,5-dimethoxybenzyl)-5-oxo-2,5-dihydro-1H-pyrrol-3-ylamino]-benzonitrile

The title compound was prepared from 1-(2,5-dimethoxybenzyl)-4-methoxy-1,5-dihydro-pyrrol-2-one (6.08 g, 23.1 mmol) and 2-amino-3-bromobenzonitrile (5.69 g, 28.9 mmol) as described for Precursor 1 (3.46 g, 35%). ¹H NMR (300.132 MHz, DMSO) δ 9.13 (s, 1H), 8.08 (dd, J=8.1, 1.3 Hz, 1H), 7.93 (dd, J=7.9, 1.2 Hz, 1H), 6.93 (t, J=8.6 Hz, 1H), 6.84-6.78 (m, 1H), 6.58 (dd, J=11.0, 3.0 Hz, 2H), 4.43-4.39 (m, 2H), 3.98 (s, 1H), 3.76 (s, 3H), 3.74 (s, 2H), 3.67 (d, J=1.3 Hz, 2H).

### 1-(2,5-Dimethoxybenzyl)-4-methoxy-1,5-dihydropyrrol-2-one

The title compound was prepared from 2,5-dimethoxybenzyl amine (9.94 mL, 65.9 mmol) and (E)-4-chloro-3-methoxy-but-2-enoic acid methyl ester (8.69 g, 52.8 mmol) as described for Precursor 1 (8.69 g, 62%). ¹H NMR (300.132 MHz, DMSO) δ 6.92 (d, J=9.0 Hz, 1H), 6.80 (dd, J=9.0, 3.1 Hz, 1H), 6.57 (d, J=3.0 Hz, 1H), 5.17 (s,1H), 4.3 (s, 2H), 3.86 (s, 2H), 3.75 (d, J=5.9 Hz, 6H), 3.67 (s, 3H). MS APCI, m/z=264 (M+H). HPLC 1.73 min.

### Precursor 3

### 9-Amino-5-bromo-2-propyl-2,3-dihydropyrrolo[3,4-b]quinolin-1-one

The title compound was prepared from 3-bromo-2-(5-oxo-1-propyl-2,5-dihydro-1H-pyrrol-3-ylamino)-benzonitrile (12.4 g, 38.73 mmol) as described for Precursor 1 (7.6 g, 61%). ¹H NMR (300.132 MHz, MeOD) δ 8.21 (dd, J=8.4, 1.1 Hz, 1H), 8.08 (dd, J=7.5, 1.2 Hz, 1H), 7.35 (t, J=8.0 Hz, 1H), 4.48 (s, 2H), 3.57 (t, J=7.2 Hz, 2H), 1.81-1.67 (m, 2H), 0.99 (t, J=7.4 Hz, 3H). MS APCI, m/z=320/322 (M+H). HPLC 1.13 min.

The intermediate compounds were prepared as follows:

### 3-Bromo-2-(5-oxo-1-propyl-2,5-dihydro-1H-pyrrol-3-ylamino)-benzonitrile

The title compound was prepared from 2-amino-3-bromobenzonitrile (16.2 g, 82.2 mmol) and 4-methoxy-1-propyl-1,5-dihydropyrrol-2-one (12.8 g, 82.5 mmol) as described for Precursor 1 (10.5 g, 40%). ¹H NMR (300.132 MHz, MeOD) δ 8.02 (dd, J=8.1, 1.3 Hz, 1H), 7.81 (dd, J=7.8, 1.3 Hz, 1H), 7.38 (t, J=8.0 Hz, 1H), 4.53 (s, 1H), 4.17 (s, 2H), 3.36 (t, J=7.2 Hz, 2H), 1.69-1.55 (m, 2H), 0.93 (t, J=7.4 Hz, 3H). MS APCI, m/z=320/322 (M+H). HPLC 1.91 min.

### 4-Methoxy-1-propyl-1,5-dihydropyrrol-2-one

The title compound was prepared from n-propyl amine (21 mL, 256.3 mmol) and (E)-4-chloro-3-methoxy-but-2-enoic acid methyl ester (31.4 g, 191.5 mmol) as described for Precursor 1 (23.2 g, 78%). ¹H NMR (300.132 MHz, CDCl3) δ 5.05 (s, 1H), 3.82 (s, 2H), 3.78 (s, 3H), 3.34 (t, J=7.3 Hz, 2H), 1.56 (sextet, J=7.4 Hz, 2H), 0.91 (t, J=7.4 Hz, 3H). MS APCI, m/z=156 (M+H). HPLC 1.42 min.

### Precursor 4

### 9-Amino-5-bromo-2-(3,4-dimethoxybenzyl)-2,3-dihydropyrrolo[3,4-b]quinolin-1-one

A white slurry of 3-bromo-2-[1-(3,4-dimethoxybenzyl)-5-oxo-2,5-dihydro-1H-pyrrol-3-ylamino]-benzonitrile (4.97 g, 11.61 mmol) in t-butanol (140 mL) was warmed to 45° C. and treated with sodium t-butoxide (1.34 g, 13.93 mmol). The resulting green solution was heated at 45° C. for 3 hours. The reaction was cooled to room temperature, partitioned between methylene chloride and water and saturated aqueous sodium bicarbonate (125 mL each) and extracted with methylene chloride (4×175 mL). The organic layers were washed with brine, dried over sodium sulfate, filtered, and concentrated under reduced pressure. The light tan solid was used without further purification (4.75 g, 95%). ¹H NMR (300.132 MHz, DMSO) δ 8.39 (dd, J=8.4, 1.0 Hz, 1H), 8.08 (dd, J=7.5, 1.0 Hz, 1H), 7.36 (t, J=7.9 Hz, 1H), 6.93 (t, J=1.9 Hz, 1H), 6.92 (d, J=4.3 Hz, 1H), 6.84 (dd, J=8.1, 1.8 Hz, 1H), 4.63 (s, 2H), 4.36 (s, 2H), 3.75 (s, 3H), 3.73 (s, 3H). APCI, m/z=429 (M+1). HPLC 1.46 min.

The intermediate compounds were prepared as follows:

### 3-Bromo-2-[1-(3,4-dimethoxybenzyl)-5-oxo-2,5-dihydro-1H-pyrrol-3-ylamino]-benzonitrile

The title compound was prepared from 1-(3,4-dimethoxybenzyl)-4-methoxy-1,5-dihydro-pyrrol-2-one (5.0 g, 19.0 mmol) and 2-amino-3-bromobenzonitrile (4.68 g, 23.0 mmol) as described for Precursor 1 (2.57 g, 32%). ¹H NMR (300.132 MHz, DMSO) δ 9.11 (s, 1H), 8.08 (d, J=8.1 Hz, 1H), 7.92 (d, J=7.8 Hz, 1H), 7.40 (t, J=7.9 Hz, 1H), 6.92 (d, J=8.1 Hz, 1H), 6.80 (d, J=1.5 Hz, 1H), 6.74 (dd, J=8.1, 1.5 Hz, 1H), 4.44 (s, 1H), 4.40 (s, 2H), 3.90 (s, 2H), 3.74 (s, 3H), 3.72 (s, 3H). MS APCI, m/z=428/430 (M+1). HPLC 1.81 min.

### 1-(3,4-Dimethoxybenzyl)-4-methoxy-1,5-dihydropyrrol-2-one

The title compound was prepared from 3,4-dimethoxybenzyl amine (25 g, 149.5 mmol) and (E)-4-chloro-3-methoxy-but-2-enoic acid methyl ester (19.69 g, 1 19.6 mmol) as described for Precursor 1 (12.75 g, 40%). ¹H NMR (300.132 MHz, DMSO) δ 6.90 (d, J=8.1 Hz, 1H), 6.80 (d, J=1.9 Hz, 1H), 6.71 (dd, J=8.2, 1.9 Hz, 1H), 5.16 (s, 1H), 4.37 (s, 2H), 3.75 (s, 3H), 3.72 (s, 6H). MS APCI, m/z=264 (M+H). HPLC 1.40 min.

### Precursor 5

### 9-Amino-2-benzo[1,3]dioxol-5-ylmethyl-5-bromo-2,3-dihydro-pyrrolo[3,4-b]quinolin-1-one

The title compound was prepared from 2-(1-benzo[1,3]dioxol-5-ylmethyl-5-oxo-2,5-dihydro-1H-pyrrol-3-yl-amino)-3-bromobenzonitrile (5.73 g, 13.9 mmol) as described for Precursor 1 (2.59 mg, 45%). ¹H NMR (300.132 MHz, DMSO) δ 8.40 (dd, J=8.4, 1.1 Hz, 1H), 8.08 (dd, J=7.4, 1.0 Hz, 1H), 7.36 (t, J=7.9 Hz, 1H), 6.90-6.87 (m, 2H), 6.81 (dd, J=7.9, 1.6 Hz, 2H), 5.99 (s, 2H), 4.61 (s, 2H), 4.36 (s, 2H). MS APCI, m/z=412/414 (M). HPLC 1.6 min.

The intermediate compounds were prepared as follows:

### 2-(1-Benzo[1,3]dioxol-5-ylmethyl-5-oxo-2,5-dihydro-1H-pyrrol-3-ylamino)-3-bromo-benzonitrile

The title compound was prepared from 1-benzo[1,3]dioxol-5-yl-methyl-4-methoxy-1,5-dihydropyrrol-2-one (6.68 g, 27.0 mmol) and 2-amino-3-bromobenzonitrile (6.66 g, 33.5 mmol) as described for Precursor 1 (5.86 g, 53%). ¹H NMR (300.132 MHz, DMSO) δ 9.12 (s, 1H), 8.08 (dd, J=8.1, 1.3 Hz, 1H), 7.93 (dd, J=7.8, 1.3 Hz, 1H), 7.41 (t, J=7.9 Hz, 1H), 6.88 (d, J=7.9 Hz, 1H), 6.77 (d, J=1.5 Hz, 1H), 6.71 (dd, J=7.9, 1.6 Hz, 1H), 6.00 (s, 2H), 4.43 (s, 1H), 4.37 (s, 2H), 3.91 (s, 2H). MS APCI, m/z=412/414 (M+H). HPLC 1.92 min.

### 1-Benzo[1,3]dioxol-5-y-lmethyl-4-methoxy-1,5-dihydropyrrol-2-one

The title compound was prepared from C-benzo[1,3]dioxol-5-yl-methylamine amine (8.42 mL, 67.6 mmol) and (E)-4-chloro-3-methoxy-but-2-enoic acid methyl ester (8.9 g, 54.1 mmol) as described for Precursor 1 (7.67 g, 57%). ¹H NMR (300.132 MHz, DMSO) δ 6.85 (d, J=7.9 Hz, 1H), 6.74 (d, J=1.4 Hz, 1H), 6.67 (dd, J=8.0, 1.3 Hz, 1H), 5.98 (s, 2H), 5.16 (s, 1H), 4.35 (s, 2H), 3.81 (s, 2H), 3.75 (s, 3H). MS APCI, m/z=248 (M+H). HPLC 1.64 min.

### Precursor 6

### 9-Amino-5-bromo-2-cyclopropyl-2,3-dihydro-pyrrolo[3,4-b]quinolin-1-one

The title compound was prepared from 3-bromo-2-(1-cyclopropyl-5-oxo-2,5-dihydro-1H-pyrrol-3-yl-amino)-benzonitrile (2.89 g, 9.09 mmol) as described for Precursor 4 (1.20 g, 42%). ¹H NMR (500.333 MHz, DMSO) δ 8.37 (d, J=8.3 Hz, 1H), 8.07 (d, J=7.5 Hz, 1H), 7.35 (t, J=7.8 Hz, 1H), 4.39 (s, 2H), 3.29 (s, 2H), 2.91 (septet, J=3.6 Hz, 1H), 0.89-0.86 (m, 2H), 0.81-0.77 (m, 2H). MS APCI, m/z=318 (M). HPLC 1.05 min.

The intermediate compounds were prepared as follows:

### 3-Bromo-2-(1-cyclopropyl-5-oxo-2,5-dihydro-1H-pyrrol-3-yl-amino)-benzonitrile

The title compound was prepared from 1-cyclopropyl-4-methoxy-1,5-dihydropyrrol-2-one (4.1 g, 26.8 mmol) and 2-amino-3-bromobenzonitrile (6.66 g, 33.5 mmol) as described for Precursor 1 (2.95 g, 35%). ¹H NMR (500.333 MHz, DMSO) δ 9.11 (s, 1H), 8.08 (dd, J=8.0, 1.2 Hz, 1H), 7.92 (dd, J=7.9, 1.2 Hz, 1H), 7.41 (t, J=8.1 Hz, 1H), 4.30 (s, 1H), 3.96 (s, 2H), 2.58 (septet, J=3.6 Hz, 1H), 0.68-0.61 (m, 4H). APCI, m/z=318/320 (M+H). HPLC 1.54 min.

### 1-Cyclopropyl-4-methoxy-1,5-dihydropyrrol-2-one

Cyclopropylamine (12.63 mL, 182.3 mmol) and triethylamine (10 mL, 76.3 mmol) were dissolved in acetonitrile (90 mL) at room temperature. A solution of (E)-4-chloro-3-methoxy-but-2-enoic acid methyl ester (10.0 g, 60.8 mmol) in acetonitrile (90 mL) was added dropwise over 40 minutes and the reaction was stirred at room temperature overnight. The mixture (dark orange solution with a white precipitate) was refluxed for 3 hours, cooled to room temperature and diluted with 10% citric acid (200 mL). The mixture was extracted with methylene chloride (3×150 mL) and the organic layers were combined, dried over magnesium sulfate, filtered, and concentrated under reduced pressure. After drying under high vacuum the title compound was obtained as a pure solid (4.13 g, 44%). ¹H NMR (500.333 MHz, DMSO) δ 5.04 (s, 1H), 3.84 (s, 2H), 3.73 (s, 3H), 2.62-2.58 (m, 1H), 0.64-0.62 (m, 4H). MS APCI, m/z=154 (M+H). HPLC 0.96 min.

### Precursor 7

### 9-Amino-5-bromo-2-cyclobutyl-2,3-dihydro-pyrrolo[3,4-b]quinolin-1-one

The title compound was prepared from 3-bromo-2-(1-cyclobutyl-5-oxo-2,5-dihydro-1H-pyrrol-3-yl-amino)-benzonitrile (4.24 g, 12.8 mmol) as described for Precursor 4 (3.52 g, 83%). ¹H NMR (500.333 MHz, DMSO) δ 8.37 (dd, J=8.5, 1.1 Hz, 1H), 8.08 (dd, J=7.3, 1.2 Hz, 1H), 7.75 (bs, 1H), 7.35 (t, J=7.9 Hz, 1H), 4.75 (quintet, J=8.7 Hz, 1H), 4.57 (s, 2H), 2.37 (quintet of doublets, J=9.5, 2.4 Hz, 2H), 2.18-2.11 (m, 2H), 1.72 (septet, J=5.3 Hz, 2H). MS APCI, m/z=332/334 (M+H). HPLC 1.26 min.

The intermediate compounds were prepared as follows:

### 3-Bromo-2-(1-cyclobutyl-5-oxo-2,5-dihydro-1H-pyrrol-3-yl-amino)-benzonitrile

The title compound was prepared from 1-cyclobutyl-4-methoxy-1,5-dihydropyrrol-2-one (3.58 g, 21.4 mmol) and 2-amino-3-bromobenzonitrile (5.28 g, 26.8 mmol) as described for Precursor 1 to give a white solid (4.24 g, 61%). ¹H NMR (500.333 MHz, DMSO) δ 9.16 (s, 1H), 8.08 (dd, J=8.0, 1.2 Hz, 1H), 7.93 (dd, J=7.9, 1.1 Hz, 1H), 7.41 (t, J=8.0 Hz, 1H), 4.49 (quintet, J=8.6 Hz, 1H), 4.33 (s, 1H), 4.10 (s, 2H), 2.18 (quintet of doublets, J=9.5, 1.8 Hz, 2H), 2.07-2.02 (m, 2H), 1.64-1.57 (m, 2H). APCI, mz=332/334 (M+H). HPLC 1.75 min.

### 1-Cyclobutyl-4-methoxy-1,5-dihydropyrrol-2-one

The title compound was prepared from cyclobutylamine (10.0 g, 140.6 mmol) and (E)-4-chloro-3-methoxy-but-2-enoic acid methyl ester (10.7 g, 65.0 mmol) as described for Precursor 6 to give an off-white solid (7.02 g, 65%). ¹H NMR (500.333 MHz, DMSO) δ 5.06 (s, 1H), 4.50 (quintet, J=8.7 Hz, 1H), 4.01 (s, 2H), 3.75 (s, 3H), 2.14 (quintet of doublets, J=9.5, 2.5 Hz, 2H), 2.05-1.98 (m, 2H), 1.62-1.56 (m, 2H). MS APCI, m/z=168 (M+H). HPLC 1.35 min.

### Precursor 8

### 9-Amino-5-bromo-2-ethyl-2,3-dihydro-pyrrolo[3,4-b]quinolin-1-one

The title compound was prepared from 3-bromo-2-(1-ethyl-5-oxo-2,5-dihydro-1H-pyrrol-3-yl-amino)-benzonitrile (2.6 g, 7.64 mmol) as described for Precursor 4 (2.30 g, 98%).

¹H NMR (300.132 MHz, CDCl₃) δ 8.07 (dd, J=7.5, 1.2 Hz, 1H), 7.81 (dd, J=8.3, 1.3 Hz, 1H), 7.32 (t, J=8.0 Hz, 1H), 4.51 (s, 2H), 3.68 (q, J=7.3 Hz, 2H), 1.30 (t, J=7.2 Hz, 3H). MS APCI, m/z=306/308 (M+H). HPLC 1.34 min.

The intermediate compounds were prepared as follows:

### 3-Bromo-2-(1-ethyl-5-oxo-2,5-dihydro-1H-pyrrol-3-yl-amino)-benzonitrile

The title compound was prepared from 4-methoxy-1-ethyl-1,5-dihydropyrrol-2-one (6.0 g, 42.5 mmol) and 2-amino-3-bromobenzonitrile (6.90 g, 35.0 mmol) as described for Precursor 1 (2.70 g, 25%). ¹H NMR (300.132 MHz, CDCl₃) δ 7.87 (dd, J=8.1, 1.4 Hz, 1H), 7.66 (dd, J=7.8, 1.4 Hz, 1H), 7.23 (t, J=8.0 Hz, 1H), 4.84 (s, 1H), 4.06 (s, 2H), 3.46 (q, J=7.3 Hz, 2H), 1.17 (t, J=7.3 Hz, 3H). MS APCI, m/z=306/308 (M+H). HPLC 2.50 min.

### 1-Ethyl-4-methoxy-1,5-dihydropyrrol-2-one

The title compound was prepared from ethylamine hydrochloride (7.43 g, 91.1 mmol) and (E)-4-chloro-3-methoxy-but-2-enoic acid methyl ester (10.0 g, 60.8 mmol) as described for Precursor 1 (6.84 g, 80%). ¹H NMR (300.132 MHz, CDCl₃) δ 5.04 (dd, J=1.7, 0.5 Hz, 1H), 3.83 (s, 2H), 3.78 (d, J=1.9 Hz, 3H), 3.43 (qd, J=7.2, 2.0 Hz, 2H), 1.14 (td, J=7.1, 2.0 Hz, 3H). GCMS, m/z=141 (M).

### Precursor 9

### 9-Amino-5-bromo-2-methyl-2,3-dihydro-pyrrolo[3,4-b]quinolin-1-one

The title compound was prepared from 3-bromo-2-(1-methyl-5-oxo-2,5-dihydro-1H-pyrrol-3-yl-amino)-benzonitrile (2.60 g, 8.93 mmol) as described for Precursor 4 (2.44 g, 94%). ¹H NMR (300.132 MHz, DMSO) δ 8.38 (dd, J=8.4, 1.2 Hz, 1H), 8.08 (dd, J=7.6, 1.0 Hz, 1H), 7.35 (t, J=7.9 Hz, 1H), 4.45 (s, 2H), 3.06 (s, 3H). MS APCI, mz=292/294 (M+H). HPLC 1.62 min.

The intermediate compounds were prepared as follows:

### 3-Bromo-2-(1-methyl-5-oxo-2,5-dihydro-1H-pyrrol-3-yl-amino)-benzonitrile

The title compound was prepared from 4-methoxy-1-methyl-1,5-dihydropyrrol-2-one (7.4 g, 58.2 mmol) and 2-amino-3-bromobenzonitrile (9.5 g, 48.5 mmol) as described for Precursor 1 (2.63 g, 19%). ¹H NMR (300.132 MHz, DMSO) δ 9.11 (s, 1H), 8.08 (dd, J=8.1, 1.3 Hz, 1H), 7.93 (dd, J=7.7, 1.3 Hz, 1H), 7.41 (t, J=7.9 Hz, 1H), 4.35 (s, 1H), 4.02 (s, 2H), 2.81 (s, 3H). APCI, m/z=292/294 (M+H). HPLC 1.68 min.

### 4-Methoxy-1-methyl-1,5-dihydropyrrol-2-one

The title compound was prepared from methylamine (100 mL of a 2M solution in THF, 200 mmol) and (E)-4-chloro-3-methoxy-but-2-enoic acid methyl ester (10.0 g, 60.8 mmol) as described for Precursor 1 (4.57 g, 59%). ¹H NMR (300.132 MHz, CDCl₃) δ 5.05 (s, 1H), 3.82 (s, 2H), 3.78 (s, 3H), 2.95 (s, 3H). MS APCI, m/z=128 (M+H). HPLC 1.15 min.

### Precursor 10

### 9-Amino -6-fluoro-5-iodo-2-propyl-2,3-dihydropyrrolo[3,4-b]quinolin-1-one

The title compound was prepared from 4-fluoro-3-iodo-2-(5-oxo-1-propyl-2,5-dihydro-1H-pyrrol-3-yl-amino)-benzonitrile (135 mg, 0.35 mmol) as described for Precursor 4 as a yellow solid. The material was used directly in the next reaction. ¹H NMR (500.333 MHz, CDCl₃) δ 7.82 (dd, J=9.1, 5.8 Hz, 1H), 7.23 (dd, J=9.1, 7.0 Hz, 1H), 6.46 (s, 2H), 4.50 (s, 2H), 3.58 (t, J=7.3 Hz, 2H), 1.72 (sextet, J=7.3 Hz, 2H), 0.99 (t, J=7.4 Hz, 3H). MS APCI, m/z=386 (M+H). HPLC 2.01 min.

The intermediate compounds were prepared as follows:

### 4-Fluoro-3-iodo-2-(5-oxo-1-propyl-2, 5-dihydro-1H-pyrrol-3-yl-amino)-benzonitrile

The title compound was prepared from 4-methoxy-1-propyl-1,5-dihydropyrrol-2-one (180 mg, 1.16 mmol) and 2-amino-4-fluoro-3-iodobenzonitrile (250 mg, 0.95 mmol) as described for Precursor 1 to give an off-white solid (138 mg, 38%). ¹H NMR (500.333 MHz, CDCl₃) δ 7.72-7.61 (m, 1H), 7.09-6.88 (m, 1H), 6.18 (s, 1H), 4.89 (s, 1H), 4.04 (s, 2H), 3.38 (t, J=7.4 Hz, 2H), 1.64-1.59 (m, 2H), 0.94 (t, J=7.4 Hz, 3H). MS APCI, m/z=386 (M+H). HPLC 2.05 min.

### 2-Amino-4-fluoro-3-iodobenzonitrile

A stirred solution of 6-fluoro-7-iodo-1H-indole-2,3-dione 3-oxime (2.65 g, 8.66 mmol) in dimethylformamide (200 mL) was heated at mild reflux (185-190° C.) for 10 hours, cooled, partitioned between equal volumes diethyl ether and water, extracted with ether (three times) and ethyl acetate (one time). The combined organic extracts were washed with half-saturated brine, dried, and concentrated. The crude product was purified on silica gel using a gradient of 0 to 50% ethyl acetate in hexanes as eluent. The final product was obtained as an off-white solid (1.70 g, 75%).

¹H NMR (500.333 MHz, CDCl₃) δ 7.39 (dd, J=8.7, 5.9 Hz, 1H), 6.48 (dd, J=8.5, 7.4 Hz, 1H), 5.08 (s, 2H).

### 6-Fluoro-7-iodo-1H-indole-2,3-dione 3-oxime

To a stirred suspension of 6-fluoro-7-iodo-1H-indole-2,3-dione (3.2 g, 11.0 mmol) in ethanol (52 mL) was added in one portion hydroxylamine hydrochloride (1.2 g, 17.3 mmol) in water (9 mL). The turbid mixture was warmed to 55° C. The initially orange colored mixture became mustard colored upon warming. The heat was removed immediately after 55° C. was obtained and the reaction was cooled and then partitioned between ethyl acetate and water. The organics were concentrated to give the final product as a yellow solid (3.3 g, 98%). ¹H NMR (500.333 MHz, MeOD) δ 8.04 (dd, J=8.2, 5.5 Hz, 1H), 6.83 (t, J=8.6 Hz, 1H).

### 6-Fluoro-7-iodo-1H-indole-2,3-dione

N-(3-Fluoro-2-iodo-phenyl)-2-[(Z)-hydroxyimino]-acetamide (3.4 g, 11.0 mmol) was added in portions over 10-15 minutes to well-stirred sulfuric acid (17 mL) preheated to 60-65° C. The reaction was heated to 80° C. over the next half hour, maintained for an additional 50 minutes, cooled to room temperature, added to crushed ice, and extracted with ethyl acetate (three times). The organics were washed, dried, and concentrated to give the final product was obtained as a yellow-orange solid (3.2 g, 99%). ¹H NMR (500.333 MHz, CDCl₃) δ 7.76 (s, 1H), 7.62 (dd, J=8.2, 5.3 Hz, 1H), 6.86 (t, J=8.2 Hz, 1H). MS APCI, m/z=292 (M+H). HPLC 1.81 min.

### N-(3-Fluoro-2-iodo-phenyl)-2-[(Z)-hydroxyimino]-acetamide

To a stirred solution-suspension of 2,2,2-trichloro-1-ethoxyethanol (0.75 g, 3.88 mmol) in water (9 mL) and concentrated hydrochloric acid (0.1 mL) at room temperature was added sodium sulfate (4.3 g, 30.3 mol), followed in several minutes by addition of a solution-suspension of 3-fluoro-2-iodo-phenylamine (0.88 g, 3.71 mmol) in water (5 mL) and concentrated hydrochloric acid (0.3 mL), hydroxylamine hydrochloride (0.83 g, 11.9 mmol), and ethanol (0.8 mL). The resulting mixture was heated at 80° C. for 3 hours during which time the turbidity increased. The cooled mixture was partitioned between water and chloroform and extracted with chloroform (three times). The organics were washed with water, dried, and concentrated to a crude solid. Trituration with 1:1 toluene/hexanes provided the pure product as a pale yellow solid (0.6 g, 52%). ¹H NMR (300.132 MHz, CDCl₃) δ 8.86 (s, 1H), 8.16 (d, J=8.3 Hz, 1H), 7.90 (s, 1H), 7.59 (s, 1H), 7.34 (td, J=8.3, 6.4 Hz, 1H), 6.90-6.83 (m, 1H). ¹H NMR F19 decoupled (300.132 MHz, CDCl3) δ 8.87 (s, 1H), 7.34 (t, J=8.4 Hz, 1H), 6.86 (d, J=8.0 Hz, 1H), 7.59 (s, 1H), 7.90 (s, 1H), 8.16 (d, J=8.4 Hz, 1H).

### 3-Fluoro-2-iodo-phenylamine

To a well stirred solution of the tin (II) chloride dihydrate (210 g, 0.93 mol) in concentrated aqueous hydrochloric acid (360 mL) at room temperature was added in portions over 1.5 hour the 2-iodo-3-fluoro-nitrobenzene (40 g, 0.15 mol). An exotherm reaching 42° C. was observed. The mixture was allowed to gradually cool to room temperature, chilled to 0° C. Sodium hydroxide (50% aqueous solution, 600 mL) was added dropwise until the reaction mixture was strongly basic. The mixture was extracted with diethyl ether (four times) and the combined organics washed with half-saturated brine, dried and concentrated to a tan colored solid which was used without further purification (32.0 g, 90%). ¹H NMR (500.333 MHz, CDCl₃) δ 7.06 (td, J=8.0, 6.3 Hz, 1H), 6.51 (d, J=8.1 Hz, 1H), 6.43 (td, J=7.9, 1.1 Hz, 1H), 4.26 (s, 2H). MS APCI, m/z=238 (M+H).

### Precursor 11

### 9-amino-2-cyclopropyl-5-fluoro-2,3-dihydro-1H-pyrrolo[3,4-b]quinolin-1-one

The 2-(1-cyclopropyl-5-oxo-2,5-dihydro-1H-pyrrol-3-ylamino)-3-fluorobenzonitrile (1.1 g, 4.28 mmol) was taken up in t-BuOH (50 mL) and gently heated to 45° C. to allow the slurry to freely stir. The sodium tert-butoxide (0.776 g, 8.07 mmol) was added portion wise at that temperature. The reaction was heated to 100° C. The solution changed from a tan slurry to a clear green solution briefly. The solution became opaque green with participate as it was allowed to reflux for one half hour. LC at this time showed complete disappearance of the starting material and one clean new peak in its place. The reaction was cooled to room temperature and poured into saturated aqueous sodium bicarbonate (50 mL). Water (50 ml) was added followed by methylene chloride (75 mL). The mixture was shaken and separated. The aqueous layer was extracted 2 more times with methylene chloride (50 ml). The organics were combined, dried over magnesium sulfate, filtered, and evaporated to a tan solid. This solid was dissolved in methanol/methylene chloride and absorbed on silica gel. The residue was purified via flash column eluting with methylene chloride/methanol. 9-Amino-2-cyclopropyl-5-fluoro-2,3-dihydro-1H-pyrrolo[3,4-b]quinolin-1-one was isolated as an off white solid (1.062 g, 4.13 mmol, 97%). ¹H NMR (500.333 MHz, DMSO) δ 8.15 (d, J=8.4 Hz, 1H), 7.78 (bs, 2H), 7.54 (m,1H), 7.41 (m, 1H), 4.36 (s, 2H), 2.90 (m, 1H), 0.83 (m, 4H). MS APCI, m/z=258 (M+H). HPLC 0.89 min.

The intermediate compounds were prepared as follows:

### 2-(1-cyclopropyl-5-oxo-2,5-dihydro-1H-pyrrol-3-ylamino)-3-fluorobenzonitrile

2-Amino-3-fluorobenzonitrile (1.1 g, 8.08 mmol) and 1-cyclopropyl-4-methoxy-1H-pyrrol-2(5H)-one (1.170 g, 7.64 mmol) were combined in acetic acid (10 mL) and heated to 80° C. Methanesulfonic acid (1.311 mL, 20.20 mmol) was dissolved in acetic acid (2 mL) and added dropwise via syringe over 15 minutes. The reaction was stirred for 1 hour at 80° C. and then cooled to RT and placed on a rotoevaporator under high vacuum for 15 minutes at 55° C. to remove the acetic acid. The resulting oil was dissolved in methylene chloride (80 mL) and slowly added dropwise over 20 minutes to a solution of saturated aqueous sodium bicarbonate (70 mL) mixed with 5 N sodium hydroxide (20 mL). This resultant biphasic system was separated. The aqueous was extracted 2 more times with methylene chloride (60 ml) and all organics were combined, dried over magnesium sulfate, and filtered. The filtrate was evacuated to produce 1.5 grams of tan solid. This solid was dissolved in methylene chloride and methanol. Silica gel was added (10 g) and solvent removed. The residue was purified via flash column eluting with ethyl acetate/methylene chloride to afford 2-(1-cyclopropyl-5-oxo-2,5-dihydro-1H-pyrrol-3-ylamino)-3-fluorobenzonitrile as an off white solid (87%). ¹H NMR (500.333 MHz, DMSO) δ 9.18 (s, 1H), 7.70 (m,1H), 7.45 (m, 1H), 4.49 (s, 1H), 3.99 (s, 2H), 2.57 (m, 1H), 0.65 (m, 4H). MS APCI, m/z=258 (M+H). HPLC 1.66 min.

### 2-amino-3-fluorobenzonitrile

To a round-bottomed flask was added (Z)-7-fluoro-3-(hydroxyimino)indolin-2-one (3.12 g, 17.32 mmol) in dimethyl formamide (150 mL) to give a brown/amber solution. The solution was heated to 180° C., which produced a steady reflux. Internal temperature was monitored to be 152° C. The reaction was heated at that temperature for 3 hours and then stirred at room temperature overnight. The reaction was diluted with water (125 mL), saturated aqueous sodium bicarbonate (125 mL) and ethyl acetate (250 mL), shaken and separated. The aqueous layers were extracted 2 more times with ethyl acetate. The organics were combined and back extracted once with an equal volume of water. The organics were then dried over magnesium sulfate, filtered and evacuated. The residue was purified via flash column eluting with methylene chloride to afford 2-amino-3-fluorobenzonitrile as a green tinted white solid (2.36 g, 17.34 mmol, 78%). ¹H NMR (500.333 MHz, DMSO) δ 7.29 (m, 2H), 6.60 (ddd, J=4.7, 7.9, 7.9 Hz, 1H), 6.09 (s, 2H). HPLC 1.20 min

### 7-fluoro-3-(hydroxyimino)indoline-2-one

7-fluoroindoline-2,3-dione (5 g, 30.28 mmol) was taken up in ethanol (70 mL). Hydroxylamine hydrochloride (3.13 g, 45.04 mmol) was added in one portion and this mixture was heated in a 105° C. oil bath. Reflux at that temperature was continued for 2.5 hours. The mixture was cooled to room temperature and poured into 5 times its volume of water. The resulting yellow precipitate was filtered and washed with water. This solid was dried at 70° C. under vacuum. Reducing the volume of filtrate in a rotoevaporator and allowing the liquor to stand overnight at room temperature formed a second crop. The resulting solid was filtered and washed with water. This second sample was dried at 70° C. under vacuum. These two crops were combined to afford the 7-fluoro-3-(hydroxyimino)indoline-2-one as a yellow solid (4.19 g, 23.26 mmol, 77% yield). ¹H NMR (500.333 MHz, DMSO) δ 13.50 (s, 1H), 11.18 (s, 1H), 7.80 (d, J=7.5 Hz, 1H), 7.29 (dd, J=9.2, 9.4 Hz, 1H), 7.04 (m, 1H). MS APCI, m/z=181 (M+H). HPLC 1.20 min.

### Precursor 12

### 9-Amino-5-bromo-2-cyclopentyl-2,3-dihydro-pyrrolo[3,4-b]quinolin-1-one

The title compound was prepared from 3-bromo-2-(1-cyclopentyl-5-oxo-2,5-dihydro-1H-pyrrol-3-yl-amino)-benzonitrile (4.36 g, 12.6 mmol) as described for Precursor 11 and obtained as an off-white solid after purification on silica gel using a gradient of 100:0 to 0:100 ethyl acetate:methylene chloride followed by trituration in methylene chloride (2.35 g, 54%). ¹H NMR (500.333 MHz, DMSO) δ 8.37 (dd, J=8.4, 1.2 Hz, 1H), 8.08 (dd, J=7.5, 1.2 Hz, 1H), 7.75 (bs, 2H), 7.35 (dd, J=8.4, 7.5 Hz, 1H), 4.58 (quintet, J=7.8 Hz, 1H), 4.47 (s, 2H), 1.92-1.81 (m, 2H), 1.81-1.67 (m, 4H), 1.66-1.56 (m, 2H). MS APCI, m/z=346/348 (M+H). HPLC 1.70 min.

The intermediate compounds were prepared as follows:

### 3-Bromo-2-(1-cyclopentyl-5-oxo-2, 5-dihydro-1H-pyrrol-3-yl-amino)-benzonitrile

To a pale yellow solution of 2-amino-3-bromobenzonitrile (2.48 g, 12.59 mmol) and methanesulfonic acid (4.1 ml, 63.14 mmol) in acetonitrile (50 mL) at reflux, was added dropwise a dark yellow solution of 1-cyclopentyl-4-methoxy-1H-pyrrol-2(5H)-one (4.56 g, 25.16 mmol) in acetonitrile (16 mL) over 1 hour. The light golden brown solution was refluxed for an additional 3 hours and then stirred at room temperature overnight. The light golden brown solution was partitioned between chloroform (100 mL), saturated sodium bicarbonate (100 mL) and water (50 mL). The aqueous layer was extracted with chloroform (3×100 mL), dried over magnesium sulfate, filtered, concentrated, and dried under high vacuum to afford the crude product as a brown oil which was carried forward without further purification (˜3.81 g, 87%). ¹H NMR (500.333 MHz, DMSO) δ 9.09 (s, 1H), 8.08 (dd, J=8.1, 1.4 Hz, 1H), 7.92 (dd, J=7.8, 1.4 Hz, 1H), 7.40 (dd, J=8.1, 7.8 Hz, 1H), 5.07 (s, 1H), 4.31 (septet, J=8.5 Hz, 1H), 3.99 (s, 2H), 1.80-1.60 (m, 4H), 1.60-1.44 (m, 4H). APCI, m/z=346/348 (M+H). HPLC 2.13 min.

### 1-Cyclopentyl-4-methoxy-1,5-dihydropyrrol-2-one

The title compound was prepared from cyclopentylamine (18.1 ml, 183.2 mmol) and (E)-4-chloro-3-methoxy-but-2-enoic acid methyl ester (10.04 g, 61.0 mmol) as described for Precursor 1 except that the triethylamine was added to the solution of cyclopropylamine in acetonitrile prior to the addition of the (E)-4-chloro-3-methoxy-but-2-enoic acid methyl ester and a gradient of 100:0 to 0:100 hexanes:ethyl acetate was used for the chromotography. The title compound was isolated as an off-white, waxy solid (8.46 g, 77%). ¹H NMR (500.333 MHz, DMSO) δ 5.07 (s, 1H), 4.31 (quintet, J=7.8 Hz, 1H), 3.89 (s, 2H), 3.75 (s, 3H), 1.77-1.69 (m, 2H), 1.69-1.59 (m, 2H), 1.56-1.44 (m, 4H). MS APCI, m/z=182.1 (M+H). HPLC 1.79 min.

### Precursor 13

### 9-Amino-5-bromo-6-fluoro-2-ethyl-2,3-dihydro-pyrrolo[3,4-b]quinolin-1-one

The title compound was prepared from 3-bromo-4-fluoro-2-(1-ethyl-5-oxo-2,5-dihydro-1H-pyrrol-3-yl-amino)-benzonitrile (650 mg, 2.01 mmol) as described for Precursor 14 (535 mg, 82.3%). ¹H NMR (500 MHz, CHLOROFORM-d) δ ppm 7.80 (dd, J=9.2, 5.6 Hz, 1 H) 7.30 (dd, J=9.1, 7.7 Hz, 1 H) 6.47 (br. s., 2 H) 4.51 (s, 2 H) 3.68 (q, J=7.2 Hz, 2 H) 1.30 (t, J=7.3 Hz, 3 H). MS APCI, m/z=324.2/326.2 (M+H). HPLC 1.52 min.

The intermediate compounds were prepared as follows:

### 3-Bromo-4-fluoro-2-(1-ethyl-5-oxo-2,5-dihydro-1H-pyrrol-3-yl-amino)-benzonitrile

The title compound was prepared from 4-methoxy-1-ethyl-1,5-dihydropyrrol-2-one (637 mg, 4.51 mmol) and 2-amino-3-bromo-4-fluorobenzonitrile (970 mg, 4.51 mmol) as described for Precursor 1 (420 mg, 28.7%). ¹H NMR (500 MHz, MeOD) δ ppm 7.86 (dd, J=8.7, 5.6 Hz, 1 H) 7.35 (t, J=8.3 Hz, 1 H) 4.61 (s, 1 H) 4.19 (s, 2 H) 3.44 (q, J=7.2 Hz, 2 H) 1.18 (t, J=7.2 Hz, 3 H). MS APCI, m/z=324.2/326.2 (M+H). HPLC 1.88 min.

### Precursor 14

### 9-Amino-5-bromo-2,3-dihydro-2-isopropylpyrrolo[3,4-b]quinolin-1-one

3-Bromo-2-(1-isopropyl-5-oxo-2,5-dihydro-1H-pyrrol-3-yl-amino)-benzonitrile (2.20 g, 6.87 mmol) was dissolved in t-BuOH (50 mL) at 65° C. Sodium tert-butoxide (1.32 g, 13.74 mmol) was added portion wise at that temperature. The reaction was stirred at 65° C. for one hour. The solution changed from a clear brown to a clear green solution briefly, and then became opaque green with participate as it was allowed to reflux for one half hour. The reaction was cooled to room temperature, quenched with water (100 mL), and the solvent (t-BuOH) was removed to obtained a white suspension. The white suspension stayed at 4° C. overnight and filtered to give an off-white solid as title compound (1.89 g, 86%). ¹H NMR (300 MHz, DMSO-d₆) δ ppm 8.38 (dd, J=8.3, 1.1 Hz, 1 H) 8.08 (dd, J=7.5, 1.2 Hz, 1 H) 7.35 (t, J=7.9 Hz, 1 H) 4.43 (s, 2H) 4.32-4.57 (m, 1 H) 1.24 (d, J=6.7 Hz, 6 H). MS APCI, m/z=320.2/322.2 (M+1). HPLC 1.45 min.

The intermediate compounds were prepared as follows:

### 3-Bromo-2-(1-isopropyl-5-oxo-2,5-dihydro-1H-pyrrol-3-yl-amino)-benzonitrile

2-Amino-3-bromobenzonitrile (1.50 g, 7.61 mmol) and methanesulfonic acid (3.90 g, 20.58 mmol) in acetic acid (10 mL) were heated to 80° C. 1-Isopropyl-4-methoxy-1,5-dihydropyrrol-2-one (3.40 g, 21.9 mmol) in acetic acid (12.5 mL) was added dropwise at the same temperature. Thirty minutes after the addition, all of acetic acid was removed from the reaction solution. The residue was diluted with methylene chloride (200 mL), washed with saturated NaHCO_{3(aq)}, dried through dried through MgSO₄, filtrated and evaporated to dry. The crude material was added to a silica gel column and was eluted with 15-100% ethyl acetate in hexane to give a tan solid as the title compound (2.00 g, 82.1%). ¹H NMR (300.132 MHz, DMSO) δ 9.11 (s, 1H), 8.08(dd, J=8.1, 1.3 Hz, 1H), 7.93 (dd, J=7.7, 1.3 Hz, 1H), 7.40 (t, J=7.9 Hz, 1H), 4.35 (s, 1H), 4.15 (septet, J=6.7 Hz, 1H) 3.95 (s, 2H), 1.11 (d, J=6.8 Hz, 6H). MS APCI, m/z=320.2/322.2 (M+H). HPLC 1.97 min.

### 4-Methoxy-1-isopropyl-1,5-dihydropyrrol-2-one

The title compound was prepared from isopropyl amine (11.49 g, 194.4 mmol) and (E)-4-chloro-3-methoxy-but-2-enoic acid methyl ester (8.0 g, 48.6 mmol) as described for Precursor 1 (7.90 g, 100%) except the reaction ran at room temperature overnight. ¹H NMR (300 MHz, CHLOROFORM-d) δ ppm 5.03 (s, 1 H) 4.45 (septet, J=6.8 Hz, 1 H) 3.78 (s, 5 H) 1.16 (d, J=6.8 Hz, 6 H). MS APCI, m/z=156.3 (M+H). HPLC 1.38 min.

### Precursor 15

### 9-Amino-5-bromo-6-fluoro-2-methyl-2,3-dihydro-pyrrolo[3,4-b]quinolin-1-one

The title compound was prepared from 3-bromo-4-fluoro-2-(1-methyl-5-oxo-2,5-dihydro-1H-pyrrol-3-yl-amino)-benzonitrile (260 mg, 0.84 mmol) as described for Precursor 14 (210 mg, 81%). ¹H NMR (300 MHz, CHLOROFORM-d) □ ppm 7.81 (dd, J=9.2, 5.6 Hz, 1 H) 7.30 (dd, J=9.2, 7.7 Hz, 1 H) 6.47 (br. s., 2 H) 4.50 (s, 2 H) 3.20 (s, 3 H). MS APCI, m/z=310.2/312.2 (M+H). HPLC 1.44 min.

The intermediate compounds were prepared as follows:

### 3-Bromo-4-fluoro-2-(1-methyl-5-oxo-2,5-dihydro-1H-pyrrol-3-yl-amino)-benzonitrile

1-Methyl-4-methoxy-1,5-dihydropyrrol-2-one (106 mg, 0.42 mmol) and 2-amino-3-bromo-4-fluorobenzonitrile (90 mg, 0.42 mmol) and methanesulfonic acid (161 mg, 1.68 mmol) in acetic acid (1 mL) were heated at 80° C. After twenty minutes, all of acetic acid was removed from the reaction solution. The residue was diluted with methylene chloride (100 mL), washed with saturated NaHCO_{3(aq)}, dried through dried through MgSO₄, filtrated and evaporated to dry. The crude material was added to a silica gel column and was eluted with 30-100% ethyl acetate in hexane to give a peach solid as the title compound (100 mg, 76.8%). ¹H NMR (300 MHz, CHLOROFORM-d) δ ppm 7.67 (dd, J=8.7, 5.6 Hz, 1 H) 7.14 (dd, J=8.7, 7.3 Hz, 1 H) 6.07 (br. s., 1 H) 4.97 (s, 1 H) 4.07 (s, 2 H) 3.00 (s, 3 H). MS APCI, m/z=310.2/312.2 (M+H). HPLC 1.75 min.

### Precursor 16 (Alternate Procedure)

### 9-amino-5-bromo-2-((1s,3s)-3-methylcyclobutyl)-2,3-dihydro-1H-pyrrolo[3,4-b]quinolin-1-one

The title compound was prepared from 3-bromo-2-(1-((1s,3s)-3-methylcyclobutyl)-5-oxo-2,5-dihydro-1H-pyrrol-3-ylamino)benzonitrile (5.15 g, 14.13 mmol) as described for Precursor 14 (4.24 g, 86.6%). ¹H NMR (300 MHz, CHLOROFORM-d) δ ppm 8.07 (dd, J=7.6, 1.3 Hz, 1 H) 7.79 (dd, J=8.2, 1.3 Hz, 1 H) 7.30 (t, J=8.0 Hz, 1 H) 6.43 (br. s., 2 H) 4.65-4.79 (m, 1 H) 4.56 (s, 2 H) 2.42-2.54 (m, 2 H) 2.08-2.24 (m, 1 H) 1.78-1.91 (m, 2 H) 1.14 (d, J=6.3 Hz, 3 H). MS APCI, m/z=346.1/348.1 HPLC 1.75 min.

The intermediate compounds were prepared as follows:

### 3-Bromo-2-(1-((1s,3s)-3-methylcyclobutyl)-5-oxo-2,5-dihydro-1H-pyrrol-3-ylamino)benzonitrile

4-Methoxy-1-((1s,3s)-3-methylcyclobutyl)-1H-pyrrol-2(5H)-one (4.20 g, 22.02 mmol) and 2-amino-3-bromobenzonitrile (5.94 g, 30.16 mmol) in acetic acid (15 mL) were warmed to 80° C. to give an amber solution. Methanesulfonic acid (3.57 mL, 55.04 mmol) in acetic acid (4 mL) was added drop wise into the reaction at 80° C. over one hour. After the addition, the reaction was stirred for another 30 minutes at 80° C. to complete. All of acetic acid was removed under high vacuum at 50° C. The residual was diluted in methylene chloride (150 mL) and then titrated into a half-saturated NaHCO₃ aqueous solution at 0° C. slowly. The organic layer was dried through MgSO₄, filtrated and evaporated to dry. The crude material was added to a silica gel column and was eluted with 0-10% methanol in methylene chloride to give an orange-yellow solid as the title compound (5.27 g, 68%) ¹H NMR(300 MHz, CHLOROFORM-d) δ ppm 7.88 (dd, J=8.1, 1.4 Hz, 1 H) 7.67 (dd, J=7.7, 1.4 Hz, 1 H) 7.23 (t, J=8.01 Hz, 1 H) 6.02 (s, 1 H) 4.85 (s, 1 H) 4.39-4.63 (m, 1 H) 4.11 (s, 2 H) 2.29-2.50 (m, 2 H) 1.90-2.20 (m, 1 H) 1.59-1.79 (m, 2 H) 1.09 (d, J=6.5 Hz, 3 H). MS APCI, m/z=346.1/348.1 HPLC 2.19 min.

### 4-Methoxy-1-((1s,3s)-3-methylcyclobutyl)-1H-pyrrol-2(5H)-one

(1s,3s)-3-Methylcyclobutanamine hydrochloride (4.98 g, 36.82 mmol) and triethylamine (12.83 mL, 92.05 mmol) were stirred in acetonitrile (50 mL) to give a white suspension at room temperature for 30 minutes. (E)-Methyl-4-chloro-3-methoxybut-2-enoate (5.05 g, 30.68 mmol) in acetonitrile (40 mL) was added drop wise. After the addition, the reaction was stirred at room temperature overnight followed by heating at 85° C. for four hours to complete. The reaction was cooled to RT, filtered out the triethylamine-HCl salt and all of the solvent was evaporated. The crude material was purified through a silica gel column using 0-10% methanol in methylene chloride to give a yellow wax-like sold (4.3 g, 77%) as the title compound. ¹H NMR (300 MHz, CHLOROFORM-d) δ ppm 5.01 (s, 1 H) 4.52 (tt, J=9.8, 7.7 Hz, 1 H) 3.86 (s, 2 H) 3.78 (s, 3 H) 2.30-2.43 (m, 2 H) 1.96-2.11 (m, 1 H) 1.56-1.68 (m, 2 H) 1.07 (d, J=6.5 Hz, 3 H). MS APCI, m/z=182.2 (M+CH₃CN). HPLC 1.88 min.

### (1s,3s)-3-Methylcyclobutanamine hydrochloride

tert-Butyl (1s,3s)-3-methylcyclobutylcarbamate (10.81 g, 55.41 mmol) was diluted in MeOH (92 mL), concentrated hydrogen chloride (23.09 mL, 277.06 mmol) was added and the reaction was stirred at room temperature overnight. Then all of the solvent was evaporated to give a brown gum. The brown gum was crystallized from ether to give an off-white needle crystal as the desired product (5.32 g, 79%). ¹H NMR (500 MHz, DMSO-d₆) δ ppm 3.45 (tt, J=8.9, 7.6 Hz, 1 H) 2.25-2.35 (m, 2 H) 2.01-2.15 (1 H) 1.65 1.75 (m, 2 H) 1.04 (d, J=6.7 Hz, 3 H).

### tert-Butyl (1s,3s)-3-methylcyclobutylcarbamate

(1s,3s)-3-Methylcyclobutanecarboxylic acid (2.99 g, 26.20 mmol), diphenyl phosphorazidate (6.23 mL, 28.81 mmol), and triethylamine (4.38 mL, 31.43 mmol) in t-BuOH (35 mL) were refluxed at 85° C. overnight. The reaction was cooled to room temperature, quenched with half saturated NaHCO_{3(aq)}, and all of t-BuOH was evaporated. The residue was extracted with ether (100 mL×3). The combined ether extracts were washed with water (100 mL×3), dried through MgSO₄ and evaporated to give a wax-like white solid (4.2 g, 91%) as the title compound. ¹H NMR (500 MHz, DMSO-d₆) δ ppm 6.95 (d, J=7.3 Hz, 1 H) 3.63-3.78 (m, 1 H) 2.18-2.33 (m, 2 H) 1.80-1.97 (m, 1 H) 1.35-1.50 (m, 2 H) 1.36 (s, 9 H) 0.98 (d, J=6.7 Hz, 3 H).

### (1s,3s)-3-Methylcyclobutanecarboxylic acid

The title compound was prepared from (E)-1-(prop-1-enyl)piperidine through 4 steps as described in the literature (Liebigs Annalen der Chemie 1990, 5, 411 & J. of Organic Chemistry 1964, 29, 801). ¹H NMR (500 MHz, BENZENE-d₆) δ ppm 2.81 (quintet, J=8.9 Hz, 1 H) 2.10-2.20 (m, 2 H) 1.96-2.07 (m, 1 H) 1.87-1.96 (m, 2 H) 0.93 (d, J=6.4 Hz, 3 H).

### (E)-1-(Prop-1-enyl)piperidine

The title compound was prepared from 1-allylpiperidine as described in the literature (J. of Molecular Catalysis A: Chemical 2005, 237, 17). ¹H NMR (300 MHz, CHLOROFORM-d) δ ppm 5.83 (dd, J=13.8, 1.4 Hz, 1 H) 4.37 (dq, J=13.7, 6.6 Hz, 1 H) 2.69-2.76 (m, 4 H) 1.62 (dd, J=6.4, 1.4 Hz, 3 H) 1.41-1.60 (m, 6 H overlapped with water peak).

### Precursor 17

### 9-amino-5-bromo-2-((1s,3s)-3-methylcyclobutyl)-2,3-dihydro-1H-pyrrolo[3,4-b]quinolin-1-one

The title compound was prepared from 3-bromo-2-(1-((1s,3s)-3-methylcyclobutyl)-5-oxo-2,5-dihydro-1H-pyrrol-3-ylamino)benzonitrile (0.774 g, 2.24 mmol) as described for Precursor 11 and obtained as an off-white solid after purification on silica gel using ethyl acetate in methylene chloride (0.442 g, 57.1%). ¹H NMR (500 MHz, DMSO-d₆) δ ppm 8.37 (dd, J=8.35, 1.34 Hz, 1 H) 8.08 (dd, J=7.48, 1.30 Hz, 1 H) 7.35(dd, J=8.05, 7.82 Hz, 1 H) 4.50-4.61 (m, 3 H) 2.27-2.38 (m, 2 H) 2.04-2.16 (m, 1 H) 1.89-2.01 (m, 2 ) 1.11 (d, J=6.56 Hz, 3 H). MS APCI, m/z=346 (M+H). HPLC 1.75 min.

The intermediate compounds were prepared as follows:

### 3-bromo-2-(1-((1s,3s)-3-methylcyclobutyl)-5-oxo-2,5-dihydro-1H-pyrrol-3-ylamino)benzonitrile

2-amino-3-bromobenzonitrile (0.548 g, 2.78 mmol) and 4-methoxy-1-((1s,3s)-3-methylcyclobutyl)-1H-pyrrol-2(5H)-one (0.605 g, 3.34 mmol) were combined in 5 ml of acetic acid and heated to 80° C. Methanesulfonic acid (1.311 mL, 20.20 mmol) was dissolved in 2 ml of acetic acid and added via syringe dropwise over 5 minutes. Continued to stir for 1.5 hours @ 80° C. Cooled to RT. Diluted with 50 ml of CH2Cl2 and added dropwise to 60 ml of saturated NaHCO3 (aqu) with 20 ml of 5 N NaOH. Cooled mixture by adding a few chunks of ice. Let stir for 15 minutes and separated. Extracted 3 times with CH2Cl2 and combined organic extracts. Dried with MgSO4, filtered and evaporated. The title compound was obtained as an off-white solid after purification on silica gel using ethyl acetate in methylene chloride (0.774 g, 80%). ¹H NMR (500 MHz, DMSO-d6) d ppm 9.12 (s, 1 H) 8.08 (dd, 1 H) 7.93 (dd, J=7.70, 0.71 Hz, 1 H) 7.41(dd, J=15.91, 0.15 Hz, 1 H) 4.24-4.36 (m, 2 H) 4.08 (s, 2 H) 2.17-2.29 (m, 2 H) 1.91-2.05 (m, 1 H) 1.66-1.81 (m, 2 H) 1.06 (d, J=6.52 Hz, 3 H). MS APCI, m/z=346 (M+H). HPLC 2.23 min.

### 4-ethoxy-1-((1s,3s)-3-methylcyclobutyl)-1H-pyrrol-2(5H)-one

3-methylcyclobutanamine, HCl (2.000 g, 16.45 mmol) was diluted with acetonitrile (22 mL) and formed a white slurry. The N,N-diisopropylethylamine (7.18 mL, 41.12 mmol) was added all at once and this mixture was allowed to stir at RT for 10 minutes. (Z)-methyl 4-chloro-3-methoxybut-2-enoate (2.256 g, 13.71 mmol) was diluted with acetonitrile (22 mL) and added to the amine mixture over 20 minutes dropwise through a syringe. Allowed to stir @ RT for 3 hours. Then heated to reflux on a timer for a total of 10 hours. Allowed to sit at RT overnight. Next day the material was absorbed directly onto silica gel and run through an Isco 80 gram column of normal phase silica gel. Eluted with ethyl acetate hexane. This crude semi-solid was separated by chiral SFC chromatography to afford the title compound as a white solid (0.656 g, 26%). ¹H NMR (500 MHz, DMSO-d₆) δ ppm 5.05 (s, 1 H) 4.25-4.37 (m, 1 H) 3.98 (s, 2H) 3.75 (s, 3 H) 2.11-2.24 (m, 2 H) 1.90-2.02 (m, 1 H) 1.63-1.76 (m, 2 H) 1.04 (d, J=6.6 Hz, 3 H). MS APCI, m/z=182 (M+H). HPLC 1.90 min.

### 3-methylcyclobutanamine HCl

In a three-necked flask equipped with a solid addition funnel and a thermometer were added 3-Methylcyclobutanecarboxylic acid (14.8 g, 0.13 mol), H₂SO₄ (40 mL) and CHCl₃ (150 mL). The solution was heated at 45-50° C. and NaN₃ (16.9 g, 0.26 mol) was added in portions at such a rate to maintain a low gas evolution (the addition was done in about 2 h). After 6 h at this temperature, the mixture was cooled to r.t. and stirred at this temperature overnight. The mixture was quenched with water (185 mL) and the aqueous phase was washed with Et₂O (caution: HN₃, this phase was treated in basic conditions for safety). The acidic aqueous phase was cooled in an ice bath then 50% NaOH was added to obtain pH=13. The product was extracted with Et₂O. The pH was checked after the first two or three extractions to maintain it near 13. The extraction was stopped when TLC indicated that no more amine was present in the aqueous phase (ninhydrin). The combined organic phases were dried with Na₂SO₄. HCl (5-6 M in isopropanol) was added to the organic phase until no more white cloud was formed then the mixture was concentrated under reduced pressure. The product was triturated in MeOH/Et₂O to give ammonium salt 6 (13.0 g, 82%) as a white solid. ¹H NMR (300 MHz, CD₃OD): δ 1.11 and 1.17 (2d, J=6.6, 6.9 Hz, 3H), 1.68-1.79 (m, 1H), 1.99-2.07 (m, 1H), 2.12-2.36 (m, 1.5H), 2.43-2.60 (m, 1.5H), 3.58 (qu, J=8.4 Hz, 0.5H), 3.85 (qu, J=7.2 Hz, 0.5H); ¹³C NMR (75 MHz, CD₃OD): δ 21.3, 21.8, 25.1, 25.4, 34.5, 36.4, 42.9, 45.3.

### 3-Methylcyclobutanecarboxylic acid

The title compound was prepared as described in the literature (Wu and Grubbs; Organic synthesis, Coll. Vol. 5, p. 273 (1973); Vol. 47, p. 28, (1967).). Distillation under reduced pressure gave the title compound, (bp=88-92° C., 10 Torr) as a colorless oil. ¹H NMR (300 MHz, CDCl₃): δ 1.05 (d, J=6.0 Hz, 1.5H), 1.11 (d, J=6.3 Hz, 1.5H), 1.82-1.92 (m, 2H), 2.26-2.54 (m, 3H), 2.91-3.03 (m, 0.5H), 3.10-3.20 (m, 0.5H).

### Precursor 18

### 9-Amino-2-(3-chloro-4-methoxybenzyl)-5-bromo 2,3-dihydropyrrolo[3,4-b]quinolin-1-one

The title compound was prepared from 3-bromo-2-(3-chloro-4-methoxybenzyl)-5-oxo-2,5-dihydro-1H-pyrrol-3-ylamino)benzonitrile (0.370 g, 0.86 mmol) as described for Precursor 11 and obtained as an off-white solid after purification on silica gel using ethyl acetate in hexane (0.356 g, 95%). ¹H NMR (500 MHz, DMSO-d₆) δ ppm 8.40 (dd, J=8.37, 1.32 Hz, 1 H) 8.08 (dd, J=7.48, 1.30 Hz, 1 H) 7.32-7.41 (m, 2 H) 7.27 (dd, J=2.17, 0.08 Hz, 1 H) 7.13 (d, J=8.58 Hz, 1 H) 4.64 (s, 2 H) 4.38 (s, 2 H) 3.83 (s, 3 H). MS APCI, m/z=432 (M+H). HPLC 1.51 min.

The intermediate compounds were prepared as follows:

### 3-bromo-2-(3-chloro-4-methoxybenzyl)-5-oxo-2,5-dihydro-1H-pyrrol-3-ylamino)benzonitrile

2-amino-3-bromobenzonitrile (0.549 g, 2.79 mmol), 3-chloro-4-methoxybenzyl-1H-pyrrol-2(5H)-one (0.595 g, 2.23 mmol) and p-toluenesulfonic acid (0.339 g, 1.78 mmol) were combined as solids in a round bottom flask. Heated to 125° C. for 30 minutes and cooled to RT, forming an amber glass. This material was taken up in methylene chloride and washed with saturated sodium bicarbonate and confirmed to still be pH=7. Separated and the aqueous was washed three more times with an equal volume of methylene chloride. All organics were combined and dried with MgSO4, filtered and evacuated. This material was purification on silica gel using ethyl acetate in methylene chloride (0.400 g, 33%). ¹H NMR (500 MHz, DMSO-d₆) δ ppm 9.14 (s, 1 H) 8.03-8.15 (m, 1 H) 7.89-7.97 (m, 1 H) 7.36-7.48 (m, 1 H) 7.26-7.33 (m, 1 H) 7.09-7.21 (m, 2 H) 4.32-4.50 (m, 3 H) 3.92 (s, 2 H) 3.81-3.88 (m, 3 H). MS APCI, m/z=432 (M+H). HPLC 1.45 min.

### 3-chloro-4-methoxybenzyl-1H-pyrrol-2(5H)-one

The title compound was prepared from 3-chloro-4-methoxybenzylamine (2.90 g, 16.90 mmol) and (E)-4-chloro-3-methoxy-but-2-enoic acid methyl ester (2.22 g, 13.5 mmol) as described for Precursor 1 except that the triethylamine was added to the solution of 3-chloro-4-methoxybenzylamine in acetonitrile prior to the addition of the (E)-4-chloro-3-methoxy-but-2-enoic acid methyl ester and a gradient of 100:0 to 0:100 hexanes:ethyl acetate was used for the chromotography. The title compound was isolated as an off-white, waxy solid (2.09 g, 58%). ¹H NMR (500 MHz, DMSO-d₆) δ ppm 7.25 (d, J=2.12 Hz, 1 H) 7.12-7.16 (m, 1 H) 7.08-7.12 (m, 1 H) 5.17 (s, 1 H) 4.38 (s, 2 H) 3.82-3.86 (m, 5 H) 3.76 (s, 3 H). MS APCI, m/z=268 (M+H). HPLC 1.95 min.

### Precursor 19

### (R)-9-amino-5-bromo-6-fluoro-2-(tetrahydrofuran-3-yl)-2,3-dihydro-1H-pyrrolo[3,4-b]quinolin-1-one

The title compound was prepared from (R)-3-bromo-4-fluoro-2-(5-oxo-1-(tetrahydrofuran-3-yl)-2,5-dihydro-1H-pyrrol-3-ylamino)benzonitrile (1.4 g, 3.8 mmol) as described for Precursor 11 and obtained as a tan solid (0.98 g, 70%). 1H NMR (300 MHz, CHLOROFORM-d) δ ppm 7.80 (dd, J=9.2, 5.6 Hz, 1 H) 7.29 (dd, J=9.2, 7.6 Hz, 1 H) 6.49 (br. s., 2 H) 5.06-5.16 (m, 1 H) 4.58 (d, J=17.6 Hz, 1 H) 4.56 (d, J=17.6 Hz, 1 H) 4.11 (td, J=8.5, 6.1 Hz, 1 H)3.80-3.96 (m, 3 H) 2.31-2.46 (m, 1 H) 1.98-2.12(m, 1 H). MS APCI, m/z=366/368. (M+H).

The intermediate compounds were prepared as follows:

### (R)-3-bromo-4-fluoro-2-(5-oxo-1-(tetrahydrofuran-3-yl)-2,5-dihydro-1H-pyrrol-3-ylamino)benzonitrile

The title compound was prepared from 2-amino-3-bromo-4-fluorobenzonitrile (0.9 g, 4.2 mmol) and (R)-4-methoxy-1-(tetrahydrofuran-3-yl)-1H-pyrrol-2(5H)-one (1.2 g, 6.7 mmol) as described for Precursor 11 and obtained as a pale green solid (1.4 g, 90%). 1H NMR (300 MHz, MeOD) δ ppm 7.86 (dd, J=8.7, 5.6 Hz, 1 H) 7.35 (dd, J=8.7, 7.9 Hz, 1 H) 4.79-4.83 (m, 1 H) 4.62 (m, 1H) 4.24 (s, 2 H) 4.05 (td, J=8.5, 5.9 Hz, 1 H) 3.74-3.86 (m, 3 H) 2.27-2.34 (m, 1 H) 1.99-2.07 (m, 1 H). MS APCI, m/z=366/368. (M+H). MS APCI, m/z=366/368. (M+H).

### (R)-4-methoxy-1-(tetrahydrofuran-3-yl)-1H-pyrrol-2(5H)-one

The title compound was prepared from (E)-methyl 4-chloro-3-methoxybut-2-enoate (3.0 g, 18.2 mmol) and R(+)-3-aminotetrahydrofuran toluene-4-sulfonate (6.0 g, 23.1 mmol) as described for Precursor 11, except that N,N-diisopropylamine (6.5 g, 50 mmol) was substituted for triethylamine, and obtained as an amber syrup (2.45 g, 73.4%). 1H NMR (300 MHz, CHLOROFORM-d) δ ppm 5.04 (s, 1 H) 4.87-4.98 (m, 1 H) 3.97-4.06 (m, 1 H) 3.89 (d, J=17.4 Hz, 1 H) 3.84 (d, J=17.4 Hz, 1 H) 3.79 (s, 3 H) 3.73-3.78 (m, 3 H) 2.20-2.35 (m, 1 H) 1.80-1.95 (m, 1 H). MS APCI, m/z=184. (M+H).

### Precursor 20

### (S)-9-amino-5-bromo-6-fluoro-2-(tetrahydrofuran-3-yl)-2,3-dihydro-1H-pyrrolo[3,4-b]quinolin-1-one

The title compound was prepared from (S)-3-bromo-4-fluoro-2-(5-oxo-1-(tetrahydrofuran-3-yl)-2,5-dihydro-1H-pyrrol-3-ylamino)benzonitrile (1.3 g, 3.5 mmol) as described for Precursor 11 and obtained as a tan solid (0.9 g, 69%). 1H NMR (300 MHz, CHLOROFORM-d) ε ppm 7.80 (dd, J=9.2, 5.6 Hz, 1 H) 7.29 (dd, J=9.2, 7.8 Hz, 1 H) 6.46 (br. s., 2 H) 5.05-5.19 (m, 1 H) 4.58 (d, J=17.6 Hz, 1 H) 4.55 (d, J=17.6 Hz, 1 H) 4.11 (td, J=8.5, 6.1 Hz, 1 H) 3.81-3.94 (m, 3 H) 2.29-2.46 (m, 1 H) 1.97-2.13 (m, 1 H). MS APCI, m/z=366/368. (M+H).

The intermediate compounds were prepared as follows:

### (S)-3-bromo-4-fluoro-2-(5-oxo-1-(tetrahydrofuran-3-yl)-2,5-dihydro-1H-pyrrol-3-ylamino)benzonitrile

The title compound was prepared from 2-amino-3-bromo-4-fluorobenzonitrile (0.9 g, 4.2 mmol) and (S)-4-methoxy-1-(tetrahydrofuran-3-yl)-1H-pyrrol-2(5H)-one (1.2 g, 6.7 mmol) as described for Precursor 11 and obtained as a pale green solid (1.3 g, 85%). 1H NMR (300 MHz, MeOD) δ ppm 7.86 (dd, J=8.7, 5.6 Hz, 1 H) 7.35 (dd, J=8.7, 7.9 Hz, 1 H) 4.78-4.83 (m, 1 H) 4.62 (s, 1 H) 4.24 (s, 2 H) 4.02-4.09 (m, 1 H) 3.74-3.85 (m, 3 H) 2.24-2.37 (m, 1 H) 1.94-2.02 (m, 1 H). MS APCI, m/z=366/368. (M+H).

### (S)-4-methoxy-1-(tetrahydrofuran-3-yl)-1H-pyrrol-2(5H)-one

The title compound was prepared from (E)-methyl 4-chloro-3-methoxybut-2-enoate (5.0 g, 30.4 mmol) and S(−)-3-aminotetrahydrofuran hydrochloride (5.0 g, 40.5 mmol) as described for Precursor 11, except that N,N-diisopropylamine (11.1 g, 86 mmol) was substituted for triethylamine, and obtained as an amber syrup (3.7 g, 66.5%). 1H NMR (300 MHz, CHLOROFORM-d) δ ppm 5.04 (s, 1 H) 4.88-4.97 (m, 1 H) 3.97-4.06 (m, 1 H) 3.92 (d, J=17.5 Hz, 1 H) 3.86 (d, J=17.5 Hz, 1 H) 3.79 (s, 3 H) 3.73-3.78 (m, 3 H) 2.20-2.34 (m, 1 H) 1.85-1.94 (m, 1 H). MS APCI, m/z=184. (M+H).

### Detailed Synthesis Methods/Procedures:

Method A: The quinoline-halide, arylboronic acid, heteroaryl boronic acid, or a boron compound 1-2 of Scheme 1 (1-4 molar equivalents), tetrakis(triphenylphosphine)palladium (0) (0.05-0.15 molar equivalents), and cesium carbonate or potassium carbonate (2.5 molar equivalents) were dissolved in a 7:2:1 mixture of 1,2-dimethoxyethane:ethanol:water (40 mL/mmol quinoline-halide) under nitrogen at ambient temperature. The resulting mixture was heated at reflux for 2-24 h. The reaction was then cooled to ambient temperature and extracted with ethyl acetate or methylene chloride. The residue from the organic extracts was purified by flash chromatography on silica gel eluting with increasingly polar gradient of ethyl acetate in hexanes or methanol in methylene chloride (for more polar compounds) to afford the desired pure compound. When necessary, compounds were further purified using Reverse Phase HPLC with a C8 column and a gradient of 20 to 90% CH₃CN:H₂O (both containing 0.1% TFA) over 30 minutes.

Method B: A solution of the quinoline-halide in 1,2-dimethoxyethane (20 mL/mmol quinoline-halide) and ethanol (6 mL/mmol quinoline-halide) under nitrogen at ambient temperature was added to a round-bottomed flask charged with FibreCat1032 (0.05-0.15 molar equivalents) and an arylboronic acid, heteroaryl boronic acid, or a boron compound 1-2 of Scheme 1 (1-4 molar equivalents). A solution of potassium carbonate (3.5 molar equivalents) in water (3 mL/mmol halide) was added. The resulting mixture was heated at reflux for 2-24 h. The reaction was then cooled to ambient temperature, filtered, and the filtrate extracted with ethyl acetate or methylene chloride. The residue from the organic extracts was purified by flash chromatography on silica gel eluting with increasingly polar gradient of ethyl acetate in hexanes or by Reverse Phase HPLC with a C8 column and a gradient of 20 to 90% CH₃CN:H₂O (both containing 0.1% TFA) over 30 minutes to afford the desired compound.

Method C: The quinoline-halide, arylboronic acid, heteroaryl boronic acid, or a boron compound 1-2 of Scheme 1 (1-4 molar equivalents), tetrakis(triphenylphosphine)palladium (0) (0.05-0.15 molar equivalents), were dissolved in tetrahydrofuran (40 mL/mmol quinoline-halide) under nitrogen at ambient temperature followed by addition of sodium carbonate (1M aqueous solution, 1-2.5 molar equivalents). The resulting mixture was heated at reflux for 2-24 h. The reaction was then cooled to ambient temperature and extracted with ethyl acetate or methylene chloride. The residue from the organic extracts was purified by flash chromatography on silica gel eluting with increasingly polar gradient of ethyl acetate in hexanes or methanol in methylene chloride (for more polar compounds) to afford the desired pure compound. When necessary, compounds were further purified using Reverse Phase HPLC with a C8 column and a gradient of 20 to 90% CH₃CN:H₂O (both containing 0.1% TFA) over 30 minutes.

Method D: The quinoline-halide, arylboronic acid, heteroaryl boronic acid, or a boron compound 1-2 of Scheme 1 (1-4 molar equivalents), tetrakis(triphenylphosphine)palladium (0) (0.05-0.15 molar equivalents), and potassium carbonate (2.5 molar equivalents) were dissolved in a 1:1:1 mixture of tetrahydrofuran:ethanol:water (20 mL/mmol quinoline-halide) under nitrogen at ambient temperature. The resulting mixture was heated at reflux for 2-24 h. The reaction was then cooled to ambient temperature and extracted with ethyl acetate, methylene chloride, or chloroform. The residue from the organic extracts was purified by flash chromatography on silica gel eluting with increasingly polar gradient of methanol in methylene chloride or methanol with ammonia in chloroform (for more polar compounds) to afford the desired pure compound. When necessary, compounds were further purified using Reverse Phase HPLC with a C8 column and a gradient of 20 to 90% CH₃CN:H₂O (both containing 0.1% TFA) over 30 minutes.

Method E: The quinoline-halide, arylstannane or heteroarylstannane (1-4 molar equivalents), tetrakis(triphenylphosphine)palladium (0) (0.10-0.15 molar equivalents), copper(I) iodide (0.10-0.15 molar equivalents) were dissolved in DMF (5 mL/mmol quinoline-halide) under nitrogen at ambient temperature. The resulting mixture was heated at 100 C for 2-24 h. The reaction was then cooled to ambient temperature, concentrated to a residue, and purified by flash chromatography on silica gel eluting with increasingly polar gradient of ethyl acetate in methylene chloride, methanol in methylene chloride, or methanol with ammonia in chloroform (for more polar compounds) to afford the desired pure compound. When necessary, compounds were further purified using Reverse Phase HPLC with a C8 column and a gradient of 20 to 90% CH₃CN:H₂O (both containing 0.1% TFA) over 30 minutes or a C18 column at pH 10 (ammonium bicarbonate) with acetonitrile/water as the mobile phase.

Method F: The quinoline-halide, arylboronic acid (typically 2-3 molar equivalents), cesium carbonate (2-3 molar equivalents) and bis(triphenylphosphine)palladium(II) dichloride (0.05 molar equivalents) were placed in a microwave reaction vessel and dissolved in 7:3:2 (v/v/v) 1,2-dimethoxyethane: water: ethanol (10 mL/mmol cinnoline-halide) at ambient temperature. The reaction vessel was capped, the head-space purged with dry nitrogen and the stirred mixture was heated on a Biotage Optimizer (300 W) microwave system maintaining a reaction temperature of 150° C. for 20-60 minutes, reaction pressures of 7 bar were typically observed. The reaction was then cooled to ambient temperature and extracted with ethyl acetate. The residue from the organic extracts was purified by flash chromatography on silica gel eluting with increasingly polar gradient of ethyl acetate in hexanes to afford the desired compound.

Method G: The quinoline-halide was taken up in 2:1:1 tetrahydrafuran:water:ethanol (12 mL/mmol quinoline-halide) and the arylboronic acid, heteroaryl boronic acid, or a boron compound 1-2 of Scheme 1 (1-4 molar equivalents), 2-dicyclohexylphosphino-2′,6′-dimethoxybiphenyl (0.05-0.15 molar equivalents), tris(dibenzylideneacetone)dipalladium (0.05-0.15 molar equivalents), and potassium phosphate (3 molar equivalents) were added respectively. The resulting mixture was heated at 90° C. for 2-24 h. The reaction was then cooled to ambient temperature, diluted with aqueous 10% sodium carbonate and extracted with ethyl acetate, methylene chloride, or chloroform. The residue from the organic extracts was purified by flash chromatography on silica gel eluting with increasingly polar gradient of methanol in methylene chloride or methanol with ammonia in chloroform (for more polar compounds) to afford the desired pure compound. When necessary, compounds were further purified using Reverse Phase HPLC with a C8 column and a gradient of 20 to 90% CH₃CN:H₂O (both containing 0.1% TFA) over 30 minutes.

Method H: The pyridyl-quinoline-fluoride was taken up in 20% sodium methoxide (50 molar equivalents) and diluted with methanol (1.5 mL/mmol pyridyl-quinoline-fluoride). Placed in a smith microwave for 20 minutes @ a temperature setting of 120° C. Let cool to RT. Taken up in methylene chloride and aqueous 10% sodium carbonate. Organics separated, combined, dried with magnesium sulfate, filtered and concentrated. The residue from the organic extracts was purified by flash chromatography on silica gel eluting with increasingly polar gradient of methanol in methylene chloride or methanol with ammonia in chloroform (for more polar compounds) to afford the desired pure compound. When necessary, compounds were further purified using Reverse Phase HPLC with a C8 column and a gradient of 20 to 90% CH₃CN:H₂O (both containing 0.1% TFA) over 30 minutes.

Method J: The quinoline-halide was taken up in THF (10 mL/mmol of quinoline-halide) and the arylboronic acid, heteroaryl boronic acid, or a boron compound 1-2 of Scheme 1 (1-4 molar equivalents), Tri-tert-butylphosphine tetrafluoroborate (0.05-0.15 molar equivalents), Tris(dibenzylideneacetone)dipalladium (0.05-0.15 molar equivalents), and Potassium fluoride (3 molar equivalents) were added respectively. Heated to 90° C. for 2-24 hours. The reaction was then cooled to ambient temperature, diluted with aqueous 10% sodium carbonate and extracted with ethyl acetate, methylene chloride, or chloroform. The residue from the organic extracts was purified by flash chromatography on silica gel eluting with increasingly polar gradient of methanol in methylene chloride or methanol with ammonia in chloroform (for more polar compounds) to afford the desired pure compound. When necessary, compounds were further purified using Reverse Phase HPLC with a C8 column and a gradient of 20 to 90% CH₃CN:H₂O (both containing 0.1% TFA) over 30 minutes.

### Intermediates:

### 2-Methoxy-4-methyl-5-(4,4,5,5-tetramethyl-[1,3,2]dioxaborolan2-yl)-pyridine

A mixture of 5-bromo-2-methoxy-4-methyl-pyridine (0.26 g, 1.29 mmol), 4,4,5,5,4′,4′,5′,5′-octamethyl-[2,2′]bi[[1,3,2]dioxaborolanyl] (0.36 g, 1.42 mmol), potassium acetate (0.39 g, 4.0 mmol), and palladium acetate (9.0 mg, 2.8 mol %) in dimethylformamide (5 mL) was heated at 90° C. for 3 hours. The reaction was allowed to cool to room temperature, filtered, filtrate concentrated to dryness to give the crude title compound which was used directly in the Suzuki coupling reaction.

### Reagent 1

### 3,6-Dimethoxy-4-(tributylstannyl)pyridazine

3,6-Dimethoxypyridazine (2.00 g, 12.42 mmol) in ether (100 mL)/THF (25 mL) was treated with n-BuLi (6.5 mL, 16.14 mmol) slowly at −75° C. After the reaction was stirred at −75° C. for twenty minutes, tributylchlorostannane (4.85 g, 14.90 mmol) was added and stirred at −75° C. for another forty-five minutes. The reaction was queched with a mixture of wet ether (50 mL)/saturated NH₄Cl (50 mL) and warmed to RT. The reaction was diluted with ether (300 mL) and washed with half-saturated NH₄Cl once. The organic layer was dried through MgSO₄, filtrated and evaporated to dry to give a yellow oil. The yellow oil was added to a silica gel column and was eluted with pure hexane to give a pale-yellow liquid (1.96 g, 36.8% yield) as the title compound. ¹H NMR (300 MHz, CHLOROFORM-d) δ ppm 7.00 (s, 1 H) 4.02 (s, 1 H) 4.00 (s, 1 H) 1.44-1.56 (m, 6 H) 1.31 (sextet, J=7.3 Hz, 6 H) 1.04-1.13 (m, 6 H) 0.88 (t, J=7.3 Hz, 9 H). MS APCI, m/z=427/429/431 (M+H). HPLC 3.88 min.

The intermediate compounds were prepared as follows:

### 3,6-Dimethoxypyridazine

3,6-Dichloropyridazine (10.0 g, 67.12 mmol) and sodium methoxide (9.79 g, 181.23 mmol) in methanol (39 mL) were heated at 70° C. overnight. The reaction was cooled to room temperature and diluted with methylene chloride (200 mL), washed with water (100 mL'2), dried through MgSO₄ and evaporated to dry to give a white solid as the title compound (9.46 g, 101% yield). The crude material was used for next step without further purification. ¹H NMR (300 MHz, CHLOROFORM-d) δ ppm 6.91 (s, 2 H) 4.05 (s, 6 H). MS APCI, m/z=182 (M+H). HPLC 1.19 min.

### Reagent 2

### 5-Methyl-2-(tributylstannyl)pyridine

2-Bromo-5-methylpyridine (2.00 g, 11.63 mmol) in ether (100 mL) was treated with n-BuLi (6.1 mL, 15.11 mmol) slowly at −75° C. After five minutes, tributylchlorostannane (4.54 g, 13.95 mmol) was added and stirred at −75° C. for another forty-five minutes. The reaction was queched with a mixture of wet ether (50 mL)/saturated NH₄Cl (50 mL), warmed to RT, diluted with ether (300 mL) and washed with half-saturated NH₄C once. The organic layer was dried through MgSO₄, filtrated and evaporated to dry to give a yellow-brown oil. The crude material was added to a silica gel column and was eluted with 0-20% ethyl acetate in hexane to give a yellow oil (1.93 g, 43.9% yield, 85% purity) as the title compound. ¹H NMR (300 MHz, CHLOROFORM-d) δ ppm 8.59 (s, 1H) 7.30-7.32 (m, 2 H) 2.28 (s, 3 H) 1.49-1.59 (m, 6 H) 1.24-1.40 (m, 6 H) 1.05-1.15 (m, 6 H) 0.87 (t, J=7.3 Hz, 9 H). MS APCI, m/z=380/382/384 (M+H). HPLC 2.96 min.

### Reagent 3

### 6-(Tributylstannyl)nicotinonitrile

6-Bromonicotinonitrile (1.00 g, 5.46 mmol), 1,1,1,2,2,2-hexabutyldistannane (4.75 g, 8.20 mmol) and tetrakis(triphenylphosphine)palladium(0) (567 mg, 0.49 mmol) were heated in 1,2-dimethoxyethane (5 mL) at 100° C. for two days. The reaction was cooled to room temperature, diluted with methylene chloride (100 mL), washed with water (100 mL×3), dried through MgSO₄ and evaporated to dry. The crude material was added to a silica gel column and was eluted with 0-20% ethyl acetate in hexane to give a yellow liquid as the title compound (220 mg, 10.33% yield, 90% purity). ¹H NMR (300 MHz, CHLOROFORM-d) δ ppm 8.95 (dd, J=2.1, 0.8 Hz, 1 H) 7.71 (dd, J=7.8, 2.1 Hz, 1 H) 7.54 (dd, J=7.6, 0.8 Hz, 1 H) 1.49-1.69 (m, 6H) 1.24-1.41 (m, 6 H) 1.09-1.20 (m, 6 H) 0.88 (t, J=7.2 Hz, 9 H). MS APCI, m/z=391/393/395 (M+H). HPLC 3.64 min.

### Reagent 4

### 5-(Trimethylstannyl)nicotinonitrile

5-Bromonicotinonitrile (1.70 g, 9.29 mmol) and 1,1,1,2,2,2-hexamethyldistannane (4.57 g, 13.93 mmol) in 1,2-dimethoxyethane (12 mL) were heated at 100° C. overnight. The reaction was cooled to room temperature, diluted with methylene chloride (100 mL), washed with water (100 mL×3), dried through MgSO₄ and evaporated to dry. The crude material was added to a silica gel column and was eluted with 0-20% ethyl acetate in hexane to give a pale-yellow liquid as the title compound (1.89 g, 76% yield). ¹H NMR (300 MHz, CHLOROFORM-d) δ ppm 8.80 (d, J=1.48 Hz, 1 H) 8.79 (d, J=2.32 Hz, 1 H) 8.02 (dd, J=2.1, 1.5 Hz, 1 H) 0.40 (s, 9 H). MS APCI, m/z=265/267/269 (M+H). HPLC 2.46 min.

### Reagent 5

### 3-Methoxy-4-(tributylstannyl)pyridazine

2,2,6,6-Tetramethylpiperidine (10.4 mL, 61.51 mmol) in ether (125 mL) was cooled to −30° C. and treated with n-BuLi (24.6 mL, 61.51 mmol). The reaction solution was warmed to room temperature for 30 minutes, and then cooled to −75° C. 3-Methoxypyridazine (3.10 g, 26.75 mmol) in ether (10 mL) was added slowly at −75° C. After ten minutes, tributylchlorostannane (10.45 g, 32.09 mmol) was added all at once and stirred at −75° C. for another forty-five minutes. The reaction was queched with a mixture of wet ether (50 mL)/saturated NH₄Cl (50 mL), warmed to RT, diluted with ether (1000 mL) and washed with half-saturated NH₄C twice The organic layer was dried through MgSO₄, filtrated and evaporated to dry to give a yellow oil. The crude material was added to a silica gel column and was eluted with 0-20% ethyl acetate in hexane to give a blue liquid (2.09 g, 19.58% yield) as the title compound. ¹H NMR (300 MHz, CHLOROFORM-d) δ ppm 8.68 (d, J=4.2 Hz, 1 H) 7.43 (d, J=4.2 Hz, 1 H) 4.09 (s, 3 H) 1.44-1.57 (m, 6 H) 1.31 (sextet, J=7.3 Hz, 6 H) 0.99-1.23 (m, 6 H) 0.88 (t, J=7.2 Hz, 8 H. MS APCI, m/z=397/399/401 (M+H). HPLC 4.04 min.

The intermediate compound was prepared as follows:

### 3-Methoxypyridazine

3-chloro-6-methoxypyridazine (3.60 g, 24.90 mmol), 10% Pd/C (1.590 g, 1.49 mmol) and ammonium formate (3.14 g, 49.81 mmol) were stirred in methanol (20 mL) at room temperature for thirty minutes. The reaction mixture was filtered through Celite to get rid of Pd/C, and the filtrate was evaporated to dry. The residue was dissolved in methylene chloride, washed with water once, dried through MgSO₄, filtrated and evaporated to dry to give a brown liquid as the title compound (2.41 g, 88% yield, 95% purity). The crude material was used for next step without further purification. ¹H NMR (300 MHz, CHLOROFORM-d) δ ppm 8.83 (dd, J=4.4, 1.3 Hz, 1 H) 7.35 (dd, J=8.9, 4.4 Hz, 1 H) 6.97 (dd, J=8.9, 1.3 Hz, 1 H) 4.14 (s, 3 H. MS APCI, m/z=152 (M+ACN+H). HPLC 0.43 min.

### Reagent 6

### 4-Methoxy-5-(tributylstannyl)pyrimidine

2,2,6,6-Tetramethylpiperidine (4.2 mL, 24.52 mmol) in ether (75mL) was cooled to −30° C. and treated with n-BuLi (9.8 mL, 24.52 mmol). The reaction solution was warmed to room temperature for 30 minutes, and then cooled to −75° C. 4-Methoxypyrimidine (1.8 g, 16.35 mmol) in ether (10 mL) was added slowly at −75° C. After ten minutes, tributylchlorostannane (6.39 g, 19.62 mmol) was added all at once and stirred at −75° C. for another forty-five minutes. The organic layer was separated from the aqueous layer, and the aqueous layer was extracted with methylene chloride (100 mL×3). The combined organic layer was dried through MgSO₄, filtrated and evaporated to dry to give a yellow oil/solid mixture. The crude material was added to a silica gel column and was eluted with 0-100% ethyl acetate in hexane to give a brown-yellow liquid as the title compound (1.95 g, 29.9%yield, 90% purity). ¹H NMR (300 MHz, CHLOROFORM-d) δ ppm 8.72 (s, 1 H) 8.36 (s, 1 H) 3.35 (s, 3 H)1.44-1.59 (m, 6 H) 1.23-1.38 (m, 6 H) 1.02-1.17 (m, 6 H) 0.88 (t, J=7.3 Hz, 9 H). MS APCI, m/z=397/399/401 (M+H). HPLC 4.12 min.

The intermediate compound was prepared as follows:

### 4-Methoxypyrimidine

5-Bromo-2-chloro-4-methoxypyrimidine (5.00 g, 22.38 mmol) and 10% Pd/C (2.381 g, 2.24 mmol) and ammonium formate (8.47 g, 134.26 mmol) were stirred in methanol (50 mL) at room temperature for three hours. The reaction mixture was filtered through Celite to get rid of Pd/C, and the filtrate was evaporated to dryness. The residue was dissolved in methylene chloride, washed with water once, dried through MgSO₄, filtrated and evaporated to dryness to give a yellow liquid as the title compound (2.25 g, 91.1%). The crude material was used as such without further purification.

¹H NMR (300 MHz, CHLOROFORM-d) δ ppm 8.79 (s, 1 H) 8.41 (d, J=5.7 Hz, 1 H) 6.73 (dd, J=5.8, 1.2 Hz, 1 H) 3.99 (s, 3 H). MS APCI, m/z=152 (M+ACN+H). HPLC 0.73 min.

### Reagent 7

### 3-Fluoro-2-(tributylstannyl)pyridine

2,2,6,6-Tetramethylpiperidine (5.21 mL, 30.90 mmol) in ether (125mL) was cooled to −30° C. and treated with n-BuLi (12.36 mL, 30.90 mmol). The reaction solution was warmed to room temperature for 30 minutes, and then cooled to −75° C. 3-Fluoropyridine (2 g, 20.60 mmol) was added slowly at −75° C. After ten minutes, tributylchlorostannane (8.05 g, 24.72 mmol) was added all at once and stirred at −75° C. for another forty-five minutes. The reaction was queched with a mixture of wet ether (50 mL)/saturated NH₄Cl (50 mL), warmed to RT, diluted with ether (300 mL) and washed with half-saturated NH₄Cl twice The organic layer was dried through MgSO₄, filtrated and evaporated to dryness to give an orange oil (10.09 g, ˜35% purity based on NMR) as the title compound along with its undesired isomer. The crude material was used for next step without further purification. (partially) ¹H NMR (300 MHz, CHLOROFORM-d) δ ppm 8.58 (ddd, J=4.4, 3.0, 1.5 Hz, 1 H) 7.04-7.23 (m, 2 H) in the aromatic region. MS APCI, m/z=384/386/388 (M+H). HPLC 3.01 min.

### Reagent 8

### 5-Fluoro-2-(tributylstannyl)benzonitrile

2-bromo-5-fluorobenzonitrile (1.5 g, 7.50 mmol) in ether (50 mL) was treated with n-BuLi (4.5 mL, 11.25 mmol) slowly at −75° C. After ten minutes, tributylchlorostannane (2.93 g, 9.00 mmol) was added and stirred at −75° C. for another forty-five minutes. The reaction was queched with a mixture of wet ether (50 mL)/saturated NH₄Cl (50 mL), warmed to RT, diluted with ether (300 mL) and washed with half-saturated NH₄Cl once. The organic layer was dried over MgSO₄, filtered and evaporated to dryness. The crude material was added to a silica gel column and was eluted with 0-20% ethyl acetate in hexane to give a light-yellow oil (3.2 g, 104% yield, 70% purity) as the title compound.

¹H NMR (300 MHz, CHLOROFORM-d) δ ppm 7.51 (dd, J=8.22, 6.32 Hz, 1H) 7.37 (dd, J=8.85, 2.53 Hz, 1H) 7.21 (dd, J=8.64, 2.53 Hz, 1H) 1.10-1.60 (m, 18 H) 0.89 (t, J=7.3 Hz, 9 H).

### Reagent 9

### 4-Fluoro-2-(tributylstannyl)benzonitrile

2-bromo-5-fluorobenzonitrile (1.5 g, 7.50 mmol) in ether (50 mL) was treated with n-BuLi (4.5 mL, 11.25 mmol) slowly at −75° C. After ten minutes, tributylchlorostannane (2.93 g, 9.00 mmol) was added and stirred at −75° C. for another forty-five minutes. The reaction was queched with a mixture of wet ether (50 mL)/saturated NH₄Cl (50 mL), warmed to RT, diluted with ether (300 mL) and washed with half-saturated NH₄Cl once. The organic layer was dried over MgSO₄, filtered and evaporated to dryness. The crude material was added to a silica gel column and was eluted with 0-20% ethyl acetate in hexane to give a light-yellow oil (3.1 g, 101% yield, 70% purity) as the title compound.

¹H NMR (300 MHz, CHLOROFORM-d) δ ppm 7.66 (dd, J=8.5, 5.0 Hz, 1 H) 7.23 (dd, J=7.8, 2.7 Hz, 0 H) 7.04 (td, J=8.4, 2.7 Hz, 1 H) 1.41-1.67 (m, 6 H) 1.19-1.41 (m, 12 H) 0.89 (t, J=7.3 Hz, 9 H).

### Reagent 10

### 5-Fluoro-2-methoxy-4-(tributylstannyl)pyridine

Diisopropylamine (1.75 g, 17.31 mmol) in ether (50 mL) was cooled to −30° C. and treated with n-BuLi (6.92 mL, 17.31 mmol). The reaction solution was warmed to room temperature for 30 minutes, and then cooled to −75° C. 5-Fluoro-2-methoxypyridine (1 g, 7.87 mmol) was added slowly at −75° C. After ten minutes, tributylchlorostannane (8.05 g, 24.72 mmol) was added all at once and stirred at −75° C. for another forty-five minutes. The reaction was queched with a mixture of wet ether (50 mL)/saturated NH₄Cl (50 mL), warmed to RT, diluted with ether (300 mL) and washed with half-saturated NH₄Cl twice. The organic layer was dried through MgSO₄, filtered and evaporated to dryness to give an orange oil (2.18 g, ˜35% purity based on NMR) as the title compound along with its undesired isomer. The crude material was used as such without further purification. (partially) ¹H NMR (300 MHz, CHLOROFORM-d) δ ppm 7.87 (m, 1H) 6.75 (m, 1H) 3.90 (s, 3 H) in the aromatic region. MS APCI, m/z=414/416/418 (M+H). HPLC 3.98 min.

### Reagent 11

### 6-Methoxy-4-(tributylstannyl)nicotinonitrile

Using the method of REAGENT 5 6-methoxynicotinonitrile (2.68 g, 20.0 mmol), 2,2,6,6-tetramethylpiperidine (4.23 g, 30.0 mmol), n-butyl lithium (18.7 ml, 30.0 mmol), and tributylchlorostannae (7.8 g, 24.0 mmol) were reacted to afford a mixture of the title compound (1.35 g, 16.0%) and 6-Methoxy-5-(tributylstannyl)nicotinonitrile (0.65 g, 7.0%) as a colorless oil which was used as such in Example 30.

### Reagent 12 Same Experimental as on Page 135

### 2-Methoxy-4-methyl-5-(4,4,5,5-tetramethyl-[1,3,2]dioxaborolan2-yl)-pyridine

A mixture of 5-bromo-2-methoxy-4-methyl-pyridine (0.26 g, 1.29 mmol), 4,4,5,5,4′,4′,5′,5′-octamethyl-[2,2′]bi[[1,3,2]dioxaborolanyl] (0.36 g, 1.42 mmol), potassium acetate (0.39 g, 4.0 mmol), and palladium acetate (9.0 mg, 2.8 mol %) in dimethylformamide (5 mL) was heated at 90° C. for 3 hours. The reaction was allowed to cool to room temperature and used directly in the Suzuki coupling reaction.

### Reagent 13

### 6-methoxy-2-methyl-3-(4,4,5,5-tetramethyl-1,3,2-dioxaborolan-2-yl)pyridine

3-bromo-6-methoxy-2-methylpyridine (10 g, 49.49 mmol), Bis(pinacolato)diboron (17.60 g, 69.29 mmol), 1,1′-Bis(diphenylphosphino)ferroccene-palldium dichloride (2.51 g, 3.46 mmol), and anhydrous potassium acetate (14.57 g, 148.48 mmol) were taken up in dioxane (120 mL) and DMSO (20 mL) which had been premixed. This mixture was heated overnight at 80° C. Initially brownish in color this mixture turned black within several minutes @ 80° C. The whole amount was diluted with water (200 mL). The aqueous layer was extracted with methylene chloride (3×200 mL), dried over magnesium sulfate, filtered, concentrated, and dried under high vacuum to afford a dark brown/black crude product. The residue was purified via flash column eluting with ethyl acetate/hexane to afford the title compound as a clear oil. ¹H NMR (500 MHz, DMSO-d₆) δ ppm 7.82 (d, J=8.28 Hz, 1 H) 6.59 (d, J=7.67 Hz, 1 H) 3.85 (s, 3 H) 2.57 (s, 3 H) 1.29 (s, 12 H).

### Reagent 14

### 2-Fluoro-3-(tributylstannyl)pyrazine

A clear solution of 2,2,6,6-tetramethylpiperidine (4.1 ml, 24.29 mmol) in tetrahydrofuran (150 mL) was cooled to −30 C and treated with n-butyl lithium (9.0 ml, 22.50 mmol). The internal temperature (IT) rose from −37 to −26 C. The reaction mixture was stirred at room temperature (IT=15 C) for 0.5 hours and then placed in a N₂(1)/MeOH bath and cooled to an internal temperature of −122 C. A solution of 2-fluoropyrazine (2.0889 g, 21.30 mmol) in tetrahydrofuran (50 ml) was added via cannula over 4 minutes (IT=103). After 5 min, the tributyltin chloride (7 ml, 25.81 mmol) was added and the mixture was maintained at −100 C for 1 hr 40 minutes. The dark brown solution was quenched with 1:4:5 35% aqueous HCl:EtOH:THF, allowed to warm to room temperature over 35 minutes, made slightly basic with sodium bicarbonate, concentrated to a residue, and then partitioned between methylene chloride and water. The aqueous layer was extracted with methylene chloride (3×150 mL). The combined organic layers were dried over magnesium sulfate, filtered, and concentrated to afford the crude product as a light brown oil which was purified on silica gel using a gradient of 100:0 to 60:40 hexanes:ethyl acetate over 35 minutes to afford the desired product as a clear oil (2.26 g, 27%).

### Reagent 15

### 2,5-Dimethoxy-3-(trimethylstannyl)pyridine

The title compound was prepared from 3-bromo-2,5-dimethoxypyridine (1.0 g, 4.6 mmol) and hexamethylditin (3.0 g, 9.15 mmol) as described for REAGENT 4 and obtained as a pale yellow oil (1.2 g, 87%). 1H NMR (300 MHz, CHLOROFORM-d) δ ppm 7.74 (d, J=3.1 Hz, 1 H) 7.28 (d, J=3.1 Hz, 1 H) 3.86 (s, 3 H) 3.80 (s, 3 H) 0.28 (s, 9 H). MS APCI, m/z=300/302/304. (M+H).

The intermediate compound was prepared as follows:

### 3-Bromo-2,5-dimethoxypyridine

A stirred mixture of 3-bromo-5-fluoro-2-methoxypyridine (2.7 g, 13.1 mmol) and a solution of sodium methoxide in MeOH (6.0 ml, 25% wt) was subjected to 130 C under microwave conditions for 50 minutes. The cooled mixture was concentrated, partitioned between water and ether, and extracted with ether. The combined organics were washed with brine, dried, and concentrated to give the title compound as a white solid (1.0 g, 35%). 1H NMR (300 MHz, CHLOROFORM-d) δ ppm 7.77 (d, J=2.7 Hz, 1 H) 7.47 (d, J=2.7 Hz, 1 H) 3.96 (s, 3 H) 3.81 (s, 3 H). MS APCI, m/z=218/220. (M+H).

### EXAMPLE 1

### 9-Amino-5-(2-fluoro-6-methoxyphenyl)-2-(4-methoxybenzyl)-2,3-dihydropyrrolo[3,4-b]quinolin-1-one

Using Method A, 9-amino-5-bromo-2-(4-methoxybenzyl)-2,3-dihydropyrrolo[3,4-b]quinolin-1-one (180 mg, 0.45 mmol) and 2-fluoro-6-methoxyphenyl boronic acid (96 mg, 0.57 mmol) were reacted to afford the title compound as a white solid (70 mg, 35%).

¹H NMR (300.132 MHz, DMSO) δ 8.39 (dd, J=8.1, 1.6 Hz, 1H), 7.69 (bs, 2H), 7.55 (dd, J=7.2, 1.5 Hz, 1H), 7.51 (q, J=7.3 Hz, 1H), 7.37 (dt, J=7.0, 8.3 Hz, 1H), 7.22 (d, J=8.6 Hz, 2H), 6.93 (d, J=8.6 Hz, 1H), 6.89 (d, J=8.7 Hz, 2H), 6.83 (t, J=8.7 Hz, 1H), 4.58 (s, 2H), 4.14 (s, 2H), 3.72 (s, 3H), 3.61 (s, 3H). MS APCI, m/z=444 (M+H). HPLC 1.77 min.

### EXAMPLE 2

### 9-Amino-5-(2,5-difluorophenyl)-2-(4-methoxybenzyl)-2,3-dihydropyrrolo[3,4-b]quinolin-1-one

Using Method A, 9-amino-5-bromo-2-(4-methoxybenzyl)-2,3-dihydropyrrolo[3,4-b]quinolin-1-one (95 mg, 0.24 mmol) and 2,5-difluorophenyl boronic acid (114 mg, 0.72 mmol) were reacted to afford the title compound as a white solid (47 mg, 45%). ¹H NMR (300.132 MHz, DMSO) δ 8.46 (d, J=7.9 Hz, 1H), 7.83 (bs, 2H), 7.69 (d, J=6.8 Hz, 1H), 7.54 (t, J=7.7 Hz, 1H), 7.26 (dd, J=17.6, 8.6 Hz, 4H), 6.90 (d, J=8.2 Hz, 2H), 4.60 (s, 2H), 4.19 (s, 2H), 3.72 (s, 3H). MS APCI, m/z=432 (M+H). HPLC 1.79 min.

### EXAMPLE 3

### 9-Amino-2-(4-methoxybenzyl)-5-(2-methoxypyridin-3-yl)-2,3-dihydropyrrolo[3,4-b]quinolin-1-one

Using Method A, 9-amino-5-bromo-2-(4-methoxybenzyl)-2,3-dihydropyrrolo[3,4-b]quinolin-1-one (200 mg, 0.50 mmol) and 2-methoxypyridine-3-boronic acid (115 mg, 0.75 mmol) were reacted to afford the title compound as a white solid (105 mg, 49%). ¹H NMR (300.132 MHz, DMSO) δ 8.38 (dd, J=8.3, 1.0 Hz, 1H), 8.17 (dd, J=5.1, 1.8 Hz, 1H), 7.70 (bs, 2H), 7.64-7.56 (m, 2H), 7.50 (t, J=7.6 Hz, 1H), 7.22 (d, J=8.5 Hz, 2H), 7.04 (dd, J=7.3, 4.9 Hz, 1H), 6.89 (d, J=8.6 Hz, 2H), 4.59 (s, 2H), 4.15 (s, 2H), 3.71 (d, J=2.6 Hz, 6H). MS APCI, m/z=427 (M+H). HPLC 1.51 min.

### EXAMPLE 4

### 9-Amino-2-(2,5-dimethoxybenzyl)-5-(2-methoxypyridin-3-yl)-2,3-dihydropyrrolo[3,4-b]quinolin-1-one

Using Method A, 9-amino-5-bromo-2-(2,5-dimethoxybenzyl)-2,3-dihydropyrrolo[3,4-b]quinolin-1-one (125 mg, 0.29 mmol) and 2-methoxypyridine-3-boronic acid (56 mg, 0.37 mmol) were reacted to afford the title compound as a white solid (98 mg, 74%). ¹H NMR (300.132 MHz, DMSO) δ 8.38 (dd, J=8.3, 1.2 Hz, 1H), 8.18 (dd, J=5.0, 1.9 Hz, 1H), 7.64-7.58 (m, 2H), 7.68 (bs, 2H), 7.50 (dd, J=8.3, 7.3 Hz, 1H), 7.05 (dd, J=7.3, 5.0 Hz, 1H), 6.94 (d, J=8.9 Hz, 1H), 6.82 (dd, J=8.9, 3.1 Hz, 1H), 6.69 (d, J=3.1 Hz, 1H), 4.60 (s, 2H), 4.21 (s, 2H), 3.76 (s, 3H), 3.72 (s, 3H), 3.65 (s, 3H). MS APCI, m/z=457 (M+H). HPLC 1.54 min.

### EXAMPLE 5

### 9-Amino-5-(2-fluoro-6-methoxyphenyl)-2-propyl-2,3-dihydropyrrolo[3,4-b]quinolin-1-one

Using Method B, 9-amino-5-bromo-2-propyl-2,3-dihydropyrrolo[3,4-b]quinolin-1-one (84.2 mg, 0.26 mmol) and 2-fluoro-6-methoxyphenyl boronic acid (157.8 mg, 0.93 mmol) were reacted to afford the title compound as a white solid (41.1 mg, 43%). ¹H NMR (300.132 MHz, DMSO) δ 8.37 (dd, J=8.1, 1.5 Hz, 1H), 7.62 (s, 2H), 7.57-7.46 (m, 2H), 7.38 (dt, J=7.0, 8.3 Hz, 1H), 6.95 (d, J=8.3 Hz, 1H), 6.86 (t, J=8.6 Hz, 1H), 4.28 (s, 2H), 3.64 (s, 3H), 3.41 (t, J=7.1 Hz, 2H), 1.59 (q, J=7.3 Hz, 2H), 0.86 (t, J=7.4 Hz, 3H). MS APCI, m/z=366.2 (M+H). HPLC 1.63 min.

### EXAMPLE 6

### 9-Amino-5-(2,3-dimethylphenyl)-2-propyl-2,3-dihydropyrrolo[3,4-b]quinolin-1-one

Using Method A, 9-amino-5-bromo-2-propyl-2,3-dihydropyrrolo[3,4-b]quinolin-1-one (53.4 mg, 0.15 mmol) and 2,3-dimethylphenyl boronic acid (57.9 mg, 0.39 mmol) were reacted to afford the title compound as a beige solid (39.2 mg, 78%). ¹H NMR (300.132 MHz, DMSO) δ 8.37 (q, J=3.4 Hz, 1H), 8.35 (q, J=3.3 Hz, 1H), 7.63 (bs, 1H), 7.52-7.47 (m, 1H), 7.16 (d, J=7.3 Hz, 1H), 7.11 (t, J=7.4 Hz, 1H), 6.98 (dd, J=7.3, 1.0 Hz, 1H), 4.27 (s, 2H), 3.41 (t, J=7.2 Hz, 2H), 2.30 (s, 3H), 1.82 (s, 3H), 1.59 (sextet, J=7.3 Hz, 2H), 0.86 (t, J=7.3 Hz, 3H). MS APCI, m/z=346 (M+H). HPLC 1.75 min.

### EXAMPLE 7

### 9-Amino-5-(3,5-dimethylphenyl)-2-propyl-2,3-dihydropyrrolo[3,4-b]quinolin-1-one

Using Method C, 9-amino-5-bromo-2-propyl-2,3-dihydropyrrolo[3,4-b]quinolin-1-one (74.2 mg, 0.20 mmol) and 3,5-dimethylphenyl boronic acid (120.9 mg, 0.81 mmol) were reacted to afford the title compound as a cream colored solid (54.8 mg, 79%). ¹H NMR (300.132 MHz, DMSO) δ 8.33 (dd, J=8.2, 0.9 Hz, 1H), 7.61 (dd, J=7.2, 1.1 Hz, 1H), 7.49 (t, J=7.6 Hz, 1H), 7.14 (s, 2H), 6.99 (s, 1H), 4.33 (s, 2H), 3.43 (t, J=7.4 Hz, 2H), 2.32 (s, 6H), 1.62 (sextet, J=7.2 Hz, 2H), 0.88 (t, J=7.3 Hz, 3H). MS APCI, m/z=346 (M+H). HPLC 1.84 min.

### EXAMPLE 8

### 9-Amino-5-(6-chloropyridin-3-yl)-2-(3,4-dimethoxybenzyl)-2,3-dihydropyrrolo[3,4-b]quinolin-1-one

Using Method A, 9-amino-5-bromo-2-(3,4-dimethoxybenzyl)-2,3-dihydropyrrolo[3,4-b]quinolin-1-one (125 mg, 0.29 mmol) and 2-chloro-5-(4,4,5,5-tetramethyl-1,3,2-dioxaborolan-2-yl)pyridine (239 mg, 0.58 mmol) were reacted to afford the title compound as a solid (40 mg, 30%). ¹H NMR (300.132 MHz, DMSO) δ 8.61 (d, J=2.4 Hz, 1H), 8.45 (dd, J=8.4, 1.0 Hz, 1H), 8.09 (dd, J=8.3, 2.5 Hz, 1H), 7.79 (dd, J=7.2, 1.0 Hz, 1H), 7.59-7.54 (m, 2H), 6.93-6.90 (m, 2H), 6.83 (dd, J=8.2, 1.8 Hz, 1H), 4.60 (s, 2H), 4.24 (s, 2H), 3.74 (s, 3H), 3.72 (s, 3H). MS APCI, m/z=461 (M+H). HPLC 1.57 min.

### EXAMPLE 9

### 9-Amino-5-(2,6-dimethoxypyridin-3-yl)-2-propyl-2,3-dihydropyrrolo[3,4-b]quinolin-1-one

Using Method D, 9-amino-5-bromo-2-propyl-2,3-dihydropyrrolo[3,4-b]quinolin-1-one (250 mg, 0.78 mmol) and 2,6-dimethoxypyridine-3-boronic acid (0.31 mg, 16.9 mmol) were reacted to afford the title compound as a white solid (205.1 mg, 69%). ¹H NMR (300.132 MHz, CDCl₃) δ 7.82 (dd, J=8.3, 1.3 Hz, 1H), 7.73 (dd, J=7.3, 1.4 Hz, 1H), 7.64 (d, J=8.1 Hz, 1H), 7.49 (dd, J=7.6, 8.2 Hz, 1H), 6.43 (d, J=8.1 Hz, 1H), 4.33 (s, 2H), 3.99 (s, 3H), 3.88 (s, 3H), 3.55 (t, J=7.3 Hz, 2H), 1.68 (sextet, J=7.3 Hz, 2H), 0.97 (t, J=7.4 Hz, 3H). MS APCI, m/z=379 (M+H). HPLC 1.93 min.

### EXAMPLE 10

### 9-Amino-5-(6-methylpyridin-3-yl)-2-propyl-2,3-dihydropyrrolo[3,4-b]quinolin-1-one

Using Method D, 9-amino-5-bromo-2-propyl-2,3-dihydropyrrolo[3,4-b]quinolin-1-one (76.0 mg, 0.24 mmol) and 6-methylpyridine-3-boronic acid mono hydrate (98.0 mg, 0.63 mmol) were reacted to afford the title compound as a white solid (71.8 mg, 91%). ¹H NMR (300.132 MHz, CDCl₃) δ 8.82 (d, J=2.0 Hz, 1H), 7.96 (dd, J=8.0, 2.2 Hz, 1H), 7.87 (dd, J=8.4, 1.4 Hz, 1H), 7.73 (dd, J=7.2, 1.4 Hz, 1H), 7.54 (dd, J=7.2, 8.3 Hz, 1H), 7.28 (s, 1H), 6.42 (bs, 2H), 4.36 (s, 2H), 3.56 (t, J=7.2 Hz, 2H), 2.65 (s, 3H), 1.70 (sextet, J=7.3 Hz, 2H), 0.98 (t, J=7.4 Hz, 3H). MS APCI, m/z=333 (M+H). HPLC 1.16 min.

### EXAMPLE 11

### 9-Amino-2-(3,4-dimethoxybenzyl)-5-(2,5-dimethoxyphenyl)-2,3-dihydropyrrolo[3,4-b]quinolin-1-one

Using Method A, 9-amino-5-bromo-2-(3,4-dimethoxybenzyl)-2,3-dihydropyrrolo[3,4-b]quinolin-1-one (205 mg, 0.48 mmol) and 2,5-dimethoxyphenyl boronic acid (109 mg, 0.60 mmol) were reacted to afford the title compound as a solid (100 mg, 43%). ¹H NMR (300.132 MHz, DMSO) δ 8.34 (dd, J=8.2, 1.5 Hz, 1H), 7.65 (bs, 1H), 7.55 (dd, J=7.3, 1.4 Hz, 1H), 7.47 (t, J=7.6 Hz, 1H), 6.99 (d, J=9.0 Hz, 1H), 6.92-6.87 (m, 3), 6.81 (dd, J=8.1, 1.8 Hz, 1H), 6.74 (d, J=3.1 Hz, 1H), 4.58 (s, 2H), 4.18 (s, 2H), 3.72 (s, 3H), 3.71 (s, 3H), 3.70 (s, 3H), 3.53 (s, 3H). MS APCI, m/z=486 (M+H). HPLC 1.69 min.

### EXAMPLE 12

### 9-Amino-5-(6-methoxy-4-methylpyridin-3-yl)-2-propyl-2,3-dihydropyrrolo[3,4b]quinolin-1-one

Using Method D, 9-amino-5-bromo-2-propyl-2,3-dihydropyrrolo[3,4-b]quinolin-1-one (80 mg, 0.25 mmol) and 2-methoxy-4-methyl-5-(4,4,5,5-tetramethyl-[1,3,2]dioxaborolan-2-yl)-pyridine (160 mg, 0.64 mmol) were reacted to afford the title compound as a white solid (47.6 mg, 53%). ¹H NMR (300.132 MHz, CDCl₃) δ 8.03 (s, 1H), 7.89 (dd, J=8.2, 1.6 Hz, 1H), 7.60-7.50 (m, 2H), 6.70 (s, 1H), 6.42 (bs, 1H), 4.33 (s, 2H), 3.98 (s, 3H), 3.54 (t, J=7.3 Hz, 2H), 2.03 (s, 3H), 1.68 (sextet, J=7.3 Hz, 2H), 0.97 (t, J=7.4 Hz, 3H). MS APCI, m/z=363 (M+H). HPLC 1.78 min.

### EXAMPLE 13

### 9-Amino-5-(2-fluoropyridin-3-yl)-2-propyl-2,3-dihydropyrrolo[3,4b]quinolin-1-one

Using Method D, 9-amino-5-bromo-2-propyl-2,3-dihydropyrrolo[3,4-b]quinolin-1-one (76 mg, 0.24 mmol) and 2-fluoropyridine-3-boronic acid (95 mg, 0.67 mmol) were reacted to afford the title compound as a white solid (47.4 mg, 60%). ¹H NMR (300.132 MHz, CDCl₃) δ 8.27 (dq, J=4.8, 1.0 Hz, 1H), 7.96 (d, J=1.9 Hz, 1H), 7.93 (t, J=1.6 Hz, 1H), 7.91 (d, J=1.3 Hz, 1H), 7.73 (dt, J=7.1, 1.1 Hz, 1H), 7.60-7.26 (m, 1H), 6.42 (s, 2H), 4.33 (s, 2H), 3.55 (t, J=7.2 Hz, 2H), 1.69 (sextet, J=7.4 Hz, 2H), 0.97 (t, J=7.4 Hz, 3H). MS APCI, m/z=337 (M+H). HPLC 1.60 min.

### EXAMPLE 14

### 9-Amino-2-benzo[1,3]dioxol-5-yl-methyl-5-(2-methoxy-5-methylphenyl)-2,3-dihydropyrrolo[3,4-b]quinolin-1-one

Using Method A, 9-amino-2-benzo[1,3]dioxol-5-yl-methyl-5-bromo-2,3-dihydro-pyrrolo[3,4-b]quinolin-1-one (135 mg, 0.33 mmol) and 2-methoxy-5-methylphenyl boronic acid (68 mg, 0.41 mmol) were reacted to afford the title compound as a solid (90 mg, 61%). ¹H NMR (300.132 MHz, DMSO) δ 8.33 (dd, J=8.1, 1.7 Hz, 1H), 7.64 (bs, 1H), 7.52 (dd, J=7.1, 1.7 Hz, 1H), 7.47 (q, J=7.7 Hz, 1H), 7.12 (dd, J=8.3, 2.0 Hz, 1H), 6.96-6.84 (m, 4H), 6.77 (dd, J=7.8, 1.3 Hz, 1H), 5.97 (s, 2H), 4.56 (s, 2H), 4.16 (s, 2H), 3.55 (s, 3H), 2.25 (s, 3H). MS APCI, m/z=454 (M+H). HPLC 1.86 min.

### EXAMPLE 15

### 9-Amino-5-(2-chloro-6-methylpyridin-3-yl)-2-propyl-2,3-dihydropyrrolo[3,4-b]quinolin-1-one

Using Method D, 9-amino-5-bromo-2-propyl-2,3-dihydropyrrolo[3,4-b]quinolin-1-one (75 mg, 0.23 mmol) and 2-chloro-6-methylpyridine-3-boronic acid (170 mg, 0.99 mmol) were reacted to afford the title compound as a white solid (73.6 mg, 86%). ¹H NMR (300.132 MHz, CDCl₃) δ 7.91 (dd, J=8.3, 1.5 Hz, 1H), 7.66 (dd, J=7.2, 1.5 Hz, 1H), 7.63 (d, J=7.6 Hz, 1H), 7.53 (dd, J=8.0, 7.2 Hz, 1H), 7.19 (d, J=7.6 Hz, 1H), 6.42 (bs, 1H), 4.31 (s, 2H), 3.54 (t, J=7.2 Hz, 2H), 2.63 (s, 3H), 1.68 (sextet, J=7.4 Hz, 2H), 0.97 (t, J=7.4 Hz, 3H). MS APCI, m/z=367 (M+H). HPLC 1.73 min.

### EXAMPLE 16

### 9-Amino-2-cyclopropyl-5-(2-fluoro-6-methoxyphenyl)-2,3-dihydropyrrolo[3,4-b]quinolin-1-one

Using Method A, 9-amino-5-bromo-2-cyclopropyl-2,3-dihydro-pyrrolo[3,4-b]quinolin-1-one (159 mg, 0.50 mmol) and 2-fluoro-6-methoxyphenyl boronic acid (170 mg, 1.0 mmol) were reacted to afford the title compound as a white solid (100 mg, 55%). ¹H NMR (500.333 MHz, DMSO) δ 8.36 (dd, J=8.3, 1.2 Hz, 1H), 7.63 (bs, 1H), 7.54 (dd, J=7.0, 1.2 Hz, 1H), 7.49 (t, J=7.6 Hz, 1H), 7.38 (dt, J=7.4, 8.3 Hz, 1H), 6.95 (d, J=8.5 Hz, 1H), 6.85 (t, J=8.6 Hz, 1H), 4.21 (d, J=1.4 Hz, 2H), 3.63 (s, 3H), 2.88 (septet, J=3.8 Hz, 1H), 0.82 (q, J=3.3 Hz, 2H), 0.76-0.72 (m, 2H). MS APCI, m/z=364 (M+H). HPLC 1.42 min.

### EXAMPLE 17

### 9-Amino-2-ethyl-5-(2-fluoro-6-methoxyphenyl)-2,3-dihydro-pyrrolo[3,4-b]quinolin-1-one

Using Method A, 9-amino-5-bromo-2-ethyl-2,3-dihydropyrrolo[3,4-b]quinolin-1-one (249 mg, 0.81 mmol) and 2-fluoro-6-methoxyphenyl boronic acid (414 mg, 2.43 mmol) were reacted to afford the title compound as a solid (174 mg, 61%). ¹H NMR (300.132 MHz, CDCl₃) δ 7.87 (dd, J=8.3, 1.2 Hz, 1H), 7.66 (dd, J=7.0, 1.2 Hz, 1H), 7.52 (dd, J=8.1, 7.4 Hz, 1H), 7.34 (dt, J=6.8, 8.2 Hz, 1H), 6.85-6.79 (m, 2H), 6.35 (bs, 2H), 4.31 (d, J=4.1 Hz, 2H), 3.70 (s, 3H), 3.62 (dq, J=4.3, 7.2 Hz, 2H), 1.24 (t, J=7.2 Hz, 3H). MS APCI, m/z=352 (M+H). HPLC 1.65 min.

### EXAMPLE 18

### 9-Amino-2-cyclobutyl-5-(2-fluoro-6-methoxyphenyl)-2,3-dihydropyrrolo[3,4-b]quinolin-1-one

Using Method A, 9-amino-5-bromo-2-cyclobutyl-2,3-dihydropyrrolo[3,4-b]quinolin-1-one (200 mg, 0.60 mmol) and 2-fluoro-6-methoxyphenyl boronic acid (204 mg, 1.20 mmol) were reacted to afford the title compound as a solid (149 mg, 66%). ¹H NMR (500.333 MHz, DMSO) δ 8.37 (dd, J=8.3, 1.2 Hz, 1H), 7.63 (bs, 1H), 7.55 (dd, J=6.9, 1.2 Hz, 1H), 7.49 (dd, J=8.3, 6.9 Hz, 1H), 7.39 (dt, J=6.8, 8.5 Hz, 1H), 6.96 (d, J=8.4 Hz, 1H), 6.86 (t, J=8.6 Hz, 1H), 4.72 (quintet, J=8.6 Hz, 1H), 4.39 (d, J=2.5 Hz, 2H), 3.64 (s, 3H), 2.31 (sextet of triplets, J=9.6, 2.0 Hz, 2H), 2.14-2.06 (m, 2H), 1.73-1.62 (m, 2H). MS APCI, m/z=378 (M+H). HPLC 1.50 min.

### EXAMPLE 19

### 9-Amino-2-ethyl-5-(2-methoxypyridin-3-yl)-2,3-dihydropyrrolo[3,4-b]quinolin-1-one

Using Method A, 9-amino-5-bromo-2-ethyl-2,3-dihydropyrrolo[3,4-b]quinolin-1-one (200 mg, 0.65 mmol) and 2-methoxypyridine-3-boronic acid (220 mg, 1.44 mmol) were reacted to afford the title compound as a beige solid (165 mg, 76%). ¹H NMR (300.132 MHz, DMSO) δ 8.36 (dd, J=8.3, 1.3 Hz, 1H), 8.19 (dd, J=5.1, 1.9 Hz, 1H), 7.64-7.58 (m, 3H), 7.49 (dd, J=8.0, 7.1 Hz, 1H), 7.07 (dd, J=7.5, 5.0 Hz, 1H), 4.29 (s, 2H), 3.74 (s, 3H), 3.49 (q, J=7.1 Hz, 2H), 1.15 (t, J=7.3 Hz, 3H). MS APCI, m/z=335 (M+H). HPLC 1.50 min.

### EXAMPLE 20

### 9-Amino-5-(2-fluoro-6-methoxy-phenyl)-2-methyl-2,3-dihydropyrrolo[3,4-b]quinolin-1-one

Using Method A, 9-amino-5-bromo-2-methyl-2,3-dihydropyrrolo[3,4-b]quinolin-1-one (250 mg, 0.86 mmol) and 2-fluoro-6-methoxyphenyl boronic acid (436.5 mg, 2.57 mmol) were reacted to afford the title compound as a solid (185 mg, 64%). ¹H NMR (300.132 MHz, CDCl₃) δ 7.87 (dd, J=8.3, 1.2 Hz, 1H), 7.66 (dd, J=7.0, 1.3 Hz,1H), 7.52 (dd, J=8.4, 7.4 Hz, 1H), 7.34 (dt, J=6.6, 8.3 Hz, 1H), 6.87-6.78 (m, 2H), 6.35 (bs, 2H), 4.29 (d, J=2.4 Hz, 2H), 3.70 (s, 3H), 3.14 (s, 3H). MS APCI, m/z=338 (M+H). HPLC 1.56 min.

### EXAMPLE 21

### 9-Amino-2-cyclopropyl-5-(2,5-dimethoxyphenyl)-2,3-dihydropyrrolo[3,4-b]quinolin-1-one

Using Method A, 9-amino-5-bromo-2-cyclopropyl-2,3-dihydro-pyrrolo[3,4-b]quinolin-1-one (200 mg, 0.62 mmol) and 2,5-dimethoxyphenyl boronic acid (229 mg, 1.26 mmol) were reacted to afford the title compound as a solid (125 mg, 53%). ¹H NMR (500.333 MHz, DMSO) δ 8.31 (dd, J=8.3, 1.0 Hz, 1H), 7.60 (bs, 1H), 7.54 (dd, J=7.1, 1.3 Hz, 1H), 7.46 (dd, J=8.4, 7.4 Hz, 1H), 7.00 (d, J=9.1 Hz, 1H), 6.90 (dd, J=8.7, 3.0 Hz, 1H), 6.76 (d, J=3.0 Hz, 1H), 4.21 (s, 2H), 3.72 (s, 3H), 3.55 (s, 3H), 2.89 (septet, J=3.7 Hz, 1H), 0.84-0.73 (m, 4H). MS APCI, m/z=376 (M+H). HPLC 1.50 min.

### EXAMPLE 22

### 9-Amino-2-cyclopropyl-5-(2-fluoro-3-methoxyphenyl)-2,3-dihydropyrrolo[3,4-b]quinolin-1-one

Using Method A, 9-amino-5-bromo-2-cyclopropyl-2,3-dihydro-pyrrolo[3,4-b]quinolin-1-one (200 mg, 0.63 mmol) and 2-fluoro-3-methoxyphenyl boronic acid (214 mg, 1.26 mmol) were reacted to afford the title compound as a solid (165 mg, 72%). ¹H NMR (500.333 MHz, DMSO) δ 8.40 (dd, J=8.4, 0.9 Hz, 1H), 7.68 (bs, 1H), 7.62 (dd, J=7.3, 0.9 Hz, 1H), 7.51 (dd, J=8.6, 7.4 Hz, 1H), 7.20-7.14 (m, 2H), 6.95-6.88 (m, 1H), 4.24 (s, 2H), 3.88 (s, 3H), 2.89 (septet, J=3.7 Hz, 1H), 0.86-0.81 (m, 2H), 0.77-0.72 (m, 2H). MS APCI, m/z=364 (M+H). HPLC 1.41 min.

### EXAMPLE 23

### 9-Amino-5-(2-chloro-6-methylpyridin-3-yl)-2-(3,4-dimethoxybenzyl)-2,3-dihydropyrrolo[3,4-b]quinolin-1-one

Using Method A, 9-amino-5-bromo-2-(3,4-dimethoxybenzyl)-2,3-dihydropyrrolo[3,4-b]quinolin-1-one (150 mg, 0.43 mmol) and 2-chloro-6-methylpyridine-3-boronic acid (111 mg, 0.65 mmol) were reacted to afford the title compound as a solid (69 mg, 34%).

¹H NMR (500.333 MHz, DMSO) δ 8.44 (dd, J=8.4, 1.2 Hz, 1H), 7.75 (bs, 1H), 7.67 (d, J=7.7 Hz, 1H), 7.63 (dd, J=7.1, 1.1 Hz, 1H), 7.53 (dd, J=8.3, 7.1 Hz, 1H), 7.32 (d, J=7.6 Hz, 1H), 6.91 (d, J=2.5 Hz, 1H), 6.90 (d, J=3.8 Hz, 1H), 6.81 (dd, J=8.3, 1.7 Hz, 1H), 4.58 (s, 2H), 4.17 (s, 2H), 3.73 (s, 3H), 3.72 (s, 3H), 2.52 (s, 3H). MS APCI, m/z=475 (M+H). HPLC 1.51 min.

### EXAMPLE 24

### 9-Amino-5-(2,6-dimethoxypyridin-3-yl)-6-fluoro-2-propyl-2,3-dihydro-pyrrolo[3,4-b]quinolin-1-one

Using Method D, 9-amino-6-fluoro-5-iodo-2-propyl-2,3-dihydro-pyrrolo[3,4-b]quinolin-1-one (130 mg, 0.34 mmol) and 2,6-dimethoxypyridin-3-boronic acid (165 mg, 0.90 mmol) were reacted to afford the title compound as a white solid (97.0 mg, 72%). ¹H NMR (300.132 MHz, CDCl₃) δ 7.82 (dd, J=9.2, 5.8 Hz, 1H), 7.55 (d, J=8.0 Hz, 1H), 7.31 (t, J=8.9 Hz, 1H), 6.45 (d, J=7.9 Hz, 1H), 6.36 (bs, 2H), 4.31 (s, 2H), 3.99 (s, 3H), 3.88 (s, 3H), 3.54 (td, J=7.2, 2.9 Hz, 2H), 1.67 (sextet, J=7.4 Hz, 2H), 0.96 (t, J=7.4 Hz, 3H). MS APCI, m/z=397 (M+H). HPLC 1.93 min.

### EXAMPLE 25

### 2-(9-Amino-2-ethyl-1-oxo-2,3-dihydro-1H-pyrrolo[3,4-b]quinolin-5-yl)-benzonitrile

Using Method A, 9-amino-5-bromo-2-ethyl-2,3-dihydropyrrolo[3,4-b]quinolin-1-one (250 mg, 0.82 mmol) and 2-cyano-phenyl-boronic acid (240 mg, 1.62 mmol) were reacted to afford the title compound as a solid (65.4 mg, 24.3%).

¹H NMR (500.333 MHz, DMSO) δ 8.47 (d, J=8.6 Hz, 1H), 7.90 (d, J=7.6 Hz, 1H), 7.77 (t, J=7.8 Hz, 1H), 7.71 (d, J=7.0 Hz, 1H), 7.54˜7.61 (m, 3H), 4.31 (s, 2H), 3.50 (q, J=7.2 Hz, 2H), 1.16 (t, J=7.2 Hz, 3H). MS APCI, m/z=329 (M+H). HPLC 1.60 min.

### EXAMPLE 26

### 9-Amino-5-(2,6-dimethoxypyridin-3-yl)-2-ethyl-6-fluoro-2,3-dihydropyrrolo[3,4b]quinolin-1-one

Using Method A, 9-amino-5-bromo-6-fluoro-2-ethyl-2,3-dihydropyrrolo[3,4-b]quinolin-1-one (175 mg, 0.54 mmol) and 2,6-dimethoxy-pyridine-3-boronic acid (198 mg, 1.08 mmol) were reacted to afford the title compound as a solid (95 mg, 46.1%).

¹H NMR (500.333 MHz, CDCl₃) δ 7.83 (dd, J=9.2, 5.5 Hz, 1H), 7.55 (d, J=7.9 Hz, 1H), 7.31 (t, J=8.6 Hz, 1H), 6.45 (d, J=8.5 Hz, 1H), 6.36 (br, 2H), 4.32 (s, 2H), 3.99 (s, 3H), 3.88 (s, 3H), 3.63 (q, J=6.9 Hz, 2H), 1.25 (t, J=7.1 Hz, 3H). MS APCI, m/z=383 (M+H). HPLC 1.88 min.

### EXAMPLE 27

### 9-Amino-5-(2,6-dimethoxypyridin-3-yl)-2-ethyl-2,3-dihydropyrrolo[3,4-b]quinolin-1-one

Using Method A, 9-amino-5-bromo-2-ethyl-2,3-dihydropyrrolo[3,4-b]quinolin-1-one (200 mg, 0.65 mmol) and 2,6-dimethoxy-pyridine-3-boronic acid (264 mg, 1.44 mmol) were reacted to afford the title compound as a solid (142 mg, 59.6%).

¹H NMR (500.333 MHz, DMSO) δ 8.32 (dd, J=7.8, 1.2 Hz, 1H), 7.60 (dd, J=7.0, 1.2 Hz, 1H), 7.57 (d, J=7.9 Hz, 1H), 7.47 (t, J=7.7 Hz, 1H) 6.46 (d, J=7.9 Hz, 1H), 4.30 (s, 2H), 3.93 (s, 3H), 3.77 (s, 3H), 3.50 (q, J=7.2 Hz, 2H), 1.16 (t, J=7.3 Hz, 3H). MS APCI, m/z=365 (M+H). HPLC 1.81 min.

### EXAMPLE 28

### 9-amino-2-cyclopropyl-5-(2,4-dimethoxyphenyl)-6-fluoro-2,3-dihydro-1H-pyrrolo[3,4-b]quinolin-1-one

Using Method D, 9-amino-2-cyclopropyl-6-fluoro-5-iodo-2,3-dihydro-1H-pyrrolo[3,4-b]quinolin-1-one (150 mg, 0.39 mmol) and 2,4-dimethoxyphenylboronic acid (200 mg, 1.15 mmol) were reacted to afford the title compound as a white solid (110.5 mg, 71.7%).

¹H NMR (300.132 MHz, CDCl3) δ 7.80 (dd, J=9.2, 5.8 Hz, 1H), 7.30 (t, J=8.8 Hz, 1H), 7.23-7.15 (m, 1H), 6.65-6.60 (m, 2H), 6.36 (s, 2H), 4.24 (s, 2H), 3.88 (s, 3H), 3.71 (s, 3H), 2.92-2.83 (m, 1H), 0.93-0.79 (m, 4H). MS APCI, m/z=393 (M+H). HPLC 1.75 min.

### EXAMPLE 29

### 9-Amino-2-ethyl-5-(3,4-dimethoxyphenyl)-2,3-dihydropyrrolo[3,4-b]quinolin-1-one

Using Method A, 9-amino-5-bromo-2-ethyl-2,3-dihydropyrrolo[3,4-b]quinolin-1-one (250 mg, 0.82 mmol) and 3,4-dimethoxy-phenyl-boronic acid (297 mg, 1.63 mmol) were reacted to afford the title compound as a white solid (159.2 mg, 53.5%).

¹H NMR (500.333 MHz, DMSO) δ 8.30 (dd, J=8.6, 1.3 Hz, 1H), 7.69 (dd, J=7.2, 1.4 Hz, 1H), 7.50 (dd, J=8.4, 7.2 Hz, 1H), 7.26 (d, J=2.1 Hz, 1H), 7.14 (dd, J=8.3, 1.9 Hz, 1H), 7.01 (d, J=8.2 Hz, 1H), 4.36 (s, 2H), 3.82 (s, 3H), 3.78 (s, 3H), 3.52 (q, J=7.2 Hz, 2H), 1.18 (t, J=7.2 Hz, 3H). MS APCI, m/z=364 (M+H). HPLC 1.67 min.

### EXAMPLE 30

### 9-amino-5-(2,5-dimethoxyphenyl)-2-ethyl-6-fluoro-2,3-dihydro-1H-pyrrolo[3,4-b]quinolin-1-one

Using Method D, 9-amino-2-ethyl-6-fluoro-5-iodo-2,3-dihydro-1H-pyrrolo[3,4-b]quinolin-1-one (150 mg, 0.4 mmol) and 2,5-dimethoxyphenylboronic acid (250 mg, 1.37 mmol) were reacted to afford the title compound as a white solid (10.3 mg, 6.7%).

¹H NMR (300.132 MHz, DMSO) δ 8.42 (dd, J=9.3, 5.8 Hz, 1H), 7.67 (s, 2H), 7.42 (t, J=8.9 Hz, 1H), 7.04 (d, J=8.9 Hz, 1H), 6.95 (dd, J=8.9, 3.5 Hz, 1H), 6.74 (d, J=3.0 Hz, 1H), 4.30 (s, 2H), 3.72 (s, 3H), 3.58 (s, 3H), 3.48 (q, J=7.2 Hz, 2H), 1.14 (t, J=7.3 Hz, 3H). MS APCI, m/z=382 (M+H). HPLC 1.80 min.

### EXAMPLE 31

### 9-Amino-5-(2,6-dimethoxypyridin-3-yl)-2-methyl-2,3-dihydropyrrolo[3,4-b]quinolin-1-one

Using Method A, 9-amino-5-bromo-2-methyl-2,3-dihydropyrrolo[3,4-b]quinolin-1-one (200 mg, 0.68 mmol) and 2,6-dimethoxy-pyridine-3-boronic acid (251 mg, 1.37 mmol) were reacted to afford the title compound as a white solid (151 mg, 63.4%).

¹H NMR (500.333 MHz, CDCl₃) δ 7.81 (dd, J=8.3, 1.3 Hz, 1H), 7.73 (dd, J=7.2, 1.4 Hz, 1H), 7.64 (d, J=7.9 Hz, 1H), 7.50 (dd, J=8.2, 7.3 Hz, 1H), 6.43 (d, J=8.0 Hz, 1H), 6.34 (br, 2H), 4.33 (s, 2H), 3.99 (s, 3H), 3.88 (s, 3H), 3.16 (s, 3H). MS APCI, m/z=351 (M+H). HPLC 1.71 min.

### EXAMPLE 32

### 9-Amino-2-ethyl-5-(4-fluoro-2-methoxyphenyl)-2,3-dihydropyrrolo[3,4-b]quinolin-1-one

Using Method A, 9-amino-5-bromo-2-ethyl-2,3-dihydropyrrolo[3,4-b]quinolin-1-one (250 mg, 0.82 mmol) and 4-fluoro-2-methoxy-phenyl-boronic acid (278 mg, 1.83 mmol) were reacted to afford the title compound as a solid (245 mg, 85.1%).

¹H NMR (300.132 MHz, CDCl₃) δ 7.84 (dd, J=8.3, 1.5 Hz, 1H), 7.65 (dd, J=7.0, 1.4 Hz, 1H), 7.50 (dd, J=8.3, 7.2 Hz, 1H), 7.25˜7.30 (m, 1H), 6.70˜6.80 (m, 2H), 6.36 (br, 1H), 4.32 (s, 2H), 3.70 (s, 3H), 3.64 (q, J=7.4 Hz, 2H), 1.25 (t, J=7.1 Hz, 3). MS APCI, m/z=352 (M+H). HPLC 1.77 min.

### EXAMPLE 33

### 9-Amino-2-ethyl-5-(2-fluoro-3-methoxyphenyl)-2,3-dihydropyrrolo[3,4-b]quinolin-1-one

Using Method A, 9-amino-5-bromo-2-ethyl-2,3-dihydropyrrolo[3,4-b]quinolin-1-one (250 mg, 0.82 mmol) and 2-fluoro-3-methoxy-phenyl-boronic acid (278 mg, 1.63 mmol) were reacted to afford the title compound as a solid (220 mg, 76.5%).

¹H NMR (300.132 MHz, CDCl₃) δ 7.89 (d, J=8.3 Hz, 1H), 7.70 (d, J=6.8 Hz, 1H), 7.51 (t, J=7.7 Hz, 1H), 7.15 (t, J=7.4 Hz, 1H), 7.03 (d, J=7.4 Hz, 2H), 6.39 (br, 1H), 4.35 (s, 2H), 3.94 (s, 3H), 3.64 (q, J=7.3 Hz, 2H), 1.25 (t, J=7.4 Hz, 3H). MS APCI, m/z=352 (M+H). HPLC 1.74 min.

### EXAMPLE 34

### 9-amino-5-(2,4-dimethoxypyrimidin-5-yl)-2-ethyl-6-fluoro-2,3-dihydro-1H-pyrrolo[3,4-b]quinolin-1-one

Using Method D, 9-amino-2-ethyl-6-fluoro-5-iodo-2,3-dihydro-1H-pyrrolo[3,4-b]quinolin-1-one (150 mg, 0.40 mmol) and 2,4-dimethoxypyrimidin-5-ylboronic acid (500 mg, 2.72 mmol) were reacted to afford the title compound as a white solid (81.1 mg, 52.3%). ¹H NMR (500.333 MHz, DMSO) δ 8.48 (dd, J=9.6, 6.5 Hz, 1H), 8.26 (s, 1H), 7.55 (s, 2H), 7.47 (t, J=13.6 Hz, 1H), 4.32 (s, 2H), 3.98 (s, 3H), 3.83 (s, 3H), 3.49 (q, J=9.4 Hz, 2H), 2.50 (t, J=6.5 Hz, 3H). MS APCI, m/z=383 (M+H). HPLC 1.62 min.

### EXAMPLE 35

### 9-amino-2-cyclopropyl-6-fluoro-5-(2-fluoro-6-methoxyphenyl)-2,3-dihydro-1H-pyrrolo[3,4-b]quinolin-1-one

Using Method D, 9-amino-5-bromo-2-cyclopropyl-6-fluoro-2,3-dihydro-1H-pyrrolo[3,4-b]quinolin-1-one (180 mg, 0.54 mmol) and 2-fluoro-6-methoxyphenylboronic acid (1.0 g, 5.88 mmol) were reacted to afford the title compound as an off-white solid (45.9 mg, 22.5%). ¹H NMR (500.333 MHz, DMSO) δ 8.44 (dd, J=9.3, 6.2 Hz, 1H), 7.52 (s, 2H), 7.42 (m, 2H), 6.96 (d, J=8.4 Hz, 1H), 6.86 (t, J=8.5 Hz, 1H), 4.20 (d, J=1H), 4.16 (d, J=17.9 Hz, 1H), 3.65 (s, 3H), 2.89-2.86 (m, 1H), 0.84-0.71 (m, 4H). MS APCI, m/z=382 (M+H). HPLC 1.77 min.

### EXAMPLE 36

### 9-amino-2-ethyl-6-fluoro-5-(4-methoxypyridin-3-yl)-2,3-dihydro-1H-pyrrolo[3,4-b]quinolin-1-one

Using Method D, 9-amino-2-ethyl-6-fluoro-5-iodo-2,3-dihydro-1H-pyrrolo[3,4-b]quinolin-1-one (150 mg, 0.40 mmol) and 4-methoxypyridin-3-ylboronic acid (750 mg, 4.90 mmol) were reacted to afford the title compound as an off-white solid (76.2 mg, 53.5%). ¹H NMR (500.333 MHz, CDCl3) δ 8.57 (d, J=5.8 Hz, 1H), 8.42 (s, 1H), 7.88 (dd, J=9.2, 5.8 Hz, 1H), 7.33 (t, J=8.8 Hz, 1H), 6.96 (d, J=5.8 Hz, 1H), 6.39 (s, 2H), 4.29 (dd, J=21.4, 17.3 Hz, 2H), 3.81 (s, 3H), 3.63 (q, J=7.3 Hz, 2H), 1.25 (t, J=7.3 Hz, 3H). MS APCI, m/z=352 (M+H). HPLC 1.19 min.

### EXAMPLE 37

### 9-amino-2-cyclopropyl-5-(2,5-dimethoxyphenyl)-6-fluoro-2,3-dihydro-1H-pyrrolo[3,4-b]quinolin-1-one

Using Method D, 9-amino-2-cyclopropyl-6-fluoro-5-iodo-2,3-dihydro-1H-pyrrolo[3,4-b]quinolin-1-one (150 mg, 0.39 mmol) and 2,5-dimethoxyphenylboronic acid (200 mg, 1.10 mmol) were reacted to afford the title compound as an off-white solid (63.1 mg, 41.0%). ¹H NMR (300.132 MHz, CDCl3) δ 7.83 (dd, J=9.7, 5.8 Hz, 1H), 7.31 (t, J=8.8 Hz, 1H), 7.01-6.93 (m, 2H), 6.86 (d, J=2.1 Hz, 1H), 6.37 (s, 2H), 4.24 (s, 2H), 3.79 (s, 3H), 3.67 (s, 3H), 2.92-2.83 (m, 1H), 0.93-0.79 (m, 4H). MS APCI, m/z=394 (M+H). HPLC 1.72 min.

### EXAMPLE 38

### 9-amino-2-cyclopropyl-6-fluoro-5-(2-methoxypyridin-3-yl)-2,3-dihydro-1H-pyrrolo[3,4-b]quinolin-1-one

Using Method D, 9-amino-5-bromo-2-cyclopropyl-6-fluoro-2,3-dihydro-1H-pyrrolo[3,4-b]quinolin-1-one (200 mg, 0.59 mmol) and 2-methoxypyridin-3-ylboronic acid (800 mg, 5.23 mmol) were reacted to afford the title compound as a pale yellow solid (50.2 mg, 23.2%). ¹H NMR (300.132 MHz, CDCl3) δ 8.27 (dd, J=5.2, 2.1 Hz, 1H), 7.84 (dd, J=9.3, 6.0 Hz, 1H), 7.60 (dd, J=7.2, 1.9 Hz, 1H), 7.32 (t, J=8.8 Hz, 1H), 7.02 (dd, J=7.3, 5.1 Hz, 1H), 6.34 (s, 2H), 4.21 (s, 2H), 3.85 (s, 3H), 2.93-2.84 (m, 1H), 0.94-0.80 (m, 4H). MS APCI, m/z=365 (M+H). HPLC 1.52 min.

### EXAMPLE 39

### 9-amino-2-(2,5-dimethoxybenzyl)-5-(4-methoxypyridin-3-yl)-2,3-dihydro-1H-pyrrolo[3,4-b]quinolin-1-one

Using Method A, 9-amino-5-bromo-2-(2,5-dimethoxybenzyl)-2,3-dihydro-1H-pyrrolo[3,4-b]quinolin-1-one (205 mg, 0.48 mmol) and 4-methoxypyridin-3-ylboronic acid boronic acid (92 mg, 0.60 mmol) were reacted to afford the title compound as a white solid (70 mg, 32%). ¹H NMR (300.132 MHz, DMSO) δ 8.43 (m, 2H), 8.25 (s, 1H), 7.61 (d, J=6.5 Hz,1H), 7.52 (dd, J=8.2, 8.3 Hz,1H), 7.14 (d, J=5.7 Hz,1H), 6.94 (d, J=8.8 Hz,1H), 6.70 (d, J=2.7 Hz,1H), 4.60 (s, 2H), 4.22 (s, 2H), 3.76 (s, 3H), 3.70 (s, 3H), 3.65 (s, 3H), 6.83 (dd, J=3.2, 9.0 Hz,1H), MS APCI, m/z=457 (M+H). HPLC 1.20 min.

### EXAMPLE 40

### 9-Amino-2-propyl-5-pyridin-3-yl-2,3-dihydropyrrolo[3,4-b]quinolin-1-one

Using Method A, 9-amino-5-iodo-2-propyl-2,3-dihydropyrrolo[3,4-b]quinolin-1-one (74.6 mg, 0.20 mmol) and 3-pyridyl boronic acid (77.8 mg, 0.63 mmol) were reacted to afford the title compound as a white solid (61.4 mg, 95%). ¹H NMR (300.132 MHz, DMSO) δ 8.79 (d, J=1.7 Hz, 1H), 8.56 (dd, J=4.7, 1.4 Hz, 1H), 8.41 (dd, J=8.3, 1.0 Hz, 1H), 8.01 (dt, J=7.9, 1.9 Hz, 1H), 7.76 (dd, J=7.1, 1.1 Hz, 1H), 7.70 (bs, 2H), 7.56 (dd, J=8.7, 7.3 Hz, 1H), 7.47 (dd, J=7.7, 4.8 Hz, 1H), 4.35 (s, 2H), 3.44 (t, J=7.1 Hz, 2H), 1.61 (sextet, J=7.3 Hz, 2H), 0.87 (t, J=7.3 Hz, 3H). MS APCI, m/z=319.0 (M+H). HPLC 1.12 min.

### EXAMPLE 41

### 9-amino-2-cyclopropyl-6-fluoro-5-(4-methoxypyridin-3-yl)-2,3-dihydro-1H-pyrrolo[3,4-b]quinolin-1-one

Using Method D, 9-amino-5-bromo-2-cyclopropyl-6-fluoro-2,3-dihydro-1H-pyrrolo[3,4-b]quinolin-1-one (200 mg, 0.59 mmol) and 4-methoxypyridin-3-ylboronic acid (600 mg, 3.92 mmol) were reacted to afford the title compound as a pale yellow solid (45.2 mg, 20.8%). ¹H NMR (300.132 MHz, CDCl3) δ 8.56 (d, J=5.8 Hz, 1H), 8.38 (s, 1H), 7.87 (dd, J=9.2, 5.8 Hz, 1H), 7.33 (t, J=8.8 Hz, 1H), 6.96 (d, J=5.9 Hz, 1H), 6.34 (s, 2H), 4.23 (s, 2H), 3.80 (s, 3H), 2.94-2.81 (m, 1H), 0.92-0.81 (m, 4H). MS APCI, m/z=365 (M+H). HPLC 1.18 min.

### EXAMPLE 42

### 9-amino-2-cyclobutyl-5-(2,5-dimethoxyphenyl)-2,3-dihydro-1H-pyrrolo[3,4-b]quinolin-1-one

Using Method A, 9-amino-5-bromo-2-cyclobutyl-2,3-dihydro-1H-pyrrolo[3,4-b]quinolin-1-one (250 mg, 0.75 mmol) and 2,5-dimethoxyphenylboronic acid boronic acid (171 mg, 0.94 mmol) were reacted to afford the title compound as a white solid (101 mg, 34%). ¹H NMR (500.333 MHz, DMSO) δ 8.32 (d, J=8.2 Hz, 1H), 7.58 (bs, 2H), 7.55 (dd, J=7.2, 1.2 Hz,1H), 7.46 (dd, J=8.3, 7.1 Hz,1H), 7.02 (d, J=9.0 Hz,1H), 6.91 (dd, J=9.0, 3.1 Hz,1H), 6.78 (d, J=3.1 Hz,1H), 4.72 (q, J=8.6 Hz, 1H), 4.40 (s, 2H), 3.72 (s, 3H), 3.56 (s, 3H), 2.31 m, 2H), 2.11 m 2H), 1.68 (m, 2H). MS APCI, m/z=390 (M+H). HPLC 1.56 min.

### EXAMPLE 43

### 9-Amino-2-butyl-5-(2,6-dimethoxypyridin-3-yl)-2,3-dihydropyrrolo[3,4-b]quinolin-1-one

Using Method A, 9-amino-5-bromo-2-butyll-2,3-dihydropyrrolo[3,4-b]quinolin-1-one (200 mg, 0.60 mmol) and 2,6-dimethoxy-pyridine-3-boronic acid (242 mg, 1.32 mmol) were reacted to afford the title compound as a solid (130 mg, 55.3%).

¹H NMR (300.132 MHz, CDCl₃) δ 7.82 (dd, J=8.4, 1.5 Hz, 1H), 7.74 (dd, J=7.2, 1.5 Hz, 1H), 7.64 (d, J=8.0 Hz, 1H), 7.50 (dd, J=8.3, 7.2 Hz, 1H), 6.43 (d, J=8.0 Hz, 1), 6.35 (br, 2H), 4.33 (s, 2H), 3.99 (s, 3H), 3.88 (s, 3H), 3.59 (t, J=7.2 Hz, 2), 1.64 (m, 2H), 1.38 (m, 2H), 0.96 (t, J=7.3 Hz, 3H). MS APCI, m/z=393 (M+H). HPLC 1.96 min.

### EXAMPLE 44

### 9-Amino-2-cyclopropyl-5-(2,4-dimethoxypyrimidin-5-yl)-6-fluoro-2,3-dihydropyrrolo[3,4-b]quinolin-1-one

Using Method D, 9-amino-2-cyclopropyl-6-fluoro-5-iodo-2,3-dihydro-1H-pyrrolo[3,4-b]quinolin-1-one (150 mg, 0.39 mmol) and 2,4-dimethoxypyrimidin-5-ylboronic acid (450 mg, 2.45 mmol) were reacted to afford the title compound as a white solid (71.1 mg, 46%). ¹H NMR (300.132 MHz, CDCl3) δ 8.26 (s, 1H), 7.86 (dd, J=9.2, 5.8 Hz, 1H), 7.31 (t, J=8.8 Hz, 1H), 6.41 (s, 2H), 4.58 (s, 2H), 4.08 (s, 3H), 4.08 (s, 2.93-2.84 (m, 1H), 0.95-0.81 (m, 4H). MS APCI, m/z=396 (M+H). HPLC 1.27 min.

### EXAMPLE 45

### 9-Amino-2-ethyl-6-fluoro-5-(2-methoxyphenyl)-2,3-dihydropyrrolo[3,4-b]quinolin-1-one

Using Method D, 9-amino-5-bromo-2-ethyl-6-fluoro-2,3-dihydro-1H-pyrrolo[3,4-b]quinolin-1-one (180 mg, 0.56 mmol) and 2-methoxyphenylboronic acid (300 mg, 1.97 mmol) were reacted to afford the title compound as a off-white solid (73.1 mg, 37.5%).

¹H NMR (300.132 MHz, CDCl3) δ 7.84 (dd, J=9.2, 5.8 Hz, 1H), 7.47-7.29 (m, 2H), 7.21-7.04 (m, 3H), 6.34 (s, 2H), 4.29 (s, 2H), 4.21 (s, 3H), 3.66-3.58 (m, 2H), 1.24 (t, J=7.3 Hz, 3H). MS APCI, m/z=352 (M+H). HPLC 1.73 min.

### EXAMPLE 46

### 9-Amino-2-ethyl-6-fluoro-5-(5-fluoro-2-methoxyphenyl)-2,3-dihydropyrrolo[3,4-b]quinolin-1-one

Using Method D, 9-amino-5-bromo-2-ethyl-6-fluoro-2,3-dihydro-1H-pyrrolo[3,4-b]quinolin-1-one (180 mg, 0.56 mmol) and 5-fluoro-2-methoxyphenylboronic acid (300 mg, 1.77 mmol) were reacted to afford the title compound as a pale yellow solid (93.1 mg, 45.4%). ¹H NMR (300.132 MHz, CDCl3) δ 7.85 (dd, J=9.2, 5.8 Hz, 1H), 7.31 (t, J=8.7 Hz, 1H), 7.14-6.94 (m, 3H), 6.36 (s, 2H), 4.37-4.24 (m, 2H), 3.70 (s, 3H), 3.63 (qd, J=7.2, 2.2 Hz, 2H), 1.25 (t, J=7.2 Hz, 3H). MS APCI, m/z=370 (M+H). HPLC 1.84 min.

### EXAMPLE 47

### 9-Amino-5-(3,4-dimethoxyphenyl)-2-ethyl-6-fluoro-2,3-dihydropyrrolo[3,4-b]quinolin-1-one

Using Method D, 9-amino-5-bromo-2-ethyl-6-fluoro-2,3-dihydro-1H-pyrrolo[3,4-b]quinolin-1-one (185 mg, 0.57 mmol) and 3,4-dimethoxyphenylboronic acid (350 mg, 1.92 mmol) were reacted to afford the title compound as an off-white solid (96.4 mg, 44.2%). ¹H NMR (300.132 MHz, CDCl3) δ 7.81 (dd, J=9.2, 5.7 Hz, 1H), 7.33 (t, J=9.0 Hz, 1H), 7.13-7.08 (m, 2H), 7.00 (d, J=8.2 Hz, 1H), 6.37 (s, 2H), 4.35 (s, 2H), 3.95 (s, 3H), 3.89 (s, 3H), 3.64 (q, J=7.2 Hz, 2H), 1.26 (t, J=7.2 Hz, 3H). MS APCI, m/z=382 (M+H). HPLC 1.68 min.

### EXAMPLE 48

### 9-Amino-2-cyclobutyl-5-(2-methoxypyridin-3-yl)-2,3-dihydropyrrolo[3,4-b]quinolin-1-one

Using Method A, 9-amino-5-bromo-2-cyclobutyl-2,3-dihydro-pyrrolo[3,4-b]quinolin-1-one (100 mg, 0.30 mmol) and 2-methoxy-pyridin-3-boronic acid (72 mg, 0.47 mmol) were reacted to afford the title compound as a solid (99.6 mg, 86.5%). ¹H NMR (300.132 MHz, CDCl₃) δ 8.23 (dd, J=5.0, 1.7 Hz, 1H), 7.86 (dd, J=8.5, 1.3 Hz, 1H), 7.69 (dd, J=7.0, 1.5 Hz, 1H), 7.66 (dd, J=7.2, 2.1 Hz, 1H), 7.48 (t, J=7.6 Hz, 1H), 7.00 (dd, J=7.2, 5.1 Hz, 1H), 6.45 (bs, 2H), 4.89 (quintet, J=8.7 Hz, 1H), 4.38 (s, 2H), 3.87 (s, 3H), 2.25 (q, J=7.7 Hz, 4H), 1.76 (quintet, J=8.1 Hz, 2H). MS APCI, m/z=361 (M+H). HPLC 1.67 min.

### EXAMPLE 49

### 9-Amino-5-(5-chloro-2-methoxyphenyl)-2-cyclobutyl-2,3-dihydropyrrolo[3,4-b]quinolin-1-one

Using Method A, 9-amino-5-bromo-2-cyclobutyl-2,3-dihydro-pyrrolo[3,4-b]quinolin-1-one (100 mg, 0.30 mmol) and 2-methoxy-5-chloro-phenyl-boronic acid (75 mg, 0.47 mmol) were reacted to afford the title compound as a solid (92.0 mg, 78.8%). ¹H NMR (300.132 MHz, CDCl₃) δ 7.84 (dd, J=8.5, 1.3 Hz, 1H), 7.64 (dd, J=7.2, 1.3 Hz, 1H), 7.47 (t, J=7.8 Hz, 1H), 7.35˜7.30 (m, 1H), 7.30 (s, 1H), 6.95˜6.92 (m, 1H), 6.40 (bs, 2H), 4.89 (quintet, J=8.7 Hz, 1H), 4.39 (s, 2H), 3.68 (s, 3H), 2.26 (q, J=7.9 Hz, 4H), 1.76 (quintet, J=8.0 Hz, 2H). MS APCI, m/z=394 (M+H). HPLC 2.00 min.

### EXAMPLE 51

### 9-amino-2-cyclopropyl-5-(3,4-dimethoxyphenoxy)-2,3-dihydro-1H-pyrrolo[3,4-b]quinolin-1-one

3,4-Dimethoxyphenol (0.539 g, 3.5 mmol) was dissolved in dimethylformamide (2 mL). To the brown stirring suspension was added sodium hydride (0.96 g, 4.0 mmol) in portions. Let stir at room temperature for 5 minutes. To this clear brown solution was added 2-(1-cyclopropyl-5-oxo-2,5-dihydro-1H-pyrrol-3-ylamino)-3-fluorobenzonitrile (0.300 g, 1.17 mmol), washed in with an additional 1 mL of DMF. The mixture was immediately heated to 160° C. and stirred for 6 hours. After cooling to RT, the reaction was diluted with saturated aqueous sodium bicarbonate (100 mL) and extracted 3 times with 75 mL of methylene chloride. The organics were combined and evacuated under high vacuum to remove the dimethylformamide. The residue was dissolved in 15 ml of DMSO and 0.5 ml of TFA. This solution was injected onto a Gilson reverse phase 2 inch C8 column for separation in 5 equal injections. Fractions containing product were pooled, reduced in volume by half in a rotoevaporator and basified with 5N sodium hydroxide to pH=12. A white solid instantly dropped out of solution. This thick white precipitate was filtered and dried under high vacuum to afford 110 mg of sample was dissolved in methylene chloride and methanol, reabsorbed onto silica gel, and purified using 10% methanol/methylene chloride as the eluent to give 9-Amino-5-(3,4-dimethoxyphenoxy)-2-ethyl-2,3-dihydro-1H-pyrrolo[3,4-b]quinolin-1-one as a white solid (14.0%). ¹H NMR (500.333 MHz, DMSO) δ 8.05 (m, 1H), 7.64 (s, 2H), 7.35 (dd, J=8.0, 8.1 Hz, 1H), 7.10 (dd, J=1.0, 7.7 Hz, 1H), 6.90 (d, J=8.8 Hz, 1H), 6.76 (d, J=2.8 Hz, 1H), 6.40 (dd, J=2.8, 8.7 Hz, 1H), 4.32 (s, 2H), 3.73 (s, 3H), 3.72 (s, 3H), 2.89 (m,1H), 0.82 (m, 4H). MS APCI, m/z=392 (M+H). HPLC 1.31 min.

### EXAMPLE 52

### 9-amino-2-cyclobutyl-5-(2,6-dimethoxypyridin-3-yl)-6-fluoro-2,3-dihydro-1H-pyrrolo[3,4-b]quinolin-1-one

Using Method D, 9-amino-5-bromo-2-cyclobutyl-6-fluoro-2,3-dihydro-1H-pyrrolo[3,4-b]quinolin-1-one (180 mg, 0.51 mmol), and 2,6-dimethoxypyridin-3-ylboronic acid (250 mg, 1.37 mmol) were reacted to afford the title compound as a white solid (63.0 mg, 28.5%). ¹H NMR (300.132 MHz, MeOD) δ 8.25 (dd, J=8.8, 6.3 Hz, 1H), 7.48 (d, J=7.5 Hz, 1H), 7.33 (t, J=9.0 Hz, 1H), 6.46 (d, J=7.5 Hz, 1H), 4.40 (s, 2H), 4.00 (s, 3H), 3.85 (s, 3H), 3.33-3.29 (m, 1H), 2.44-2.18 (m, 4H), 1.86-1.73 (m, 2H). MS APCI, m/z=408 (M+H). HPLC 1.69 min.

### EXAMPLE 53

### 9-amino-2-cyclobutyl-5-(2,4-dimethoxyphenyl)-2,3-dihydro-1H-pyrrolo[3,4-b]quinolin-1-one

Using Method A, 9-amino-5-bromo-2-cyclobutyl-2,3-dihydro-1H-pyrrolo[3,4-b]quinolin-1-one (83 mg, 0.25 mmol) and 2,4-dimethoxyphenylboronic acid (68 mg, 0.375 mmol) were reacted to afford the title compound as a white solid (61 mg, 62%). ¹H NMR (500.333 MHz, DMSO) δ 8.28 (dd, J=8.3, 1.2 Hz, 1H), 7.57 (s, 2H), 7.52 (dd, J=7.1, 1.4 Hz, 1H), 7.46-7.43 (m, 1H), 7.10 (d, J=8.4 Hz,1H), 6.66 (d, J=2.3 Hz, 1H), 6.58 (dd, J=8.3, 2.4 Hz, 1H), 4.76-4.69 (m, 1H), 4.38 (s, 2H), 3.83 (s, 3H), 3.62 (s, 3H), 2.36-2.27 (m, 2H), 2.14-2.07 (m, 2H), 1.71-1.64 (m, 2H). MS APCI, m/z=390 (M+H). HPLC 1.53 min.

### EXAMPLE 54

### 9-Amino-2-cyclopropyl-5-(2,6-dimethoxy-pyridin-3-yl)-6-fluoro-2,3-dihydro-pyrrolo[3,4-b]quinolin-1-one

Using Method D, 9-amino-2-cyclopropyl-6-fluoro-5-iodo-2,3-dihydro-1H-pyrrolo[3,4-b]quinolin-1-one (150 mg, 0.39 mmol), and 2,6-dimethoxypyridin-3-ylboronic acid (210 mg, 1.15 mmol) were reacted to afford the title compound as a white solid (84.4 mg, 54.8%). ¹H NMR (300.132 MHz, CDCl3) δ 7.84-7.78 (m, 1H), 7.54 (d, J=8.0 Hz, 1H), 7.33-7.27 (m, 1H), 6.45 (d, J=8.1 Hz, 1H), 6.37 (s, 2H), 4.25 (s, 2H), 3.99 (s, 3H), 3.88 (s, 3H), 2.93-2.84 (m, 1H), 0.94-0.80 (m, 4H). MS APCI, m/z=394 (M+H). HPLC 1.93 min.

### EXAMPLE 55

### 9-Amino-6-fluoro-5-(2-fluoro-6-methoxy-phenyl)-2-propyl-2,3-dihydro-pyrrolo[3,4-b]quinolin-1-one

Using Method D, 9-amino-6-fluoro-5-iodo-2-propyl-2,3-dihydro-1H-pyrrolo[3,4-b]quinolin-1-one (230 mg, 0.60 mmol), and 2-fluoro-6-methoxyphenylboronic acid (500 mg, 2.94 mmol) were reacted to afford the title compound as a white solid (29.4 mg, 12.8%). ¹H NMR (300.132 MHz, CDCl3) δ 7.88 (dd, J=9.2, 5.8 Hz, 1H), 7.43-7.28 (m, 2H), 6.87-6.80 (m, 2H), 6.39 (s, 2H), 4.29 (dd, J=20.5, 17.4 Hz, 2H), 3.73 (s, 3H), 3.58-3.46 (m, 2H), 1.72-1.60 (m, 2H), 0.95 (t, J=7.4 Hz, 3H). MS APCI, m/z=383 (M+H). HPLC 1.98 min.

### EXAMPLE 56

### 9-Amino-5-(2,6-difluoro-phenyl)-2-propyl-2,3-dihydro-pyrrolo[3,4-b]quinolin-1-one

Using Method D, 9-amino-5-bromo-2-propyl-2,3-dihydro-1H-pyrrolo[3,4-b]quinolin-1-one (130 mg, 0.41 mmol), and 2,6-difluorophenylboronic acid (240 mg, 1.52 mmol) were reacted to afford the title compound as a white solid (27.5 mg, 19.2%).

¹H NMR (500.333 MHz, DMSO) δ 8.47 (dd, J=8.4, 1.3 Hz, 1H), 7.72 (s, 2H), 7.70 (d, J=6.1 Hz, 1H), 7.55 (dd, J=8.3, 7.2 Hz, 1H), 7.49 (ddd, J=15.0, 8.4, 6.6 Hz, 1H), 7.17 (t, J=7.7 Hz, 2H), 5.03 (s, 2H), 3.41 (t, J=7.2 Hz, 2H), 1.64-1.55 (m, 2H), 0.86 (t, J=7.4 Hz, 3H). MS APCI, m/z=354 (M+H). HPLC 1.54 min.

### EXAMPLE 57

### 9-Amino-2-ethyl-5-(4-methoxy-pyridin-3-yl)-2,3-dihydro-pyrrolo[3,4-b]quinolin-1-one

Using Method A, 9-amino-5-bromo-2-ethyl-2,3-dihydro-pyrrolo[3,4-b]quinolin-1-one (250 mg, 0.82 mmol) and 4-methoxy-pyridin-3-boronic acid (429 mg, 2.80 mmol) were reacted to afford the title compound as a solid (180 mg, 65.7%). ¹H NMR (500.333 MHz, CDCl₃) δ 8.54 (d, J=5.8 Hz, 1H), 8.47 (s, 1H), 7.88 (dd, J=8.8, 1.4 Hz, 1H), 7.69 (dd, J=7.0, 1.5 Hz, 1H), 7.52 (dd, J=8.2, 7.3 Hz, 1H), 6.94 (d, J=5.8 Hz, 1H), 6.35 (bs, 2H), 4.30 (s, 2H), 3.78 (s, 3H), 3.64 (q, J=7.3 Hz, 2H), 1.26 (t, J=7.4 Hz, 3H). MS APCI, m/z=335 (M+H). HPLC 0.68 min.

### EXAMPLE 58

### 9-Amino-2-ethyl-6-fluoro-5-(2-methoxy-pyridin-3-yl)-2,3-dihydro-pyrrolo[3,4-b]quinolin-1-one

Using Method D, 9-amino-2-ethyl-6-fluoro-5-iodo-2,3-dihydro-1H-pyrrolo[3,4-b]quinolin-1-one (150 mg, 0.40 mmol), and 2-methoxypyridin-3-ylboronic acid (400 mg, 2.62 mmol) were reacted to afford the title compound as a white solid (63.4 mg, 44.6%).

¹H NMR (500.333 MHz, DMSO) δ 8.46 (dd, J=9.1, 6.2 Hz, 1H), 8.24 (dd, J=5.1, 1.9 Hz, 1H), 7.63 (dd, J=7.3, 2.0 Hz, 1H), 7.45 (t, J=10.5 Hz, 1H), 7.10 (dd, J=6.9, 5.0 Hz, 1H), 4.29 (s, 2H), 3.75 (s, 3H), 3.48 (q, J=7.3 Hz, 2H), 1.14 (t, J=7.2 Hz, 3H). MS APCI, m/z=353 (M+H). HPLC 1.58 min.

### EXAMPLE 59

### 9-amino-2-cyclopropyl-5-(2,5-dichlorophenyl)-2,3-dihydro-1H-pyrrolo[3,4-b]quinolin-1-one

Using Method A, 9-amino-5-bromo-2-cyclopropyl-2,3-dihydro-1H-pyrrolo[3,4-b]quinolin-1-one (175 mg, 0.55 mmol) and 2,5-dichlorophenylboronic acid (157 mg, 0.875 mmol) were reacted to afford the title compound as a white solid (173 mg, 82%).

¹H NMR (500.333 MHz, DMSO) δ 8.43 (dd, J=1.4, 8.3 Hz,1H), 7.72 (s, 2H), 7.61-7.44 (m, 4H), 7.42 (d, J=2.5 Hz, 1H), 4.23 (s, 2H), 3.29 (s, 3H) 2.91-2.86 (m, 1H), 0.84-0.70 (m, 4H). MS APCI, m/z=384 (M+H). HPLC 1.55 min.

### EXAMPLE 60

### 9-amino-2-cyclopropyl-5-(2-fluoro-5-methoxyphenyl)-2,3-dihydro-1H-pyrrolo[3,4-b]quinolin-1-one

Using Method A, 9-amino-5-bromo-2-cyclopropyl-2,3-dihydro-1H-pyrrolo[3,4-b]quinolin-1-one (150 mg, 0.47 mmol) and 2-fluoro-5-methoxyphenylboronic acid (120 mg, 0.708 mmol) were reacted to afford the title compound as a white solid (119 mg, 70%). ¹H NMR (500.333 MHz, DMSO) δ 8.40 (dd, J=8.4, 1.4 Hz, 1H), 7.70 (s, 2H), 7.65 (d, J=7.4 Hz, 1H), 7.51 (dd, J=8.9, 6.6 Hz,1H), 7.16 (dd, J=9.2, 8.9 Hz, 1H), 6.97-6.90 (m, 2H), 4.24 (s, 2H), 3.76 (s, 3H), 2.91-2.86 (m, 1H), 0.85-0.71 (m, 4H). MS APCI, m/z=364 (M+H). HPLC 1.37 min.

### EXAMPLE 61

### 9-Amino-2-cyclopropyl-6-fluoro-5-(5-fluoro-2-methoxy-phenyl)-2,3-dihydro-pyrrolo[3,4-b]quinolin-1-one

Using Method D, 9-amino-5-bromo-2-cyclopropyl-6-fluoro-2,3-dihydro-1H-pyrrolo[3,4-b]quinolin-1-one (190 mg, 0.57 mmol) and 5-fluoro-2-methoxyphenylboronic acid (650 mg, 3.82 mmol) were reacted to afford the title compound as a white solid (68.3 mg, 31.6%). ¹H NMR (500.333 MHz, DMSO) δ 8.44 (dd, J=9.3, 6.2 Hz, 1H), 7.63 (s, 2H), 7.43 (t, J=9.0 Hz, 1H), 7.21 (td, J=8.7, 3.2 Hz, 1H), 7.11 (dd, J=9.2, 4.6 Hz, 1H), 7.03 (dd, J=9.0, 3.2 Hz, 1H), 4.22 (s, 2H), 3.62 (s, 3H), 2.90-2.85 (m, 1H), 0.84-0.71 (m, 4H). MS APCI, m/z=382 (M+H). HPLC 1.48 min.

### EXAMPLE 62

### 9-Amino-2-cyclobutyl-5-(4-methoxy-pyridin-3-yl)-2,3-dihydro-pyrrolo[3,4-b]quinolin-1-one

Using Method A, 9-amino-5-bromo-2-cyclobutyl-2,3-dihydro-pyrrolo[3,4-b]quinolin-1-one (100 mg, 0.30 mmol) and 4-methoxy-pyridin-3-boronic acid (100 mg, 0.65 mmol) were reacted to afford the title compound as a solid (85 mg, 73.8%). ¹H NMR (300.132 MHz, CDCl₃) δ 8.54 (d, J=5.9 Hz, 1H), 8.46 (s, 1H), 7.89 (dd, J=8.4, 1.3 Hz, 1H), 7.68 (d, J=6.8 Hz, 1H), 7.50 (t, J=7.8 Hz, 1H), 6.94 (d, J=5.5 Hz, 1H), 6.44 (bs, 2H), 4.89 (quintet, J=8.6 Hz, 1H), 4.38 (s, 2H), 3.78 (s, 3H), 2.26 (q, J=7.9 Hz, 4H), 1.76 (quintet, J=7.8 Hz, 2H). MS APCI, m/z=361 (M+H). HPLC 1.22 min.

### EXAMPLE 63

### 9-Amino-2-cyclobutyl-5-(2-methoxy-phenyl)-2,3-dihydro-pyrrolo[3,4-b]quinolin-1-one

Using Method A, 9-amino-5-bromo-2-cyclobutyl-2,3-dihydro-pyrrolo[3,4-b]quinolin-1-one (100 mg, 0.30 mmol) and 2-methoxy-phenyl-boronic acid (71 mg, 0.47 mmol) were reacted to afford the title compound as a solid (113.5 mg, 98.7%). ¹H NMR (300.132 MHz, CDCl₃) δ 8.23 (dd, J=8.4, 1.3 Hz, 1H), 7.67 (dd, J=7.2, 1.3 Hz, 1H), 7.49 (t, J=7.8 Hz, 1H), 7.39 (td, J=7.8, 1.6 Hz, 1H), 7.33 (dd, J=7.6, 1.7 Hz, 1H), 7.06 (t, J=7.4 Hz, 1H), 7.04 (d, J=8.4 Hz, 1H), 6.36 (bs, 2H), 4.89 (quintet, J=8.7 Hz, 1H), 4.38 (s, 2H), 3.71 (s, 3H), 2.25 (q, J=7.9 Hz, 4H), 1.76 (quintet, J=8.0 Hz, 2H). MS APCI, m/z=360 (M+H). HPLC 1.84 min.

### EXAMPLE 64

### 9-Amino-2-cyclobutyl-5-(2,6-dimethoxy-pyridin-3-yl)-2,3-dihydro-pyrrolo[3,4-b]quinolin-1-one

Using Method A, 9-amino-5-bromo-2-cyclobutyl-2,3-dihydro-pyrrolo[3,4-b]quinolin-1-one (150 mg, 0.45 mmol) and 2,6-dimethoxy-pyridin-3-boronic acid (124 mg, 0.68 mmol) were reacted to afford the title compound as a solid (153 mg, 86.9%). ¹H NMR (300.132 MHz, CDCl₃) δ 7.81 (dd, J=8.7, 1.3 Hz, 1H), 7.73 (dd, J=7.2, 1.5 Hz, 1H), 7.64 (d, J=8.1 Hz, 1H), 7.49 (dd, J=8.3, 7.4 Hz, 1H), 6.44 (d, J=8.0 Hz, 1H), 6.35 (bs, 2H), 4.90 (quintet, J=8.7 Hz, 1H), 4.41 (s, 2H), 3.99 (s, 3H), 3.88 (s, 3H), 2.27 (q, J=7.8 Hz, 4H), 1.77 (quintet, J=7.5 Hz, 2H). MS APCI, m/z=391 (M+H). HPLC 1.95 min.

### EXAMPLE 65

### 9-Amino-2-(3,4-dimethoxybenzyl)-5-(2-fluoro-6-methoxyphenyl)-2,3-dihydro-1H-pyrrolo[3,4-b]quinolin-1-one

Using Method A, 9-amino-5-bromo-2-(3,4-dimethoxybenzyl)-2,3-dihydro-1H-pyrrolo[3,4-b]quinolin-1-one (300 mg, 0.70 mmol) and 2-fluoro-5-methoxyphenylboronic acid (357 mg, 2.10 mmol) were reacted to afford the title compound as an off white solid (98 mg, 30%). ¹H NMR (300.132 MHz, DMSO) δ 8.40 (d, J=7.6 Hz, 1H), 7.69 (s, 2H), 7.52 (m, 2H), 7.41-7.33 (m, 1H), 6.87 (m, 5H), 4.58 (s, 2H), 4.17 (s, 2H), 3.72 (s, 3H), 3.71 (s, 3H), 3.62 (s, 3H). MS APCI, m/z=474 (M+H). HPLC 1.66 min.

### EXAMPLE 66

### 9-amino-2-cyclopropyl-5-(2-methoxypyridin-3-yl)-2,3-dihydro-1H-pyrrolo[3,4-b]quinolin-1-one

Using Method A, 9-amino-5-bromo-2-cyclopropyl-2,3-dihydro-1H-pyrrolo[3,4-b]quinolin-1-one (250 mg, 0.786 mmol) and 2-methoxypyridin-3-ylboronic acid (180 mg, 1.18 mmol) were reacted to afford the title compound as an off white solid (150 mg, 55%). ¹H NMR (500.333 MHz, DMSO) δ 8.46 (d, J=6.0 Hz, 1H), 8.37 (dd, J=8.6, 1.1 Hz,1H), 8.24 (s, 1H), 7.65 (s, 2H), 7.61 (dd, J=7.1, 1.1 Hz,1H), 7.49 (dd, J=8.9, 8.3 Hz,1H), 7.15 (d, J=5.7 Hz,1H), 4.21 (s, 2H), 3.72 (s, 3H), 2.91-2.86 (m, 1H), 0.84-0.72 (m, 4H) MS APCI, m/z=347. (M+H). HPLC 1.14 min.

### EXAMPLE 67

### 9-Amino-2-ethyl-6-fluoro-5-(2-fluoro-6-methoxyphenyl)-2,3-dihydropyrrolo[3,4-b]quinolin-1-one

Using Method A, 9-amino-5-bromo-2-ethyl-6-fluoro-2,3-dihydropyrrolo[3,4-b]quinolin-1-one (175 mg, 0.54 mmol) and 2-fluoro-6-methoxyphenyl boronic acid (275 mg, 1.62 mmol) were reacted to afford the title compound as an off-white solid (25 mg, 6.8%). ¹H NMR (500 MHz, CHLOROFORM-d) δ ppm 7.88 (dd, J=9.2, 5.8 Hz, 1 H) 7.39 (td, J=7.8, 6.6 Hz, 1 H) 7.33 (t, J=8.7 Hz, 1 H) 6.84 (d, J=8.4 Hz, 1 H) 6.84 (t, J=8.2 Hz, 1 H) 6.36 (br. s., 2 H) 4.32 (d, J=25.4 Hz, 1 H) 4.30 (d, J=25.4 Hz, 1 H) 3.73 (s, 3 H) 3.61 (ddd, J=15.9, 7.2, 7.1 Hz, 2 H) 1.24 (t, J=7.2 Hz, 3 H). MS APCI, m/z=370.3 (M+H). HPLC 1.81 min.

### EXAMPLE 68

### 9-Amino-5-(2,4-dimethoxy-phenyl)-2-ethyl-6-fluoro-2,3-dihydropyrrolo[3,4-b]quinolin-1-one

Using Method A, 9-amino-5-bromo-2-ethyl-6-fluoro-2,3-dihydropyrrolo[3,4-b]quinolin-1-one (175 mg, 0.54 mmol) and 2,4-dimethoxyphenyl boronic acid (198 mg, 1.08 mmol) were reacted to afford the title compound as a white solid (141 mg, 13.1%).

¹H NMR (500 MHz, CHLOROFORM-d) δ ppm 7.81 (dd, J=9.2, 6.1 Hz, 1 H) 7.30 (t, J=8.9 Hz, 1 H) 7.20 (d, J=9.2 Hz, 1 H) 6.58-6.67 (m, 2 H) 6.36 (br. s., 2 H) 4.32 (d, J=18.9 Hz, 1H) 4.32 (d, J=18.9 Hz, 1H) 3.88 (s, 3 H) 3.71 (s, 3 H) 3.54-3.68 (m, J=7.32 Hz, 2 H) 1.24 (t, J=7.3 Hz, 3 H). MS APCI, m/z=383.3 (M+H). HPLC 1.89 min.

### EXAMPLE 69

### 9-Amino-5-(2-fluoro-6-methoxyphenyl)-2-isopropyl-2,3-dihydropyrrolo[3,4-b]quinolin-1-one

Using Method A, 9-amino-5-bromo-2-isopropyl-2,3-dihydropyrrolo[3,4-b]quinolin-1-one (250 mg, 0.78 mmol) and 2-fluoro-6-methoxyphenyl boronic acid (265 mg, 1.56 mmol) were reacted to afford the title compound as an off-white solid (90 mg, 43.5%). 1H NMR (300 MHz, CHLOROFORM-d) δ ppm 7.88 (dd, J=8.3, 1.4 Hz, 1 H) 7.65 (dd, J=7.1, 1.4 Hz, 1 H) 7.52 (dd, J=8.2, 7.2 Hz, 1 H) 7.30-7.41 (m, 1 H) 6.83 (dd, J=8.4, 1.6 Hz, 2 H) 6.20-6.51 (m, 2 H) 4.63 (dt, J=13.5, 6.7 Hz, 1 H) 4.28 (d, J=17.3 Hz, 1H) 4.27 (d, J=17.3 Hz, 1H) 3.71 (s, 3 H) 1.26 (d, J=6.7 Hz, 6 H). MS APCI, m/z=366.4 (M+H). HPLC 1.82 min.

### EXAMPLE 70

### 9-Amino-6-fluoro-5-(2-fluoro-6-methoxyphenyl)-2-methyl-2,3-dihydropyrrolo[3,4-b]quinolin-1-one

Using Method A, 9-amino-5-bromo-2-methyl-6-fluoro-2,3-dihydropyrrolo[3,4-b]quinolin-1-one (205 mg, 0.66 mmol) and 2-fluoro-6-methoxyphenyl boronic acid (450 mg, 2.64 mmol) were reacted to afford the title compound as an off-white solid (30 mg, 8.5%). ¹H NMR (500 MHz, DMSO-d₆) δ ppm 8.48 (dd, J=9.3, 6.3 Hz, 1 H) 7.40-7.48 (m, 2 H) 6.98 (d, J=8.2 Hz, 1 H) 6.90 (t, J=8.7 Hz, 1 H) 4.27 (s, 2 H) 3.66 (s, 3 H) 2.99 (s, 3 H). MS APCI, m/z=356.1 (M+H). HPLC 4.72 min.

### EXAMPLE 71

### 9-Amino-5-(2-fluoro-3-methoxyphenyl)-2-methyl-2,3-dihydropyrrolo[3,4-b]quinolin-1-one

Using Method A, 9-amino-5-bromo-2-methyl-2,3-dihydropyrrolo[3,4-b]quinolin-1-one (255 mg, 0.87 mmol) and 2-fluoro-3-methoxyphenyl boronic acid (297 mg, 1.75 mmol) were reacted to afford the title compound as an off-white solid (244 mg, 82.9%).

¹H NMR (300 MHz, CHLOROFORM-d) δ ppm 7.88 (dd, J=8.3, 1.5 Hz, 1 H) 7.70 (d, J=6.8 Hz, 1 H) 7.52 (dd, J=8.3, 7.2 Hz, 1 H) 7.11-7.19 (m, 1 H) 7.03 (t, J=7.2 Hz, 2 H) 6.36 (br. s., 1 H) 4.34 (s, 2 H) 3.95 (s, 3 H) 3.15 (s, 3 H). MS APCI, m/z=338.4 (M+H). HPLC 1.63 min.

### EXAMPLE 72

### 9-Amino-2-ethyl-5-(2-fluoro-5-methoxyphenyl)-2,3-dihydropyrrolo[3,4b]quinolin-1-one

Using Method A, 9-amino-5-bromo-2-ethyl-2,3-dihydropyrrolo[3,4-b]quinolin-1-one (250 mg, 0.82 mmol) and 2-fluoro-5-methoxyphenyl boronic acid (278 mg, 1.63 mmol) were reacted to afford the title compound as an off-white solid (218.5 mg, 75.9%).

¹H NMR (300 MHz, CHLOROFORM-d) δ ppm 7.88 (dd, J=8.5, 1.3 Hz, 1 H) 7.71 (d, J=6.8 Hz, 1 H) 7.52 (dd, J=8.3, 7.2 Hz, 1 H) 7.08 (t, J=9.0 Hz, 1 H) 7.00 (dd, J=5.7, 3.0 Hz, 1H) 6.85-6.95 (m, 1H) 6.38 (br. s, 2 H) 4.36 (s, 2 H) 3.82 (s, 3 H) 3.64 (q, J=7.2 Hz, 2 H) 1.26 (t, J=7.3 Hz, 3 H). MS APCI, m/z=352.2 (M+H). HPLC 1.76 min.

### EXAMPLE 73

### 9-Amino-2-ethyl-5-(5-fluoro-2-methoxyphenyl)-2,3-dihydropyrrolo[3,4-b]quinolin-1-one

Using Method A, 9-amino-5-bromo-2-ethyl-2,3-dihydropyrrolo[3,4-b]quinolin-1-one (250 mg, 0.82 mmol) and 5-fluoro-2-methoxyphenyl boronic acid (278 mg, 1.63 mmol) were reacted to afford the title compound as an off-white solid (190 mg, 66.0%).

¹H NMR (300 MHz, CHLOROFORM-d) δ ppm 7.85 (dd, J=8.3, 1.5 Hz, 1 H) 7.67 (dd, J=7.2, 1.5 Hz, 1 H) 7.50 (dd, J=8.3, 7.2 Hz, 1 H) 7.00-7.15 (m, 2 H) 6.95 (dd, J=8.7, 4.1 Hz, 1 H) 6.37 (br. s., 2 H) 4.33 (s, 2 H) 3.68 (s, 3 H) 3.64 (q, J=7.2 Hz, 2 H) 1.26 (t, J=7.2 Hz, 3 H). MS APCI, m/z=352.2 (M+H). HPLC 1.76 min.

### EXAMPLE 74

### 9-Amino-2-ethyl-5-(4-fluoro-3-methoxyphenyl)-2,3-dihydropyrrolo[3,4-b]quinolin-1-one

Using Method A, 9-amino-5-bromo-2-ethyl-2,3-dihydropyrrolo[3,4-b]quinolin-1-one (250 mg, 0.82 mmol) and 4-fluoro-3-methoxyphenyl boronic acid (278 mg, 1.63 mmol) were reacted to afford the title compound as an off-white solid (145 mg, 50.4%).

¹H NMR (300 MHz, CHLOROFORM-d) δ ppm 7.84 (dd, J=8.3, 1.5 Hz, 1 H) 7.72 (dd, J=7.2, 1.5 Hz, 1 H) 7.52 (dd, J=8.3, 7.2 Hz, 1 H) 7.34 (dd, J=7.2, 1.9 Hz, 1 H) 7.11-7.20 (m, 2 H) 6.40 (br. s., 2 H) 4.38 (s, 2 H) 3.93 (s, 3 H) 3.66 (q, J=7.3 Hz, 2 H) 1.28 (t, J=7.3 Hz, 3 H). MS APCI, m/z=352.2 (M+H). HPLC 1.78 min.

### EXAMPLE 75

### 9-Amino-2-ethyl-5-(4-methylpyridin-3-yl)-2,3-dihydropyrrolo[3,4-b]quinolin-1-one

Using Method A, 9-amino-5-bromo-2-ethyl-2,3-dihydropyrrolo[3,4-b]quinolin-1-one (250 mg, 0.82 mmol) and 4-methylpyridin-3-yl-3-boronic acid (224 mg, 1.63 mmol) were reacted to afford the title compound as an off-white solid (166 mg, 63.7%). ¹H NMR (300 MHz, CHLOROFORM-d) δ ppm 8.51 (d, J=4.9 Hz, 1 H) 8.46 (s, 1 H) 7.91 (dd, J=8.1, 1.7 Hz, 1 H) 7.60 (dd, J=7.0, 1.7 Hz, 1 H) 7.54 (t, J=7.5 Hz, 1 H) 7.23 (d, J=4.9 Hz, 1 H) 6.42 (br. s., 2 H) 4.30 (s, 2 H) 3.64 (q, J=7.2 Hz, 2 H) 2.08 (s, 3 H) 1.26 (t, J=7.3 Hz, 3 H). MS APCI, m/z=319.3 (M+H). HPLC 1.05 min.

### EXAMPLE 76

### 2-(9-Amino-2-cyclobutyl-1-oxo-2,3-dihydro-1H-pyrrolo[3,4-b]quinolin-5-yl)benzonitrile

Using Method A, 9-amino-5-bromo-2-cyclobutyl-2,3-dihydro-1H-pyrrolo[3,4-b]quinolin-1-one (100 mg, 0.32 mmol) and 2-cyanophenylboronic acid (95 mg, 10.65 mmol) were reacted to afford the title compound as a white solid (20.3 mg, 5.7%). ¹H NMR (500 MHz, DMSO-d₆) δ ppm 8.47 (dd, J=8.4, 1.4 Hz, 1 H) 7.91 (d, J=7.9 Hz, 1 H) 7.77 (td, J=7.7, 1.4 Hz, 1 H) 7.71 (dd, J=7.0, 1.2 Hz, 1 H) 7.53-7.62 (m, 3 H) 4.73 (quintet, J=8.2 Hz, 1 H) 4.42 (s, 2 H) 2.32 (m, 2 H) 2.11 (m, 2 H) 1.68 (m, 2 H). MS APCI, m/z=355.1 (M+H). HPLC 1.42 min.

### EXAMPLE 77

### 9-Amino-2-cyclobutyl-5-(4-fluoro-2-methoxyphenyl)-2,3-dihydropyrrolo[3,4-b]quinolin-1-one

Using Method A, 9-amino-5-bromo-2-cyclobutyl-2,3-dihydro-1H-pyrrolo[3,4-b]quinolin-1-one (250 mg, 0.75 mmol) and 4-fluoro-2-methoxyphenyl boronic acid (256 mg, 1.51 mmol) were reacted to afford the title compound as an off-white solid (237 mg, 84%). ¹H NMR (300 MHz, CHLOROFORM-d) δ ppm 7.83 (dd, J=8.3, 1.4 Hz, 1 H) 7.65 (dd, J=7.1, 1.4 Hz, 1 H) 7.49 (dd, J=8.2, 7.2 Hz, 1 H) 7.28 (m, 1 H) 6.70-6.82 (m, 2 H) 6.35 (br. s., 2 H) 4.90 (quintet, J=8.5 Hz, 1 H) 4.39 (s, 2 H) 3.70 (s, 3 H) 2.17-2.34 (m, 4 H) 1.76 (quintet, J=7.7 Hz, 2 H). MS APCI, m/z=378.1 (M+H). HPLC 1.90 min.

### EXAMPLE 78

### 9-Amino-2-cyclobutyl-5-(2-fluoro-3-methoxyphenyl)-2,3-dihydropyrrolo[3,4-b]quinolin-1-one

Using Method A, 9-amino-5-bromo-2-cyclobutyl-2,3-dihydro-1H-pyrrolo[3,4-b]quinolin-1-one (250 mg, 0.75 mmol) and 2-fluoro-3-methoxyphenyl boronic acid (256 mg, 1.51 mmol) were reacted to afford the title compound as a pink solid (238 mg, 84%).

¹H NMR (300 MHz, CHLOROFORM-d) δ ppm 7.87 (dd, J=8.3, 1.4 Hz, 1 H) 7.70 (br. d., J=7.0 Hz, 1 H) 7.51 (dd, J=8.2, 7.2 Hz, 1 H) 7.14 (dd, J=7.3, 1.2 Hz, 1 H) 7.03 (t, J=6.8 Hz, 1 H) 6.97-7.08 (m, 1 H) 6.38 (br. s., 2 H) 4.82-4.98 (quintet, J=8.7 Hz, 1 H) 4.43 (s, 2 H) 3.95 (s, 3 H) 2.26 (q, J=7.6 Hz, 4 H) 1.76 (quintet, J=7.8 Hz, 2 H). MS APCI, m/z=378.1 (M+H). HPLC 1.91 min.

### EXAMPLE 79

### 9-Amino-2-cyclobutyl-5-(2-fluoro-5-methoxyphenyl)-2,3-dihydropyrrolo[3,4-b]quinolin-1-one

Using Method A, 9-amino-5-bromo-2-cyclobutyl-2,3-dihydro-1H-pyrrolo[3,4-b]quinolin-1-one (250 mg, 0.75 mmol) and 2-fluoro-5-methoxyphenyl boronic acid (128 mg, 0.75 mmol) were reacted to afford the title compound as a pink solid (248 mg, 87%).

¹H NMR (300 MHz, CHLOROFORM-d) δ ppm 7.88 (dd, J=8.3, 1.4 Hz, 1 H) 7.71 (br. d., J=6.7 Hz, 1 H) 7.52 (dd, J=8.2, 7.2 Hz, 1 H) 7.08 (t, J=9.0 Hz, 1 H) 7.00 (dd, J=5.7, 3.2 Hz, 1 H) 6.92 (dt, J=9.1, 3.5 Hz, 1 H) 6.39 (br. s., 2 H) 4.90 (quintet, J=8.8 Hz, 1 H) 4.44 (s, 2 H) 3.82 (s, 3 H) 2.27 (q, J=7.7 Hz, 4 H) 1.77 (quintet, J=7.6 Hz, 2 H). MS APCI, m/z=378.1 (M+H). HPLC 1.92 min.

### EXAMPLE 80

### 9-Amino-2-cyclobutyl-5-pyrazin-2-yl-2,3-dihydropyrrolo[3,4-b]quinolin-1-one

Using Method E, 9-amino-5-bromo-2-cyclobutyl-2,3-dihydro-1H-pyrrolo[3,4-b]quinolin-1-one (350 mg, 1.05 mmol) and 2-tributylstannyl-pyrazine (488 mg, 1.26 mmol) were reacted to afford the title compound as a yellow solid (220 mg, 63.0%). ¹H NMR 1H NMR (300 MHz, CHLOROFORM-d) δ ppm 9.43 (d, J=1.3 Hz, 1 H) 8.70 (d, J=2.3 Hz, 1 H) 8.54 (d, J=2.5 Hz, 1 H) 8.18 (dd, J=7.2, 1.3 Hz, 1 H) 7.95 (dd, J=8.3, 1.4 Hz, 1 H) 7.61 (dd, J=8.2, 7.4 Hz, 1 H) 6.45 (br. s., 2 H) 4.92 (quintet, J=8.5 Hz, 1 ) 4.47 (s, 2 H) 2.30 (q, J=8.1 Hz, 4 H) 1.80 (quintet, J=8.0 Hz, 2 H). MS APCI, m/z=332.1 (M+H). HPLC 1.49 min.

### EXAMPLE 81

### 9-Amino-2-cyclobutyl-5-(3-methoxypyridin-4-yl)-2,3-dihydropyrrolo[3, 4-b]quinolin-1-one

Using Method A, 9-amino-5-bromo-2-cyclobutyl-2,3-dihydro-1H-pyrrolo[3,4-b]quinolin-1-one (250 mg, 0.75 mmol) and 3-methoxy-pyridin-4-boronic acid (230 mg, 1.51 mmol) were reacted to afford the title compound as a pale-peach solid (187 mg, 68.9%). ¹H NMR (300 MHz, CHLOROFORM-d) δ ppm 8.43 (s, 1 H) 8.35 (d, J=4.8 Hz, 1 H) 7.88 (dd, J=8.3, 1.4 Hz, 1 H) 7.67 (dd, J=7.1, 1.4 Hz, 1 H) 7.53 (dd J=8.2, 7.4 Hz, 1 H) 7.29 (d, J=4.6 Hz, 1 H) 6.39 (br. s., 2 H) 4.90 (quintet, J=8.7 Hz, 1 H) 4.39 (s, 2 H) 3.81 (s, 3 H) 2.26 (q, J=7.6 Hz, 4 H) 1.77 (quintet, J=7.7 Hz, 2 H). MS APCI, m/z=361.2 (M+H). HPLC 1.38 min.

### EXAMPLE 82

### 9-Amino-2-cyclobutyl-5-pyridin-4-yl-2,3-dihydropyrrolo[3,4-b]quinolin-1-one

Using Method E, 9-amino-5-bromo-2-cyclobutyl-2,3-dihydro-1H-pyrrolo[3,4-b]quinolin-1-one (350 mg, 1.05 mmol) and 4-tributylstannyl-pyridine (500 mg, 1.36 mmol) were reacted to afford the title compound as an off-white solid (193 mg, 55.5%).

¹H NMR (500 MHz, DMSO-d₆) δ ppm 8.69 (br. s., 2 H) 8.47 (dd, J=8.2, 1.2 Hz, 1 H) 7.80 (dd, J=7.0, 1.2 Hz, 1 H) 7.73 (d, J=4.6 Hz, 2 H) 7.58 (dd, J=8.2, 7.3 Hz, 1 H) 4.74 (t, J=8.2 Hz, 1 H) 4.48 (s, 2 H) 2.34 (quintet-doublet, J=9.6, 2.4 Hz, 2 H) 2.12 (m, 2 H) 1.69 (m, 2 H). MS APCI, m/z=331.2 (M+H). HPLC 4.59 min.

### EXAMPLE 83

### 9-Amino-2-cyclobutyl-5-pyridin-2-yl-2,3-dihydropyrrolo[3,4-b]quinolin-1-one

Using Method E, 9-amino-5-bromo-2-cyclobutyl-2,3-dihydro-1H-pyrrolo[3,4-b]quinolin-1-one (350 mg, 1.05 mmol) and 2-tributylstannyl-pyridine (490 mg, 1.33 mmol) were reacted to afford the title compound as a white solid (136 mg, 39.2%). ¹H NMR (500 MHz, DMSO-d₆) δ ppm 8.68 (d, J=4.0 Hz, 1 H) 8.42 (dd, J=8.4, 1.4 Hz, 1 H) 8.07 (d, J=7.9 Hz, 1 H) 8.02 (dd, J=7.2, 1.4 Hz, 1 H) 7.83 (td, J=7.8, 1.8 Hz, 1 H) 7.57 (d, J=8.2 Hz, 1 H) 7.37 (ddd, J=7.3, 4.9, 1.2 Hz, 1 H) 4.75 (quintet, J=8.6 Hz, 1 H) 4.49 (s, 2 H) 2.35 (quintet-doublet, J=9.5, 2.4 Hz, 2 H) 2.13 (m, 2 H) 1.70 (m, 2 H). MS APCI, m/z=331.1 (M+H). HPLC 1.77 min.

### EXAMPLE 84

### 9-Amino-2-cyclobutyl-5-(3,6-dimethoxypyridazin-4-yl)-2,3-dihydropyrrolo[3,4-b]quinolin-1-one

Using Method E, 9-amino-5-bromo-2-cyclobutyl-2,3-dihydro-1H-pyrrolo[3,4-b]quinolin-1-one (350 mg, 1.05 mmol) and 3,6-dimethoxy-4-tributylstannyl-pyridazine (904 mg, 2.11 mmol) were reacted to afford the title compound as an off-white solid (326 mg, 79%). ¹H NMR (300 MHz, CHLOROFORM-d) δ 7.91 (dd, J=8.3, 1.4 Hz, 1 H) 7.68 (dd, J=7.2, 1.5 Hz, 1 H) 7.52 (dd, J=8.2, 7.2 Hz, 1 H) 7.03 (s, 1 H) 6.41 (br. s., 2 H) 4.90 (quintet, J=8.8 Hz, 1 H) 4.39 (s, 2 H) 4.12 (s, 3 H) 2.28 (q, J=7.7 Hz, 4 H) 1.78 (quintet, J=7.7 Hz, 2 H). MS APCI, m/z=392.2 (M+H). HPLC 1.71 min.

### EXAMPLE 85

### 9-Amino-2-cyclobutyl-5-(6-methoxypyridin-2-yl)-2,3-dihydropyrrolo[3,4-b]quinolin-1-one

Using Method E, 9-amino-5-bromo-2-cyclobutyl-2,3-dihydro-1H-pyrrolo[3,4-b]quinolin-1-one (350 mg, 1.05 mmol) and 6-methoxy-2-tributylstannyl-pyridine (839 mg, 2.11 mmol) were reacted to afford the title compound as a white solid (188 mg, 49.5%). ¹H NMR (300 MHz, CHLOROFORM-d) δ ppm 8.26 (dd, J=7.3, 1.4 Hz, 1 H) 7.87 (dd, J=8.3, 1.4 Hz, 1 H) 7.81 (d, J=7.4 Hz, 1 H) 7.66 (t, J=7.8 Hz, 1 H) 7.57 (dd, J=8.2, 7.6 Hz, 1 H) 6.75 (d, J=8.0 Hz, 1 H) 6.39 (br. s., 2 H) 4.92 (quintet, J=8.6 Hz, 1 H) 4.49 (s, 2 H) 4.02 (s, 3 H) 2.22-2.36 (m, 4 H) 1.79 (quintet, J=8.0 Hz, 2 H). MS APCI, m/z=361.1 (M+H). HPLC 1.94 min.

### EXAMPLE 86

### 9-Amino-2-cyclobutyl-3-hydroxy-5-(6-methylpyridin-2-yl)-2,3-dihydropyrrolo[3,4-b]quinolin-1-one

Using Method E, 9-amino-5-bromo-2-cyclobutyl-2,3-dihydro-1H-pyrrolo[3,4-b]quinolin-1-one (350 mg, 1.05 mmol) and 6-methy-2-tributylstannyl-pyridine (805 mg, 2.11 mmol) were reacted to afford the title compound as a white solid (26 mg, 6.85%).

¹H NMR (500 MHz, DMSO-d₆) δ ppm 8.40 (dd, J=8.5, 1.2 Hz, 1 H) 8.03 (dd, J=7.2, 1.1 Hz, 1 H) 7.92 (d, J=7.9 Hz, 1 H) 7.72 (t, J=7.8 Hz, 1 H) 7.58 (d, J=7.3 Hz, 1 H) 7.23 (d, J=7.6 Hz, 1 H) 6.50 (d, J=9.5 Hz, 1 H) 5.79 (d, J=9.5 Hz, 1 H) 4.49 (quintet, J=8.7 Hz, 1 H) 2.61 (quintet, J=10.4 Hz, 2 H) 2.52-2.58 (one proton was buried under the solvent) 2.10-2.20 (m, 2 H) 1.63-1.77 (m, 2 H). MS APCI, m/z=361.3 (M+H). HPLC 1.64 min.

### EXAMPLE 87

### 9-Amino-2-cyclobutyl-5-(5-methylpyridin-2-yl)-2,3-dihydropyrrolo[3,4-b]quinolin-1-one

Using Method E, 9-amino-5-bromo-2-cyclobutyl-2,3-dihydro-1H-pyrrolo[3,4-b]quinolin-1-one (350 mg, 1.05 mmol) and 5-methy-2-tributylstannyl-pyridine (805 mg, 2.11 mmol) were reacted to afford the title compound as a pale-yellow solid (201 mg, 55.4%). ¹H NMR (300 MHz, CHLOROFORM-d) δ ppm 8.60 (d, J=2.1 Hz, 1 H) 8.11 (dd, J=7.3, 1.4 Hz, 1 H) 7.96 (d, J=8.2 Hz, 1 H) 7.90 (dd, J=8.2, 1.3 Hz, 1 H) 7.56 (dd, J=8.2, 7.4 Hz, 1 H) 7.59 (dd, J=8.3, 2.0 Hz, 1 H) 6.44 (br. s., 2 H) 4.91 (quintet, J=8.9, 8.6 Hz, 1 H) 4.48 (s, 2 H) 2.42 (s, 3 H) 2.28 (q, J=7.7 Hz, 4 H) 1.78 (quintet, J=6.9 Hz, 2 H). MS APCI, m/z=345.1 (M+H). HPLC 1.89 min.

### EXAMPLE 88

### 9-Amino-2-cyclobutyl-5-pyrimidin-2-yl-2,3-dihydropyrrolo[3,4-b]quinolin-1-one

9-amino-5-bromo-2-cyclobutyl-2,3-dihydro-1H-pyrrolo[3,4-b]quinolin-1-one (306 mg, 0.92 mmol), CombiPhos-Pd6 (46.1 mg, 0.09 mmol), 2-(tributylstannyl)-pyrimidine (680 mg, 1.84 mmol) and N,N-dicyclohexylmethylamine (252 mg, 1.29 mmol) in DMF (5 mL) were heated at 100° C. for 48 hours. Then, the reaction mixture was cooled to room temperature, diluted with methylene chloride (100 ml), washed with water, dried through magnesium sulfate and evaporated to dry. The crude product was purified by column chromatography three times eluted with 20-100% ethyl acetate in hexane, 0-100% CAN in chloroform and 0-5% methanol in methylene chloride to afford the title compound as a yellow solid (26 mg, 8.5%). ¹H NMR (500 MHz, CHLOROFORM-d) δ ppm 8.93 (d, J=4.9 Hz, 2 H) 7.96 (t, J=7.2 Hz, 2 H) 7.56 (dd, J=8.2, 7.3 Hz, 1 H) 7.33 (t, J=5.0 Hz, 1 H) 6.41 (br. s., 2 H) 4.90 (quintet, J=8.7 Hz, 1 H) 4.46 (s, 2 H) 2.19-2.30 (m, 4 H) 1.71-1.83 (m, 2 H). MS APCI, m/z=332.3 (M+H). HPLC 1.63 min.

### EXAMPLE 89

### 6-(9-Amino-2-cyclobutyl-1-oxo-2,3-dihydro-1H-pyrrolo[3,4-b]quinolin-5-yl)-nicotinonitrile

Using Method E, 9-amino-5-bromo-2-cyclobutyl-2,3-dihydro-1H-pyrrolo[3,4-b]quinolin-1-one (186 mg, 0.56 mmol) and 6-tributylstannyl-nicotinonitrile (220 mg, 0.56 mmol) were reacted to afford the title compound as an off-white solid (44 mg, 22.1%).

¹H NMR (300 MHz, CHLOROFORM-d) δ ppm 9.01 (d, J=1.32 Hz, 1H) 8.38 (d, J=8.9 Hz, 1 H) 8.22 (dd, J=7.3, 1.1 Hz, 1 H) 8.01 (dd, J=8.3, 2.3 Hz, 1 H) 7.96 (dd, J=8.38 (d, J=8.9 Hz, 1 H) 7.61 (t, J=7.8 Hz, 1 H) 6.46 (br. s., 2 H) 4.92 (quintet, J=8.6 Hz, 1 H) 4.47 (s, 2 H) 2.30 (q, J=7.7 Hz, 4 H) 1.80 (quintet, J=7.4 Hz, 2 H). MS APCI, m/z=356.1 (M+H). HPLC 1.78 min.

### EXAMPLE 90

### 5-(9-Amino-2-cyclobutyl-1-oxo-2,3-dihydro-1H-pyrrolo[3,4-b]quinolin-5-yl)-nicotinonitrile

Using Method E, 9-amino-5-bromo-2-cyclobutyl-2,3-dihydro-1H-pyrrolo[3,4-b]quinolin-1-one (350 mg, 1.05 mmol) and 5-trimethylstannyl-nicotinonitrile (562 mg, 2.11 mmol) were reacted to afford the title compound as an off-white solid (228 mg, 60.8%). ¹H NMR (300 MHz, CHLOROFORM-d) δ ppm 9.08 (d, J=2.1 Hz, 1 H) 8.88 (d, J=2.0 Hz, 0 H) 8.38 (t, J=2.0 Hz, 1 H) 7.95 (dd, J=8.4, 1.2 Hz, 1 H) 7.75 (dd, J=7.2, 1.3 Hz, 1 H) 7.58 (dd, J=8.2, 7.4 Hz, 1 H) 6.48 (br. s., 2 H) 4.92 (quintet, J=8.6 Hz, 1 H) 4.46 (s, 1 H) 2.18-2.44 (m, 4 H) 1.80 (t, J=8.0 Hz, 2 H). MS APCI, m/z=356.1 (M+H). HPLC 1.79 min.

### EXAMPLE 91

### 9-Amino-2-cyclobutyl-5-(3-methoxypyridazin-4-yl)-2,3-dihydropyrrolo[3,4-b]quinolin-1-one

Using Method E, 9-amino-5-bromo-2-cyclobutyl-2,3-dihydro-1H-pyrrolo[3,4-b]quinolin-1-one (350 mg, 1.05 mmol) and 3-methoxy-4-tributylstannyl-pyridazine (841 mg, 2.11 mmol) were reacted to afford the title compound as a peach solid (271 mg, 71.2%). ¹H NMR (300 MHz, CHLOROFORM-d) δ ppm 8.94 (d, J=4.5 Hz, 1 H) 7.93 (dd, J=8.4, 1.2 Hz, 1 H) 7.71 (dd, J=7.1, 1.2 Hz, 1 H) 7.53 (dd, J=8.1, 7.3 Hz, 1 H) 7.46 (d, J=4.7 Hz, 1 H) 6.44 (br. s., 2 H) 4.90 (quintet, J=8.7 Hz, 1 H) 4.38 (s, 2 H) 4.07 (s, 3 H) 2.27 (q, J=7.7 Hz, 4 H) 1.78 (quintet, J=7.7 Hz, 2 H). MS APCI, m/z=362.1 (M+H). HPLC 1.55 min.

### EXAMPLE 92

### 9-Amino-2-cyclobutyl-5-(4-methoxy-pyrimidin-5-yl)-2,3-dihydropyrrolo[3,4-b]quinolin-1-one

Using Method E, 9-amino-5-bromo-2-cyclobutyl-2,3-dihydro-1H-pyrrolo[3,4-b]quinolin-1-one (350 mg, 1.05 mmol) and 4-methoxy-5-tributylstannyl-pyrimidine (715 mg, 1.79 mmol) were reacted to afford the title compound as a yellow solid (265 mg, 69.6%). ¹H NMR (300 MHz, CHLOROFORM-d) δ ppm 8.84 (br. s., 1 H) 8.56 (br. s., 1 H) 7.90 (dd, J=8.2, 1.3 Hz, 1 H) 7.72 (dd, J=7.2, 1.3 Hz, 1 H) 7.52 (dd, J=8.1, 7.5 Hz, 1 H) 6.43 (br. s., 2 H) 4.90 (quintet, J=8.6 Hz, 1 H) 4.39 (s, 2 H) 3.94 (s, 3 H) 2.27 (q, J=7.7 Hz, 4 H) 1.78 (quintet, J=7.7 Hz, 2 H). MS APCI, m/z=362.1 (M+H). HPLC 1.62 min.

### EXAMPLE 93

### 9-Amino-2-cyclobutyl-5-(3-fluoropyridin-2-yl)-2,3-dihydropyrrolo[3,4-b]quinolin-1-one

Using Method E, 9-amino-5-bromo-2-cyclobutyl-2,3-dihydro-1H-pyrrolo[3,4-b]quinolin-1-one (350 mg, 1.05 mmol) and 3-fluoro-2-tributylstannyl-pyridine (1.54 g, 50% wt, 1.99 mmol) were reacted to afford the title compound as a peach solid (260 mg, 70.8%). ¹H NMR (300 MHz, CHLOROFORM-d) δ ppm 8.57 (dt, J=4.4, 1.3 Hz, 1 H) 7.94 (dd, J=8.2, 1.3 Hz, 1 H) 7.84 (dd, J=7.1, 1.2 Hz, 1H) 7.56 (dd, J=8.1, 7.5 Hz, 1 H) 7.52 (td, J=8.75, 1.3 Hz, 1 H) 7.39 (td, J=8.2, 4.2 Hz, 1 H) 6.41 (br. s., 2 H) 4.89 (quintet, J=8.8 Hz, 1 H) 4.41 (s, 2 H) 2.26 (q, J=7.6 Hz, 4 H) 1.76 (quintet, J=8.4 Hz, 2 H). MS APCI, m/z=349.2 (M+H). HPLC 1.53 min.

### EXAMPLE 94

### 2-(9-Amino-2-cyclobutyl-1-oxo-2,3-dihydro-1H-pyrrolo[3,4-b]quinolin-5-yl)-5-fluorobenzonitrile

Using Method E, 9-amino-5-bromo-2-cyclobutyl-2,3-dihydro-1H-pyrrolo[3,4-b]quinolin-1-one (300 mg, 0.90 mmol) and 5-fluoro-2-tributylstannyl-benzonitrile (900 mg, 70% wt, 1.54 mmol) were reacted to afford the title compound as a pale-peach solid (196 mg, 58.3%). ¹H NMR (300 MHz, CHLOROFORM-d) δ ppm 7.93 (dd, J=8.3, 1.4 Hz, 1 H) 7.70 (dd, J=7.2, 1.3 Hz, 1 H) 7.51-7.59 (m, 2 H) 7.48 (dd, J=8.2, 2.7 Hz, 1 ) 7.39 (td, J=8.3, 2.7 Hz, 1 H) 6.43 (br. s., 2 H) 4.89 (quintet, J=9.0, 8.7 Hz, 1 H) 4.42 (s, 2 H) 2.19-2.35 (m, 4 H) 1.77 (quintet, J=7.8 Hz, 2 H). MS APCI, m/z=373.3 (M+H). HPLC 1.85 min.

### EXAMPLE 95

### 2-(9-Amino-2-cyclobutyl-1-oxo-2,3-dihydro-1H-pyrrolo[3,4-b]quinolin-5-yl)-4-fluorobenzonitrile

Using Method E, 9-amino-5-bromo-2-cyclobutyl-2,3-dihydro-1H-pyrrolo[3,4-b]quinolin-1-one (350 mg, 1.05 mmol) and 4-fluoro-2-tributylstannyl-benzonitrile (1.24 g, 70% wt, 2.11 mmol) were reacted to afford the title compound as a white solid (227 mg, 57.9%). ¹H NMR (300 MHz, CHLOROFORM-d) δ ppm 7.95 (dd, J=8.4, 1.5 Hz, 1 H) 7.78 (dd, J=8.5, 5.6 Hz, 1 H) 7.72 (dd, J=7.2, 1.3 Hz, 1 H) 7.55 (dd, J=8.3, 7.3 Hz, 1 H) 7.31 (dd, J=9.2, 2.6 Hz, 1 H) 7.19 (dd, J=8.1, 5.6 Hz, 1 H) 6.44 (br. s., 2 H) 4.90 (quintet, J=8.8 Hz, 1 H) 4.43 (s, 2 H) 2.21-2.33 (td, J=8.2, 7.5 Hz, 4 H) 1.77 (quintet, J=7.9 Hz, 2 H). MS APCI, m/z=373.3 (M+H). HPLC 1.93 min.

### EXAMPLE 96

### 4-(9-Amino-2-cyclobutyl-1-oxo-2,3-dihydro-1H-pyrrolo[3,4-b]quinolin-5-yl)-6-methoxynicotinonitrile

Using Method E, 9-amino-5-bromo-2-cyclobutyl-2,3-dihydro-1H-pyrrolo[3,4-b]quinolin-1-one (500 mg, 1.51 mmol) and 6-methoxy-4-tributylstannyl-nicotinonitrile (2.0 g, 71% wt, 3.36 mmol) were reacted to afford the title compound as a white solid (188.3 mg, 32.5%). ¹H NMR (500 MHz, DMSO-d₆) δ ppm 8.74 (s, 1H) 8.51 (d, J=8.5 Hz, 1 H) 7.75 (d, J=7.3 Hz, 1 H) 7.58 (t, J=7.6 Hz, 1 H) 7.05 (s, 1 H) 4.73 (quintet, J=8.6 Hz, 1 H) 4.43 (s, 2 H) 4.01 (s, 3 H) 2.28-2.38 (m, 2 H) 2.05-2.15 (m, 2 H) 1.61-1.74 (m, 2 H). MS APCI, m/z=386.0 (M+H). HPLC 7.83 min.

### EXAMPLE 97

### 9-Amino-5-(1,3-dimethyl-1H-pyrazol-4-yl)-6-fluoro-2-(R)-tetrahydro-furan-3-yl-2,3-dihydropyrrolo[3,4-b]quinolin-1-one

Using Method A, 9-amino-5-bromo-6-fluoro-2-[(R)-tetrahydro-furan-3-yl]-2,3-dihydropyrrolo[3,4-b]quinolin-1-one (250 mg, 0.68 mmol) and 1,3-dimethyl-4-(4,4,5,5-tetramethyl-[1,3,2]dioxaborolan2-yl)-1H-pyrazole (354 mg, 2.31 mmol) were reacted to afford the title compound as a peach solid (81.3 mg, 31.2%). ¹H NMR (300 MHz, CHLOROFORM-d) δ ppm 7.79 (dd, J=9.3, 5.7 Hz, 1 H) 7.56 (s, 1 H) 7.32 (t, J=9.0 Hz, 1 H) 6.39 (br. s., 2 H) 5.11 (sextet, J=4.3 Hz, 1 H) 4.47 (d, J=12.2 Hz, 1 H) 4.41 (d, J=12.4 Hz, 1 H) 4.09 (td, J=8.5, 5.9 Hz, 1 H) 3.94 (s, 3 H) 3.79-3.91 (m, 1 H) 3.89 (d, J=4.8 Hz, 2 H) 2.28-2.45 (m, 1 H) 2.18 (s, 3 H) 1.93-2.10 (m, 1 H). MS APCI, m/z=382.2 (M+H). HPLC 1.40 min.

### EXAMPLE 98

### 9-Amino-2-cyclobutyl-5-(5-fluoro-2-methoxypyridin-4-yl)-2,3-dihydropyrrolo[3,4-b]quinolin-1-one

Using Method E, 9-amino-5-bromo-2-cyclobutyl-2,3-dihydro-1H-pyrrolo[3,4-b]quinolin-1-one (500 mg, 1.51 mmol) and 5-fluoro-2-methoxy-4-tributylstannyl-pyridine (2.1 g, 35% wt, 1.34 mmol) were reacted to afford the title compound as a white solid (251.9 mg, 44.2%). ¹H NMR (500 MHz, DMSO-d₆) δ ppm 8.47 (dd, J=8.5, 1.2 Hz, 1 H) 8.18 (d, J=1.2 Hz, 1 H) 7.72 (dd, J=7.0, 0.9 Hz, 1 H) 7.56 (dd, J=8.4, 7.2 Hz, 1 H) 6.90 (d, J=4.6 Hz, 1 H) 4.73 (quintet, J=8.6 Hz, 1 H) 4.45 (s, 2 H) 3.90 (s, 3 H) 2.33 (quintet-doublet, J=9.5, 2.4 Hz, 2 H) 2.05-2.17 (m, 2 H) 1.63-1.75 (m, 2 H). MS APCI, m/z=379.2 (M+H). HPLC 8.52 min.

### EXAMPLE 99

### 9-Amino-2-cyclobutyl-5-(5-fluoro-2-methoxyphenyl)-2,3-dihydropyrrolo[3,4-b]quinolin-1-one

Using Method A, 9-amino-5-bromo-2-cyclobutyl-2,3-dihydro-1H-pyrrolo[3,4-b]quinolin-1-one (250 mg, 0.75 mmol) and 5-fluoro-2-methoxyphenylboronic acid (256 mg, 1.51 mmol) were reacted to afford the title compound as a white solid (251.5 mg, 89%). ¹H NMR (300 MHz, CHLOROFORM-d) δ ppm 7.84 (dd, J=8.3, 1.4 Hz, 1 H) 7.67 (dd, J=7.2, 1.5 Hz, 1 H) 7.50 (dd, J=8.2, 7.2 Hz, 1 H) 7.02-7.13 (m, 2 H) 6.90-7.00 (m, 1 H) 6.37 (br. s., 2 H) 4.90 (quintet, J=8.5 Hz, 1 H) 4.40 (s, 2 H) 3.68 (s, 3 H) 2.26 (q, J=7.6 Hz, 4 H) 1.77 (quintet, J=7.7 Hz, 2 H. MS APCI, m/z=378.1 (M+H). HPLC 1.93 min.

### EXAMPLE 100

### 9-Amino-2-cyclobutyl-5-(2,4-dimethoxyphenyl)-6-fluoro-2,3-dihydro-1H-pyrrolo[3,4-b]quinolin-1-one

Using Method F, 9-amino-5-bromo-2-cyclobutyl-6-fluoro-2,3-dihydro-1H-pyrrolo[3,4-b]quinolin-1-one (150 mg, 0.43 mmol), and 2,4-Dimethoxyphenylboronic acid (234 mg, 1.29 mmol) were reacted to afford the title compound as a white solid (104 mg, 60% yield). 1H NMR (500 MHz, DMSO-d₆) δ ppm 8.56-8.16 (m, 1H), 7.67 (br, 2H), 7.39 (t, J=9 Hz, 1H), 7.08 (d, J=9 Hz, 1H), 6.70-6.67 (m, 2H), 4.71 (m, 1H), 4.38 (s, 2H), 3.84 (s, 3H), 3.64 (s, 3H), 2.30 (m, 2H), 2.11 (m, 2H), 1.67 (m, 2H). MS APCI, m/z=408 (M+H).

### EXAMPLE 101

### 9-Amino-2-cyclobutyl-6-fluoro-5-(6-methylpyridin-3-yl)-2,3-dihydro-1H-pyrrolo[3,4-b]quinolin-1-one

Using Method F, 9-amino-5-bromo-2-cyclobutyl-6-fluoro-2,3-dihydro-1H-pyrrolo[3,4-b]quinolin-1-one (150 mg, 0.43 mmol), and 6-methylpyridine-3-boronic acid (176 mg, 1.29 mmol) were reacted to afford the title compound as a white solid (109 mg, 70% yield). 1H NMR (500 MHz, DMSO-d₆) δ ppm 8.49 (m, 2H), 7.77 (m, 3H), 7.51 (t, J=9 Hz, 1H), 7.38 (d, J=8 Hz, 1H), 4.71 (m, 1H), 4.43 (s, 2H), 2.56 (s, 3H), 2.30 (m, 2H), 2.11 (m, 2H), 1.67 (m, 2H). MS APCI, m/z=363 (M+H).

### EXAMPLE 102

### 9-Amino-2-cyclobutyl-6-fluoro-5-(2-fluoropyridin-3-yl)-2,3-dihydro-1H-pyrrolo[3,4-b]quinolin-1-one

Tetrakis(triphenylphosphine)palladium (0) (74.2 mg, 0.06 mmol) was added to a mixture of 9-amino-5-bromo-2-cyclobutyl-6-fluoro-2,3-dihydro-1H-pyrrolo[3,4-b]quinolin-1-one (150 mg, 0.43 mmol), 2-fluoropyridine-3-boronic acid (181 mg, 1.29 mmol), and cesium carbonate (517 mg, 1.59 mmol) in DME (2 mL), ethanol (0.571 mL), and water (0.857 mL) under nitrogen at 25° C. in a septum-capped microwave reaction vial. The mixture was heated by microwave at 110° C. for 20 minutes, cooled to room temperature and diluted with ethyl acetate. The organic phase was separated, filtered and evaporated. The organic residue was purified by flash chromatography on silica gel eluting with an increasingly polar gradient of acetonitrile in chloroform (10-60%). The product was crystallized from a small volume of acetonitrile to afford the title compound (50 mg, 32% yield) as a white solid. 1H NMR (500 MHz, DMSO-d₆) δ ppm 8.68 (m, 1H), 8.41 (m, 1H), 8.08 (m, 1H), 7.79 (br, 2H) 7.67 (m, 1H), 7.56 (m, 1H), 4.70 (m, 1H), 4.53 (s, 2H), 2.30 (m, 2H), 2.11 (m, 2H), 1.69 (m, 2H). MS APCI, m/z=367 (M+H).

### EXAMPLE 103

### 9-Amino-2-cyclobutyl-6-fluoro-5-(4-fluoro-2-methoxyphenyl)-2,3-dihydro-1H-pyrrolo[3,4-b]quinolin-1-one

Using Method F, 9-amino-5-bromo-2-cyclobutyl-6-fluoro-2,3-dihydro-1H-pyrrolo[3,4-b]quinolin-1-one (150 mg, 0.43 mmol), and 4-fluoro-2-methoxyphenylboronic acid (182 mg, 1.07 mmol) were reacted to afford the title compound as a white solid (78 mg, 46% yield). 1H NMR (500 MHz, DMSO-d₆) δ ppm 8.42 (m, 1H), 7.67 (br, 2H), 7.42 (t, J=9 Hz, 1H), 7.18 (m, 1H), 7.03 (dd, 1H), 6.85 (m, 1H), 4.71 (m, 1H), 4.38 (s, 2H), 3.66 (s, 3H), 2.30 (m, 2H), 2.11 (m, 2H), 1.67 (m, 2H). MS APCI, m/z=396 (M+H).

### EXAMPLE 104

### 9-Amino-2-cyclobutyl-6-fluoro-5-(pyrimidin-5-yl)-2,3-dihydro-1H-pyrrolo[3,4-b]quinolin-1-one

Using Method F, 9-amino-5-bromo-2-cyclobutyl-6-fluoro-2,3-dihydro-1H-pyrrolo[3,4-b]quinolin-1-one (150 mg, 0.43 mmol), and pyrimidine-5-boronic acid (212 mg, 1.71 mmol) were reacted to afford the title compound as a white solid (25 mg, 13% yield). 1H NMR (500 MHz, DMSO-d₆) δ ppm 9.21 (s, 1H), 8.94 (s, 2H), 8.54 (m, 1H), 7.72 (br, 2H), 7.57 (t, J=9 Hz, 1H), 4.72 (m, 1H), 4.46 (s, 2H), 2.30 (m, 2H), 2.11 (m, 2H), 1.67 (m, 2H). MS APCI, m/z=350 (M+H).

### EXAMPLE 105

### 9-Amino-2-cyclobutyl-6-fluoro-5-(3-methoxypyridin-4-yl)-2,3-dihydro-1H-pyrrolo[3,4-b]quinolin-1-one

Using Method F, 9-amino-5-bromo-2-cyclobutyl-6-fluoro-2,3-dihydro-1H-pyrrolo[3,4-b]quinolin-1-one (200 mg, 0.57 mmol), and 3-methoxypyridine-4-boronic acid (262 mg, 1.71 mmol) were reacted to afford the title compound as a white solid (51 mg, 24% yield). 1H NMR (500 MHz, DMSO-d₆) δ ppm 8.50 (m, 2H), 8.29 (d, J=5 Hz, 1H), 7.46 (m, 1H), 7.72 (br, 2H), 7.25 (d, 1H), 4.72 (m, 1H), 4.40 (s, 2H), 3.78 (s, 3H), 2.30 (m, 2H), 2.11 (m, 2H), 1.67 (m, 2H). MS APCI, m/z=379 (M+H).

### EXAMPLE 106

### 9-Amino-2-cyclobutyl-6-fluoro-5-(2-fluoro-3-methoxyphenyl)-2,3-dihydro-1H-pyrrolo[3,4-b]quinolin-1-one

To a solution of 9-amino-5-bromo-2-cyclobutyl-6-fluoro-2,3-dihydro-1H-pyrrolo[3,4-b]quinolin-1-one (150 mg, 0.43 mmol) in acetonitrile (2 mL) and water (2 mL) was added 2-fluoro-3-methoxyphenylboronic acid (146 mg, 0.86 mmol), potassium carbonate (148 mg, 1.07 mmol) and bis(di-tert-butyl(4-dimethylaminophenyl)phosphine)-dichloropalladium(II) (15.16 mg, 0.02 mmol). The suspension was heated at 140° C. in a sealed tube by microwave for 20 minutes then cooled and diluted with ethyl acetate. The organic phase was separated, evaporated and the residue purified by flash chromatography on silica gel eluting with acetonitrile in chloroform 10-50% gradient. Product containing fractions were pooled and evaporated to afford the title compound (169 mg, 47% yield) as a white solid. 1H NMR (500 MHz, DMSO-d₆) δ ppm 8.51 (m, 1H), 7.75 (br, 2H), 7.49 (m, 1H), 7.22 (m, 2H), 6.93 (m, 1H), 4.71 (m, 1H), 4.42 (m, 2H), 3.90 (s, 3H), 2.30 (m, 2H), 2.10 (m, 2H), 1.67 (m, 2H). MS APCI, m/z=396 (M+H).

### EXAMPLE 107

### 9-Amino-2-cyclobutyl-6-fluoro-5-(6-methoxypyridin-3-yl)-2,3-dihydro-1H-pyrrolo[3,4-b]quinolin-1-one

Using Method F, 9-amino-5-bromo-2-cyclobutyl-6-fluoro-2,3-dihydro-1H-pyrrolo[3,4-b]quinolin-1-one (150 mg, 0.43 mmol), and 4-methoxy-3-pyridinyl boronic acid (164 mg, 1.07 mmol) were reacted to afford the title compound as a white solid (65 mg, 40% yield). 1H NMR (500 MHz, DMSO-d₆) δ ppm 8.46 (m, 1H), 8.24 (s, 1H), 7.79 (m, 1H), 7.72 (br, 2H), 7.49 (t, 1H), 6.93 (d, 1H), 4.72 (m, 1H), 4.44 (s, 2H), 3.93 (s, 3H), 2.32 (m, 2H), 2.12 (m, 2H), 1.68 (m, 2H). MS APCI, m/z=379 (M+H).

### EXAMPLE 108

### 9-Amino-2-cyclobutyl-6-fluoro-5-(2-vinylphenyl)-2,3-dihydro-1H-pyrrolo[3,4-b]quinolin-1-one

Using Method F, 9-amino-5-bromo-2-cyclobutyl-6-fluoro-2,3-dihydro-1H-pyrrolo[3,4-b]quinolin-1-one (300 mg, 0.86 mmol), and 2-vinylbenzeneboronic acid (190 mg, 1.29 mmol) were reacted to afford the title compound as a white solid (215 mg,67% yield). 1H NMR (500 MHz, DMSO-d₆) δ ppm 8.49 (m, 1H), 7.75 (d, 1H), 7.70 (br, 2H), 7.49-7-40 (m, 2H), 7.36 (m, 1H), 7.19 (d, 1H), 6.20 (m, 1H), 5.68 (d, J=17 Hz, 1H), 5.03 (d, J=12 Hz, 1H), 4.70 (m, 1H), 4.37 (s, 2H), 2.28 (m, 2H), 2.09 (m, 2H), 1.68 (m, 2). MS APCI, m/z=374 (M+H).

### EXAMPLE 109

### 9-Amino-2-(3-chloro-4-methoxybenzyl)-5-(2-fluoro-6-methoxyphenyl) 2,3-dihydropyrrolo[3,4-b]quinolin-1-one

Using Method A, 9-Amino-2-(3-chloro-4-methoxybenzyl)-5-bromo 2,3-dihydropyrrolo[3,4-b]quinolin-1-one (0.175 g, 0.412 mmol) and 2-fluoro-6-methoxyphenylboronic acid (0.212 g, 1.25 mmol) were reacted to afford the title compound as an off-white solid (0.101 g, 51%). ¹H NMR (500 MHz, DMSO-d₆) δ ppm 3.62 (s, 3 H), 3.82 (s, 3 H), 4.18 (s, 2 H), 4.53-4.63 (m, 2 H), 6.83 (dd, J=8.6 Hz, 1 H), 6.93 (d, J=8.4 Hz, 1 H), 7.10 (d, J=8.5 Hz, 1 H), 7.23 (dd, J=8.5, 2.0 Hz, 1 H), 7.32-7.40 (m, 2 H), 7.50 (dd, J=7.7 Hz, 1 H), 7.53-7.58 (m, 1 H), 7.68 (br. s., 2 H), 8.39 (dd, J=8.3, 1.4 Hz, 1 H). MS APCI, m/z=478 (M+H). HPLC 1.86 min.

### EXAMPLE 110

### 2-(9-Amino-2-cyclopropyl-1-oxo-2,3-dihydro-1H-pyrrolo[3,4-b]quinolin-5-yl)-benzonitrile

Using Method A, 9-Amino-2-cyclopropyl-5-bromo 2,3-dihydropyrrolo[3,4-b]quinolin-1-one (0.200 g, 0.629 mmol) and 2-cyanophenylboronic acid (0.073 g, 0.943 mmol) were reacted to afford the title compound as an off-white solid (0.040 g, 19%). ¹H NMR (500 MHz, DMSO-d₆) δ ppm 0.70-0.79 (m, 2 H), 0.79-0.86 (m, 2 H), 2.85-2.93 (m, 1 H), 4.23 (s, 2 H), 7.51-7.61 (m, 3 H), 7.70 (dd, J=7.0, 1.3 Hz, 1 H), 7.76 (dd, J=7.7 Hz, 1 H), 7.90 (d, J=7.7 Hz, 1H), 8.47 (dd, J=8.4, 1.3 Hz, 1 H). MS APCI, m/z=341 (M+H). HPLC 1.55 min.

### EXAMPLE 111

### 9-Amino-2-cyclopropyl-5-(6-methyl-pyridin-3-yl)-2,3-dihydropyrrolo[3,4-b]quinolin-1-one

Using Method A, 9-Amino-2-cyclopropyl-5-bromo 2,3-dihydropyrrolo[3,4-b]quinolin-1-one (0.200 g, 0.629 mmol) and 6-methylpyridine-3-boronic acid (0.129 g, 0.943 mmol) were reacted to afford the title compound as an off-white solid (0.148 g, 71%). ¹H NMR (500 MHz, DMSO-d₆) δ ppm 0.72-0.87 (m, 4 H), 2.53 (s, 3 H), 2.84-2.95 (m, J=7.1, 7.1, 3.8,3.6 Hz, 1 H), 4.27 (s, 2 H), 7.31 (d, J=7.9 Hz, 1 H), 7.53 (dd, J=7.7 Hz, 1 H), 7.69-7.75 (m, 1 H), 7.88 (dd, J=8.0, 2.4 Hz, 1 H), 8.35-8.41 (m, 1 H), 8.64 (d, J=2.4 Hz, 1 H). MS APCI, m/z=331 (M+H). HPLC 2.50 min (polar method).

### EXAMPLE 112

### 9-Amino-2-cyclopropyl-5-(2,5-difluoro-phenyl)-2,3dihydropyrrolo[3,4-b]quinolin-1-one

Using Method A, 9-Amino-2-cyclopropyl-5-bromo 2,3-dihydropyrrolo[3,4-b]quinolin-1-one (0.150 g, 0.472 mmol) and 2,5-difluorophenylboronic acid (0.112 g, 0.708 mmol) were reacted to afford the title compound as an off-white solid (0.128 g, 77%). ¹H NMR (500 MHz, DMSO-d₆) δ ppm 0.71-0.86 (m, 4 H), 2.85-2.93 (m, 1 H), 4.25 (s, 2 H), 7.23-7.34 (m, 3 H), 7.53 (dd, J=8.2, 7.2 Hz, 1 H), 7.68 (d, J=6.9 Hz, 1 H), 8.43 (dd, J=8.4, 1.3 Hz, 1 H). MS APCI, m/z=352 (M+H). HPLC 1.41 min.

### EXAMPLE 113

### 9-Amino-2-cyclopropyl-5-(2-fluorophenyl)-2,3-dihydropyrrolo[3,4-b]quinolin-1-one

Using Method A, 9-Amino-2-cyclopropyl-5-bromo 2,3-dihydropyrrolo[3,4-b]quinolin-1-one (0.191 g, 0.600 mmol) and 2-fluorophenylboronic acid (0.126 g, 0.900 mmol) were reacted to afford the title compound as an off-white solid (0.123 g, 61%). ¹H NMR (500 MHz, DMSO-d₆) δ ppm 0.69-0.92 (m, 4 H), 2.89-2.90 (m, 1 H), 4.23 (s, 2 H), 7.20-7.30 (m, 2 H), 7.36-7.46 (m, 2 H), 7.53 (dd, J=8.2 Hz, 1 H), 7.64 (d, J=7.0 Hz, 1 H), 8.40 (dd, J=8.4, 1.2 Hz, 1 H). MS APCI, m/z=334 (M+H). HPLC 1.32 min.

### EXAMPLE 114

### 9-Amino-2-cyclopropyl-5-(2,6-difluoro-phenyl)-2,3dihydropyrrolo[3,4-b]quinolin-1-one

Using Method G, 9-Amino-2-cyclopropyl-5-bromo 2,3-dihydropyrrolo[3,4-b]quinolin-1-one (0.250 g, 0.786 mmol) and 2,6-difluorophenylboronic acid (0.493 g, 3.144 mmol) were reacted to afford the title compound as an off-white solid (0.030 g, 11%). ¹H NMR (500 MHz, DMSO-d₆) δ ppm 0.73-0.83 (m, J=6.8, 1.9 Hz, 4 H), 2.87-2.90 (m, 1H), 4.24 (s, 2 H), 7.12-7.21 (m, 2 H), 7.44-7.51 (m, 1 H), 7.51-7.58 (m, 1 H), 7.69 (dd, J=7.0, 1.2 Hz, 1 H), 8.46 (dd, J=8.4, 1.4 Hz, 1 H). MS APCI, m/z=352 (M+H). HPLC 1.38 min.

### EXAMPLE 115

### 9-Amino-2-cyclopropyl-5-(2-fluoro-4-methoxy-phenyl)-2,3-dihydropyrrolo[3,4-b]quinolin-1-one

Using Method A, 9-Amino-2-cyclopropyl-5-bromo 2,3-dihydropyrrolo[3,4-b]quinolin-1-one (0.191 g, 0.600 mmol) and 2-fluoro-4-methoxyphenylboronic acid (0.204 g, 1.200 mmol) were reacted to afford the title compound as an off-white solid (0.128 g, 59%). ¹H NMR (500 MHz, DMSO-d₆) δ ppm 0.71-0.79 (m, 2 H), 0.79-0.86 (m, 2 H), 2.85-2.92 (m, 1 H), 3.83 (s, 3 H), 4.23 (s, 2 H), 6.80-6.89 (m, 2 H), 7.31 (dd, J=8.4 Hz, 1 H), 7.50 (dd, J=8.3, 7.1 Hz, 1 H), 7.61 (dd, J=7.1, 0.9 Hz, 1 H), 8.37 (dd, J=8.4, 1.4 Hz, 1 H). MS APCI, m/z=364 (M+H). HPLC 1.42 min.

### EXAMPLE 116

### 9-Amino-5-(2-chloro-5-methoxyphenyl)-2-cyclopropyl-2,3-dihydropyrrolo[3,4-b]quinolin-1-one

Using Method A, 9-Amino-2-cyclopropyl-5-bromo 2,3-dihydropyrrolo[3,4-b]quinolin-1-one (0.150 g, 0.472 mmol) and 2-chloro-5-methoxyphenylboronic acid (0.132 g, 0.708 mmol) were reacted to afford the title compound as an off-white foam (0.128 g, 72%). ¹H NMR (500 MHz, DMSO-d₆) δ ppm 0.69-0.78 (m, 2 H), 0.78-0.85 (m, 2 H), 2.84-2.92 (m, 1 H), 3.77 (s, 3 H), 4.22 (s, 2 H), 6.86-6.91 (m, 1 H), 6.95-7.00 (m, 1 H), 7.41 (d, J=8.8 Hz, 1 H), 7.51 (dd, J=7.6 Hz, 1 H), 7.53-7.58 (m, 1 H), 8.39 (dd, J=8.3, 1.4 Hz, 1 H). MS APCI, m/z=380 (M+H). HPLC 1.44 min.

### EXAMPLE 117

### 9-Amino-2-cyclopropyl-5-(2,6-difluoro-4-methoxyphenyl)-2,3-dihydropyrrolo[3,4-b]quinolin-1-one

Using Method G, 9-Amino-2-cyclopropyl-5-bromo 2,3-dihydropyrrolo[3,4-b]quinolin-1-one (0.250 g, 0.786 mmol) and 2,6-difluoro-4-methoxyphenylboronic acid (0.443 g, 2.36 mmol) were reacted to afford the title compound as an off-white solid (0.015 g, 5%). ¹H NMR (500 MHz, DMSO-d₆) δ ppm 0.71-0.78 (m, 2 H), 0.79-0.84 (m, 2 H), 2.85-2.91 (m, 1 H), 3.85 (s, 3 H), 4.24 (s, 2 H), 6.80 (d, J=9.3 Hz, 2 H), 7.52 (dd, J=8.4, 7.1 Hz, 1 H), 7.65 (dd, J=7.0, 1.2 Hz, 1 H), 8.42 (dd, J=8.5, 1.4 Hz, 1 H). MS APCI, m/z=382 (M+H). HPLC 1.50 min.

### EXAMPLE 118

### 9-Amino-2-cyclopropyl-5-(2,3-difluorophenyl)-2,3-dihydropyrrolo[3,4-b]quinolin-1-one

Using Method A, 9-Amino-2-cyclopropyl-5-bromo 2,3-dihydropyrrolo[3,4-b]quinolin-1-one (0.151 g, 0.470 mmol) and 2-3-difluorophenylboronic acid (0.111 g, 0.705 mmol) were reacted to afford the title compound as an off-white solid (0.143 g, 87%). ¹H NMR (500 MHz, DMSO-d₆) δ ppm 0.70-0.79 (m, 2 H), 0.79-0.86 (m, 2 H), 2.86-2.92 (m, 1 H), 4.25 (s, 2 H), 7.18-7.30 (m, 2 H), 7.40-7.48 (m, 1 H), 7.54 (dd, J=8.3, 7.1 Hz, 1 H), 7.68 (d, J=7.0 Hz, 1 H), 8.44 (dd, J=8.4, 1.3 Hz, 1 H). MS APCI, m/z=352 (M+H). HPLC 1.39 min.

### EXAMPLE 119

### 9-Amino-2-cyclopropyl-5-(2,4-difluorophenyl)-2,3-dihydropyrrolo[3,4-b]quinolin-1-one

Using Method A, 9-Amino-2-cyclopropyl-5-bromo 2,3-dihydropyrrolo[3,4-b]quinolin-1-one (0.162 g, 0.509 mmol) and 2-4-difluorophenylboronic acid (0.121 g, 0.764 mmol) were reacted to afford the title compound as an off-white solid (0.150 g, 84%). ¹H NMR (500 MHz, DMSO-d₆) δ ppm 0.72-0.85 (m, 4 H), 2.85-2.93 (m, J=7.3, 7.3, 3.9, 3.7 Hz, 1 H), 4.24 (s, 2 H), 7.11-7.18 (m, J=8.5, 5.9, 0.5, 0.5 Hz, 1 H), 7.27 (td, J=9.7, 2.6 Hz, 1 H), 7.40-7.49 (m, 1H), 7.52 (t, J=7.7 Hz, 1 H), 7.65 (dd, J=7.1, 1.5 Hz, 1 H), 8.41 (dd, J=8.4, 1.5 Hz, 1 H). MS APCI, m/z=352 (M+H). HPLC 1.39 min.

### EXAMPLE 120

### 9-Amino-2-cyclopropyl-5-(2-fluoro-6-methylpyridin-3-yl)-2,3-dihydropyrrolo[3,4-b]quinolin-1-one

Using Method G, 9-Amino-2-cyclopropyl-5-bromo 2,3-dihydropyrrolo[3,4-b]quinolin-1-one (0.180 g, 0.566 mmol) and 2-fluoro-6-methylpyridine-3-boronic acid (0.201 g, 0.0849 mmol) were reacted to afford the title compound as an off-white solid (0.129 g, 65%). ¹H NMR (500 MHz, DMSO-d₆) δ ppm 0.70-0.86 (m, 4 H), 2.50 (s, 3 H), 2.85-2.92 (m, 1 H), 4.24 (s, 2H), 7.28 (dd, J=7.6, 1.8 Hz, 1 H), 7.53 (dd, J=8.4, 7.1 Hz, 1 H), 7.66-7.72 (m, 1 H), 7.83 (dd, J=9.9, 7.3 Hz, 1 H), 8.42 (dd, J=8.4, 1.5 Hz, 1 H). MS APCI, m/z=349 (M+H). HPLC 1.19 min.

### EXAMPLE 121

### 9-Amino-2-cyclobutyl-5-(6-methylpyridin-3-yl)-2,3-dihydropyrrolo[3,4-b]quinolin-1-one

Using Method A, 9-Amino-2-cyclobutyl-5-bromo-2,3-dihydropyrrolo[3,4-b]quinolin-1-one (0.100 g, 0.301 mmol) and 6-methylpyridine-3-boronic acid (0.062 g, 0.452 mmol) were reacted to afford the title compound as an off-white solid (0.043 g, 41%). ¹H NMR (500 MHz, DMSO-d₆) δ ppm 1.63-1.75 (m, 2 H), 2.08-2.16 (m, 2 H), 2.27-2.39 (m, 2 H), 2.54 (s, 3 H), 4.46 (s, 2 H), 4.69-4.79 (m, 1 H), 7.32 (d, J=7.9 Hz, 1 H), 7.54 (dd, J=8.3, 7.2 Hz, 1 H), 7.73 (dd, J=7.2, 1.4 Hz, 1 H), 7.90 (dd, J=7.9, 2.3 Hz, 1 H), 8.38 (dd, J=8.4, 1.4 Hz, 1 H), 8.66 (d, J=1.8 Hz, 1 H). MS APCI, m/z=345 (M+H). HPLC 1.00 min.

### EXAMPLE 122

### 9-Amino-2-cyclobutyl-5-(2,6-difluoro-4-methoxyphenyl)-2,3-dihydropyrrolo[3,4-b]quinolin-1-one

Using Method G, 9-Amino-2-cyclobutyl-5-bromo-2,3-dihydropyrrolo[3,4-b]quinolin-1-one (0.250 g, 0.753 mmol) and 2,6-difluoro-4-methoxyphenylboronic acid (0.424 g, 2.26 mmol) were reacted to afford the title compound as an off-white solid (0.007 g, 2%). ¹H NMR (500 MHz, DMSO-d₆) δ ppm 1.62-1.73 (m, 2 H), 2.05-2.18 (m, 2 H), 2.26-2.39 (m, 2 H), 3.86 (s, 3 H), 4.43 (s, 2 H), 4.67-4.77 (m, 1 H), 6.76-6.86 (m, 2 H), 7.53 (dd, J=8.1 Hz, 1 H), 7.67 (d, J=6.4 Hz, 1 H), 8.43 (dd, J=8.4, 0.8 Hz, 1 H). MS APCI, m/z=396 (M+H). HPLC 1.66 min.

### EXAMPLE 123

### 9-Amino-2-cyclobutyl-5-(2,4-dimethoxy-pyrimidin-5-yl)-2,3-dihydropyrrolo[3,4-b]quinolin-1-one

Using Method A, 9-Amino-2-cyclobutyl-5-bromo-2,3-dihydropyrrolo[3,4-b]quinolin-1-one (0.080 g, 0.24 mmol) and 2,6-dimethoxy-5-(4,4,5,5-tetramethyl-1,3,2-dioxaborolan-2-yl)pyrimidine (0.096 g, 0.361 mmol) were reacted to afford the title compound as an off-white solid (0.052 g, 55%). ¹H NMR (500 MHz, DMSO-d₆) δ ppm 1.63-1.76 (m, 2 H), 2.05-2.16 (m, 2 H), 2.26-2.38 (m, 2 H), 3.82 (s, 3 H), 3.98 (s, 3 H), 4.42 (s, 2 H), 4.67-4.77 (m, 1 H), 7.50 (dd, J=8.4, 7.1 Hz, 1 H), 7.67 (dd, J=7.1, 1.4 Hz, 1 H), 8.25 (s, 1 H), 8.37 (dd, J=8.4, 1.4 Hz, 1 H). MS APCI, m/z=392 (M+H). HPLC 1.31 min.

### EXAMPLE 124

### 9-Amino-2-cyclobutyl-5-(2-fluorophenyl)-2,3-dihydro-pyrrolo[3,4-b]quinolin-1-one

Using Method A, 9-Amino-2-cyclobutyl-5-bromo-2,3-dihydropyrrolo[3,4-b]quinolin-1-one (0.082 g, 0.246 mmol) and 2-fluorophenylboronic acid (0.043 grams, 0.321 mmol) were reacted to afford the title compound as an off-white solid (0.048 g, 56%). ¹H NMR (500 MHz, DMSO-d₆) δ ppm 1.62-1.73 (m, 2 H), 2.05-2.16 (m, 2 H), 2.25-2.37 (m, 2 H), 4.42 (s, 2 H), 4.67-4.77 (m, 1 H), 7.22-7.30 (m, 2 H), 7.38-7.47 (m, 2 H), 7.53 (dd, J=8.4, 7.0 Hz, 1 H), 7.65 (dd, J=1.6, 0.2 Hz, 1 H), 8.41 (dd, J=8.4, 1.4 Hz, 1 H). MS APCI, m/z=348 (M+H). HPLC 1.41 min.

### EXAMPLE 125

### 5-(9-Amino-2-cyclobutyl-1-oxo-2,3-dihydro-1H-pyrrolo[3,4-b]quinolin-5-yl)-pyridine-2-carbonitrile

Using Method A, 9-Amino-2-cyclobutyl-5-bromo-2,3-dihydropyrrolo[3,4-b]quinolin-1-one (0.300 g, 0.90 mmol) and 5-(4,4,5,5-tetramethyl-1,3,2-dioxaborolan-2-yl)picolinonitrile (0.312 g, 1.35 mmol) were reacted to afford the title compound as an off-white solid (0.217 g, 68%). ¹H NMR (500 MHz, DMSO-d₆) δ ppm 1.64-1.76 (m, 2 H), 2.07-2.18 (m, 2 H), 2.28-2.39 (m, 2 H), 4.47 (s, 2 H), 4.70-4.78 (m, 1 H), 7.59 (dd, J=8.3, 7.3 Hz, 1 H), 7.86 (dd, J=7.2, 1.4 Hz, 1 H), 8.13 (dd, J=8.0, 0.2 Hz, 1 H), 8.29 (dd, J=8.0, 2.3 Hz, 1 H), 8.48 (dd, J=8.4, 1.4 Hz, 1 H), 8.98-9.05 (m, 1 H). MS APCI, mz=356 (M+H). HPLC 1.77 min.

### EXAMPLE 126

### 9-Amino-2-cyclobutyl-5-(6-fluoro-2-methylpyridin-3-yl)-2,3-dihydropyrrolo[3,4-b]quinolin-1-one

Using Method A, 9-Amino-2-cyclobutyl-5-bromo-2,3-dihydropyrrolo[3,4-b]quinolin-1-one (0.310 g, 0.93 mmol) and 6-fluoro-2-methylpyridin-3-ylboronic acid (0.289 g, 1.87 mmol) were reacted to afford the title compound as an off-white solid (0.267 g, 67%). ¹H NMR (500 MHz, DMSO-d₆) δ ppm 1.62-1.75 (m, 2 H), 2.03-2.16 (m, 2 H), 2.24-2.37 (m, 2 H),2.46-2.54 (m, 3 H), 4.43 (s, 2 H), 4.67-4.79 (m, 1 H), 7.45 (ddd, J=7.2, 5.0, 1.9 Hz, 1 H), 7.56 (dd, J=8.4, 0.2 Hz, 1 H), 7.73 (dd, J=7.2, 1.3 Hz, 1 H), 7.95-8.03 (m, 1 H), 8.45 (dd, J=7.7, 0.8 Hz, 1 H). MS APCI, m/z=363 (M+H). HPLC 1.62 min.

### EXAMPLE 127

### 9-Amino-2-cyclobutyl-5-(2-fluoropyridin-3-yl)-2,3-dihydropyrrolo[3,4-b]quinolin-1-one

Using Method A, 9-Amino-2-cyclobutyl-5-bromo-2,3-dihydropyrrolo[3,4-b]quinolin-1-one (0.205 g, 0.62 mmol) and 2-fluoropyridin-3-ylboronic acid (0.174 g, 1.23 mmol) were reacted to afford the title compound as an off-white solid (0.156 g, 72.6%). ¹H NMR (500 MHz, DMSO-d₆) δ ppm 1.62-1.74 (m, 2 H), 2.09-2.17 (m, 2 H), 2.25-2.40 (m, 2 H), 4.43 (s, 2 H), 4.66-4.78 (m, 1 H), 7.45 (ddd, J=7.2, 5.0, 1.9 Hz, 1 H), 7.55 (dd, J=8.4, 7.1 Hz, 1 H), 7.73 (dd, J=7.0, 1.1 Hz, 1 H), 7.99 (ddd, J=9.5, 7.4, 1.9 Hz, 1 H), 8.24-8.29 (m, 1 H), 8.45 (dd, J=8.4, 1.3 Hz, 1 H). MS APCI, m/z=349 (M+H). HPLC 1.62 min.

### EXAMPLE 128

### 9-Amino-2-cyclobutyl-5-(6-methoxy-5-methylpyridin-3-yl)-2,3-dihydropyrrolo[3,4-b]quinolin-1-one

Where the starting 9-amino-2-cyclobutyl-5-(6-fluoro-5-methylpyridin-3-yl)-2,3-dihydro-1H-pyrrolo[3,4-b]quinolin-1-one was prepared by Method A. Using Method H, 9-amino-2-cyclobutyl-5-(6-fluoro-5-methylpyridin-3-yl)-2,3-dihydro-1H-pyrrolo[3,4-b]quinolin-1-one (0.38 g, 1.05 mmol) was reacted to afford the title compound as a white solid(0.255 g, 65%). ¹H NMR (500 MHz, DMSO-d₆) δ ppm 1.64-1.71 (m, 2 H), 2.08-2.17 (m, 2 H), 2.22 (s, 3 H), 2.29-2.40 (m, 2 H), 3.95 (s, 3 H), 4.47 (s, 2 H), 4.69-4.79 (m, 1 H), 7.51 (dd, J=8.4, 7.1 Hz, 1 H), 7.71 (dd, J=7.1, 1.4 Hz, 1 H), 7.80 (dd, J=2.4, 0.8 Hz, 1 H), 8.22 (dd, J=1.5, 0.9 Hz, 1 H), 8.34 (dd, J=8.4, 1.4 Hz, 1 H). MS APCI, m/z=375 (M+H). HPLC 1.91 min.

### EXAMPLE 129

### 9-Amino-2-cyclobutyl-5-((P)2-fluoro-6-methoxyphenyl)-2,3-dihydropyrrolo[3,4-b]quinolin-1-one

Using Method A, 9-Amino-2-cyclobutyl-5-bromo-2,3-dihydropyrrolo[3,4-b]quinolin-1-one (0.807 g, 2.14 mmol) and 2-fluoro-6-methoxyphenylboronic acid (1.177 g, 6.92 mmol) were reacted to afford a mixture of atropisomers that were separated by chiral Super Critical Fluid chromatography as a white solid as a single atropisomer (0.130 g, 16%). Vibrational Circular Dichroic Analysis (VCD) used to determine the absolute axial chirality, as the plus (P) isomer. ¹H NMR (500 MHz, DMSO-d₆) δ ppm 1.62-1.72 (m, 2 H), 2.05-2.15 (m, 2 H), 2.24-2.37 (m, 2 H), 3.64 (s, 3 H), 4.33-4.45 (m, 2 H), 4.67-4.78 (m, 1 H), 6.82-6.89 (m, 1 H), 6.93-6.98 (m, 1 H), 7.33-7.43 (m,1 H), 7.49 (dd, J=8.3, 7.1 Hz, 1 H), 7.53-7.58 (m, 1 H), 8.37 (dd, J=8.4, 1.5 Hz, 1 H). MS APCI, m/z=378 (M+H). HPLC 1.91 min.

### EXAMPLE 130

### 9-Amino-2-cyclobutyl-5-((M)2-fluoro-6-methoxyphenyl)-2,3-dihydropyrrolo[3,4-b]quinolin-1-one

Using Method A, 9-Amino-2-cyclobutyl-5-bromo-2,3-dihydropyrrolo[3,4-b]quinolin-1-one (0.807 g, 2.14 mmol) and 2-fluoro-6-methoxyphenylboronic acid (1.177 g, 6.92 mmol) were reacted to afford a mixture of atropisomers that were separated by Super Critical Fluid chromatography as a white solid as a single atropisomer. (0.141 g, 17%). Vibrational Circular Dichroic Analysis (VCD) used to determine the absolute axial chirality, as the minus (M) isomer. ¹H NMR (500 MHz, DMSO-d₆) δ ppm 1.62-1.74 (m, 2 H), 2.03-2.15 (m, 2 H), 2.24-2.37 (m, 2 H), 3.64(s, 3 H), 4.32-4.46 (m, 2 H), 4.65-4.77 (m, 1 H), 6.83-6.90 (m, 1 H), 6.96 (dd, J=8.5, 0.8 Hz, 1 H), 7.34-7.42 (m, 1 H), 7.49 (dd, J=8.3, 7.0 Hz, 1 H), 7.56 (dd, J=7.0, 1.5 Hz, 1 H), 8.37 (dd, J=8.4, 1.6 Hz, 1 H). MS APCI, m/z=378 (M+H). HPLC 1.88 min.

### EXAMPLE 131

### 2-(9-Amino-2-cyclobutyl-1-oxo-2,3-dihydro-1H-pyrrolo[3,4-b]quinolin 5-yl)-6-methoxybenzonitrile

Using Method I, 9-amino-5-bromo-2-cyclobutyl-2,3-dihydro-1H-pyrrolo[3,4-b]quinolin-1-one (0.3 g, 0.90 mmol) and 2-(5,5-dimethyl-1,3,2-dioxaborinan-2-yl)-6-methoxybenzonitrile (0.332 g, 1.35 mmol) were reacted to afford the title compound as an off-white solid (0.228 g, 66%). ¹H NMR (500 MHz, DMSO-d₆) δ ppm 1.61-1.76 (m, 2 H), 2.06-2.16 (m, 2 H), 2.26-2.38 (m, 2 H), 3.98 (s, 3 H), 4.42 (s, 2 H), 4.67-4.78 (m, 1 H), 7.09 (dd, J=6.9, 0.2 Hz, 1 H), 7.26 (dd, J=8.0, 0.2 Hz, 1 H), 7.55 (dd, J=8.4, 7.0 Hz, 1 H), 7.68-7.73 (m, 2 H), 8.45 (dd, J=8.4, 1.5 Hz, 1 H). MS APCI, m/z=385 (M+H). HPLC 1.88 min.

### EXAMPLE 132

### 2-(9-Amino-2-cyclobutyl-1-oxo-2,3-dihydro-1H-pyrrolo[3,4-b]quinolin-5-yl)-3-methoxybenzonitrile

Using Method I, 9-amino-5-bromo-2-cyclobutyl-2,3-dihydro-1H-pyrrolo[3,4-b]quinolin-1-one (0.3 g, 0.90 mmol) and 2-(5,5-dimethyl-1,3,2-dioxaborinan-2-yl)-3-methoxybenzonitrile (0.332 g, 1.35 mmol) were reacted to afford the title compound as an off-white solid (0.124 g, 36%). ¹H NMR (500 MHz, DMSO-d₆) δ ppm 1.60-1.74 (m, 2 H), 2.04-2.16 (m, 2 H), 2.24-2.37 (m, 2 H), 3.66 (s, 3 H), 4.33-4.43 (m, 2 H), 4.67-4.77 (m, 1 H), 7.45-7.48 (m, 2 H), 7.50-7.58 (m, 2 H), 7.59-7.63 (m,1 H), 8.43 (dd, J=7.6, 0.8 Hz, 1 H). MS APCI, m/z=385 (M+H). HPLC 1.84 min.

### EXAMPLE 133

### 9-Amino-2-cyclobutyl-5-(2,6-difluoropyridin-3-yl)-2,3-dhydropyrrolo[3,4-b]quinolin-1-one

Using Method I, 9-amino-5-bromo-2-cyclobutyl-2,3-dihydro-1H-pyrrolo[3,4-b]quinolin-1-one (0.25 g, 0.75 mmol) and 2,6-difluoropyridin-3-ylboronic acid (0.239 g, 1.51 mmol) were reacted to afford the title compound as an off-white solid (0.031 g, 11%). ¹H NMR (500 MHz, DMSO-d₆) δ ppm 1.63-1.74 (m, 2 H), 2.08-2.17 (m, 2 H), 2.26-2.39 (m, 2 H), 4.44(s, 2 H), 4.66-4.79 (m, 1 H), 7.27 (dd, J=8.0, 2.5 H), 7.56 (dd, J=8.4, 7.1 Hz, 1 H), 7.76 (dd, J=6.3,0.8 Hz, 1 H), 8.17-8.25 (m, 1 H), 8.46 (dd, J=8.4, 1.4 Hz, 1 H). MS APCI, m/z=367 (M+H). HPLC 7.89 min (polar method).

### EXAMPLE 134

### 9-Amino-5-(2-fluoro-6-methoxyphenyl)-2-(3-methylcyclobutyl)-2,3-dihydropyrrolo[3,4-b]quinolin-1-one

Using Method A, 9-amino-5-bromo-2-(3-methylcyclobutyl)-2,3-dihydro-1H-pyrrolo[3,4-b]quinolin-1-one (0.242 g, 0.70 mmol) and 2-fluoro-6-methoxyphenylboronic acid (0.267 g, 1.57 mmol) were reacted to afford the title compound as an off-white solid (0.209 g, 76%). ¹H NMR (500 MHz, DMSO-d₆) δ ppm 1.03-1.21 (m, 3 H), 1.78-1.94 (m, 2 H), 2.01-2.10 (m, 1 H), 2.22-2.34 (m, 2 H), 3.64 (s, 3 H), 4.32-4.46 (m, 2 H), 4.48-4.95 (m, 1H), 6.81-6.90 (m, 1 H), 6.96 (d, J=8.4 Hz, 1 H), 7.33-7.44 (m, 1 H), 7.46-7.52 (m, 1H), 7.56 (dd, J=7.0, 1.5 Hz, 1 H), 8.36 (dd, J=8.3, 1.6 Hz, 1 H). MS APCI, m/z=392 (M+H). HPLC 1.00 min.

### EXAMPLE 135

### 9-Amino-2-cyclobutyl-5-(1-methyl-1H-pyrazol-4-yl)-2,3-dihydropyrrolo[3,4-b]quinolin-1-one

Using Method A, 9-amino-5-bromo-2-cyclobutyl-2,3-dihydro-1H-pyrrolo[3,4-b]quinolin-1-one (0.230 g, 0.69 mmol) and 1-methyl-4-(4,4,5,5-tetramethyl-1,3,2-dioxaborolan-2-yl)-1H-pyrazole (0.144 g, 0.69 mmol) were reacted to afford the title compound as an off-white solid (0.145 g, 62%). ¹H NMR (500 MHz, DMSO-d₆) δ ppm 1.72 (dt, J=9.8, 4.9 Hz, 2 H), 2.16 (ddd, J=8.0, 2.4, 2.3 Hz, 2 H),2.38 (td, J=9.5, 2.4 Hz, 2 H), 3.92 (s, 3 H), 4.57 (s, 2 H), 4.72-4.84 (m, 1 H), 7.43 (dd, J=7.9, 7.6 Hz, 1 H), 7.98 (dd, J=8.0, 0.7 Hz, 1 H), 8.16-8.21 (m, 2 H), 8.58 (s, 1 H). MS APCI, m/z=334 (M+H). HPLC 1.56 min.

### EXAMPLE 136

### 9-amino-5-(6-methoxy-2-methylpyridin-3-yl)-2-((1s,3s)-3-methylcyclobutyl)-2,3-dihydro-1H-pyrrolo[3,4-b]quinolin-1-one

Using Method A, 9-amino-5-bromo-2-((1s,3 s)-3-methylcyclobutyl)-2,3-dihydro-1H-pyrrolo[3,4-b]quinolin-1-one (0.110 g, 0.32 mmol) and 6-methoxy-2-methyl-3-(4,4,5,5-tetramethyl-1,3,2-dioxaborolan-2-yl)pyridine (0.119 g, 0.48 mmol) were reacted to afford the title compound as an off-white solid (0.111 g, 90%). ¹H NMR (500 MHz, DMSO-d6) δ ppm 1.00-1.12 (m, 3 H), 1.83-1.90 (m, 1 H), 2.00-2.13 (m, 4 H), 2.21-2.33 (m, 2 H), 2.45-2.55 (m, 1 H), 3.90 (s, 3 H), 4.43 (s, 2H), 4.47-4.61 (m, 1 H), 6.65-6.73 (m, 2 H), 7.42-7.54 (m, 1 H), 7.55-7.59 (m, 1 H), 8.37 (dd, J=8.5, 1.7 Hz, 1 H). MS APCI, m/z=389 (M+H). HPLC 1.87 min.

### EXAMPLE 137

### 9-amino-5-(2-fluoro-6-methoxyphenyl)-2-((1s,3s)-3-methylcyclobutyl)-2,3-dihydro-1H-pyrrolo[3,4-b]quinolin-1-one

Using Method A, 9-amino-5-bromo-2-((1s,3s)-3-methylcyclobutyl)-2,3-dihydro-1H-pyrrolo[3,4-b]quinolin-1-one (0.110 g, 0.32 mmol) and 2-fluoro-6-methoxyphenylboronic acid (0.108 g, 0.64 mmol) were reacted to afford the title compound as an off-white solid (0.102 g, 82%). ¹H NMR (500 MHz, DMSO-d6) δ ppm 1.06 (d, J=6.5 Hz, 3 H), 1.83-1.94 (m, 2 H), 2.00-2.11 (m, 1 H), 2.22-2.32 (m, 2 H), 3.64 (s, 3 H), 4.33-4.42 (m, 2 H), 4.49-4.59 (m, 1 H), 6.86 (dd, J=8.8 Hz, 1 H), 6.96 (d, J=8.4 Hz, 1 H), 7.36-7.42 (m, 1 H), 7.49 (dd, J=8.2, 7.1 Hz, 1 H), 7.56 (dd, J=7.1, 1.4 Hz, 1 H), 7.64 (br. s., 1 H), 8.36 (dd, J=8.3, 1.4 Hz, 2 H). MS APCI, m/z=392 (M+H). HPLC 0.91 min.

### EXAMPLE 138

### 9-amino-5-(2-methoxypyridin-3-yl)-2-((1s,3s)-3-methylcyclobutyl)-2,3-dihydro-1H-pyrrolo[3,4-b]quinolin-1-one

Using Method A, 9-amino-5-bromo-2-((1s,3 s)-3-methylcyclobutyl)-2,3-dihydro-1H-pyrrolo[3,4-b]quinolin-1-one (0.100 g, 0.29 mmol) and 2-methoxypyridin-3-ylboronic acid (0.088 g, 0.58 mmol) were reacted to afford the title compound as an off-white solid (0.095 g, 88%). ¹H NMR (500 MHz, DMSO-d6) δ ppm 8.36 (d, J=8.4 Hz, 1 H) 8.20 (dd, J=5.0, 1.9 Hz, 1 H) 7.68 (br. s., 2 H) 7.59-7.64 (m, 2 H) 7.49 (dd, J=7.7 Hz, 1 H) 7.07 (dd, J=7.2, 5.0 Hz, 1 H) 4.47-4.59 (m, 1 H) 4.38 (s, 2 H) 3.74 (s, 3 H) 2.28 (qd, J=7.8, 2.7 Hz, 2 H) 1.99-2.12 (m, 1 H) 1.80-1.94 (m, 2 H) 1.07 (d, J=6.6 Hz, 3 H). MS APCI, m/z=375 (M+H). HPLC 1.75 min.

### EXAMPLE 139

### 2-(9-Amino-2-cyclopropyl-1-oxo-2,3-dihydro-1H-pyrrolo[3,4-b]quinolin-5-yl)-3-methoxybenzonitrile

Using Method I, 9-amino-5-bromo-2-cyclopropyl-2,3-dihydro-1H-pyrrolo[3,4-b]quinolin-1-one (0.380 g, 1.19 mmol) and 2-(5,5-dimethyl-1,3,2-dioxaborinan-2-yl)-3-methoxybenzonitrile (0.439 g, 1.79 mmol) were reacted to afford the title compound as an off-white solid (0.083 g, 19%). ¹H NMR (500 MHz, DMSO-d₆) δ ppm 8.42 (dd, J=8.32, 1.37 Hz, 1 H) 7.58-7.63 (m, 3 H) 7.40-7.48 (m,2 H) 4.20 (s, 2 H) 3.65 (s, 3) 2.82-2.94 (m, 1 H) 0.68-0.87 (m, 4 H). MS APCI, m/z=371 (M+H). HPLC 1.66 min.

### EXAMPLE 140

### 9-Amino-2-cyclopropyl-5-((P)2-fluoro-6-methoxyphenyl)-2,3-dihydropyrrolo[3,4-b]quinolin-1-one

Using Method A, 9-Amino-2-cyclopropyl-5-bromo-2,3-dihydropyrrolo[3,4-b]quinolin-1-one (0.800 g, 2.52 mmol) and 2-fluoro-6-methoxyphenylboronic acid (0.855 g, 5.03 mmol) were reacted to afford a mixture of atropisomers that were separated by Super Critical Fluid chromatography as a white solid as a single atropisomer (0.240 g, 52%). Vibrational Circular Dichroic Analysis (VCD) used to determine the absolute axial chirality, as the plus (P) isomer. ¹H NMR (300 MHz, DMSO-d₆) δ ppm 8.36 (dd, J=8.11, 1.84 Hz, 1 H) 7.45-7.57 (m, 2 H) 7.28-7.43 (m,1 H) 6.95 (d, J=8.38 Hz, 1) 6.78-6.90 (m, 1 H) 4.21 (s, 2 H) 3.63 (s,3 H) 2.80-2.94 (m, 1 H) 0.63-0.80 (m, 4 H). MS APCI, m/z=364 (M+H). HPLC 1.35 min.

### EXAMPLE 141

### 9-Amino-2-cyclobutyl-5-(2-fluoro-6-methylpyridin-3-yl)-2,3-dihydropyrrolo[3,4-b]quinolin-1-one

Using Method G, 9-amino-5-bromo-2-cyclobutyl-2,3-dihydro-1H-pyrrolo[3,4-b]quinolin-1-one (0.107 g, 0.32 mmol) and 2-fluoro-6-methylpyridin-3-ylboronic acid (0.075 g, 0.48 mmol) (0.144 g, 0.69 mmol) were reacted to afford the title compound as an off-white solid (0.117 g, 56%). ¹H NMR (500 MHz, DMSO-d₆) δ ppm 8.43 (dd, J=8.43, 1.49 Hz, 1 H) 7.85 (dd, J=9.84, 7.40 Hz, 1 H) 7.70 (dd, J=7.10, 1.53 Hz, 1 H) 7.53 (dd, J=8.39, 7.02 Hz, 1 H) 7.26-7.31 (m, 1 H) 4.67-4.78 (m, 1 H) 4.43 (s,2 H) 2.25-2.38 (m, J=9.52, 9.52, 9.52, 9.52, 2.67 Hz, 2 H) 2.04-2.17 (m, 2 H) 1.62-1.75 (m, 2 H). MS APCI, m/z=363 (M+H). HPLC 1.67 min.

### EXAMPLE 142

### 9-Amino-2-cyclobutyl-5-(6-methoxy-2-methylpyridin-3-yl)-2,3-dihydropyrrolo[3,4-b]quinolin-1-one

Using Method A, 9-amino-5-bromo-2-cyclobutyl-2,3-dihydro-1H-pyrrolo[3,4-b]quinolin-1-one (8.62 g, 25.94 mmol) and 6-methoxy-2-methyl-3-(4,4,5,5-tetramethyl-1,3,2-dioxaborolan-2-yl)pyridine (8.4 g, 33.72 mmol) were reacted to afford the title compound as an off-white solid (5.98 g, 62%). ¹H NMR (500 MHz, DMSO-d₆) δ ppm 8.37 (dd, J=8.32, 1.60 Hz, 1 H) 7.54-7.60 (m, 1 H) 7.42-7.53 (m,2 H) 6.69 (dd, J=8.28, 0.19 Hz, 1 H) 4.66-4.79 (m, 1 H) 4.42 (s, 2 H) 3.90 (s, 3 H) 2.24-2.38 (m, 2 H) 2.05-2.16 (m, 5 H) 1.61-1.73 (m, 2 H). MS APCI, m/z=375 (M+H). HPLC 1.82 min.

### EXAMPLE 143

### 9-Amino-2-cyclobutyl-5-(1,3-dimethyl-1H-pyrazol-4-yl)-2,3-dihydropyrrolo[3,4-b]quinolin-1-one

Using Method A, 9-amino-5-bromo-2-cyclobutyl-2,3-dihydro-1H-pyrrolo[3,4-b]quinolin-1-one (0.240 g, 0.72 mmol) and (2-(1,3-dimethyl-1H-pyrazol-4-yl)-4,5,5-trimethyl-1,3,2-dioxaborolan-4-yl)methylium (0.208 g, 0.94 mmol) were reacted to afford the title compound as an off-white solid (0.139 g, 54%). ¹H NMR (500 MHz, DMSO-d₆) δ ppm 8.24 (dd, J=8.39, 1.53 Hz, 1 H) 7.86 (s, 1 H) 7.60-7.66 (m, 1 H)7.45 (dd, JJ=8.28, 7.21 Hz, 1 H) 4.69-4.80 (m, 1 H) 4.48 (s, 2 H) 3.83 (s, 3 H) 2.28-2.41 (m, 2 H) 2.08-2.19 (m, 5 H) 1.63-1.76 (m, 2 H). MS APCI, m/z=348 (M+H). HPLC 1.57 min.

### EXAMPLE 144

### 9-Amino-2-cyclobutyl-5-(6-fluoro-5-methylpyridin-3-yl)-2,3-dihydropyrrolo[3,4-b]quinolin-1-one

Using Method A, 9-amino-5-bromo-2-cyclobutyl-2,3-dihydropyrrolo[3,4-b]quinolin-1-one (0.2655 g, 0.80 mmol) and 6-fluoro-5-methylpyridin-3-ylboronic acid (0.31176 g, 2.01 mmol) were reacted to afford the title compound as an off-white solid (0.1458 g, 50.3%). ¹H NMR (500.333 MHz, DMSO) δ 8.40 (d, J=8.4 Hz, 1H), 8.25 (s, 1H), 8.06 (d, J=9.5 Hz, 1H), 7.76 (dd, J=7.2, 1.2 Hz, 1H), 7.54 (dd, J=7.2, 8.4 Hz, 1H), 7.70 (bs, 1H), 4.74 (quintet, J=8.6 Hz, 1H), 4.47 (s, 2H), 2.37-2.29 (m, 2H), 2.16-2.08 (m, 2H), 1.69 (m Hz, 2H), 2.33 (s, 3H). MS APCI, m/z=363.3 (M+H). HPLC 1.87 min. MS TOF, Theor m/z=363.16157 (M+H), Expl m/z=363.16202, Error=1.24 ppm.

### EXAMPLE 145

### 9-Amino-2-cyclopentyl-5-(2-fluoro-6-methoxyphenyl)-2,3-dihydropyrrolo[3,4-b]quinolin-1-one

Using Method A, 9-amino-5-bromo-2-cyclopentyl-2,3-dihydropyrrolo[3,4-b]quinolin-1-one (213.7 mg, 0.62 mmol) and 2-fluoro-6-methoxyphenyl boronic acid (326.8 mg, 1.92 mmol) were reacted to afford the title compound as an off-white solid (180.5 mg, 75%). ¹H NMR (500.333 MHz, DMSO) δ 8.37 (dd, J=8.3, 1.2 Hz, 1H), 7.63 (bs, 2H), 7.55 (dd, J=7.0, 1.3 Hz, 1H), 7.49 (dd, J=8.1, 7.0 Hz, 1H), 7.38 (dt, J=6.8, 8.2 Hz, 1H), 6.95 (d, J=8.4 Hz, 1H), 6.85 (t, J=8.6 Hz, 1H), 4.55 (quintet, J=7.7 Hz, 1H). 4.28 (s, 2H), 3.64 (s, 3H), 1.86-1.78 (m, 2H), 1.76-1.51 (m, 6H). MS APCI, m/z=392.1 (M+H). HPLC 1.98 min.

### EXAMPLE 146

### 2-(9-Amino-2-cyclopentyl-1-oxo-2,3-dihydro-1H-pyrrolo[3,4-b]quinolin-5-yl)-benzonitrile

Using Method A, 9-amino-5-bromo-2-cyclopentyl-2,3-dihydropyrrolo[3,4-b]quinolin-1-one (229.0 mg, 0.66 mmol) and 2-cyanophenyl boronic acid (211.3 mg, 1.44 mmol) were reacted to afford the title compound as a white solid (91.3 mg, 37%). ¹H NMR (500.333 MHz, DMSO) δ 8.47 (dd, J=8.4, 1.3 Hz, 1H), 7.90 (dd, J=7.7, 0.9 Hz, 1H), 7.76 (td, J=7.7, 1.3 Hz, 1H), 7.70 (dd, J=7.1, 1.3 Hz, 1H), 7.60-7.55 (m, 3H), 4.56 (quintet, J=7.8 Hz, 1H), 4.30 (s, 2H), 1.87-1.79 (m, 2H), 1.76-1.62 (m, 4H), 1.62-1.53 (m, 2H). MS APCI, m/z=369.2 (M+H). HPLC 1.88 min. MS TOF, Theor m/z=369.17099 (M+H), Expl m/z=369.17166, Error=1.82 ppm.

### EXAMPLE 147

### 9-Amino-2-cyclopentyl-5-(6-methoxypyridin-3-yl)-2,3-dihydropyrrolo[3,4-b]quinolin-1-one

Using Method A, 9-amino-5-bromo-2-cyclopentyl-2,3-dihydropyrrolo[3,4-b]quinolin-1-one (229.0 mg, 0.66 mmol) and 6-methoxypyridin-3-ylboronic acid (211.0 mg, 1.38 mmol) were reacted to afford the title compound as an off-white solid (140.2 mg, 57%). ¹H NMR (500.333 MHz, DMSO) δ 8.39 (d, J=2.2 Hz, 1H), 8.36 (d, J=8.4 Hz, 1H), 7.97 (dd, J=8.5, 2.4 Hz, 1H), 7.73 (dd, J=7.0, 1.2 Hz, 1H), 7.52 (dd, J=7.2, 8.3 Hz, 1H), 6.89 (d, J=8.6 Hz, 1H), 4.57 (quintet, J=7.8 Hz, 1H), 4.36 (s, 2H), 3.92 (s, 3H), 1.88-1.80 (m, 2H), 1.77-1.64 (m, 4H), 1.63-1.54 (m, 2H). MS APCI, m/z=375.2 (M+H). HPLC 1.80 min. MS TOF, Theor m/z=375.18155 (M+H) Expl m/z=375.18237, Error=2.19 ppm.

### EXAMPLE 148

### 9-Amino-2-cyclobutyl-5-(6-morpholin-4-yl-pyridin-3-yl)-2,3-dihydropyrrolo[3,4-b]quinolin-1-one

Using Method A, 9-amino-5-bromo-2-cyclobutyl-2,3-dihydropyrrolo[3,4-b]quinolin-1-one (352.2 mg, 1.06 mmol) and 4-(5-(4,4,5,5-tetramethyl-1,3,2-dioxaborolan-2-yl)pyridin-2-yl)morpholine (774.2 mg, 2.67 mmol) were reacted to afford the title compound as an off-white solid (310.3 mg, 70.4%). ¹H NMR (500.333 MHz, DMSO) δ 8.41 (d, J=2.2 Hz, 1H), 8.31 (dd, J=8.5, 1.2 Hz, 1H), 7.88 (dd, J=8.7, 2.5 Hz, 1H), 7.70 (dd, J=7.0, 1.2 Hz, 2H), 6.90 (d, J=8.8 Hz, 1H), 4.74 (quintet, J=8.7 Hz, 1H), 4.46 (s, 2H), 3.74 (dd, J=6.0, 4.5 Hz, 4H), 3.51 (t, J=4.9 Hz, 4H), 3.28 (s, 3H), 2.34 (quintetd, J=9.5, 2.2 Hz, 2H), 2.17-2.09 (m, 2H), 1.74-1.66 (m, 2H). MS APCI, m/z=416.2 (M+H). HPLC 1.54 min. MS TOF, Theor m/z=416.20810 (M+H), Expl m/z=416.20816, Error=0.14 ppm.

### EXAMPLE 149

### 9-Amino-2-cyclobutyl-5-(6-methoxy-pyridin-3-yl)-2,3-dihydropyrrolo[3,4-b]quinolin-1-one

Using Method A, 9-amino-5-bromo-2-cyclobutyl-2,3-dihydropyrrolo[3,4-b]quinolin-1-one (350.4 mg, 1.05 mmol) and 6-methoxypyridin-3-ylboronic acid (430.3 mg, 2.81 mmol) were reacted to afford the title compound as a white solid (253.0 mg, 67%). ¹H NMR (500.333 MHz, DMSO) δ 8.40 (d, J=2.4 Hz, 1H), 8.36 (dd, J=8.3, 1.2 Hz, 1H), 7.98 (dd, J=8.4, 2.4 Hz, 1H), 7.73 (dd, J=7.0, 1.2 Hz, 1H), 7.67 (bs, 2H), 7.52 (dd, J=8.5, 7.1 Hz, 1H), 6.89 (d, J=8.6 Hz, 1H), 4.75 (quintet, J=8.7 Hz, 1H), 4.46 (s, 2H), 3.93 (s, 3H), 2.34 (quintetd, J=9.5, 2.4 Hz, 2H), 2.17-2.09 (m, 2H), 1.74-1.64 (m, 2H). MS APCI, m/z=361.2 (M+H). HPLC 7.2 min. MS TOF, Theor m/z=361.1654 (M+H), Expl m/z=361.1655, Error=−0.92 ppm.

### EXAMPLE 150

### 9-Amino-2-cyclobutyl-5-(4-methylpyridin-3-yl)-2,3-dihydropyrrolo[3,4-b]quinolin-1-one

Using Method A, 9-amino-5-bromo-2-cyclobutyl-2,3-dihydropyrrolo[3,4-b]quinolin-1-one (337.1 mg, 1.01 mmol) and (4-methyl-3-pyridyl)-boronic acid (0.3145 g, 2.30 mmol) were reacted to afford the title compound as a white solid (222.7 mg, 64%). ¹H NMR (500.333 MHz, DMSO) d 8.46-8.41 (m, H), 8.32 (s, 1H), 7.71 (bs, 2H), 7.60 (dd, J=7.1, 1.4 Hz, 1H), 7.54 (dd, J=8.6, 7.1 Hz, 1H), 7.32 (d, J=4.9 Hz, 1H), 4.73 (quintet, J=8.7 Hz, 1H), 4.42 (s, 2H), 2.38-2.24 (m, 2H), 2.14-2.06 (m, 2H), 2.01 (s, 3H), 1.72-1.63 (m, 2H). MS APCI, m/z=345.2 (M+H). HPLC 0.71 min. MS TOF, Theor m/z=345.17099 (M+H), Expl=345.17151, Error=1.51 ppm.

### EXAMPLE 151

### 9-Amino-2-cyclobutyl-5-(3-fluoropyrazin-2-yl)-2,3-dihydropyrrolo[3,4-b]quinolin-1-one

Using Method E, 9-amino-5-bromo-2-cyclobutyl-2,3-dihydro-1H-pyrrolo[3,4-b]quinolin-1-one (0.3313 g, 1.00 mmol) and 2-fluoro-3-(tributylstannyl)pyrazine (0.8472 g, 2.19 mmol) were reacted to afford the title compound as a white solid (37.9 mg, 11%).

¹H NMR(500.333 MHz, DMSO) δ 8.73 (dd, J=4.5, 2.7 Hz, 1H), 8.54 (dd, J=8.5, 1.3 Hz, 1H), 8.41 (dd, J=2.8, 1.5 Hz, 1H), 7.86 (dd, J=7.1, 1.3 Hz, 1H), 7.78 (bs, 1H), 7.61 (dd, J=8.5, 7.1 Hz, 1H), 4.72 (quintet, J=8.7 Hz, 1H), 4.43 (s, 2H), 2.32 (quintetd, J=9.7, 2.3 Hz, 2H), 2.15-2.05 (m, 2H), 1.74-1.61 (m, 2H). MS APCI, m/z=350.1 (M+H). HPLC 1.71 min. MS TOF, Theor m/z=350.14116 (M+H), Expl m/z=350.14154, Err=1.07 ppm.

### EXAMPLE 152

### 9-Amino-2-cyclobutyl-5-(5-methoxy-pyridin-3-yl)-2,3-dihydropyrrolo[3,4-b]quinolin-1-one

Using Method A, 9-amino-5-bromo-2-cyclobutyl-2,3-dihydropyrrolo[3,4-b]quinolin-1-one (352.3 mg, 1.06 mmol) and 3-methoxy-5-(4,4,5,5-tetramethyl-1,3,2-dioxaborolan-2-yl)pyridine (636.8 mg, 2.71 mmol) were reacted to afford the title compound as an off-white solid (246.8 mg, 65%). ¹H NMR (500.333 MHz, DMSO) d 8.42 (d, J=1.4 Hz, 1H), 8.40 (d, J=1.7 Hz, 1H), 8.29 (d, J=2.7 Hz, 1H), 7.0, 1.3 Hz, 1H), 7.61 (dd, J=2.9, 1.8 Hz, 1H), 7.55 (dd, J=7.0, 8.3 Hz, 1H), 4.74 (quintet, J=8.7 Hz, 1H), 4.48 (s, 2H), 3.89 (s, 3H), 2.39-2.29 (m, 2H), 2.17-2.08 (m, 2H), 1.74-1.64 (m, 2H). MS APCI, m/z=361.2 (M+H). HPLC 1.60 min. MS TOF, Theor m/z=361.16590, Expl m/z=361.16489, Error=−2.81 ppm.

### EXAMPLE 153

### 9-Amino-2-cyclopropyl-6-fluoro-5-(2-fluoro-3-methoxyphenyl)-2,3-dihydropyrrolo[3,4-b]quinolin-1-one

Using Method D, 9-amino-5-bromo-2-cyclopropyl-6-fluoro-2,3-dihydro-1H-pyrrolo[3,4-b]quinolin-1-one (180 mg, 0.54 mmol) and 2-fluoro-3-methoxyphenylboronic acid (0.7 g, 4.12 mmol) were reacted to afford the title compound as a tan solid (23.5 mg, 11.9%). 1H NMR (500 MHz, DMSO-d₆) δ ppm 8.50 (dd, J=9.6, 6.3 Hz, 1 H) 7.49 (dd, J=9.5, 9.1 Hz, 1 H) 7.17-7.24 (m, 2 H) 6.87-6.93 (m, 1 H) 4.23 (d, J=17.4 Hz, 1 H) 4.22 (d, J=17.4 Hz, 1 H) 3.89 (s, 3 H) 2.84-2.91 (m, 1 H) 0.78-0.86 (m, 2 H) 0.70-0.78 (m, 2 H). MS APCI, m/z=382. (M+H).

### EXAMPLE 154

### 9-Amino-2-cyclopropyl-5-(2,6-difluoro-3-methoxyphenyl)-6-fluoro-2,3-dihydropyrrolo[3,4-b]quinolin-1-one

Using Method D, 9-amino-5-bromo-2-cyclopropyl-6-fluoro-2,3-dihydro-1H-pyrrolo[3,4-b]quinolin-1-one (180 mg, 0.54 mmol) and 2,6-difluoro-3-methoxyphenylboronic acid (0.7 g, 3.7 mmol) were reacted to afford the title compound as a white solid (29.4 mg, 13.6%). 1H NMR (500 MHz, DMSO-d₆) δ ppm 8.57 (dd, J=9.3, 6.3 Hz, 1 H) 7.53 (t, J=9.0 Hz, 1 H) 7.28 (td, J=9.3, 5.2 Hz, 1 H) 7.14 (td, J=9.0, 1.8 Hz, 1 H) 4.24 (s, 2 H) 3.89 (s, 3 H) 2.83-2.92 (m, 1 H) 0.78-0.87 (m, 2 H) 0.69-0.78 (m, 2 H). MS APCI, m/z=400. (M+H).

### EXAMPLE 155

### 9-Amino-2-cyclopropyl-6-fluoro-5-(2-fluoro-5-methoxyphenyl)-2,3-dihydropyrrolo[3,4-b]quinolin-1-one

Using Method D, 9-amino-5-bromo-2-cyclopropyl-6-fluoro-2,3-dihydro-1H-pyrrolo[3,4-b]quinolin-1-one (180 mg, 0.54 mmol) and 2-fluoro-5-methoxyphenylboronic acid (0.7 g, 4.1 mmol) were reacted to afford the title compound as a white solid (135 mg, 68.4%). 1H NMR (300 MHz, DMSO-d₆) δ ppm 8.50 (dd, J=9.3, 6.2 Hz, 1 H) 7.49 (t, J=9.1 Hz, 1 H) 7.22 (t, J=9.1 Hz, 1 H) 7.01 (ddd, J=8.7, 3.8, 3.6 Hz, 1 H) 6.91 (dd, J=5.7, 3.1 Hz, 1 H) 4.24 (s, 2 H) 3.76 (s, 3 H) 2.83-2.95 (m, 1 H) 0.71-0.87 (m, 4 H). MS APCI, m/z=382. (M+H).

### EXAMPLE 156

### 9-Amino-2-ethyl-6-fluoro-5-(4-methylpyridin-3-yl)-2,3-dihydropyrrolo[3,4-b]quinolin-1-one

Using Method D, 9-amino-2-ethyl-6-fluoro-5-bromo-2,3-dihydro-1H-pyrrolo[3,4-b]quinolin-1-one (185 mg, 0.57 mmol) and 4-methylpyridin-3-ylboronic acid (700 mg, 5.1 mmol) were reacted to afford the title compound as an off-white solid (67.4 mg, 35%). 1H NMR (300 MHz, CHLOROFORM-d) δ ppm 8.53 (d, J=5.0 Hz, 1 H) 8.43 (s, 1 H) 7.91 (dd, J=9.2, 5.8 Hz, 1 H) 7.36 (dd, J=8.4 Hz, 1 H) 7.27-7.29 (m, 1 H) 4.28 (d, J=1.0 Hz, 2 H) 3.63 (q, J=7.2 Hz, 2 H) 2.10 (s, 3 H) 1.25 (t, J=7.3 Hz, 3 H). MS APCI, m/z=337. (M+H).

### EXAMPLE 157

### 9-Amino-2-ethyl-6-fluoro-5-(2-fluoro-5-methoxyphenyl)-2,3-dihydropyrrolo[3,4-b]quinolin-1-one

Using Method D, 9-amino-2-ethyl-6-fluoro-5-bromo-2,3-dihydro-1H-pyrrolo[3,4-b]quinolin-1-one (190 mg, 0.59 mmol) and 2-fluoro-5-methoxyphenylboronic acid (600 mg, 3.5 mmol) were reacted to afford the title compound as a white solid (73 mg, 33.5%). 1H NMR (500 MHz, DMSO-d₆) δ ppm 8.51 (dd, J=9.3, 6.2 Hz, 1 H) 7.49 (t, J=9.0 Hz, 1 H) 7.22 (t, J=9.0 Hz, 1 H) 7.02 (td, J=4.5, 3.4 Hz, 1 H) 6.93 (dd, J=5.7, 3.2 Hz, 1 H) 4.33 (s, 2 H) 3.76 (s, 3 H) 3.48 (q, J=7.2 Hz, 2 H) 1.15 (t, J=7.2 Hz, 3 H). MS APCI, m/z=370. (M+H).

### EXAMPLE 158

### 9-Amino-2-cyclobutyl-5-(2,4-dimethoxypyrimidin-5-yl)-6-fluoro-2,3-dihydropyrrolo[3,4-b]quinolin-1-one

Using Method D, 9-amino-5-bromo-2-cyclobutyl-6-fluoro-2,3-dihydro-1H-pyrrolo[3,4-b]quinolin-1-one (180 mg, 0.51 mmol), and 2,4-dimethoxy-5-(4,4,5,5-tetramethyl-1,3,2-dioxaborolan-2-yl)pyrimidine (850 mg, 3.2 mmol) were reacted to afford the title compound as a white solid (101 mg, 48%). 1H NMR (300 MHz, CHLOROFORM-d) δ ppm 8.27 (s, 1H) 7.87 (dd, J=9.2, 5.8 Hz, 1 H) 7.33 (d, J=8.6 Hz, 1 H) 6.40 (br. s., 2 H) 4.80-4.98 (m, 1 H) 4.39 (s, 2 H) 4.08 (s, 3 H) 3.94 (s, 3 H) 2.17-2.35 (m, 4 H) 1.69-1.88 (m, 2 H). MS APCI, m/z=410. (M+H).

### EXAMPLE 159

### 9-Amino-2-cyclobutyl-5-(2,5-dimethoxyphenyl)-6-fluoro-2,3-dihydropyrrolo[3,4-b]quinolin-1-one

Using Method D, 9-amino-5-bromo-2-cyclobutyl-6-fluoro-2,3-dihydro-1H-pyrrolo[3,4-b]quinolin-1-one (180 mg, 0.51 mmol), and 2,5-dimethoxyphenylboronic acid (350 mg, 1.92 mmol) were reacted to afford the title compound as a white solid (113 mg, 54%). 1H NMR (300 MHz, CHLOROFORM-d) δ ppm 7.83 (dd, J=9.2, 5.8 Hz, 1 H) 7.29 (d, J=8.5 Hz, 1 H) 6.93-7.02 (m, 2 H) 6.87 (d, J=2.6 Hz, 1 H) 6.35 (br. s., 2 H) 4.80-4.96 (m, 1 H) 4.38 (s, 2 H) 3.80 (s, 3 H) 3.68 (s, 3 H) 2.16-2.32 (m, 4 H) 1.70-1.84 (m, 2 H). MS APCI, m/z=408. (M+H).

### EXAMPLE 160

### 9-Amino-2-cyclobutyl-6-fluoro-5-(2-methoxypyridin-3-yl)-2,3-dihydropyrrolo[3,4-b]quinolin-1-one

Using Method D, 9-amino-5-bromo-2-cyclobutyl-6-fluoro-2,3-dihydro-1H-pyrrolo[3,4-b]quinolin-1-one (180 mg, 0.51 mmol), and 2-methoxypyridin-3-ylboronic acid (525 mg, 3.1 mmol) were reacted to afford the title compound as a white solid (105 mg, 49%). 1H NMR (300 MHz, MeOD) δ ppm 8.61 (dd, J=9.3, 5.4 Hz, 1 H) 8.43 (dd, J=5.1, 1.9 Hz, 1 H) 7.79 (dd, J=7.3, 1.9 Hz, 1 H) 7.67 (dd, J=9.2, 8.5 Hz, 1 H) 7.23 (dd, J=7.3, 5.1 Hz, 1 H) 4.65-4.79 (m, 1 H) 4.67 (d, J=2.4 Hz, 2 H) 3.89 (s, 3 H) 2.25-2.41 (m, 4 H) 1.74-1.89 (m, 2 H). MS APCI, m/z=379. (M+H).

### EXAMPLE 161

### 9-Amino-2-cyclopropyl-6-fluoro-5-(2-methoxyphenyl)-2,3-dihydropyrrolo[3,4-b]quinolin-1-one

Using Method D, 9-amino-5-bromo-2-cyclopropyl-6-fluoro-2,3-dihydro-1H-pyrrolo[3,4-b]quinolin-1-one (250 mg, 0.74 mmol) and 2-methoxyphenylboronic acid (0.45 g, 2.96 mmol) were reacted to afford the title compound as a white solid (108 mg, 39.8%). 1H NMR (300 MHz, DMSO-d₆) δ ppm 8.37-8.46 (m, 1 H) 7.34-7.46 (m, 2 H) 7.14 (dd, J=7.5, 1.8 Hz, 1 H) 7.12 (d, J=8.1 Hz, 1 H) 7.01 (td, 1 H) 4.19 (s, 2 H) 3.63 (s, 3 H) 2.81-2.93 (m, 1 H) 0.78-0.84 (m, 2 H) 0.69-0.77 (m, 2 H). MS APCI, m/z=364. (M+H).

### EXAMPLE 162

### 9-Amino-2-cyclobutyl-6-fluoro-5-(2-methoxyphenyl)-2,3-dihydropyrrolo[3,4-b]quinolin-1-one

Using Method D, 9-amino-5-bromo-2-cyclobutyl-6-fluoro-2,3-dihydro-1H-pyrrolo[3,4-b]quinolin-1-one (350 mg, 1.0 mmol), and 2-methoxyphenylboronic acid (510 mg, 3.36 mmol) were reacted to afford the title compound as a white solid (222 mg, 58.9%). 1H NMR (300 MHz, DMSO-d₆) δ ppm 8.42 (dd, J=9.4, 6.2 Hz, 1 H) 7.35-7.47 (m, 2 H) 7.16 (dd, J=7.4, 1.7 Hz, 1 H) 7.13 (d, J=8.1 Hz, 1 H) 7.02 (t, J=7.5 Hz, 1 H) 4.65-4.79 (m, 1 H) 4.38 (s, 2 H) 3.64 (s, 3 H) 2.24-2.36 (m, 2 H) 2.03-2.16 (m, 2 H) 1.60-1.74 (m, 2 H). MS APCI, m/z=378. (M+H).

### EXAMPLE 163

### 9-Amino-5-(5-chloro-2-methoxyphenyl)-2-ethyl-6-fluoro-2,3-dihydropyrrolo[3,4-b]quinolin-1-one

Using Method D, 9-amino-2-ethyl-6-fluoro-5-bromo-2,3-dihydro-1H-pyrrolo[3,4-b]quinolin-1-one (254 mg, 0.78 mmol) and 5-chloro-2-methoxyphenylboronic acid (480 mg, 2.6 mmol) were reacted to afford the title compound as a white solid (128 mg, 42.5%). 1H NMR (500 MHz, DMSO-d₆) δ ppm 8.45 (dd, J=9.3, 6.3 Hz, 1 H) 7.40-7.47 (m, 2 H) 7.21 (d, J=2.7 Hz, 1 H) 7.15 (d, J=8.9 Hz, 1 H) 4.31 (d, J=17.7 Hz, 1 H) 4.30 (d, J=17.7 Hz, 1 H) 3.65 (s, 3 H) 3.42-3.54 (m, 2 H) 1.14 (t, J=7.2 Hz, 3 H). MS APCI, m/z=386. (M+H).

### EXAMPLE 164

### 9-Amino-2-cyclobutyl-6-fluoro-5-(2-fluoro-5-methoxyphenyl)-2,3-dihydropyrrolo[3,4-b]quinolin-1-one

Using Method D, 9-amino-5-bromo-2-cyclobutyl-6-fluoro-2,3-dihydro-1H-pyrrolo[3,4-b]quinolin-1-one (350 mg, 1.0 mmol), and 2-fluoro-5-methoxyphenylboronic acid (510 mg, 3.0 mmol) were reacted to afford the title compound as a white solid (138 mg, 34.8%). 1H NMR (500 MHz, DMSO-d₆) δ ppm 8.51 (dd, J=9.3, 6.1 Hz, 1 H) 7.49 (t, J=9.0 Hz, 1 H) 7.22 (t, J=9.0 Hz, 1 H) 7.02 (dt, J=8.4, 6.5 Hz, 1 H) 6.94 (dd, J=5.7, 3.2 Hz, 1 H) 4.67-4.77 (m, 1 H) 4.44 (s, 2 H) 3.77 (s, 3 H) 2.25-2.37 (m, 2 H) 2.05-2.15 (m, 2 H) 1.63-1.72 (m, 2 H). MS APCI, m/z=396. (M+H).

### EXAMPLE 165

### 9-Amino-2-cyclobutyl-6-fluoro-5-(5-fluoro-2-methoxyphenyl)-2,3-dihydropyrrolo[3,4-b]quinolin-1-one

Using Method D, 9-amino-5-bromo-2-cyclobutyl-6-fluoro-2,3-dihydro-1H-pyrrolo[3,4-b]quinolin-1-one (350 mg, 1.0 mmol), and 5-fluoro-2-methoxyphenylboronic acid (500 mg, 2.9 mmol) were reacted to afford the title compound as a white solid (242 mg, 61.1%). 1H NMR (500 MHz, DMSO-d₆) δ ppm 8.44 (dd, J=9.2, 6.2 Hz, 1 H) 7.43 (t, J=9.0 Hz, 1 H) 7.21 (td, J=8.5, 3.2 Hz, 1 H) 7.12 (dd, J=9.2, 4.7 Hz, 1 H) 7.05 (dd, J=9.0, 3.2 Hz, 1 H) 4.67-4.75 (m, 1 H) 4.41 (d, J=17.6 Hz, 1 H) 4.41 (d, J=17.6 Hz, 1 H) 3.63 (s, 3 H) 2.25-2.34 (m, 2 H) 2.09-2.14 (m, 2 H) 1.63-1.72 (m, 2 H). MS APCI, m/z=396. (M+H).

### EXAMPLE 166

### 9-Amino-2-cyclobutyl-6-fluoro-5-(6-methoxy-4-methylpyridin-3-yl)-2,3-dihydropyrrolo[3,4-b]quinolin-1-one

Using Method D, 9-amino-5-bromo-2-cyclobutyl-6-fluoro-2,3-dihydro-1H-pyrrolo[3,4-b]quinolin-1-one (250 mg, 0.71 mmol), and 2-methoxy-4-methyl-5-(4,4,5,5-tetramethyl-1,3,2-dioxaborolan-2-yl)pyridine (350 mg, 1.4 mmol) were reacted to afford the title compound as an off-white solid (51.1 mg, 18%). 1H NMR (500 MHz, DMSO-d₆) δ ppm 8.49 (dd, J=9.3, 6.2 Hz, 1 H) 7.92 (s, 1 H) 7.49 (t, J=8.9 Hz, 1 H) 6.82 (s, 1 H) 4.66-4.77 (m, 1 H) 4.37-4.46 (m, 2 H) 3.90 (s, 3 H) 2.25-2.34 (m, 2 H) 2.07-2.14 (m, 2 H) 1.94 (s, 3 H) 1.67 (br. s., 2 H). MS APCI, m/z=393. (M+H).

### EXAMPLE 167

### 9-Amino-2-cyclobutyl-6-fluoro-5-(6-methoxy-2-methylpyridin-3-yl)-2,3-dihydropyrrolo[3,4-b]quinolin-1-one

Using Method D, 9-amino-5-bromo-2-cyclobutyl-6-fluoro-2,3-dihydro-1H-pyrrolo[3,4-b]quinolin-1-one (350 mg, 0.71 mmol), and 6-methoxy-2-methyl-3-(4,4,5,5-tetramethyl-1,3,2-dioxaborolan-2-yl)pyridine (850 mg, 3.4 mmol) were reacted to afford the title compound as a white solid (63.4 mg, 16.2%). 1H NMR (500 MHz, DMSO-d₆) δ ppm 8.48 (dd, J=9.3, 6.3 Hz, 1 H) 7.45-7.51 (m, 2 H) 6.72 (d, J=8.3 Hz, 1 H) 4.67-4.76 (m, 1 H) 4.42 (d, J=17.6 Hz, 1 H) 4.41 (d, J=17.6 Hz, 1 H) 3.91 (s, 3 H) 2.25-2.36 (m, 2 H) 2.09-2.14 (m, 2 H) 2.08 (s, 3 H) 1.63-1.74 (m, 2 H). MS APCI, m/z=393. (M+H).

### EXAMPLE 168

### 9-Amino-2-cyclobutyl-5-(2,5-dimethoxypyridin-3-yl)-2,3-dihydropyrrolo[3,4-b]quinolin-1-one

Using Method E, 9-amino-5-bromo-2-cyclobutyl-2,3-dihydro-1H-pyrrolo[3,4-b]quinolin-1-one (280 mg, 0.84 mmol) and 2,5-dimethoxy-3-(trimethylstannyl)pyridine (500 mg, 1.66 mmol) were reacted to afford the title compound as an off-white solid (84 mg, 25.5%). 1H NMR (300 MHz, CHLOROFORM-d) δ ppm 7.90 (d, J=3.0 Hz, 1 H) 7.85 (dd, J=8.3, 1.3 Hz, 1 H) 7.71 (dd, J=7.2, 1.3 Hz, 1 H) 7.52 (dd, J=8.2 Hz, 1 H) 7.34 (d, J=3.0 Hz, 1 H) 6.37 (ddd, J=2.0, 1.2, 1.0 Hz, 2 H) 4.82-4.97 (m, 1 H) 4.40 (s, 2 H) 3.86 (s, 3 H) 3.83 (s, 3 H) 2.24-2.35 (m, 4 H) 1.70-1.85 (m, 2 H). MS APCI, m/z=391. (M+H).

### EXAMPLE 169

### 9-Amino-6-fluoro-5-(2-methoxypyridin-3-yl)-2-(R)-tetrahydrofuran-3-yl-2,3-dihydropyrrolo[3,4-b]quinolin-1-one

Using Method D, (R)-9-amino-5-bromo-6-fluoro-2-(tetrahydrofuran-3-yl)-2,3-dihydro-1H-pyrrolo[3,4-b]quinolin-1-one (265 mg, 0.72 mmol), and 2-methoxypyridin-3-ylboronic acid (710 mg, 4.6 mmol) were reacted to afford the title compound as a pale yellow solid (148 mg, 51.7%). 1H NMR (300 MHz, CHLOROFORM-d) δ ppm 8.27 (dd, J=5.0, 1.9 Hz, 1 H) 7.86 (dd, J=9.2, 5.8 Hz, 1 H) 7.60 (dt, J=7.2, 2.1 Hz, 1 H) 7.34 (dd, J=9.0 Hz, 1 H) 7.02 (ddd, J=7.2, 5.1, 1.2 Hz, 1 H) 6.37 (br. s., 2 H) 5.04-5.14 (m, 1 H) 4.36 (d, J=17.3 Hz, 1 H) 4.34 (d, J=17.3 Hz, 1 H) 4.06 (td, J=8.5, 6.1 Hz, 1 H) 3.88 (s, 3 H) 3.76-3.92 (m, 3 H) 2.28-2.41 (m, 1 H) 1.94-2.06 (m, 1 H). MS APCI, m/z=395. (M+H).

### EXAMPLE 170

### 9-Amino-6-fluoro-5-(2-methoxypyridin-3-yl)-2-(S)-tetrahydrofuran-3-yl-2,3-dihydro-pyrrolo[3,4-b]quinolin-1-one

Using Method D, (S)-9-amino-5-bromo-6-fluoro-2-(tetrahydrofuran-3-yl)-2,3-dihydro-1H-pyrrolo[3,4-b]quinolin-1-one (302 mg, 0.82 mmol), and 2-methoxypyridin-3-ylboronic acid (950 mg, 6.1 mmol) were reacted to afford the title compound as a white solid (106 mg, 32.5%). 1H NMR (300 MHz, CHLOROFORM-d) δ ppm 8.27 (dd, J=4.9, 2.0 Hz, 1 H) 7.86 (dd, J=9.2, 5.8 Hz, 1 H) 7.60 (dt, J=7.3, 2.1 Hz, 1 H) 7.33 (dd, J=9.2, 8.8 Hz, 1 H) 7.02 (ddd, J=7.2, 5.1, 1.2 Hz, 1 H) 6.37 (br. s., 2 H) 5.02-5.15 (m, 1 H) 4.36 (d, J=17.4 Hz, 1 H) 4.34 (d, J=17.4 Hz, 1 H) 4.01-4.11 (m, 1 H) 3.82-3.89 (m, 3 H) 3.88 (s, 3 H) 2.27-2.41 (m, 1 H) 1.93-2.06 (m, 1 H). MS APCI, m/z=395. (M+H).

### EXAMPLE 171

### 9-Amino-2-cyclobutyl-5-(3,4-dimethoxyphenyl)-6-fluoro-2,3-dihydropyrrolo[3,4-b]quinolin-1-one

Using Method D, 9-amino-5-bromo-2-cyclobutyl-6-fluoro-2,3-dihydro-1H-pyrrolo[3,4-b]quinolin-1-one (180 mg, 0.51 mmol), and 3,4-dimethoxyphenylboronic acid (250 mg, 1.37 mmol) were reacted to afford the title compound as a white solid (147 mg, 70%). 1H NMR (500 MHz, DMSO-d₆) δ ppm 8.40 (dd, J=9.2, 6.1 Hz, 1 H) 7.44 (d, J=9.2 Hz, 1 H) 7.03 (d, J=8.3 Hz, 1 H) 7.02 (d, J=1.6 Hz, 1 H) 6.94 (dd, J=7.5, 1.0 Hz, 1 H) 4.68-4.77 (m, 1 H) 4.42 (s, 2 H) 3.83 (s, 3 H) 3.75 (s, 3 H) 2.26-2.37 (m, 2 H) 2.07-2.15 (m, 2 H) 1.64-1.73 (m, 2 H). MS APCI, m/z=408. (M+H).

### Method AA

Preparation of Xenopus oocytes

Xenopus laevis frogs (Xenopus I, Kalamazoo, Mich.) were anesthetized using 0.15% tricaine. Surgically removed ovarian lobes were teased out in OR2 solution (82 NaCl, 2.5 KCl, 5 HEPES, 1.5 NaH₂PO₄, 1 MgCl₂, 0.1 EDTA, in mM, pH 7.4). The oocytes were defolliculated by incubation in 25 mL OR2 containing 0.2% collagenase 1A (SIGMA) two times for about 60 minutes on a platform shaker and stored in Leibovitz's L-15 medium. Oocytes were injected the following day in 0.5× Leibovitz's L-15 medium containing 50 mg/ml gentamycin, 10 units/ml penicillin, and 10 mg/ml streptomycin.

### Method BB

Preparation and Injection of cRNA

Capped cRNAs from the linearized vectors containing human α₁, β₂ and γ₂ subunits of the GABAA receptor genes were mixed in ratio of 1:1:30. Oocytes were injected with 25-50 nL of mixed RNA with an appx molar ratio for α₁, β₂, and γ₂ as 1:1:10. Oocyte recordings were done 2-10 days after injection. The same methods apply to subtypes derived from α₂β₃γ₂, α₃β₃γ₂, and α₅β₃γ₂, except for 1:1:1 ratio was used for α, β, and γ subunits.

### Method CC

### Two-Electrode Voltage-Clamping Measurements

All measurements were done in a medium containing ND-96 (96 NaCl, 2 KCl, 1.8 CaCl₂.2H₂O, 1 MgCl₂.6H₂O, 5 HEPES, in mM, pH 7.5). Two-electrode voltage-clamp recording was carried out using OpusXpress amplifier (Axon Instruments, Foster City, Calif.), which allows simultaneous recording from 8 oocytes. Oocytes were impaled with two electrodes of 1-2 MΩ tip resistance when filled with 3M KCl. Recordings were begun when membrane potential became stable at potentials negative to −50-−60 mV. Membrane potential was held at −60 mV. Typical leak currents were between 0-40 nA, and rarely if a few cells did have a relatively high leak (>100 nA) they were not used. For the determination of the GABA EC10, a series of 30 s pulses with increasing concentrations of GABA were applied to the cells every 5 minutes. After calculating EC10 for GABA for each oocyte, a series of 30 s GABA pulses were applied at 5 minutes interval, with increasing doses of the modulator. The concentration of GABA corresponded to the EC10 value calculated for each oocyte. The modulator pulses started 30 s before the GABA pulse so as to allow preincubation with the modulator. A set of 3 pulses with just GABA without modulator was given prior to the modulator-containing pulses to define the baseline GABA response. Two oocytes per each experiment were dedicated to observe the effect of diazepam on GABA response to ensure the presence of γ₂ subunit in the GABAA pentameric complex, which imparts diazepam sensitivity to the complex.

### Method DD

### Calculation of Current Amplitude and Curve Fitting

Current amplitude (i) was measured from baseline to peak using Clampfit (Axon Inst., Foster City, Calif.). Potentiation was calculated as percent change from the baseline GABA current flux 100×(i_{mod}/i_{control})−1) where i_{mod}=current mediated by modulator+GABA and i_{control}=current mediated by GABA alone. A value of 100% potentiation means that modulator has caused the control current to double. Similarly, a value of −50% potentiation means the presence of modulator caused a 50% decrease in the control current. Various other data shown here were fitted and plotted using GraphPad Prism (GraphPad Software, Inc. San Diego, Calif.). The percentage potentiation was converted to relative potentiation by dividing it with percentage potentiation value obtained from the same assay with diazepam as a control.

### Method EE

### GABAA1 Binding Method

### Reagents

Assay and Wash Buffer: 50 mM Tris-Citrate, 200 mM NaCl, pH 7.8

Compounds at 10 mM in DMSO: Put 75 μl in column 1 of compound plate.

Flumazenil, 10 mM (for NSB)

Membranes α1, β2, γ2 receptor subunits transfected into Sf9 cells and harvested; prepared by Cell Trends, stored at −80° C.) Sonicate thawed membranes for about 5-10 seconds at setting 3 on Brinkman sonicator, then dilute membranes 1:71 in assay buffer (working conc.=100 ug/ml protein). Keep on ice.

[³H]-Flunitrazepam (Cat #NET567): Prepare 10× stock=30 nM, [F] in assay=˜3 nM

### Assay (See Below for Automation Programs.)

1. On PlateMate, prepare 1:3 serial dilutions (30 μl+60 μl) in DMSO for final assay concentrations of 10 μM to 170 pM (Automation Programs 1 and 2). Add 5 ul of 30 uM flumazenil to wells 12 D-E for 50% control wells.

2. Spot 2 μl of compound dilutions into dry plate (Automation Program 3). Manually spot 2 μl 10 mM flumazenil into wells 12 F-H for nonspecific control.

3. Make 1:100 dilution in assay buffer (2 μl into 200 μl) and dispense 25 μl compound into assay plates (Automation Program 4).

4. Dispense 200 μl membranes into assay plate (Automation Program 5).

5. Add 25 μl [³H]-Flunitrazepam (Automation Program 6). Incubate for 1 hr at 4° C.

6. Collect membranes on a cell harvester onto GF/B filter plates (pre-wet with dH₂O and wash 5×400 μl/well, with cold assay buffer. (First 3 washes are considered hot; last two are cold.)

7. Dry plates for 2-3 hours at RT.

8. Add 40 μl Microscint 40/well (Automation Program 7); seal plates. Count on a TopCount.

### Automation Programs

1. PlateMate add 60 ul DMSO for dilutions 96 w: 96/300 ul head, 5516 tips in columns 2-12, compound plate in left stacker A, DMSO reservoir on stage 2

2. PlateMate 11 pt-dilut one-third GABAA: 96/300 ul head, 5516 tips in column 1 of serial dilution magazine, compound plate in left stacker A

3. PlateMate 2 ul addition of cmpd dry new wash: 96/30 ul head, 5506 tips, compound plate in left stacker A, dilution plate in right stacker A, 100% DMSO in reservoir on stage 2, must change to fresh DMSO every 4-6 plates.

4. PlateMate tip chg mix and disp 25 ul to assay plate 96 w: 96/300 ul head, 5516 tips, dilution plate in left stacker A, assay plates in right stacker A, auto fill assay buffer reservoir on stage 2, need to change tips after every plate.

5. PlateMate add 200 ul membranes 96 w: 96/300 ul head, 5516 tips, assay plates in left stacker A, membrane reservoir on stage 2.

6. RapidPlate add 25 ul hot (number of plates): 100 μl (yellow box) tips in position 1, hot reservoir in position 2, plates beginning in position 3

7. RapidPlate add microscint 40 ul (number of plates): 200 μl (burgundy box) tips in position 1, Microscint 40 reservoir in position 2, plates beginning in position 3.

### Data Analysis

Data is analyzed by calculating percent of control, IC50, and Ki in an XLfit template. The following formula is used in the templates:

$$Ki = \frac{IC 50}{1 + [ ligand ] / {K}_{D}}$$

### Method FF

### GABAA2 Binding Method

### Reagents

Assay and Wash Buffer: 50 mM Tris-Citrate, 200 mM NaCl, pH 7.8

Compounds at 10 mM in DMSO: Put 75 μl in column 1 of compound plate.

Flumazenil, 10 mM (for NSB)

Membranes (α2, β3, γ2 receptor subunits transfected into Sf9 cells and harvested; prepared by Paragon at 12.5 mg/ml, stored at −80° C.) Sonicate thawed membranes for about 5-10 seconds at setting 3 on Brinkman sonicator, then dilute membranes 1:50 in assay buffer (working conc.=250 ug/ml protein). Keep on ice.

[³H]-Flunitrazepam (Cat #NET567): Prepare 10× stock=20 nM, [F} in assay=˜2 nM

### Assay (See Below for Automation Programs.)

1. On PlateMate, prepare 1:3 serial dilutions (30 μl+60 μl) in DMSO for final assay concentrations of 10 μM to 170 pM (Automation Programs 1 and 2). Add 5 ul of 30 uM flumazenil to wells 12 D-E for 50% control wells.

2. Spot 2 μl of compound dilutions into dry plate (Automation Program 3). Manually spot 2 μl 10 mM flumazenil into wells 12 F-H for nonspecific control.

3. Make 1:100 dilution in assay buffer (2 μl into 200 μl) and dispense 25 μl compound into assay plates (Automation Program 4).

4. Dispense 200 μl membranes into assay plate (Automation Program 5).

5. Add 25 μl [³H]-Flunitrazepam (Automation Program 6). Incubate for 1 hr at 4° C.

6. Collect membranes on a cell harvester onto GF/B filter plates (pre-wet with dH₂O and wash 5×400 μl/well, with cold assay buffer. (First 3 washes are considered hot; last two are cold.)

7. Dry plates for 2-3 hours at RT.

8. Add 40 μl Microscint 40/well (Automation Program 7); seal plates. Count on a TopCount.

### Automation Programs

1. PlateMate add 60 ul DMSO for dilutions 96 w: 96/300 ul head, 5516 tips in columns 2-12, compound plate in left stacker A, DMSO reservoir on stage 2.

2. PlateMate 11 pt-dilut one-third GABAA: 96/300 ul head, 5516 tips in column 1 of serial dilution magazine, compound plate in left stacker A.

3. PlateMate 2 ul addition of cmpd dry new wash: 96/30 ul head, 5506 tips, compound plate in left stacker A, dilution plate in right stacker A, 100% DMSO in reservoir on stage 2, must change to fresh DMSO every 4-6 plates.

4. PlateMate tip chg mix and disp 25 ul to assay plate 96 w: 96/300 ul head, 5516 tips, dilution plate in left stacker A, assay plates in right stacker A, auto fill assay buffer reservoir on stage 2, need to change tips after every plate.

5. PlateMate add 200 ul membranes 96 w: 96/300 ul head, 5516 tips, assay plates in left stacker A, membrane reservoir on stage 2.

6. RapidPlate add 25 ul hot (number of plates): 100 μl (yellow box) tips in position 1, hot reservoir in position 2, plates beginning in position 3.

7. RapidPlate add microscint 40 ul (number of plates): 200 μl (burgundy box) tips in position 1, Microscint 40 reservoir in position 2, plates beginning in position 3.

### Data Analysis

Data is analyzed by calculating percent of control, IC50, and Ki in an XLfit template. The following formula is used in the templates:

$$Ki = \frac{IC 50}{1 + [ ligand ] / {K}_{D}}$$

### Method GG

### GABAA3 Binding Method

### Reagents

Assay and Wash Buffer: 50 mM Tris-Citrate, 200 mM NaCl, pH 7.8

Compounds at 10 mM in DMSO: Put 75 ul in column 1 of compound plate.

Flumazenil, 10 mM (for NSB)

Membranes (α3, β3, γ2 receptor subunits transfected into Sf9 cells and harvested; prepared by Cell Trends, stored at −80° C.) Sonicate thawed membranes for about 5-10 seconds at setting 3 on Brinkman sonicator, then dilute membranes 1:125 to make a solution of 200 ug/mL in assay buffer. Keep on ice.

[3H]-Flunitrazepam (Cat #NET567): Prepare 10× stock=30 nM, [F} in assay=˜3 nM

### Assay (See Below for Automation Programs.)

1. On PlateMate, prepare 1:3 serial dilutions (30 μl+60 μl) in DMSO for final assay concentrations of 10 μM to 170 pM (Automation Programs 1 and 2). Add 5 μl of 30 μM flumazenil to wells 12 D-E for 50% control wells.

2. Spot 2 μl of compound dilutions into dry plate (Automation Program 3). Manually spot 2 μl 10 mM flumazenil into wells 12 F-H for nonspecific control.

3. Make 1:100 dilution in assay buffer (2 μl into 200 μl) and dispense 25 μl compound into assay plates (Automation Program 4).

4. Dispense 200 μl membranes into assay plate (Automation Program 5).

5. Add 25 μl [³H]-Flunitrazepam (Automation Program 6). Incubate for 1 hr at 4° C.

6. Collect membranes on a cell harvester onto GF/B filter plates (pre-wet with dH₂O and wash 5×400 μl/well, with cold assay buffer. (First 3 washes are considered hot; last two are cold.)

7. Dry plates for 2-3 hours at RT.

8. Add 40 μl Microscint 40/well (Automation Program 7); seal plates. Count on a TopCount.

### Automation Programs

1. PlateMate add 60 μl DMSO for dilutions 96 w: 96/300 μl head, 5516 tips in columns 2-12, compound plate in left stacker A, DMSO reservoir on stage 2.

2. PlateMate 11 pt-dilut one-third GABAA: 96/300 μl head, 5516 tips in column 1 of serial dilution magazine, compound plate in left stacker A.

3. PlateMate 2 μl addition of cmpd dry new wash: 96/30 μl head, 5506 tips, compound plate in left stacker A, dilution plate in right stacker A, 100% DMSO in reservoir on stage 2, must change to fresh DMSO every 4-6 plates.

4. PlateMate tip chg mix and disp 25 μl to assay plate 96 w: 96/300 μl head, 5516 tips, dilution plate in left stacker A, assay plates in right stacker A, auto fill assay buffer reservoir on stage 2, need to change tips after every plate.

5. PlateMate add 200 μl membranes 96 w: 96/300 μl head, 5516 tips, assay plates in left stacker A, membrane reservoir on stage 2.

6. RapidPlate add 25 μl hot (number of plates): 100 μl (yellow box) tips in position 1, hot reservoir in position 2, plates beginning in position 3.

7. RapidPlate add microscint 40 μl (number of plates): 200 μl (burgundy box) tips in position 1, Microscint 40 reservoir in position 2, plates beginning in position 3.

### Data Analysis

Data is analyzed by calculating percent of control, IC50, and Ki in an XLfit template. The following formula is used in the templates:

$$Ki = \frac{IC 50}{1 + [ ligand ] / {K}_{D}}$$

### Method HH

### GABAA5 Binding Method

### Reagents

Assay and Wash Buffer: 50 mM Tris-Citrate, 200 mM NaCl, pH 7.8

Compounds at 10 mM in DMSO: Put 75 μl in column 1 of compound plate.

Flumazenil, 10 mM (for NSB)

Membranes (α5, β3, γ2 receptor subunits transfected into Sf9 cells and harvested; prepared by Cell Trends, stored at −80° C.) Sonicate thawed membranes for about 5-10 seconds at setting 3 on Brinkman sonicator, then dilute membranes 1:31 in assay buffer (working conc.=500 ug/ml protein). Keep on ice.

[³H]-Flunitrazepam (Cat #NET567): Prepare 10× stock=20 nM, [F] in assay=˜2 nM

### Assay (See Below for Automation Programs.)

1. On PlateMate, prepare 1:3 serial dilutions (30 μl+60 μl) in DMSO for final assay concentrations of 10 μM to 170 pM (Automation Programs 1 and 2). Add 5 ul of 30 uM flumazenil to wells 12 D-E for 50% control wells.

2. Spot 2 μl of compound dilutions into dry plate (Automation Program 3). Manually spot 2 μl 10 mM flumazenil into wells 12 F-H for nonspecific control.

3. Make 1:100 dilution in assay buffer (2 μl into 200 μl) and dispense 25 μl compound into assay plates (Automation Program 4).

4. Dispense 200 μl membranes into assay plate (Automation Program 5).

5. Add 25 μl [³H]-Flunitrazepam (Automation Program 6). Incubate for 1 hr at 4° C.

6. Collect membranes on a cell harvester onto GF/B filter plates (pre-wet with dH₂O and wash 5×400 μl/well, with cold assay buffer. (First 3 washes are considered hot; last two are cold.)

7. Dry plates for 2-3 hours at RT.

8. Add 40 μl Microscint 40/well (Automation Program 7); seal plates. Count on a TopCount.

### Automation Programs

1. PlateMate add 60 ul DMSO for dilutions 96 w: 96/300 ul head, 5516 tips in columns 2-12, compound plate in left stacker A, DMSO reservoir on stage 2.

2. PlateMate 11 pt-dilut one-third GABAA: 96/300 ul head, 5516 tips in column 1 of serial dilution magazine, compound plate in left stacker A.

3. PlateMate 2 ul addition of cmpd dry new wash: 96/30 ul head, 5506 tips, compound plate in left stacker A, dilution plate in right stacker A, 100% DMSO in reservoir on stage 2, must change to fresh DMSO every 4-6 plates.

4. PlateMate tip chg mix and disp 25 ul to assay plate 96 w: 96/300 ul head, 5516 tips, dilution plate in left stacker A, assay plates in right stacker A, auto fill assay buffer reservoir on stage 2, need to change tips after every plate.

5. PlateMate add 200 ul membranes 96 w: 96/300 ul head, 5516 tips, assay plates in left stacker A, membrane reservoir on stage 2.

6. RapidPlate add 25 ul hot (number of plates): 100 μl (yellow box) tips in position 1, hot reservoir in position 2, plates beginning in position 3.

7. RapidPlate add microscint 40 ul (number of plates): 200 μl (burgundy box) tips in position 1, Microscint 40 reservoir in position 2, plates beginning in position 3.

### Data Analysis

Data is analyzed by calculating percent of control, IC50, and Ki in an XLfit template. The following formula is used in the templates:

$$Ki = \frac{IC 50}{1 + [ ligand ] / {K}_{D}}$$

Certain compounds of the invention are tested using one or more assays described above and the test results are summarized in the following Table 2.

TABLE 2

| | | Relative | Relative |
|---|---|---|---|
| | GABAA2 | Potentiation | Potentiation |
| | Binding | for | for |
| Compound Name | Ki (M) | GABAA1 | GABAA2 |
| 9-amino-2-cyclopropyl-5-(2- | 3.63E−09 | −0.053 | 0.38 |
| fluoro-6-methoxyphenyl)-2,3- | | | |
| dihydro-1H-pyrrolo[3,4- | | | |
| b]quinolin-1-one | | | |
| 9-amino-2-cyclobutyl-5-(2,6- | 7.74E−10 | 0.023 | 0.27 |
| dimethoxypyridin-3-yl)-6- | | | |
| fluoro-2,3-dihydro-1H- | | | |
| pyrrolo[3,4-b]quinolin-1-one | | | |
| 9-amino-2-cyclobutyl-5-(2- | 1.62E−09 | −0.07 | 0.2 |
| fluoro-6-methoxyphenyl)-2,3- | | | |
| dihydro-1H-pyrrolo[3,4- | | | |
| b]quinolin-1-one | | | |
| 9-amino-2-cyclobutyl-5-(4- | 1.84E−09 | 0.09 | 0.44 |
| methoxypyridin-3-yl)-2,3- | | | |
| dihydro-1H-pyrrolo[3,4- | | | |
| b]quinolin-1-one | | | |
| 9-amino-2-cyclobutyl-5-(2,4- | 2.13E−09 | 0.1 | 0.24 |
| dimethoxyphenyl)-2,3-dihydro- | | | |
| 1H-pyrrolo[3,4-b]quinolin-1-one | | | |
| 9-amino-2-cyclopropyl-6- | 2.35E−09 | 0.13 | 0.28 |
| fluoro-5-(4-methoxypyridin-3- | | | |
| yl)-2,3-dihydro-1H-pyrrolo[3,4- | | | |
| b]quinolin-1-one | | | |
| 9-amino-2-ethyl-6-fluoro-5-(4- | 2.43E−09 | 0.056 | 0.25 |
| methoxypyridin-3-yl)-2,3- | | | |
| dihydro-1H-pyrrolo[3,4- | | | |
| b]quinolin-1-one | | | |
| 9-amino-5-(5-chloro-2- | 2.53E−09 | 0.12 | 0.58 |
| methoxyphenyl)-2-cyclobutyl- | | | |
| 2,3-dihydro-1H-pyrrolo[3,4- | | | |
| b]quinolin-1-one | | | |
| 9-amino-2-cyclopropyl-6- | 2.59E−09 | 0.11 | 0.26 |
| fluoro-5-(2-methoxypyridin-3- | | | |
| yl)-2,3-dihydro-1H-pyrrolo[3,4- | | | |
| b]quinolin-1-one | | | |
| 9-amino-2-cyclopropyl-6- | 2.59E−09 | −0.075 | 0.24 |
| fluoro-5-(2-fluoro-6- | | | |
| methoxyphenyl)-2,3-dihydro- | | | |
| 1H-pyrrolo[3,4-b]quinolin-1-one | | | |
| 9-amino-5-(2,6- | 2.94E−09 | 0.15 | 0.35 |
| dimethoxypyridin-3-yl)-6- | | | |
| fluoro-2-propyl-2,3-dihydro-1H- | | | |
| pyrrolo[3,4-b]quinolin-1-one | | | |
| 9-amino-2-cyclobutyl-5-(2- | 3.01E−09 | −0.015 | 0.44 |
| methoxypyridin-3-yl)-2,3- | | | |
| dihydro-1H-pyrrolo[3,4- | | | |
| b]quinolin-1-one | | | |
| 9-amino-6-fluoro-5-(2-fluoro-6- | 3.49E−09 | 0.06 | 0.27 |
| methoxyphenyl)-2-propyl-2,3- | | | |
| dihydro-1H-pyrrolo[3,4- | | | |
| b]quinolin-1-one | | | |
| 9-amino-2-cyclobutyl-5-(2- | 3.52E−09 | 0.038 | 0.25 |
| methoxyphenyl)-2,3-dihydro- | | | |
| 1H-pyrrolo[3,4-b]quinolin-1-one | | | |
| 9-amino-2-cyclobutyl-5-(2,6- | 3.81E−09 | 0.06 | 0.31 |
| dimethoxypyridin-3-yl)-2,3- | | | |
| dihydro-1H-pyrrolo[3,4- | | | |
| b]quinolin-1-one | | | |
| 9-amino-2-ethyl-6-fluoro-5-(2- | 4.08E−09 | −0.055 | 0.28 |
| methoxypyridin-3-yl)-2,3- | | | |
| dihydro-1H-pyrrolo[3,4- | | | |
| b]quinolin-1-one | | | |
| 9-amino-5-(2-chloro-6- | 4.36E−09 | 0.11 | 0.34 |
| methylpyridin-3-yl)-2-(3,4- | | | |
| dimethoxybenzyl)-2,3-dihydro- | | | |
| 1H-pyrrolo[3,4-b]quinolin-1-one | | | |
| 9-amino-2-ethyl-5-(2-fluoro-6- | 4.36E−09 | −0.11 | 0.15 |
| methoxyphenyl)-2,3-dihydro- | | | |
| 1H-pyrrolo[3,4-b]quinolin-1-one | | | |
| 9-amino-5-(3,4- | 4.52E−09 | 0.068 | 0.27 |
| dimethoxyphenyl)-2-ethyl-6- | | | |
| fluoro-2,3-dihydro-1H- | | | |
| pyrrolo[3,4-b]quinolin-1-one | | | |
| 9-amino-2-cyclobutyl-5-(2,5- | 4.65E−09 | 0.073 | 0.4 |
| dimethoxyphenyl)-2,3-dihydro | | | |
| 1H-pyrrolo[3,4-b]quinolin-1-one | | | |
| 9-amino-5-(2,6-difluorophenyl)- | 4.98E−09 | 0.083 | 0.24 |
| 2-propyl-2,3-dihydro-1H- | | | |
| pyrrolo[3,4-b]quinolin-1-one | | | |
| 9-amino-5-(2-fluoro-6- | 5.03E−09 | 0.11 | 0.3 |
| methoxyphenyl)-2-propyl-2,3- | | | |
| dihydro-1H-pyrrolo[3,4- | | | |
| b]quinolin-1-one | | | |
| 9-amino-2-(2,5- | 5.11E−09 | 0.18 | 0.26 |
| dimethoxybenzyl)-5-(4- | | | |
| methoxypyridin-3-yl)-2,3- | | | |
| dihydro-1H-pyrrolo[3,4- | | | |
| b]quinolin-1-one | | | |
| 9-amino-2-cyclopropyl-5-(2- | 6.36E−09 | −0.034 | 0.26 |
| fluoro-6-methoxyphenyl)-2,3- | | | |
| dihydro-1H-pyrrolo[3,4- | | | |
| b]quinolin-1-one | | | |
| 9-amino-2-cyclopropyl-5-(2,4- | 6.64E−09 | 0.13 | 0.35 |
| dimethoxypyrimidin-5-yl)-6- | | | |
| fluoro-2,3-dihydro-1H- | | | |
| pyrrolo[3,4-b]quinolin-1-one | | | |
| 9-amino-5-(2-chloro-6- | 7.06E−09 | 0.29 | 0.87 |
| methylpyridin-3-yl)-2-propyl- | | | |
| 2,3-dihydro-1H-pyrrolo[3,4- | | | |
| b]quinolin-1-one | | | |
| 9-amino-2-cyclopropyl-6- | 7.40E−09 | 0.08 | 0.23 |
| fluoro-5-(5-fluoro-2- | | | |
| methoxyphenyl)-2,3-dihydro- | | | |
| 1H-pyrrolo[3,4-b]quinolin-1-one | | | |
| 9-amino-2-cyclopropyl-5-(2,4- | 8.33E−09 | 0.058 | 0.19 |
| dimethoxyphenyl)-6-fluoro-2,3- | | | |
| dihydro-1H-pyrrolo[3,4- | | | |
| b]quinolin-1-one | | | |
| 9-amino-5-(2,6- | 8.96E−09 | 0.062 | 0.18 |
| dimethoxypyridin-3-yl)-2-ethyl- | | | |
| 6-fluoro-2,3-dihydro-1H- | | | |
| pyrrolo[3,4-b]quinolin-1-one | | | |
| 9-amino-5-(2-fluoro-6- | 9.80E−09 | −0.034 | 0.2 |
| methoxyphenyl)-2-(4- | | | |
| methoxybenzyl)-2,3-dihydro- | | | |
| 1H-pyrrolo[3,4-b]quinolin-1-one | | | |
| 9-amino-2-ethyl-5-(2-fluoro-3- | 9.93E−09 | 0.01 | 0.21 |
| methoxyphenyl)-2,3-dihydro- | | | |
| 1H-pyrrolo[3,4-b]quinolin-1-one | | | |
| 9-amino-2-(3,4- | 1.10E−08 | 0.15 | 0.19 |
| dimethoxybenzyl)-5-(2,5- | | | |
| dimethoxyphenyl)-2,3-dihydro- | | | |
| 1H-pyrrolo[3,4-b]quinolin-1-one | | | |
| 9-amino-2-cyclopropyl-5-(2,5- | 1.14E−08 | 0.094 | 0.25 |
| dichlorophenyl)-2,3-dihydro- | | | |
| 1H-pyrrolo[3,4-b]quinolin-1-one | | | |
| 9-amino-2-ethyl-6-fluoro-5-(2- | 1.32E−08 | −0.11 | 0.26 |
| methoxyphenyl)-2,3-dihydro- | | | |
| 1H-pyrrolo[3,4-b]quinolin-1-one | | | |
| 9-amino-2-cyclopropyl-5-(2,5- | 1.38E−08 | 0.078 | 0.26 |
| dimethoxyphenyl)-6-fluoro-2,3- | | | |
| dihydro-1H-pyrrolo[3,4- | | | |
| b]quinolin-1-one | | | |
| 9-amino-2-cyclopropyl-5-(2- | 1.38E−08 | 0.079 | 0.33 |
| fluoro-5-methoxyphenyl)-2,3- | | | |
| dihydro-1H-pyrrolo[3,4- | | | |
| b]quinolin-1-one | | | |
| 2-(9-amino-2-ethyl-1-oxo-2,3- | 1.41E−08 | −0.11 | 0.17 |
| dihydro-1H-pyrrolo[3,4- | | | |
| b]quinolin-5-yl)benzonitrile | | | |
| 9-amino-2-cyclopropyl-5-(2,6- | 1.45E−08 | −0.051 | 0.25 |
| dimethoxypyridin-3-yl)-6- | | | |
| fluoro-2,3-dihydro-1H- | | | |
| pyrrolo[3,4-b]quinolin-1-one | | | |
| 9-amino-2-(benzo[d][1,3]dioxol- | 1.55E−08 | 0.22 | 0.37 |
| 5-ylmethyl)-5-(2-methoxy-5- | | | |
| methylphenyl)-2,3-dihydro-1H- | | | |
| pyrrolo[3,4-b]quinolin-1-one | | | |
| 9-amino-5-(2,4- | 1.67E−08 | −0.037 | 0.23 |
| dimethoxypyrimidin-5-yl)-2- | | | |
| ethyl-6-fluoro-2,3-dihydro-1H- | | | |
| pyrrolo[3,4-b]quinolin-1-one | | | |
| 9-amino-5-(2,3- | 1.73E−08 | 0.13 | 0.22 |
| dimethylphenyl)-2-propyl-2,3- | | | |
| dihydro-1H-pyrrolo[3,4- | | | |
| b]quinolin-1-one | | | |
| 9-amino-2-ethyl-6-fluoro-5-(5- | 1.73E−08 | −0.008 | 0.3 |
| fluoro-2-methoxyphenyl)-2,3- | | | |
| dihydro-1H-pyrrolo[3,4- | | | |
| b]quinolin-1-one | | | |
| 9-amino-2-cyclopropyl-5-(2- | 1.82E−08 | 0.07 | 0.14 |
| fluoro-3-methoxyphenyl)-2,3- | | | |
| dihydro-1H-pyrrolo[3,4- | | | |
| b]quinolin-1-one | | | |
| 9-amino-5-(2-fluoro-6- | 1.88E−08 | 0.046 | 0.26 |
| methoxyphenyl)-2-methyl-2,3- | | | |
| dihydro-1H-pyrrolo[3,4- | | | |
| b]quinolin-1-one | | | |
| 9-amino-2-ethyl-5-(4- | 2.14E−08 | 0.079 | 0.26 |
| methoxypyridin-3-yl)-2,3- | | | |
| dihydro-1H-pyrrolo[3,4- | | | |
| b]quinolin-1-one | | | |
| 9-amino-5-(3,4- | 2.14E−08 | 0.07 | 0.19 |
| dimethoxyphenyl)-2-ethyl-2,3- | | | |
| dihydro-1H-pyrrolo[3,4- | | | |
| b]quinolin-1-one | | | |
| 9-amino-2-(4-methoxybenzyl)- | 2.20E−08 | 0.12 | 0.68 |
| 5-(2-methoxypyridin-3-yl)-2,3- | | | |
| dihydro-1H-pyrrolo[3,4- | | | |
| b]quinolin-1-one | | | |
| 9-amino-2-ethyl-5-(4-fluoro-2- | 2.21E−08 | −0.05 | 0.2 |
| methoxyphenyl)-2,3-dihydro- | | | |
| 1H-pyrrolo[3,4-b]quinolin-1-one | | | |
| 9-amino-2-ethyl-5-(2- | 2.42E−08 | 0.022 | 0.28 |
| methoxypyridin-3-yl)-2,3- | | | |
| dihydro-1H-pyrrolo[3,4- | | | |
| b]quinolin-1-one | | | |
| 9-amino-5-(2-fluoropyridin-3- | 2.67E−08 | 0.21 | 0.37 |
| yl)-2-propyl-2,3-dihydro-1H- | | | |
| pyrrolo[3,4-b]quinolin-1-one | | | |
| 9-amino-5-(2,6- | 2.81E−08 | 0.068 | 0.31 |
| dimethoxypyridin-3-yl)-2- | | | |
| propyl-2,3-dihydro-1H- | | | |
| pyrrolo[3,4-b]quinolin-1-one | | | |
| 9-amino-2-(2,5- | 3.12E−08 | 0.013 | 0.18 |
| dimethoxybenzyl)-5-(2- | | | |
| methoxypyridin-3-yl)-2,3- | | | |
| dihydro-1H-pyrrolo[3,4- | | | |
| b]quinolin-1-one | | | |
| 9-amino-2-cyclopropyl-5-(2,5- | 3.29E−08 | 0.063 | 0.26 |
| dimethoxyphenyl)-2,3-dihydro- | | | |
| 1H-pyrrolo[3,4-b]quinolin-1-one | | | |
| 9-amino-5-(2,5-difluorophenyl)- | 4.43E−08 | 0.12 | 0.13 |
| 2-(4-methoxybenzyl)-2,3- | | | |
| dihydro-1H-pyrrolo[3,4- | | | |
| b]quinolin-1-one | | | |
| 9-amino-2-butyl-5-(2,6- | 4.50E−08 | | 0.37 |
| dimethoxypyridin-3-yl)-2,3- | | | |
| dihydro-1H-pyrrolo[3,4- | | | |
| b]quinolin-1-one | | | |
| 9-amino-5-(6-chloropyridin-3- | 4.59E−08 | 0.013 | 0.07 |
| yl)-2-(3,4-dimethoxybenzyl)- | | | |
| 2,3-dihydro-1H-pyrrolo[3,4- | | | |
| b]quinolin-1-one | | | |
| 9-amino-2-propyl-5-(pyridin-3- | 4.65E−08 | | 0.27 |
| yl)-2,3-dihydro-1H-pyrrolo[3,4- | | | |
| b]quinolin-1-one | | | |
| 9-amino-5-(2,6- | 5.01E−08 | | 0.18 |
| dimethoxypyridin-3-yl)-2-ethyl- | | | |
| 2,3-dihydro-1H-pyrrolo[3,4- | | | |
| b]quinolin-1-one | | | |
| 9-amino-5-(6-methoxy-4- | 5.06E−08 | 0.17 | 0.23 |
| methylpyridin-3-yl)-2-propyl- | | | |
| 2,3-dihydro-1H-pyrrolo[3,4- | | | |
| b]quinolin-1-one | | | |
| 9-amino-5-(6-methylpyridin-3- | 9.22E−08 | 0.092 | 0.19 |
| yl)-2-propyl-2,3-dihydro-1H- | | | |
| pyrrolo[3,4-b]quinolin-1-one | | | |
| 9-amino-5-(3,5- | 9.43E−08 | 0.16 | 0.27 |
| dimethylphenyl)-2-propyl-2,3- | | | |
| dihydro-1H-pyrrolo[3,4- | | | |
| b]quinolin-1-one | | | |
| 9-amino-2-cyclobutyl-5-(2,5- | 7.96E−10 | 0.075 | |
| dimethoxyphenyl)-6-fluoro-2,3- | | | |
| dihydro-1H-pyrrolo[3,4- | | | |
| b]quinolin-1-one | | | |
| 9-amino-2-cyclobutyl-6-fluoro- | 8.47E−10 | | |
| 5-(2-methoxypyridin-3-yl)-2,3- | | | |
| dihydro-1H-pyrrolo[3,4- | | | |
| b]quinolin-1-one | | | |
| 9-amino-2-cyclobutyl-5-(2- | 9.45E−10 | −0.016 | 0.14 |
| fluorophenyl)-2,3-dihydro-1H- | | | |
| pyrrolo[3,4-b]quinolin-1-one | | | |
| 9-amino-2-cyclobutyl-5-(2,4- | 9.53E−10 | 0.11 | |
| dimethoxypyrimidin-5-yl)-6- | | | |
| fluoro-2,3-dihydro-1H- | | | |
| pyrrolo[3,4-b]quinolin-1-one | | | |
| 2-(9-amino-2-cyclobutyl-1-oxo- | 9.73E−10 | −0.036 | 0.12 |
| 2,3-dihydro-1H-pyrrolo[3,4- | | | |
| b]quinolin-5-yl)benzonitrile | | | |
| 9-amino-2-ethyl-6-fluoro-5-(2- | 1.40E−09 | 0.005 | 0.17 |
| fluoro-5-methoxyphenyl)-2,3- | | | |
| dihydro-1H-pyrrolo[3,4- | | | |
| b]quinolin-1-one | | | |
| 9-amino-2-cyclobutyl-5-(2,6- | 1.43E−09 | −0.023 | 0.13 |
| difluoro-4-methoxyphenyl)-2,3- | | | |
| dihydro-1H-pyrrolo[3,4- | | | |
| b]quinolin-1-one | | | |
| 9-amino-2-ethyl-6-fluoro-5-(2- | 1.49E−09 | −0.04 | 0.05 |
| fluoro-4-methoxyphenyl)-2,3- | | | |
| dihydro-1H-pyrrolo[3,4- | | | |
| b]quinolin-1-one | | | |
| 9-amino-2-(3,4- | 1.50E−09 | 0.23 | 0.37 |
| dimethoxybenzyl)-5-(4- | | | |
| methoxypyridin-3-yl)-2,3- | | | |
| dihydro-1H-pyrrolo[3,4- | | | |
| b]quinolin-1-one | | | |
| 9-amino-2-ethyl-6-fluoro-5-(2- | 1.51E−09 | −0.04 | 0.038 |
| fluorophenyl)-2,3-dihydro-1H- | | | |
| pyrrolo[3,4-b]quinolin-1-one | | | |
| 9-amino-2-ethyl-6-fluoro-5-(4- | 2.01E−09 | −0.0025 | 0.094 |
| methylpyridin-3-yl)-2,3- | | | |
| dihydro-1H-pyrrolo[3,4- | | | |
| b]quinolin-1-one | | | |
| 9-amino-2-(benzo[d][1,3]dioxol- | 2.19E−09 | 0.28 | |
| 5-ylmethyl)-5-(4- | | | |
| methoxypyridin-3-yl)-2,3- | | | |
| dihydro-1H-pyrrolo[3,4- | | | |
| b]quinolin-1-one | | | |
| 9-amino-5-(2-chloro-6- | 2.22E−09 | −0.15 | 0.051 |
| methoxyphenyl)-2-cyclobutyl- | | | |
| 2,3-dihydro-1H-pyrrolo[3,4- | | | |
| b]quinolin-1-one | | | |
| 9-amino-2-(3,4- | 2.30E−09 | 0.12 | |
| dimethoxybenzyl)-5-(2- | | | |
| methoxypyridin-3-yl)-2,3- | | | |
| dihydro-1H-pyrrolo[3,4- | | | |
| b]quinolin-1-one | | | |
| 9-amino-5-(2,6-dimethylpyridin- | 2.63E−09 | 0.45 | 0.41 |
| 3-yl)-2-propyl-2,3-dihydro-1H- | | | |
| pyrrolo[3,4-b]quinolin-1-one | | | |
| 9-amino-2-(3,4- | 2.68E−09 | 0.092 | 0.2 |
| dimethoxybenzyl)-5-(2-fluoro- | | | |
| 6-methoxyphenyl)-2,3-dihydro- | | | |
| 1H-pyrrolo[3,4-b]quinolin-1-one | | | |
| 9-amino-2-cyclopropyl-6- | 2.68E−09 | 0.032 | 0.13 |
| fluoro-5-(2-fluoro-3- | | | |
| methoxyphenyl)-2,3-dihydro- | | | |
| 1H-pyrrolo[3,4-b]quinolin-1-one | | | |
| 9-amino-2-(3,4- | 2.70E−09 | 0.21 | |
| dimethoxybenzyl)-5-(2,6- | | | |
| dimethylpyridin-3-yl)-2,3- | | | |
| dihydro-1H-pyrrolo[3,4- | | | |
| b]quinolin-1-one | | | |
| 9-amino-2-cyclopentyl-5-(2- | 3.01E−09 | −0.01 | |
| fluoro-6-methoxyphenyl)-2,3- | | | |
| dihydro-1H-pyrrolo[3,4- | | | |
| b]quinolin-1-one | | | |
| 9-amino-2-cyclobutyl-5-(2,5- | 3.15E−09 | 0.16 | |
| dichlorophenyl)-2,3-dihydro- | | | |
| 1H-pyrrolo[3,4-b]quinolin-1-one | | | |
| 9-amino-2-(4-methoxybenzyl)- | 3.35E−09 | 0.32 | 0.52 |
| 5-(4-methoxypyridin-3-yl)-2,3- | | | |
| dihydro-1H-pyrrolo[3,4- | | | |
| b]quinolin-1-one | | | |
| 9-amino-2-ethyl-6-fluoro-5-(2- | 3.47E−09 | −0.12 | 0.093 |
| fluoro-6-methoxyphenyl)-2,3- | | | |
| dihydro-1H-pyrrolo[3,4- | | | |
| b]quinolin-1-one | | | |
| 9-amino-2-cyclopropyl-5-(2,6- | 3.50E−09 | −0.058 | 0.13 |
| difluorophenyl)-2,3-dihydro-1H- | | | |
| pyrrolo[3,4-b]quinolin-1-one | | | |
| 9-amino-5-(2-methoxy-5- | 3.59E−09 | 0.58 | 0 |
| methylpyridin-3-yl)-2-propyl- | | | |
| 2,3-dihydro-1H-pyrrolo[3,4- | | | |
| b]quinolin-1-one | | | |
| 9-amino-5-(2,4-difluorophenyl)- | 3.85E−09 | −0.038 | 0.064 |
| 2-ethyl-6-fluoro-2,3-dihydro- | | | |
| 1H-pyrrolo[3,4-b]quinolin-1-one | | | |
| 9-amino-2-cyclobutyl-5-(2- | 4.07E−09 | 0.19 | |
| methoxy-5-methylphenyl)-2,3- | | | |
| dihydro-1H-pyrrolo[3,4- | | | |
| b]quinolin-1-one | | | |
| 9-amino-2-cyclopropyl-5-(2,6- | 4.12E−09 | 0.062 | 0.17 |
| difluoro-3-methoxyphenyl)-6- | | | |
| fluoro-2,3-dihydro-1H | | | |
| pyrrolo[3,4-b]quinolin-1-one | | | |
| 9-amino-2-cyclobutyl-5-(2,4- | 4.23E−09 | 0.12 | |
| dimethoxypyrimidin-5-yl)-2,3- | | | |
| dihydro-1H-pyrrolo[3,4- | | | |
| b]quinolin-1-one | | | |
| 9-amino-2-cyclobutyl-5-(6- | 4.44E−09 | −0.13 | |
| methylpyridin-3-yl)-2,3- | | | |
| dihydro-1H-pyrrolo[3,4- | | | |
| b]quinolin-1-one | | | |
| 9-amino-5-(2,6-difluoro-4- | 4.50E−09 | −0.09 | −0.02 |
| methoxyphenyl)-2-ethyl-6- | | | |
| fluoro-2,3-dihydro-1H- | | | |
| pyrrolo[3,4-b]quinolin-1-one | | | |
| 9-amino-2-butyl-5-(2-fluoro-6- | 4.62E−09 | 0.25 | |
| methoxyphenyl)-2,3-dihydro- | | | |
| 1H-pyrrolo[3,4-b]quinolin-1-one | | | |
| 9-amino-2-(3,4- | 4.70E−09 | 0.26 | |
| dimethoxybenzyl)-5-(6- | | | |
| methoxy-2-methylpyridin-3-yl)- | | | |
| 2,3-dihydro-1H-pyrrolo[3,4- | | | |
| b]quinolin-1-one | | | |
| 9-amino-2-cyclopropyl-5-(2- | 4.76E−09 | 0.28 | |
| methoxy-5-methylphenyl)-2,3- | | | |
| dihydro-1H-pyrrolo[3,4- | | | |
| b]quinolin-1-one | | | |
| 9-amino-5-(2-chlorophenyl)-2- | 4.82E−09 | 0.35 | |
| propyl-2,3-dihydro-1H- | | | |
| pyrrolo[3,4-b]quinolin-1-one | | | |
| 9-amino-5-(2,4- | 4.84E−09 | 0.072 | 0.11 |
| dimethoxyphenyl)-2-ethyl-6- | | | |
| fluoro-2,3-dihydro-1H- | | | |
| pyrrolo[3,4-b]quinolin-1-one | | | |
| 9-amino-5-(2-chloro-6- | 4.85E−09 | −0.15 | 0.013 |
| methoxyphenyl)-2-cyclopropyl- | | | |
| 6-fluoro-2,3-dihydro-1H- | | | |
| pyrrolo[3,4-b]quinolin-1-one | | | |
| 9-amino-2-ethyl-6-fluoro-5-(2- | 5.29E−09 | | |
| fluoro-3-methoxyphenyl)-2,3- | | | |
| dihydro-1H-pyrrolo[3,4- | | | |
| b]quinolin-1-one | | | |
| 9-amino-2-propyl-5-(2- | 5.53E−09 | 0.35 | |
| methylphenyl)-2,3-dihydro-1H- | | | |
| pyrrolo[3,4-b]quinolin-1-one | | | |
| 9-amino-2-(benzo[d][1,3]dioxol- | 5.78E−09 | 0.24 | |
| 5-ylmethyl)-5-(2- | | | |
| methoxypyridin-3-yl)-2,3- | | | |
| dihydro-1H-pyrrolo[3,4- | | | |
| b]quinolin-1-one | | | |
| 9-amino-2-(4-methoxybenzyl)- | 6.09E−09 | 0.22 | |
| 5-(5-methoxypyridin-3-yl)-2,3- | | | |
| dihydro-1H-pyrrolo[3,4- | | | |
| b]quinolin-1-one | | | |
| 9-amino-5-(5-fluoro-2- | 6.30E−09 | 0.29 | |
| methylphenyl)-2-propyl-2,3- | | | |
| dihydro-1H-pyrrolo[3,4- | | | |
| b]quinolin-1-one | | | |
| 9-amino-5-(2-chloro-5- | 6.63E−09 | 0.44 | 0.61 |
| methylpyridin-3-yl)-2-propyl- | | | |
| 2,3-dihydro-1H-pyrrolo[3,4- | | | |
| b]quinolin-1-one | | | |
| 9-amino-5-(2-fluoro-5- | 6.77E−09 | 0.27 | |
| methylphenyl)-2-propyl-2,3- | | | |
| dihydro-1H-pyrrolo[3,4- | | | |
| b]quinolin-1-one | | | |
| 9-amino-5-(2-fluoropyridin-3- | 6.82E−09 | 0.18 | 0.32 |
| yl)-2-(4-methoxybenzyl)-2,3- | | | |
| dihydro-1H-pyrrolo[3,4- | | | |
| b]quinolin-1-one | | | |
| 9-amino-2-cyclopropyl-5-(2,5- | 6.83E−09 | −0.034 | 0.13 |
| difluorophenyl)-2,3-dihydro-1H- | | | |
| pyrrolo[3,4-b]quinolin-1-one | | | |
| 9-amino-2-ethyl-5-(2-fluoro-4- | 7.14E−09 | −0.008 | 0.036 |
| methoxyphenyl)-2,3-dihydro- | | | |
| 1H-pyrrolo[3,4-b]quinolin-1-one | | | |
| 9-amino-2-cyclopropyl-6- | 7.27E−09 | −0.036 | 0.055 |
| fluoro-5-(2-fluoro-4- | | | |
| methoxyphenyl)-2,3-dihydro- | | | |
| 1H-pyrrolo[3,4-b]quinolin-1-one | | | |
| 9-amino-2-cyclopropyl-6- | 7.54E−09 | 0.002 | 0.15 |
| fluoro-5-(2-fluoro-5- | | | |
| methoxyphenyl)-2,3-dihydro- | | | |
| 1H-pyrrolo[3,4-b]quinolin-1-one | | | |
| 9-amino-5-(2-fluoro-5- | 7.55E−09 | 0.33 | |
| methylpyridin-3-yl)-2-propyl- | | | |
| 2,3-dihydro-1H-pyrrolo[3,4- | | | |
| b]quinolin-1-one | | | |
| 9-amino-2-cyclopropyl-6- | 7.65E−09 | 0.23 | |
| fluoro-5-(2-methoxy-5- | | | |
| methylphenyl)-2,3-dihydro-1H- | | | |
| pyrrolo[3,4-b]quinolin-1-one | | | |
| 9-amino-5-(2-chloro-6- | 7.87E−09 | 0.4 | |
| methylpyridin-3-yl)-2- | | | |
| cyclopropyl-2,3-dihydro-1H- | | | |
| pyrrolo[3,4-b]quinolin-1-one | | | |
| 9-amino-5-(2-fluoro-3- | 8.03E−09 | 0.36 | |
| methoxyphenyl)-2-(4- | | | |
| methoxybenzyl)-2,3-dihydro- | | | |
| 1H-pyrrolo[3,4-b]quinolin-1-one | | | |
| 9-amino-5-(2-fluoro-5- | 8.08E−09 | 0.6 | |
| methoxypyridin-4-yl)-2-propyl- | | | |
| 2,3-dihydro-1H-pyrrolo[3,4- | | | |
| b]quinolin-1-one | | | |
| 9-amino-2-cyclopropyl-5-(4- | 8.10E−09 | 0.21 | |
| methoxypyridin-3-yl)-2,3- | | | |
| dihydro-1H-pyrrolo[3,4- | | | |
| b]quinolin-1-one | | | |
| 9-amino-6-fluoro-5-(2-fluoro-6- | 8.11E−09 | 0.02 | 0.16 |
| methoxyphenyl)-2-methyl-2,3- | | | |
| dihydro-1H-pyrrolo[3,4- | | | |
| b]quinolin-1-one | | | |
| 9-amino-2-cyclopropyl-5-(2,6- | 8.14E−09 | −0.02 | 0.088 |
| difluoro-4-methoxyphenyl)-2,3- | | | |
| dihydro-1H-pyrrolo[3,4- | | | |
| b]quinolin-1-one | | | |
| 9-amino-5-(2,4- | 8.89E−09 | 0.22 | |
| dimethoxypyrimidin-5-yl)-6- | | | |
| fluoro-2-propyl-2,3-dihydro-1H- | | | |
| pyrrolo[3,4-b]quinolin-1-one | | | |
| 9-amino-5-(5-chloro-2- | 8.95E−09 | 0.19 | |
| methoxyphenyl)-2-cyclopropyl- | | | |
| 2,3-dihydro-1H-pyrrolo[3,4- | | | |
| b]quinolin-1-one | | | |
| 9-amino-2-(3,4- | 9.18E−09 | 0.3 | |
| dimethoxybenzyl)-5-(2- | | | |
| methoxy-5-methylphenyl)-2,3- | | | |
| dihydro-1H-pyrrolo[3,4- | | | |
| b]quinolin-1-one | | | |
| 9-amino-5-(2-chloro-5- | 9.72E−09 | 0.45 | |
| methoxypyridin-3-yl)-2-propyl- | | | |
| 2,3-dihydro-1H-pyrrolo[3,4- | | | |
| b]quinolin-1-one | | | |
| 9-amino-2-(3-chloro-4- | 9.89E−09 | 0.25 | |
| methoxybenzyl)-5-(2- | | | |
| methoxypyridin-3-yl)-2,3- | | | |
| dihydro-1H-pyrrolo[3,4- | | | |
| b]quinolin-1-one | | | |
| 9-amino-5-(1,3-dimethyl-1H- | 1.03E−08 | 0.45 | 0.64 |
| pyrazol-5-yl)-2-(4- | | | |
| methoxybenzyl)-2,3-dihydro- | | | |
| 1H-pyrrolo[3,4-b]quinolin-1-one | | | |
| 9-amino-2-(3-chloro-4- | 1.03E−08 | 0.088 | 0.15 |
| methoxybenzyl)-5-(2-fluoro-6- | | | |
| methoxyphenyl)-2,3-dihydro- | | | |
| 1H-pyrrolo[3,4-b]quinolin-1-one | | | |
| 9-amino-5-(4-methoxy-2- | 1.04E−08 | 0.25 | |
| (trifluoromethyl)phenyl)-2- | | | |
| propyl-2,3-dihydro-1H- | | | |
| pyrrolo[3,4-b]quinolin-1-one | | | |
| 9-amino-5-(5-bromo-2- | 1.06E−08 | 0.53 | |
| methoxypyridin-3-yl)-2-propyl- | | | |
| 2,3-dihydro-1H-pyrrolo[3,4- | | | |
| b]quinolin-1-one | | | |
| 9-amino-5-(2-methylpyridin-3- | 1.14E−08 | 0.22 | 0.26 |
| yl)-2-propyl-2,3-dihydro-1H- | | | |
| pyrrolo[3,4-b]quinolin-1-one | | | |
| 9-amino-5-(2-fluoro-3- | 1.15E−08 | 0.2 | |
| methoxyphenyl)-2-propyl-2,3- | | | |
| dihydro-1H-pyrrolo[3,4- | | | |
| b]quinolin-1-one | | | |
| 9-amino-5-(2,5-dichlorophenyl)- | 1.18E−08 | 0.31 | 0.32 |
| 2-propyl-2,3-dihydro-1H- | | | |
| pyrrolo[3,4-b]quinolin-1-one | | | |
| 9-amino-5-(2,6-difluoro-4- | 1.22E−08 | 0.16 | |
| methoxyphenyl)-2-propyl-2,3- | | | |
| dihydro-1H-pyrrolo[3,4- | | | |
| b]quinolin-1-one | | | |
| 9-amino-5-(3,4- | 1.23E−08 | 1.1 | |
| dimethoxyphenyl)-2-propyl-2,3- | | | |
| dihydro-1H-pyrrolo[3,4- | | | |
| b]quinolin-1-one | | | |
| 9-amino-5-(2-fluoro-5- | 1.25E−08 | 0.39 | 0.39 |
| methoxyphenyl)-2-propyl-2,3- | | | |
| dihydro-1H-pyrrolo[3,4- | | | |
| b]quinolin-1-one | | | |
| 9-amino-5-(2-fluoro-4- | 1.28E−08 | 0.12 | |
| methoxyphenyl)-2-propyl-2,3- | | | |
| dihydro-1H-pyrrolo[3,4- | | | |
| b]quinolin-1-one | | | |
| 9-amino-2-cyclopentyl-5-(2,5- | 1.30E−08 | 0.2 | |
| dimethoxyphenyl)-2,3-dihydro- | | | |
| 1H-pyrrolo[3,4-b]quinolin-1-one | | | |
| 9-amino-2-ethyl-5-(2-fluoro-5- | 1.33E−08 | 0.02 | 0.12 |
| methoxyphenyl)-2,3-dihydro- | | | |
| 1H-pyrrolo[3,4-b]quinolin-1-one | | | |
| 9-amino-2-(3,4- | 1.35E−08 | 0.19 | 0.42 |
| dimethoxybenzyl)-5-(2,4- | | | |
| dimethoxypyrimidin-5-yl)-2,3- | | | |
| dihydro-1H-pyrrolo[3,4- | | | |
| b]quinolin-1-one | | | |
| 9-amino-5-(2,5- | 1.38E−08 | 0.03 | 0.16 |
| dimethoxyphenyl)-2-ethyl-6- | | | |
| fluoro-2,3-dihydro-1H- | | | |
| pyrrolo[3,4-b]quinolin-1-one | | | |
| 9-amino-5-(2,4- | 1.39E−08 | 0.39 | |
| dimethoxyphenyl)-6-fluoro-2- | | | |
| propyl-2,3-dihydro-1H- | | | |
| pyrrolo[3,4-b]quinolin-1-one | | | |
| 9-amino-5-(3,5- | 1.42E−08 | 0.34 | |
| dimethylphenyl)-2-ethyl-2,3- | | | |
| dihydro-1H-pyrrolo[3,4- | | | |
| b]quinolin-1-one | | | |
| 9-amino-2-cyclopropyl-5-(2- | 1.45E−08 | −0.05 | 0.12 |
| fluoro-4-methoxyphenyl)-2,3- | | | |
| dihydro-1H-pyrrolo[3,4- | | | |
| b]quinolin-1-one | | | |
| 9-amino-5-(2,4-difluorophenyl)- | 1.49E−08 | 0.39 | |
| 2-propyl-2,3-dihydro-1H- | | | |
| pyrrolo[3,4-b]quinolin-1-one | | | |
| 9-amino-5-(2-chloro-6- | 1.49E−08 | −0.1 | 0.052 |
| methoxyphenyl)-2-cyclopropyl- | | | |
| 2,3-dihydro-1H-pyrrolo[3,4- | | | |
| b]quinolin-1-one | | | |
| 9-amino-5-(2-ethoxypyridin-3- | 1.50E−08 | 0.56 | |
| yl)-2-propyl-2,3-dihydro-1H- | | | |
| pyrrolo[3,4-b]quinolin-1-one | | | |
| 9-amino-5-(3,5-difluorophenyl)- | 1.53E−08 | 0.24 | |
| 2-(3,4-dimethoxybenzyl)-2,3- | | | |
| dihydro-1H-pyrrolo[3,4- | | | |
| b]quinolin-1-one | | | |
| 2-(9-amino-2-cyclopropyl-1- | 1.57E−08 | | |
| oxo-2,3-dihydro-1H- | | | |
| pyrrolo[3,4-b]quinolin-5-yl)-3- | | | |
| methoxybenzonitrile | | | |
| 9-amino-5-(2-chloro-5- | 1.63E−08 | 0.38 | |
| fluoropyridin-3-yl)-2-propyl- | | | |
| 2,3-dihydro-1H-pyrrolo[3,4- | | | |
| b]quinolin-1-one | | | |
| 2-(9-amino-2-cyclopropyl-1- | 1.63E−08 | −0.064 | 0.087 |
| oxo-2,3-dihydro-1H- | | | |
| pyrrolo[3,4-b]quinolin-5- | | | |
| yl)benzonitrile | | | |
| 9-amino-5-(6-methoxy-2- | 1.64E−08 | 0.29 | |
| methylpyridin-3-yl)-2-propyl- | | | |
| 2,3-dihydro-1H-pyrrolo[3,4- | | | |
| b]quinolin-1-one | | | |
| 9-amino-2-propyl-5-(2- | 1.66E−08 | 0.48 | |
| (trifluoromethyl)phenyl)-2,3- | | | |
| dihydro-1H-pyrrolo[3,4- | | | |
| b]quinolin-1-one | | | |
| 9-amino-2-cyclopropyl-5-(2,3- | 1.70E−08 | 0.033 | 0.16 |
| difluorophenyl)-2,3-dihydro-1H- | | | |
| pyrrolo[3,4-b]quinolin-1-one | | | |
| 9-amino-2-cyclopropyl-5-(2- | 1.83E−08 | −0.024 | 0.16 |
| fluorophenyl)-2,3-dihydro-1H- | | | |
| pyrrolo[3,4-b]quinolin-1-one | | | |
| 9-amino-2-propyl-5-(quinolin-6- | 1.95E−08 | 0.29 | |
| yl)-2,3-dihydro-1H-pyrrolo[3,4- | | | |
| b]quinolin-1-one | | | |
| 9-amino-5-(2-fluoro-6- | 1.97E−08 | 0.22 | 0.37 |
| methylpyridin-3-yl)-2-propyl- | | | |
| 2,3-dihydro-1H-pyrrolo[3,4- | | | |
| b]quinolin-1-one | | | |
| 9-amino-2-butyl-5-(2- | 1.97E−08 | 0.5 | |
| methoxypyridin-3-yl)-2,3- | | | |
| dihydro-1H-pyrrolo[3,4- | | | |
| b]quinolin-1-one | | | |
| 9-amino-2-cyclopropyl-5-(2,4- | 2.00E−08 | 0.018 | 0.14 |
| difluorophenyl)-2,3-dihydro-1H- | | | |
| pyrrolo[3,4-b]quinolin-1-one | | | |
| 9-amino-5-(6′-chloro-2,3′- | 2.04E−08 | −0.048 | 0.023 |
| bipyridin-5-yl)-2-(3,4- | | | |
| dimethoxybenzyl)-2,3-dihydro- | | | |
| 1H-pyrrolo[3,4-b]quinolin-1-one | | | |
| 9-amino-5-(2-fluoro-6- | 2.09E−08 | −0.008 | |
| methoxyphenyl)-2-((R)-1-(4- | | | |
| methoxyphenyl)ethyl)-2,3- | | | |
| dihydro-1H-pyrrolo[3,4- | | | |
| b]quinolin-1-one | | | |
| 3-(9-amino-1-oxo-2-propyl-2,3- | 2.12E−08 | 0.54 | |
| dihydro-1H-pyrrolo[3,4- | | | |
| b]quinolin-5-yl)benzonitrile | | | |
| 9-amino-2-butyl-5-(2-methoxy- | 2.13E−08 | 0.56 | |
| 5-methylphenyl)-2,3-dihydro- | | | |
| 1H-pyrrolo[3,4-b]quinolin-1-one | | | |
| 9-amino-5-(1,3-dimethyl-1H- | 2.15E−08 | 0.49 | 0.57 |
| pyrazol-5-yl)-2-propyl-2,3- | | | |
| dihydro-1H-pyrrolo[3,4- | | | |
| b]quinolin-1-one | | | |
| 9-amino-5-(5-chloro-2- | 2.15E−08 | 0.49 | |
| methoxypyridin-3-yl)-2-propyl- | | | |
| 2,3-dihydro-1H-pyrrolo[3,4- | | | |
| b]quinolin-1-one | | | |
| 9-amino-5-(2,6-dichloropyridin- | 2.16E−08 | | |
| 3-yl)-2-propyl-2,3-dihydro-1H- | | | |
| pyrrolo[3,4-b]quinolin-1-one | | | |
| 9-amino-5-(6-fluoro-2- | 2.23E−08 | 1.7 | 0.48 |
| methylpyridin-3-yl)-2-propyl- | | | |
| 2,3-dihydro-1H-pyrrolo[3,4- | | | |
| b]quinolin-1-one | | | |
| 9-amino-2-ethyl-5-(3-fluoro-2- | 2.26E−08 | 0.052 | 0.068 |
| methoxyphenyl)-2,3-dihydro- | | | |
| 1H-pyrrolo[3,4-b]quinolin-1-one | | | |
| 9-amino-2-cyclopropyl-5-(2- | 2.27E−08 | 0.48 | |
| (trifluoromethyl)phenyl)-2,3- | | | |
| dihydro-1H-pyrrolo[3,4- | | | |
| b]quinolin-1-one | | | |
| 9-amino-5-(6-chloro-2- | 2.31E−08 | 1.1 | |
| methylpyridin-3-yl)-2-propyl- | | | |
| 2,3-dihydro-1H-pyrrolo[3,4- | | | |
| b]quinolin-1-one | | | |
| 9-amino-2-ethyl-5-(4- | 2.34E−08 | 0.08 | 0.14 |
| methylpyridin-3-yl)-2,3- | | | |
| dihydro-1H-pyrrolo[3,4- | | | |
| b]quinolin-1-one | | | |
| 9-amino-2-cyclopropyl-5-(2,6- | 2.39E−08 | −0.07 | 0.025 |
| dimethylphenyl)-2,3-dihydro- | | | |
| 1H-pyrrolo[3,4-b]quinolin-1-one | | | |
| 9-amino-5-(2-methoxy-6- | 2.40E−08 | 1.1 | |
| methylpyridin-3-yl)-2-propyl- | | | |
| 2,3-dihydro-1H-pyrrolo[3,4- | | | |
| b]quinolin-1-one | | | |
| 9-amino-5-(2-fluoro-5- | 2.40E−08 | 0.55 | |
| (trifluoromethyl)phenyl)-2- | | | |
| propyl-2,3-dihydro-1H- | | | |
| pyrrolo[3,4-b]quinolin-1-one | | | |
| 9-amino-5-(2,3- | 2.45E−08 | 0.3 | |
| dimethoxyphenyl)-2-propyl-2,3- | | | |
| dihydro-1H-pyrrolo[3,4- | | | |
| b]quinolin-1-one | | | |
| 9-amino-2-propyl-5-(pyridin-4- | 2.46E−08 | 0.43 | |
| yl)-2,3-dihydro-1H-pyrrolo[3,4- | | | |
| b]quinolin-1-one | | | |
| 9-amino-2-ethyl-5-(4-fluoro-3- | 2.55E−08 | 0.09 | |
| methoxyphenyl)-2,3-dihydro- | | | |
| 1H-pyrrolo[3,4-b]quinolin-1-one | | | |
| 9-amino-2-ethyl-5-(2-methoxy- | 2.56E−08 | 0.2 | |
| 5-methylphenyl)-2,3-dihydro- | | | |
| 1H-pyrrolo[3,4-b]quinolin-1-one | | | |
| 9-amino-5-(5-fluoro-2- | 2.58E−08 | 0.22 | |
| methylphenyl)-2-(4- | | | |
| methoxybenzyl)-2,3-dihydro- | | | |
| 1H-pyrrolo[3,4-b]quinolin-1-one | | | |
| 9-amino-5-(1H-indol-5-yl)-2- | 2.68E−08 | 0.55 | |
| propyl-2,3-dihydro-1H- | | | |
| pyrrolo[3,4-b]quinolin-1-one | | | |
| 9-amino-5-(2-fluoro-6- | 2.68E−08 | −0.16 | |
| methoxyphenyl)-2-isopropyl- | | | |
| 2,3-dihydro-1H-pyrrolo[3,4- | | | |
| b]quinolin-1-one | | | |
| 9-amino-5-(2,5- | 2.69E−08 | 0.25 | |
| dimethoxyphenyl)-2-(3- | | | |
| methoxybenzyl)-2,3-dihydro- | | | |
| 1H-pyrrolo[3,4-b]quinolin-1-one | | | |
| 9-amino-5-(3,5- | 2.72E−08 | | |
| dimethoxyphenyl)-2-ethyl-2,3- | | | |
| dihydro-1H-pyrrolo[3,4- | | | |
| b]quinolin-1-one | | | |
| 9-amino-2-ethyl-5-(5-fluoro-2- | 2.74E−08 | 0.054 | 0.17 |
| methoxyphenyl)-2,3-dihydro- | | | |
| 1H-pyrrolo[3,4-b]quinolin-1-one | | | |
| 9-amino-2-(3-chlorobenzyl)-5- | 2.78E−08 | | |
| (2-fluoro-6-methoxyphenyl)- | | | |
| 2,3-dihydro-1H-pyrrolo[3,4- | | | |
| b]quinolin-1-one | | | |
| 9-amino-5-(6-chloro-2- | 2.80E−08 | 0.16 | |
| methylpyridin-3-yl)-2- | | | |
| cyclopropyl-2,3-dihydro-1H- | | | |
| pyrrolo[3,4-b]quinolin-1-one | | | |
| 9-amino-2-cyclopropyl-5-(6- | 2.99E−08 | −0.11 | 0.1 |
| methylpyridin-3-yl)-2,3- | | | |
| dihydro-1H-pyrrolo[3,4- | | | |
| b]quinolin-1-one | | | |
| 9-amino-2-(benzo[d][1,3]dioxol- | 3.02E−08 | 0.4 | |
| 5-ylmethyl)-5-(2,4- | | | |
| dimethoxypyrimidin-5-yl)-2,3- | | | |
| dihydro-1H-pyrrolo[3,4- | | | |
| b]quinolin-1-one | | | |
| 9-amino-5-(6-fluoro-5- | 3.03E−08 | 0.31 | 0.32 |
| methylpyridin-3-yl)-2-propyl- | | | |
| 2,3-dihydro-1H-pyrrolo[3,4- | | | |
| b]quinolin-1-one | | | |
| 2-(9-amino-1-oxo-2-propyl-2,3- | 3.07E−08 | 0.15 | 0.21 |
| dihydro-1H-pyrrolo[3,4- | | | |
| b]quinolin-5-yl)benzonitrile | | | |
| 9-amino-2-propyl-5-(thiophen- | 3.08E−08 | 0.19 | |
| 3-yl)-2,3-dihydro-1H- | | | |
| pyrrolo[3,4-b]quinolin-1-one | | | |
| 9-amino-5-(4-fluoro-2- | 3.12E−08 | 0.22 | |
| methoxyphenyl)-2-(4- | | | |
| methoxybenzyl)-2,3-dihydro- | | | |
| 1H-pyrrolo[3,4-b]quinolin-1-one | | | |
| 9-amino-2-cyclopropyl-5-(4- | 3.19E−08 | 0.5 | |
| methoxy-2- | | | |
| (trifluoromethyl)phenyl)-2,3- | | | |
| dihydro-1H-pyrrolo[3,4- | | | |
| b]quinolin-1-one | | | |
| 9-amino-5-(2,4- | 3.21E−08 | 0.25 | |
| dimethoxyphenyl)-2-(4- | | | |
| methoxybenzyl)-2,3-dihydro- | | | |
| 1H-pyrrolo[3,4-b]quinolin-1-one | | | |
| 9-amino-5-(2-fluoro-3- | 3.25E−08 | 0.09 | 0.11 |
| methoxyphenyl)-2-methyl-2,3- | | | |
| dihydro-1H-pyrrolo[3,4- | | | |
| b]quinolin-1-one | | | |
| 9-amino-5-(2-chloro-5- | 3.32E−08 | 0.068 | 0.14 |
| methoxyphenyl)-2-cyclopropyl- | | | |
| 2,3-dihydro-1H-pyrrolo[3,4- | | | |
| b]quinolin-1-one | | | |
| 9-amino-2-(4-methoxybenzyl)- | 3.41E−08 | | |
| 5-(2-(trifluoromethyl)phenyl)- | | | |
| 2,3-dihydro-1H-pyrrolo[3,4- | | | |
| b]quinolin-1-one | | | |
| 9-amino-2-ethyl-5-(3-fluoro-5- | 3.43E−08 | | |
| methoxyphenyl)-2,3-dihydro- | | | |
| 1H-pyrrolo[3,4-b]quinolin-1-one | | | |
| 9-amino-5-(2,5-difluorophenyl)- | 3.48E−08 | 0.21 | 0.33 |
| 2-propyl-2,3-dihydro-1H- | | | |
| pyrrolo[3,4-b]quinolin-1-one | | | |
| 9-amino-2-cyclopropyl-5-(2- | 3.51E−08 | 0.056 | 0.33 |
| methoxypyridin-3-yl)-2,3- | | | |
| dihydro-1H-pyrrolo[3,4- | | | |
| b]quinolin-1-one | | | |
| 9-amino-5-(6-methoxy-5- | 3.65E−08 | 0.32 | |
| methylpyridin-3-yl)-2-propyl- | | | |
| 2,3-dihydro-1H-pyrrolo[3,4- | | | |
| b]quinolin-1-one | | | |
| 9-amino-2-cyclopropyl-5-(2,5- | 3.67E−08 | | |
| dimethoxyphenyl)-6- | | | |
| (methylthio)-2,3-dihydro-1H- | | | |
| pyrrolo[3,4-b]quinolin-1-one | | | |
| 9-amino-2-cyclopropyl-5-(2,5- | 3.68E−08 | | |
| difluoro-4-methoxyphenyl)-2,3- | | | |
| dihydro-1H-pyrrolo[3,4- | | | |
| b]quinolin-1-one | | | |
| 9-amino-5-(3- | 3.74E−08 | 0.27 | |
| (dimethylamino)phenyl)-2- | | | |
| propyl-2,3-dihydro-1H- | | | |
| pyrrolo[3,4-b]quinolin-1-one | | | |
| 9-amino-5-(furan-3-yl)-2- | 3.82E−08 | 0.13 | |
| propyl-2,3-dihydro-1H- | | | |
| pyrrolo[3,4-b]quinolin-1-one | | | |
| 9-amino-5-(4-methoxypyridin- | 3.92E−08 | 0.39 | 0.62 |
| 3-yl)-2-propyl-2,3-dihydro-1H- | | | |
| pyrrolo[3,4-b]quinolin-1-one | | | |
| 9-amino-2-cyclobutyl-5-(3,5- | 3.93E−08 | | |
| difluorophenyl)-2,3-dihydro-1H- | | | |
| pyrrolo[3,4-b]quinolin-1-one | | | |
| 9-amino-5-(5-chloro-2- | 3.96E−08 | 0.36 | 0.54 |
| methoxyphenyl)-2-propyl-2,3- | | | |
| dihydro-1H-pyrrolo[3,4- | | | |
| b]quinolin-1-one | | | |
| 9-amino-5-(2-methoxypyridin- | 4.21E−08 | 0.36 | 0.58 |
| 3-yl)-2-propyl-2,3-dihydro-1H- | | | |
| pyrrolo[3,4-b]quinolin-1-one | | | |
| 9-amino-2-ethyl-5-(3-fluoro-4- | 4.25E−08 | 0.012 | 0.065 |
| methoxyphenyl)-2,3-dihydro- | | | |
| 1H-pyrrolo[3,4-b]quinolin-1-one | | | |
| 9-amino-2-cyclopropyl-5-(2- | 4.34E−08 | 0 | 0.18 |
| fluoro-6-methylpyridin-3-yl)- | | | |
| 2,3-dihydro-1H-pyrrolo[3,4- | | | |
| b]quinolin-1-one | | | |
| 9-amino-5-phenyl-2-propyl-2,3- | 4.38E−08 | 0.51 | |
| dihydro-1H-pyrrolo[3,4- | | | |
| b]quinolin-1-one | | | |
| 9-amino-2-propyl-5-(3- | 4.46E−08 | 0.38 | 0.29 |
| methylphenyl)-2,3-dihydro-1H- | | | |
| pyrrolo[3,4-b]quinolin-1-one | | | |
| 9-amino-2-(2-butyl)-5-(2-fluoro- | 4.62E−08 | | |
| 6-methoxyphenyl)-2,3-dihydro- | | | |
| 1H-pyrrolo[3,4-b]quinolin-1-one | | | |
| 9-amino-5-(5-fluoro-2- | 4.63E−08 | 0.39 | |
| methoxyphenyl)-2-(4- | | | |
| methoxybenzyl)-2,3-dihydro- | | | |
| 1H-pyrrolo[3,4-b]quinolin-1-one | | | |
| 9-amino-5-(3,5- | 4.67E−08 | 0.6 | |
| dimethoxyphenyl)-2-propyl-2,3- | | | |
| dihydro-1H-pyrrolo[3,4- | | | |
| b]quinolin-1-one | | | |
| 9-amino-5-(2-methoxy-5- | 4.72E−08 | 0.17 | 0.37 |
| methylphenyl)-2-propyl-2,3- | | | |
| dihydro-1H-pyrrolo[3,4- | | | |
| b]quinolin-1-one | | | |
| 9-amino-5-(3,4- | 4.77E−08 | 0.61 | |
| dimethoxyphenyl)-2-(4- | | | |
| methoxybenzyl)-2,3-dihydro | | | |
| 1H-pyrrolo[3,4-b]quinolin-1-one | | | |
| 9-amino-5-(2,5-difluoro-4- | 4.84E−08 | 0.29 | |
| methoxyphenyl)-2-(4- | | | |
| methoxybenzyl)-2,3-dihydro- | | | |
| 1H-pyrrolo[3,4-b]quinolin-1-one | | | |
| 9-amino-5-(2,4- | 4.98E−08 | 0.25 | 0.31 |
| dimethoxyphenyl)-2-propyl-2,3- | | | |
| dihydro-1H-pyrrolo[3,4- | | | |
| b]quinolin-1-one | | | |
| 9-amino-2-propyl-5-(2,3,4- | 4.98E−08 | 0.55 | |
| trimethoxyphenyl)-2,3-dihydro- | | | |
| 1H-pyrrolo[3,4-b]quinolin-1-one | | | |
| 9-amino-5-(2-methoxy-5- | 5.11E−08 | | |
| (pyridin-4-yl)phenyl)-2-propyl- | | | |
| 2,3-dihydro-1H-pyrrolo[3,4- | | | |
| b]quinolin-1-one | | | |
| 4-(9-amino-1-oxo-2-propyl-2,3- | 5.21E−08 | | |
| dihydro-1H-pyrrolo[3,4- | | | |
| b]quinolin-5-yl)benzonitrile | | | |
| 9-amino-2-cyclopropyl-5-(2- | 5.41E−08 | | |
| methoxyphenyl)-2,3-dihydro- | | | |
| 1H-pyrrolo[3,4-b]quinolin-1-one | | | |
| 9-amino-5-(2-methoxy-5- | 5.51E−08 | | |
| methylphenyl)-2-(4- | | | |
| methoxybenzyl)-2,3-dihydro- | | | |
| 1H-pyrrolo[3,4-b]quinolin-1-one | | | |
| 9-amino-2-cyclopropyl-5-(6- | 5.53E−08 | 0.088 | 0.08 |
| methoxy-5-methylpyridin-3-yl)- | | | |
| 2,3-dihydro-1H-pyrrolo[3,4- | | | |
| b]quinolin-1-one | | | |
| 9-amino-5-(4-methylpyridin-3- | 5.57E−08 | 0.36 | 0.34 |
| yl)-2-propyl-2,3-dihydro-1H- | | | |
| pyrrolo[3,4-b]quinolin-1-one | | | |
| 9-amino-5-(2-fluoro-5- | 5.58E−08 | | |
| methoxyphenyl)-2-methyl-2,3- | | | |
| dihydro-1H-pyrrolo[3,4- | | | |
| b]quinolin-1-one | | | |
| 9-amino-5-(2,5- | 5.68E−08 | 0.22 | 0.2 |
| dimethylphenyl)-2-propyl-2,3- | | | |
| dihydro-1H-pyrrolo[3,4- | | | |
| b]quinolin-1-one | | | |
| 9-amino-5-(3-fluoro-2- | 5.70E−08 | | |
| methoxyphenyl)-2-propyl-2,3- | | | |
| dihydro-1H-pyrrolo[3,4- | | | |
| b]quinolin-1-one | | | |
| 9-amino-2-cyclopropyl-5-(2,4- | 5.84E−08 | | |
| dimethoxyphenyl)-2,3-dihydro- | | | |
| 1H-pyrrolo[3,4-b]quinolin-1-one | | | |
| 9-amino-5-(2,5- | 6.01E−08 | | |
| dimethoxyphenyl)-2-ethyl-2,3- | | | |
| dihydro-1H-pyrrolo[3,4- | | | |
| b]quinolin-1-one | | | |
| 9-amino-2-(4-methoxybenzyl)- | 6.05E−08 | | |
| 5-(6-methylpyridin-3-yl)-2,3- | | | |
| dihydro-1H-pyrrolo[3,4- | | | |
| b]quinolin-1-one | | | |
| 9-amino-2-(benzo[d][1,3]dioxol- | 6.06E−08 | 0.33 | |
| 5-ylmethyl)-5-(2,5- | | | |
| dimethoxyphenyl)-2,3-dihydro- | | | |
| 1H-pyrrolo[3,4-b]quinolin-1-one | | | |
| 9-amino-5-(4-fluoro-2- | 6.08E−08 | 0.2 | 0.2 |
| methoxyphenyl)-2-propyl-2,3- | | | |
| dihydro-1H-pyrrolo[3,4- | | | |
| b]quinolin-1-one | | | |
| 9-amino-5-(3-methoxyphenyl)- | 6.29E−08 | 0.31 | 0.3 |
| 2-propyl-2,3-dihydro-1H- | | | |
| pyrrolo[3,4-b]quinolin-1-one | | | |
| 9-amino-5-(5-methoxypyridin- | 6.30E−08 | 0.23 | |
| 3-yl)-2-propyl-2,3-dihydro-1H- | | | |
| pyrrolo[3,4-b]quinolin-1-one | | | |
| 2-(9-amino-2-methyl-1-oxo-2,3- | 6.43E−08 | | |
| dihydro-1H-pyrrolo[3,4- | | | |
| b]quinolin-5-yl)benzonitrile | | | |
| 9-amino-2-cyclopropyl-5- | 6.47E−08 | | |
| (pyridin-3-yl)-2,3-dihydro-1H- | | | |
| pyrrolo[3,4-b]quinolin-1-one | | | |
| 5-(9-amino-1-oxo-2-propyl-2,3- | 6.49E−08 | 0.26 | |
| dihydro-1H-pyrrolo[3,4- | | | |
| b]quinolin-5-yl)nicotinonitrile | | | |
| 9-amino-2-methyl-5-(4- | 6.49E−08 | | |
| methylpyridin-3-yl)-2,3- | | | |
| dihydro-1H-pyrrolo[3,4- | | | |
| b]quinolin-1-one | | | |
| 9-amino-5-(3,5- | 6.55E−08 | | |
| dimethylphenyl)-2-methyl-2,3- | | | |
| dihydro-1H-pyrrolo[3,4- | | | |
| b]quinolin-1-one | | | |
| 9-amino-5-(2,5- | 7.02E−08 | | |
| dimethoxyphenyl)-2-(4- | | | |
| methoxybenzyl)-2,3-dihydro- | | | |
| 1H-pyrrolo[3,4-b]quinolin-1-one | | | |
| 9-amino-5-(2-methoxy-5- | 7.02E−08 | | |
| methylphenyl)-2-methyl-2,3- | | | |
| dihydro-1H-pyrrolo[3,4- | | | |
| b]quinolin-1-one | | | |
| 9-amino-2-(4-methoxybenzyl)- | 7.06E−08 | | |
| 5-(1-methyl-1H-pyrazol-4-yl)- | | | |
| 2,3-dihydro-1H-pyrrolo[3,4- | | | |
| b]quinolin-1-one | | | |
| 9-amino-2-cyclopropyl-5-(5- | 7.34E−08 | | |
| fluoro-2-methoxyphenyl)-2,3- | | | |
| dihydro-1H-pyrrolo[3,4- | | | |
| b]quinolin-1-one | | | |
| 9-amino-5-(3,5- | 7.41E−08 | | |
| dimethoxyphenyl)-2-methyl-2,3- | | | |
| dihydro-1H-pyrrolo[3,4- | | | |
| b]quinolin-1-one | | | |
| 5-(9-amino-1-oxo-2-propyl-2,3- | 7.45E−08 | | |
| dihydro-1H-pyrrolo[3,4- | | | |
| b]quinolin-5-yl)-2- | | | |
| fluorobenzonitrile | | | |
| 9-amino-5-(2,6-difluoropyridin- | 7.53E−08 | 0.21 | |
| 3-yl)-2-propyl-2,3-dihydro-1H- | | | |
| pyrrolo[3,4-b]quinolin-1-one | | | |
| 2-(9-amino-1-oxo-2-propyl-2,3- | 7.57E−08 | | |
| dihydro-1H-pyrrolo[3,4- | | | |
| b]quinolin-5-yl)-3- | | | |
| methoxybenzonitrile | | | |
| 9-amino-5-(2- | 7.58E−08 | | |
| methoxypyrimidin-5-yl)-2- | | | |
| propyl-2,3-dihydro-1H- | | | |
| pyrrolo[3,4-b]quinolin-1-one | | | |
| 9-amino-5-(3-chlorophenyl)-2- | 7.65E−08 | | |
| propyl-2,3-dihydro-1H- | | | |
| pyrrolo[3,4-b]quinolin-1-one | | | |
| 9-amino-2-ethyl-5-(6- | 7.76E−08 | | |
| methylpyridin-3-yl)-2,3- | | | |
| dihydro-1H-pyrrolo[3,4- | | | |
| b]quinolin-1-one | | | |
| 9-amino-5-(2-fluoro-4- | 7.78E−08 | | |
| methoxyphenyl)-2-methyl-2,3- | | | |
| dihydro-1H-pyrrolo[3,4- | | | |
| b]quinolin-1-one | | | |
| 9-amino-2-cyclopropyl-5-(2,4- | 7.82E−08 | | |
| dimethoxypyrimidin-5-yl)-2,3- | | | |
| dihydro-1H-pyrrolo[3,4- | | | |
| b]quinolin-1-one | | | |
| 9-amino-5-(4-fluorophenyl)-2- | 7.83E−08 | | |
| propyl-2,3-dihydro-1H- | | | |
| pyrrolo[3,4-b]quinolin-1-one | | | |
| 9-amino-5-(2,4- | 7.90E−08 | | |
| dimethoxyphenyl)-2-methyl-2,3- | | | |
| dihydro-1H-pyrrolo[3,4- | | | |
| b]quinolin-1-one | | | |
| 9-amino-5-(5-fluoro-2- | 8.03E−08 | 0.19 | 0.25 |
| methoxyphenyl)-2-propyl-2,3- | | | |
| dihydro-1H-pyrrolo[3,4- | | | |
| b]quinolin-1-one | | | |
| 9-amino-5-(3-fluoro-5- | 8.04E−08 | | |
| methoxyphenyl)-2-methyl-2,3- | | | |
| dihydro-1H-pyrrolo[3,4- | | | |
| b]quinolin-1-one | | | |
| 9-amino-2-propyl-5-(4- | 8.07E−08 | 0.37 | |
| methylphenyl)-2,3-dihydro-1H- | | | |
| pyrrolo[3,4-b]quinolin-1-one | | | |
| 9-amino-5-(5-fluoro-6- | 8.11E−08 | | |
| methoxypyridin-3-yl)-2-propyl- | | | |
| 2,3-dihydro-1H-pyrrolo[3,4- | | | |
| b]quinolin-1-one | | | |
| 9-amino-5-(2,3- | 8.19E−08 | | |
| dimethoxyphenyl)-2-ethyl-2,3- | | | |
| dihydro-1H-pyrrolo[3,4- | | | |
| b]quinolin-1-one | | | |
| 9-amino-5-(2,6- | 8.25E−08 | | |
| dimethoxyphenyl)-2-ethyl-2,3- | | | |
| dihydro-1H-pyrrolo[3,4- | | | |
| b]quinolin-1-one | | | |
| 9-amino-2-cyclopropyl-5-(4- | 8.31E−08 | | |
| fluoro-2-methoxyphenyl)-2,3- | | | |
| dihydro-1H-pyrrolo[3,4- | | | |
| b]quinolin-1-one | | | |
| 9-amino-5-(6-chloro-5- | 8.57E−08 | | |
| methylpyridin-3-yl)-2-propyl- | | | |
| 2,3-dihydro-1H-pyrrolo[3,4- | | | |
| b]quinolin-1-one | | | |
| 9-amino-5-(3,4- | 8.80E−08 | | |
| dimethoxyphenyl)-2-methyl-2,3- | | | |
| dihydro-1H-pyrrolo[3,4- | | | |
| b]quinolin-1-one | | | |
| 9-amino-2-propyl-5-(3,4,5- | 8.98E−08 | 0.96 | |
| trimethoxyphenyl)-2,3-dihydro- | | | |
| 1H-pyrrolo[3,4-b]quinolin-1-one | | | |
| 9-amino-2-butyl-5-(6- | 9.05E−08 | | |
| methylpyridin-3-yl)-2,3- | | | |
| dihydro-1H-pyrrolo[3,4- | | | |
| b]quinolin-1-one | | | |
| 9-amino-5-(2,3-difluorophenyl)- | 9.82E−08 | | |
| 2-(4-methoxybenzyl)-2,3- | | | |
| dihydro-1H-pyrrolo[3,4- | | | |
| b]quinolin-1-one | | | |
| 9-amino-5-(2,4- | 9.91E−08 | | |
| dimethoxyphenyl)-2-ethyl-2,3- | | | |
| dihydro-1H-pyrrolo[3,4- | | | |
| b]quinolin-1-one | | | |
| 9-amino-5-(1-methyl-1H- | 9.97E−08 | | |
| pyrazol-4-yl)-2-propyl-2,3- | | | |
| dihydro-1H-pyrrolo[3,4- | | | |
| b]quinolin-1-one | | | |
| 9-Amino-5-(2,6- | 2.90E−07 | | 0.19 |
| dimethoxypyridin-3-yl)-2- | | | |
| methyl-2,3-dihydropyrrolo[3,4- | | | |
| b]quinolin-1-one | | | |
| 9-Amino-2-ethyl-6-fluoro-5-(2- | 3.47E−09 | −0.12 | 0.093 |
| fluoro-6-methoxyphenyl)-2,3- | | | |
| dihydropyrrolo[3,4-b]quinolin- | | | |
| 1-one | | | |
| 9-Amino-5-(2,4-dimethoxy- | 4.84E−09 | 0.072 | 0.11 |
| phenyl)-2- | | | |
| ethyl-6-fluoro-2,3- | | | |
| dihydropyrrolo[3,4-b]quinolin- | | | |
| 1-one | | | |
| 9-Amino-5-(2-fluoro-6- | 2.68E−08 | −0.16 | 0.15 |
| methoxyphenyl)-2-isopropyl- | | | |
| 2,3-dihydropyrrolo[3,4- | | | |
| b]quinolin-1-one | | | |
| 9-Amino-6-fluoro-5-(2-fluoro-6- | 8.14E−09 | −0.02 | 0.088 |
| methoxyphenyl)-2-methyl-2,3- | | | |
| dihydropyrrolo[3,4-b]quinolin- | | | |
| 1-one | | | |
| 9-Amino-5-(2-fluoro-3- | 3.25E−08 | 0.09 | 0.11 |
| methoxyphenyl)-2-methyl-2,3- | | | |
| dihydropyrrolo[3,4- | | | |
| b]quinolin-1-one | | | |
| 9-Amino-2-ethyl-5-(2-fluoro-5- | 1.33E−08 | 0.02 | 0.12 |
| methoxyphenyl)-2,3- | | | |
| dihydropyrrolo[3,4b]quinolin-1- | | | |
| one | | | |
| 9-Amino-2-ethyl-5-(5-fluoro-2- | 2.74E−08 | 0.054 | 0.17 |
| methoxyphenyl)-2,3- | | | |
| dihydropyrrolo[3,4-b]quinolin- | | | |
| 1-one | | | |
| 9-Amino-2-ethyl-5-(4-fluoro-3- | 2.55E−08 | 0.09 | 0.18 |
| methoxyphenyl)-2,3- | | | |
| dihydropyrrolo[3,4-b]quinolin- | | | |
| 1-one | | | |
| 9-Amino-2-ethyl-5-(4- | 2.34E−08 | 0.08 | 0.14 |
| methylpyridin-3-yl)-2,3- | | | |
| dihydropyrrolo[3,4-b]quinolin- | | | |
| 1-one | | | |
| 2-(9-Amino-2-cyclobutyl-1-oxo- | 9.73E−10 | −0.036 | 0.12 |
| 2,3-dihydro-1H-pyrrolo[3,4- | | | |
| b]quinolin-5-yl)benzonitrile | | | |
| 9-Amino-2-cyclobutyl-5-(4- | 8.36E−09 | 0.035 | 0.26 |
| fluoro-2- | | | |
| methoxyphenyl)-2,3- | | | |
| dihydropyrrolo | | | |
| [3,4-b]quinolin-1-one | | | |
| 9-Amino-2-cyclobutyl-5-(2- | 4.19E−09 | 0.062 | 0.26 |
| fluoro-3- | | | |
| methoxyphenyl)-2,3- | | | |
| dihydropyrrolo | | | |
| [3,4-b]quinolin-1-one | | | |
| 9-Amino-2-cyclobutyl-5-(2- | 1.34E−09 | 0.1 | 0.29 |
| fluoro-5-methoxyphenyl)-2,3- | | | |
| dihydropyrrolo | | | |
| [3,4-b]quinolin-1-one | | | |
| 9-Amino-2-cyclobutyl-5- | 1.13E−08 | −0.066 | 0.11 |
| pyrazin-2-yl- | | | |
| 2,3-dihydropyrrolo[3,4- | | | |
| b]quinolin- | | | |
| 1-one | | | |
| 9-Amino-2-cyclobutyl-5-(3- | 6.40E−10 | 0.1 | 0.32 |
| methoxypyridin-4-yl)-2,3- | | | |
| dihydropyrrolo[3, | | | |
| 4-b]quinolin-1-one | | | |
| 9-Amino-2-cyclobutyl-5- | 1.35E−09 | 0.079 | 0.17 |
| pyridin-4-yl- | | | |
| 2,3-dihydropyrrolo[3,4- | | | |
| b]quinolin- | | | |
| 1-one | | | |
| 9-Amino-2-cyclobutyl-5- | 4.66E−09 | 0.061 | 0.12 |
| pyridin-2-yl- | | | |
| 2,3-dihydropyrrolo[3,4- | | | |
| b]quinolin- | | | |
| 1-one | | | |
| 9-Amino-2-cyclobutyl-5-(3,6- | 7.40E−10 | 0.088 | 0.42 |
| dimethoxypyridazin-4-yl)-2,3- | | | |
| dihydropyrrolo[3,4-b]quinolin- | | | |
| 1-one | | | |
| 9-Amino-2-cyclobutyl-5-(6- | 1.48E−09 | 0.085 | 0.16 |
| methoxypyridin-2-yl)-2,3- | | | |
| dihydropyrrolo[3,4-b]quinolin- | | | |
| 1-one | | | |
| 9-Amino-2-cyclobutyl-3- | 7.62E−09 | −0.052 | 0.1 |
| hydroxy-5-(6-methylpyridin-2- | | | |
| yl)-2,3-dihydropyrrolo[3,4- | | | |
| b]quinolin-1-one | | | |
| 9-Amino-2-cyclobutyl-5-(5- | 1.27E−08 | 0.088 | 0.13 |
| methylpyridin-2-yl)-2,3- | | | |
| dihydropyrrolo[3,4- | | | |
| b]quinolin-1-one | | | |
| 9-Amino-2-cyclobutyl-5- | 8.30E−09 | 0.013 | 0.12 |
| pyrimidin-2-yl-2,3- | | | |
| dihydropyrrolo[3,4-b]quinolin- | | | |
| 1-one | | | |
| 6-(9-Amino-2-cyclobutyl-1-oxo- | 2.39E−09 | 0.005 | 0.15 |
| 2,3-dihydro-1H-pyrrolo[3,4- | | | |
| b]quinolin-5-yl)-nicotinonitrile | | | |
| 5-(9-Amino-2-cyclobutyl-1-oxo- | 5.85E−09 | 0.048 | 0.21 |
| 2,3-dihydro-1H-pyrrolo[3,4- | | | |
| b]quinolin-5-yl)-nicotinonitrile | | | |
| 9-Amino-2-cyclobutyl-5-(3- | 4.73E−09 | 0.082 | 0.43 |
| methoxypyridazin-4-yl)-2,3- | | | |
| dihydropyrrolo[3,4-b]quinolin- | | | |
| 1-one | | | |
| 9-Amino-2-cyclobutyl-5-(4- | 6.31E−10 | 0.083 | 0.52 |
| methoxy-pyrimidin-5-yl)-2,3- | | | |
| dihydropyrrolo[3,4-b]quinolin- | | | |
| 1-one | | | |
| 9-Amino-2-cyclobutyl-5-(3- | 4.69E−09 | 0.017 | 0.19 |
| fluoropyridin-2-yl)-2,3- | | | |
| dihydropyrrolo[3,4- | | | |
| b]quinolin-1-one | | | |
| 2-(9-Amino-2-cyclobutyl-1-oxo- | 4.19E−09 | −0.024 | 0.11 |
| 2,3-dihydro-1H-pyrrolo[3,4- | | | |
| b]quinolin-5- | | | |
| yl)-5-fluorobenzonitrile | | | |
| 2-(9-Amino-2-cyclobutyl-1-oxo- | 6.20E−09 | −0.02 | 0.21 |
| 2,3-dihydro-1H-pyrrolo[3,4- | | | |
| b]quinolin-5- | | | |
| yl)-4-fluorobenzonitrile | | | |
| 4-(9-Amino-2-cyclobutyl-1-oxo- | 1.53E−08 | 0.066 | 0.36 |
| 2,3-dihydro-1H-pyrrolo[3,4- | | | |
| b]quinolin-5- | | | |
| yl)-6-methoxynicotinonitrile | | | |
| 9-Amino-5-(1,3-dimethyl-1H- | 1.04E−08 | 0.15 | 0.35 |
| pyrazol- | | | |
| 4-yl)-6-fluoro-2-(R)-tetrahydro- | | | |
| furan-3-yl-2,3- | | | |
| dihydropyrrolo[3,4-b]quinolin- | | | |
| 1-one | | | |
| 9-Amino-2-cyclobutyl-5-(5- | 6.70E−09 | 0.065 | 0.33 |
| fluoro-2- | | | |
| methoxypyridin-4-yl)-2,3- | | | |
| dihydropyrrolo | | | |
| [3,4-b]quinolin-1-one | | | |
| 9-Amino-2-cyclobutyl-5-(5- | 8.90E−09 | 0.066 | 0.36 |
| fluoro-2- | | | |
| methoxyphenyl)-2,3- | | | |
| dihydropyrrolo | | | |
| [3,4-b]quinolin-1-one | | | |
| 9-Amino-2-cyclobutyl-5-(2,4- | 2.49E−10 | 0.044 | 0.23 |
| dimethoxy-phenyl)-6-fluoro-2,3- | | | |
| dihydropyrrolo[3,4-b]quinolin- | | | |
| 1-one | | | |
| 9-Amino-2-cyclobutyl-6-fluoro- | 3.05E−10 | 0 | 0.15 |
| 5-(6- | | | |
| methylpyridin-3-yl)-2,3- | | | |
| dihydropyrrolo[3,4-b]quinolin- | | | |
| 1-one | | | |
| 9-Amino-2-cyclobutyl-6-fluoro- | 2.46E−10 | 0.018 | 0.19 |
| 5-(2- | | | |
| fluoropyridin-3-yl)-2,3- | | | |
| dihydropyrrolo[3,4-b]quinolin- | | | |
| 1-one | | | |
| 9-Amino-2-cyclobutyl-6-fluoro- | 1.20E−09 | 0.0067 | 0.15 |
| 5-(4- | | | |
| fluoro-2-methoxyphenyl)-2,3- | | | |
| dihydropyrrolo[3,4-b]quinolin- | | | |
| 1-one | | | |
| 9-Amino-2-cyclobutyl-6-fluoro- | 1.46E−09 | −0.053 | 0.09 |
| 5-pyrimidin-5-yl-2,3- | | | |
| dihydropyrrolo[3,4-b]quinolin- | | | |
| 1-one | | | |
| 9-Amino-2-cyclobutyl-6-fluoro- | 1.85E−10 | 0.02 | 0.27 |
| 5-(3- | | | |
| methoxypyridin-4-yl)-2,3- | | | |
| dihydropyrrolo[3,4-b]quinolin- | | | |
| 1-one | | | |
| 9-Amino-2-cyclobutyl-6-fluoro- | 1.22E−10 | 0.028 | 0.18 |
| 5-(2- | | | |
| fluoro-3-methoxyphenyl)-2,3- | | | |
| dihydropyrrolo[3,4-b]quinolin- | | | |
| 1-one | | | |
| 9-Amino-2-cyclobutyl-6-fluoro- | 2.63E−09 | −0.04 | 0.19 |
| 5-(6- | | | |
| methoxypyridin-3-yl)-2,3- | | | |
| dihydropyrrolo[3,4-b]quinolin- | | | |
| 1-one | | | |
| 9-Amino-2-cyclobutyl-6-fluoro- | 1.68E−09 | 0.11 | 0.11 |
| 5-(2- | | | |
| vinylphenyl)-2,3- | | | |
| dihydropyrrolo[3,4-b]quinolin- | | | |
| 1-one | | | |
| 9-Amino-2-(3-chloro-4- | 1.03E−08 | 0.088 | 0.15 |
| methoxybenzyl)-5-(2-fluoro-6- | | | |
| methoxyphenyl)-2, | | | |
| 3-dihydropyrrolo[3,4- | | | |
| b]quinolin-1- | | | |
| one | | | |
| 2-(9-Amino-2-cyclopropyl-1- | 1.63E−08 | −0.064 | 0.087 |
| oxo-2,3- | | | |
| dihydro-1H-pyrrolo[3,4- | | | |
| b]quinolin-5- | | | |
| yl)-benzonitrile | | | |
| 9-Amino-2-cyclopropyl-5-(6- | 2.99E−08 | −0.11 | 0.1 |
| methyl-pyridin-3-yl)-2,3- | | | |
| dihydropyrrolo[3,4-b]quinolin- | | | |
| 1-one | | | |
| 9-Amino-2-cyclopropyl-5-(2,5- | 6.83E−09 | −0.034 | 0.13 |
| difluoro-phenyl)-2,3- | | | |
| dihydropyrrolo[3,4-b]quinolin- | | | |
| 1-one | | | |
| 9-Amino-2-cyclopropyl-5-(2- | 1.83E−08 | −0.024 | 0.16 |
| fluorophenyl)-2,3- | | | |
| dihydropyrrolo[3,4-b]quinolin- | | | |
| 1-one | | | |
| 9-Amino-2-cyclopropyl-5-(2,6- | 3.50E−09 | −0.058 | 0.13 |
| difluoro-phenyl)-2,3- | | | |
| dihydropyrrolo[3,4-b]quinolin- | | | |
| 1-one | | | |
| 9-Amino-2-cyclopropyl-5-(2- | 1.45E−08 | −0.05 | 0.12 |
| fluoro-4-methoxy-phenyl)-2,3- | | | |
| dihydropyrrolo[3,4-b]quinolin- | | | |
| 1-one | | | |
| 9-Amino-5-(2-chloro-5- | 3.32E−08 | 0.068 | 0.14 |
| methoxyphenyl)-2-cyclopropyl- | | | |
| 2,3-dihydropyrrolo[3,4- | | | |
| b]quinolin-1-one | | | |
| 9-Amino-2-cyclopropyl-5-(2,6- | 8.14E−09 | −0.02 | 0.088 |
| difluoro-4-methoxyphenyl)-2,3- | | | |
| dihydropyrrolo[3,4-b]quinolin- | | | |
| 1-one | | | |
| 9-Amino-2-cyclopropyl-5-(2,3- | 1.70E−08 | 0.033 | 0.16 |
| difluorophenyl)-2,3- | | | |
| dihydropyrrolo[3,4- | | | |
| b]quinolin-1-one | | | |
| 9-Amino-2-cyclopropyl-5-(2,4- | 2.00E−08 | 0.018 | 0.14 |
| difluorophenyl)-2,3- | | | |
| dihydropyrrolo[3,4-b]quinolin- | | | |
| 1-one | | | |
| 9-Amino-2-cyclopropyl-5-(2- | 4.34E−08 | 0 | 0.18 |
| fluoro-6- | | | |
| methylpyridin-3-yl)-2,3- | | | |
| dihydropyrrolo | | | |
| [3,4-b]quinolin-1-one | | | |
| 9-Amino-2-cyclobutyl-5-(6- | 4.44E−09 | −0.13 | 0.21 |
| methylpyridin-3-yl)-2,3- | | | |
| dihydropyrrolo[3,4- | | | |
| b]quinolin-1-one | | | |
| 9-Amino-2-cyclobutyl-5-(2,6- | 1.43E−09 | −0.023 | 0.13 |
| difluoro-4-methoxyphenyl)-2,3- | | | |
| dihydropyrrolo[3,4-b]quinolin- | | | |
| 1-one | | | |
| 9-Amino-2-cyclobutyl-5-(2,4- | 4.23E−09 | 0.13 | 0.44 |
| dimethoxy-pyrimidin-5-yl)-2,3- | | | |
| dihydropyrrolo[3,4-b]quinolin- | | | |
| 1-one | | | |
| 9-Amino-2-cyclobutyl-5-(2- | 9.45E−10 | −0.016 | 0.14 |
| fluorophenyl)-2,3-dihydro- | | | |
| pyrrolo[3,4-b]quinolin-1-one | | | |
| 5-(9-Amino-2-cyclobutyl-1-oxo- | 1.56E−08 | −0.038 | 0.17 |
| 2,3-dihydro-1H-pyrrolo[3,4- | | | |
| b]quinolin-5-yl)-pyridine-2- | | | |
| carbonitrile | | | |
| 9-Amino-2-cyclobutyl-5-(6- | 2.39E−09 | 0.13 | 0.31 |
| fluoro-2- | | | |
| methylpyridin-3-yl)-2,3- | | | |
| dihydropyrrolo[3,4-b]quinolin- | | | |
| 1-one | | | |
| 9-Amino-2-cyclobutyl-5-(2- | 2.56E−09 | −0.002 | 0.24 |
| fluoropyridin-3-yl)-2,3- | | | |
| dihydropyrrolo[3,4- | | | |
| b]quinolin-1-one | | | |
| 9-Amino-2-cyclobutyl-5-(6- | 4.61E−09 | 0.029 | 0.16 |
| methoxy-5- | | | |
| methylpyridin-3-yl)-2,3- | | | |
| dihydropyrrolo[3,4-b]quinolin- | | | |
| 1-one | | | |
| 9-Amino-2-cyclobutyl-5-((P)-2- | 2.89E−10 | −0.11 | 0.23 |
| fluoro-6-methoxyphenyl)-2,3- | | | |
| dihydro-pyrrolo[3,4-b]quinolin- | | | |
| 1-one | | | |
| 9-Amino-2-cyclobutyl-5-((M)-2- | 9.21E−09 | 0.002 | 0.23 |
| fluoro-6-methoxyphenyl)-2,3- | | | |
| dihydro-pyrrolo[3,4-b]quinolin- | | | |
| 1-one | | | |
| 2-(9-Amino-2-cyclobutyl-1-oxo- | 9.05E−10 | −0.016 | 0.18 |
| 2,3-dihydro-1H-pyrrolo[3,4- | | | |
| b]quinolin-5- | | | |
| yl)-6-methoxybenzonitrile | | | |
| 2-(9-Amino-2-cyclobutyl-1-oxo- | 5.46E−09 | −0.043 | 0.21 |
| 2,3-dihydro-1H-pyrrolo[3,4- | | | |
| b]quinolin-5- | | | |
| yl)-3-methoxybenzonitrile | | | |
| 9-Amino-2-cyclobutyl-5-(2,6- | 7.32E−10 | 0.033 | 0.21 |
| difluoropyridin-3-yl)-2,3- | | | |
| dihydropyrrolo | | | |
| [3,4-b]quinolin-1-one | | | |
| 9-Amino-5-(2-fluoro-6- | 3.75E−09 | 0.11 | 0.36 |
| methoxyphenyl)-2-(3- | | | |
| methylcyclobutyl)-2,3- | | | |
| dihydropyrrolo[3,4-b]quinolin- | | | |
| 1-one | | | |
| 9-Amino-2-cyclobutyl-5-(1- | 3.76E−09 | 0.01 | 0.18 |
| methyl-1H- | | | |
| pyrazol-4-yl)-2,3- | | | |
| dihydropyrrolo[3,4-b]quinolin- | | | |
| 1-one | | | |
| 9-Amino-5-(6-methoxy-2- | 4.65E−09 | 0.14 | 0.3 |
| methylpyridin-3-yl)-2-((1s,3s)- | | | |
| 3-methylcyclobutyl)-2,3- | | | |
| dihydro-1H-pyrrolo[3,4- | | | |
| b]quinolin-1-one | | | |
| 9-Amino-5-(2-fluoro-6- | 9.45E−10 | 0.054 | 0.39 |
| methoxyphenyl)-2-((1s,3s)-3- | | | |
| methylcyclobutyl)-2,3-dihydro- | | | |
| 1H-pyrrolo[3,4-b]quinolin-1-one | | | |
| 9-Amino-5-(2-methoxypyridin- | 1.29E−09 | 0.085 | 0.51 |
| 3-yl)-2-((1s,3s)-3- | | | |
| methylcyclobutyl)-2,3-dihydro- | | | |
| 1H-pyrrolo[3,4-b]quinolin-1-one | | | |
| 2-(9-Amino-2-cyclopropyl-1- | 2.28E−08 | −0.078 | 0.15 |
| oxo-2,3- | | | |
| dihydro-1H-pyrrolo[3,4- | | | |
| b]quinolin-5- | | | |
| yl)-3-methoxybenzonitrile | | | |
| 9-Amino-2-cyclopropyl-5-((P)2- | 3.63E−09 | −0.018 | 0.38 |
| fluoro-6- | | | |
| methoxyphenyl)-2,3- | | | |
| dihydropyrrolo[3,4-b]quinolin- | | | |
| 1-one | | | |
| 9-Amino-2-cyclobutyl-5-(2- | 4.26E−09 | −0.017 | 0.3 |
| fluoro-6- | | | |
| methylpyridin-3-yl)-2,3- | | | |
| dihydropyrrolo[3,4-b]quinolin- | | | |
| 1-one | | | |
| 9-Amino-2-cyclobutyl-5-(6- | 5.03E−09 | −0.0033 | 0.2 |
| methoxy-2- | | | |
| methylpyridin-3-yl)-2,3- | | | |
| dihydropyrrolo[3,4-b]quinolin- | | | |
| 1-one | | | |
| 9-Amino-2-cyclobutyl-5-(1,3- | 3.91E−09 | 0.017 | 0.43 |
| dimethyl-1H-pyrazol-4-yl)-2,3- | | | |
| dihydropyrrolo[3,4-b]quinolin- | | | |
| 1-one | | | |
| 9-Amino-2-cyclobutyl-5-(6- | 8.54E−09 | 0.018 | 0.2 |
| fluoro-5- | | | |
| methylpyridin-3-yl)-2,3- | | | |
| dihydropyrrolo[3,4-b]quinolin- | | | |
| 1-one | | | |
| 9-Amino-2-cyclopentyl-5-(2- | 3.01E−09 | −0.01 | 0.3 |
| fluoro-6- | | | |
| methoxyphenyl)-2,3- | | | |
| dihydropyrrolo[3,4-b]quinolin- | | | |
| 1-one | | | |
| 2-(9-Amino-2-cyclopentyl-1- | 5.75E−09 | 0.062 | 0.27 |
| oxo-2,3- | | | |
| dihydro-1H-pyrrolo[3,4- | | | |
| b]quinolin-5- | | | |
| yl)-benzonitrile | | | |
| 9-Amino-2-cyclopentyl-5-(6- | 3.16E−08 | 0.016 | 0.23 |
| methoxy- | | | |
| pyridin-3-yl)-2,3-dihydro- | | | |
| pyrrolo[3, | | | |
| 4-b]quinolin-1-one | | | |
| 9-Amino-2-cyclobutyl-5-(6- | 8.81E−09 | −0.025 | 0.12 |
| morpholin- | | | |
| 4-yl-pyridin-3-yl)-2,3- | | | |
| dihydropyrrolo | | | |
| [3,4-b]quinolin-1-one | | | |
| 9-Amino-2-cyclobutyl-5-(6- | 6.48E−09 | −0.035 | 0.14 |
| methoxypyridin-3-yl)-2,3- | | | |
| dihydropyrrolo[3, | | | |
| 4-b]quinolin-1-one | | | |
| 9-Amino-2-cyclobutyl-5-(4- | 1.10E−09 | 0.017 | 0.13 |
| methylpyridin-3-yl)-2,3- | | | |
| dihydropyrrolo[3,4- | | | |
| b]quinolin-1-one | | | |
| 9-Amino-2-cyclobutyl-5-(3- | 2.17E−09 | −0.03 | 0.14 |
| fluoropyrazin-2-yl)-2,3- | | | |
| dihydropyrrolo[3,4- | | | |
| b]quinolin-1-one | | | |
| 9-Amino-2-cyclobutyl-5-(5- | 1.86E−09 | 0.038 | 0.26 |
| methoxy-pyridin-3-yl)-2,3- | | | |
| dihydropyrrolo[3, | | | |
| 4-b]quinolin-1-one | | | |
| 9-Amino-2-cyclopropyl-6- | 2.68E−09 | 0.032 | 0.13 |
| fluoro-5-(2- | | | |
| fluoro-3-methoxyphenyl)-2,3- | | | |
| dihydropyrrolo[3,4-b]quinolin- | | | |
| 1-one | | | |
| 9-Amino-2-cyclopropyl-5-(2,6- | 4.12E−09 | 0.062 | 0.17 |
| difluoro-3-methoxyphenyl)-6- | | | |
| fluoro-2,3-dihydropyrrolo[3,4- | | | |
| b]quinolin-1-one | | | |
| 9-Amino-2-cyclopropyl-6- | 7.54E−09 | 0.002 | 0.15 |
| fluoro-5-(2- | | | |
| fluoro-5-methoxyphenyl)-2,3- | | | |
| dihydropyrrolo[3,4-b]quinolin- | | | |
| 1-one | | | |
| 9-Amino-2-ethyl-6-fluoro-5-(4- | 2.01E−09 | −0.0025 | 0.094 |
| methylpyridin-3-yl)-2,3- | | | |
| dihydropyrrolo | | | |
| [3,4-b]quinolin-1-one | | | |
| 9-Amino-2-ethyl-6-fluoro-5-(2- | 1.40E−09 | 0.005 | 0.17 |
| fluoro-5-methoxyphenyl)-2,3- | | | |
| dihydropyrrolo[3,4-b]quinolin- | | | |
| 1-one | | | |
| 9-Amino-2-cyclobutyl-5-(2,4- | 9.53E−10 | 0.11 | 0.4 |
| dimethoxypyrimidin-5-yl)-6- | | | |
| fluoro-2,3-dihydropyrrolo[3,4- | | | |
| b]quinolin-1-one | | | |
| 9-Amino-2-cyclobutyl-5-(2,5- | 7.96E−10 | 0.075 | 0.28 |
| dimethoxyphenyl)-6-fluoro-2,3- | | | |
| dihydropyrrolo[3,4-b]quinolin- | | | |
| 1-one | | | |
| 9-Amino-2-cyclobutyl-6-fluoro- | 8.47E−10 | −0.01 | 0.28 |
| 5-(2- | | | |
| methoxypyridin-3-yl)-2,3- | | | |
| dihydropyrrolo | | | |
| [3,4-b]quinolin-1-one | | | |
| 9-Amino-2-cyclopropyl-6- | 3.72E−09 | 0.02 | 0.2 |
| fluoro-5-(2- | | | |
| methoxyphenyl)-2,3- | | | |
| dihydropyrrolo[3,4-b]quinolin- | | | |
| 1-one | | | |
| 9-Amino-2-cyclobutyl-6-fluoro- | 1.56E−09 | −0.047 | 0.2 |
| 5-(2- | | | |
| methoxyphenyl)-2,3- | | | |
| dihydropyrrolo | | | |
| [3,4-b]quinolin-1-one | | | |
| 9-Amino-5-(5-chloro-2- | 2.22E−09 | 0.033 | 0.27 |
| methoxyphenyl)-2-ethyl-6- | | | |
| fluoro-2,3-dihydropyrrolo[3,4- | | | |
| b]quinolin-1-one | | | |
| 9-Amino-2-cyclobutyl-6-fluoro- | 3.33E−10 | 0.025 | 0.26 |
| 5-(2- | | | |
| fluoro-5-methoxyphenyl)-2,3- | | | |
| dihydropyrrolo[3,4-b]quinolin- | | | |
| 1-one | | | |
| 9-Amino-2-cyclobutyl-6-fluoro- | 3.33E−10 | 0.11 | 0.26 |
| 5-(5- | | | |
| fluoro-2-methoxyphenyl)-2,3- | | | |
| dihydropyrrolo[3,4-b]quinolin- | | | |
| 1-one | | | |
| 9-Amino-2-cyclobutyl-6-fluoro- | 3.04E−09 | 0.065 | 0.17 |
| 5-(6- | | | |
| methoxy-4-methylpyridin-3-yl)- | | | |
| 2,3- | | | |
| dihydropyrrolo[3,4-b]quinolin- | | | |
| 1-one | | | |
| 9-Amino-2-cyclobutyl-6-fluoro- | 1.14E−09 | 0.042 | 0.19 |
| 5-(6- | | | |
| methoxy-2-methylpyridin-3-yl)- | | | |
| 2,3- | | | |
| dihydropyrrolo[3,4-b]quinolin- | | | |
| 1-one | | | |
| 9-Amino-2-cyclobutyl-5-(2,5- | 3.35E−09 | 0.087 | 0.47 |
| dimethoxypyridin-3-yl)-2,3- | | | |
| dihydropyrrolo[3,4-b]quinolin- | | | |
| 1-one | | | |
| 9-Amino-6-fluoro-5-(2- | 1.88E−08 | 0.097 | 0.32 |
| methoxypyridin-3-yl)-2-(R)- | | | |
| tetrahydrofuran-3-yl-2,3- | | | |
| dihydropyrrolo[3,4-b]quinolin- | | | |
| 1-one | | | |
| 9-Amino-6-fluoro-5-(2- | 5.68E−09 | −0.14 | 0.19 |
| methoxypyridin-3-yl)-2-(S)- | | | |
| tetrahydrofuran-3-yl-2,3- | | | |
| dihydro-pyrrolo[3,4-b]quinolin- | | | |
| 1-one | | | |
| 9-Amino-2-cyclobutyl-5-(3,4- | 1.00E−09 | 0.083 | 0.41 |
| dimethoxyphenyl)-6-fluoro-2,3- | | | |
| dihydropyrrolo[3,4-b]quinolin- | | | |
| 1-one | | | |

### Method II

### MT1 GTPγ³⁵S-SPA Assay

### Test Validation Standards

2-Iodomelatonin and 6-Chloromelatonin with known activities were used as validation standards during the assay development. The EC50 of 2-Iodomelatonin and 6-Chloromelatonin were ˜3E-11 M and ˜1.5E-10 M respectively in GTPγS assay of hMT1 recombinant cell membranes.

Cells and/or Microorganisms

HEK293F (human embryonic kidney 293 floating cell line) was suspension cultured in Free Style 293 Expression Medium, and expanded in house and stored in liquid nitrogen in cell freezing medium.

### Buffers, Solutions, Cell Media

| | | Make 2 |
|---|---|---|
| Lazareno GTPγS Assay Buffer: | | Liters Buffer |
| 20 mM HEPES | Sigma H-4034, FW 238.3 | 9.532 g |
| 100 mM NaCl | Sigma S-9625, FW 58.44 | 11.688 g |
| 10 mM MgCl2•6H2O | Sigma M-2670, FW 203.3 | 4.066 g |
| pH 7.4 | | Adjust with NaOH |

| | | Make 2 |
|---|---|---|
| Membrane Prepration Buffer: | | Liters Buffer |
| 20 mM HEPES | Sigma H-4034, FW 238.3 | 9.532 g |
| 3 mM MgCl2•6H2O | Sigma M-2670, FW 203.3 | 1.220 g |
| 1 mM EGTA | Sigma E-3889, FW 380.4 | 0.761 g |
| pH 7.4 | | Adjust with NaOH |

### Test Compound Preparation

Test compounds were synthesized in house. Solid compounds were solubilized at 10 mM in DMSO; then 1:3 further diluted in DMSO in 96-well U-bottom plates using PlateMate on the assay day. 2 μl of diluted compounds were transferred to Opti-assay-plates.

### Reference Compounds Preparation

Reference compound, 2-Iodomelatonin, was prepared the same way as test compound.

### Compounds Used to Normalize Experimental Results

2-Iodomelatonin for normalization was diluted in DMSO at concentration 50×3 nM (its EC100 concentration=3 nM). 2 μl of 150 nM 2-Iodomelatonin was then transferred to Opti-assay-plates.

### Cell Lines and Microorganisms

HEK293F (human embryonic kidney 293 floating cell line) cells transiently expressed human Melatonin receptor 1 (MT1) were harvested 48 hours post-transfection. The cell pellets were homogenized using Polytron; and the cell membranes were prepared for GTPγS assay.

### Preparation of Protein/Membranes Containing Target

The cell pellets were homogenized with Polytron in ice-cold buffer: 20 mM HEPES, 3 mM MgCl2, 1 mM EGTA, pH 7.4. (Freshly add protease inhibitor cocktail tables from Roche). The samples were centrifuged at 18,500 rpm for 30 mins at 4° C. in Sorvall SS-34 rotor. The membrane pellets were collected and washed with the ice-cold buffer. The samples were centrifuged at 18,500 rpm for 30 mins at 4° C. again. The membranes were resuspended in the ice-cold buffer with protease inhibitors. The protein concentration of the membrane was determined. The membranes were aliquoted and stored at −80° C.

### Test Method

### Plate Format (if Plates are Used, as Shown in the Following Table)

Numbers denote “Compound #, Dilution #, Replicate #”
Plate direction moves from highest concentration to lowest concentration
Number of compounds per plate: 8
Number of replicates per compound: 1
Number of dilutions per compound: 11
Test Plate: DR96_{—}02_C12[LR.1]

| | 1 | 2 | 3 | 4 | 5 | 6 | 7 | 8 | 9 | 10 | 11 | 12 |
|---|---|---|---|---|---|---|---|---|---|---|---|---|
| A | 1, 1, 1 | 1, 2, 1 | 1, 3, 1 | 1, 4, 1 | 1, 5, 1 | 1, 6, 1 | 1, 7, 1 | 1, 8, 1 | 1, 9, 1 | 1, 10, 1 | 1, 11, 1 | MAX |
| B | 2, 1, 1 | 2, 2, 1 | 2, 3, 1 | 2, 4, 1 | 2, 5, 1 | 2, 6, 1 | 2, 7, 1 | 2, 8, 1 | 2, 9, 1 | 2, 10, 1 | 2, 11, 1 | MAX |
| C | 3, 1, 1 | 3, 2, 1 | 3, 3, 1 | 3, 4, 1 | 3, 5, 1 | 3, 6, 1 | 3, 7, 1 | 3, 8, 1 | 3, 9, 1 | 3, 10, 1 | 3, 11, 1 | MAX |
| D | 4, 1, 1 | 4, 2, 1 | 4, 3, 1 | 4, 4, 1 | 4, 5, 1 | 4, 6, 1 | 4, 7, 1 | 4, 8, 1 | 4, 9, 1 | 4, 10, 1 | 4, 11, 1 | MAX |
| E | 5, 1, 1 | 5, 2, 1 | 5, 3, 1 | 5, 4, 1 | 5, 5, 1 | 5, 6, 1 | 5, 7, 1 | 5, 8, 1 | 5, 9, 1 | 5, 10, 1 | 5, 11, 1 | MIN |
| F | 6, 1, 1 | 6, 2, 1 | 6, 3, 1 | 6, 4, 1 | 6, 5, 1 | 6, 6, 1 | 6, 7, 1 | 6, 8, 1 | 6, 9, 1 | 6, 10, 1 | 6, 11, 1 | MIN |
| G | 7, 1, 1 | 7, 2, 1 | 7, 3, 1 | 7, 4, 1 | 7, 5, 1 | 7, 6, 1 | 7, 7, 1 | 7, 8, 1 | 7, 9, 1 | 7, 10, 1 | 7, 11, 1 | MIN |
| H | 8, 1, 1 | 8, 2, 1 | 8, 3, 1 | 8, 4, 1 | 8, 5, 1 | 8, 6, 1 | 8, 7, 1 | 8, 8, 1 | 8, 9, 1 | 8, 10, 1 | 8, 11, 1 | MIN |

MAX response (100% effect) was determined as the effect of 3 nM of 2-Iodomelatonin.
MIN response (0% effect) was determined as the effect of vehicle control.

### Description of Experimental Procedure

Human MT1/HEK293F membrane (10 μg/well) was mixed with WGA-SPA beads (300 μg/well) and GDP (10 μM) in certain volume of Lazareno assay buffer (20 mM HEPES, 100 mM NaCl, 10 mM MgCl2, pH 7.4). The membrane combo was kept on ice for 30-60 mins. Test compounds were 1:3 diluted in DMSO from 10 mM stock, and transferred 2 μl of diluted compounds to Opti assay plates-96 using PlateMate. GTPγ³⁵S was added to the membrane mixture prior to dispensing 100 μl the membrane combo to the assay plates-96. The final concentration of GTPγ³⁵S was 200 pM. The assay plates were shaking on a plate shaker for 1.5 hours at room temperature. The assay plates were spun at 2000 rpm for 5 mins in bench top centrifuge. The assay plates were measured in TopCount to capture the data within 4 hours.

### Summary of the Different Experimental Conditions (the Role of Various Results Types)

### Final Concentrations of the Constituents

| | |
|---|---|
| 10 μg/well | hMT1/HEK293F membranes |
| 300 μg/well | WGA-SPA beads |
| 10 μM | GDP |
| 200 pM | GTPγ³⁵S |
| 10 μM | Start concentration of test compound |
| 2% | DMSO |
| 20 mM | HEPES |
| 100 mM | NaCl |
| 10 mM | MgCl2 |
| pH7.4 | |

### Treatments Used in Different Experimental Conditions

The test compounds would be heated to 65° C. if they were not soluble at 10 mM in DMSO. The start concentration in general was 10 μM, but could be adjusted based on its potency. Every single batch of membranes had to be validated for its optimal assay conditions, such as, define the optimal GDP concentration, SPA beads amount and EC100 concentration of normalization compound.

### Calculation of Results

Compounds were evaluated for their agonist potency (EC50) and efficacy (Emax). Concentration-response curves were analyzed to determine the EC50 by ActivityBase using equation model #205. Compound's % activity was calculated according to the 100% and 0% activities defined on the same plate as the sample data. Wells A12-C12 were used to define 100% activity, and D12-G12 for 0% activity. More details could be found from the Plate Format above.

### Results (Dependent Variables, Dependent Measurements) and Their Calculation Method

The raw values for the replicates in the Minimum Control experimental condition were averaged. The raw values for the replicates in the Maximum Control experimental condition were averaged. The average Minimum Control was subtracted from the average Maximum Control resulting in the Data Window. The average for the Minimum Control was subtracted from each raw value in the Compound Data experimental condition resulting in the Specific Response for each data value in the Compound Data condition. Each Specific Response in the Compound Data condition was divided by the Data Window then multiplied by 100 resulting in the Percent Response. The EC50 and SlopeFactor were determined by fitting the Percent Inhibition and the concentrations of test compound to model 205 in XLfit—y=A+((B−A)/(1+((C/x)̂D))—with parameter A constrained to 0 and parameter B constrained to 100.

### Method JJ: EEG PROTOCOL

Subjects: Sprague Dawley rats weighing 300-400 g at time of surgery are used as subjects. Rats are maintained>1 week in AstraZeneca vivarium prior to surgical procedures. Rats are housed singly, maintained on a 12:12 light:dark cycle, and fed and watered ad lib for a period of>14 days following surgery. After recovery, rats are administered a restricted food diet for the duration of study as detailed below.

Surgical Procedure: Rats are initially prepared for surgery by inducing anesthesia with a 4-5% isoflurane/O₂ mixture in a small plexiglass chamber. Hair over surgical site is shaved, and the site is cleansed with alternating administrations of Betadine and alcohol. Rats are mounted in a stereotaxic frame (Koph Instruments, Tujnuga, Calif.), and an anesthesia cone (Koph Instruments) is attached to the incisor clamp and placed around rat's snout to deliver a continuous isoflurane/O₂ mixture. An ophthalmic lubricant is applied to the eyes, and eye patches are cut from sterile, opaque tissue paper and placed over the eyes to protect them from the light illuminating the surgical field. Throughout surgery, isoflurane levels are adjusted (2-4%) to maintain a respiration rate of 40-55 breaths/min., and animal's core body temperature is maintained at ˜37° C. by a thermostatically controlled homeothermic blanket.

Surgical fields are prepared using aseptic techniques, a mid-sagittal incision is made and the scalp is retracted to expose the skull over an area including both bregma and lambda landmarks and extending ˜5 mm bilaterally from midline. Holes are trephined in the skull to allow the placement of 5-6 stainless skull screws. The screws anchor the implant chronically to the animal's skull and certain screws serve as surface electrodes for EEG acquisition. Relative to bregma, electrode screw coordinates are: 1) frontal recording screw: A-P: +2.5 mm, L (left): 1.0 mm; 2) temporal recording screw: A-P: −4.5 mm, L (left): 5.5 mm; 3) occipital reference screw: A-P: −10 mm; L: 0 mm; 4) parietal reference screw: A-P: −2 mm, L (right): 2.5 mm. Individual stainless steel wire leads (50 um diameter) are stripped of Teflon insulation in the region of skull contact and are wrapped firmly around each recording or reference skull electrode. The far ends of the wires are pre-soldered to designated pins on either a 40 or 25 pin male nano-strip connector (2 rows of pines with center-to-center separation of 0.025″; Omnetics Corp., Minneapolis, Minn.). The wires and the Omnetics connector are compacted over the surgical field and potted along with the skull screws in acrylic dental cement. At the conclusion of the surgery, the wound site is treated with topical anti-infective (Neosporin), the rat is rehydrated with a10 ml sterile 0.9% NaCl solution containing Buprenex HCl (0.05 mg/kg, subcut) for analgesia, and 0.2 ml (im.) of bicillin as a prophylactic antibiotic before being returned to its home cage.

Post-surgical Training: Following an ˜14 day recovery period in which rats are given free access to both food and water, rats are placed on a restricted diet consisting of sufficient feed pellets (˜3 pellets/day) to maintain weight reached on their first post-recovery day. After 3-5 days of restricted feeding, rats are shaped and trained on a single tone auditory detection task. Behavioral training is conducted in a plexiglas operant conditioning chamber (11″ L×8.25″ W×13″ H, metal grid floor; Med Associates, St. Albans Vt.) enclosed within a larger opaque acoustical chamber. A nosepoke response receptacle containing an infrared photocell beam and detector is mounted 1.12″ above the floor grid on one side wall of the plexiglas chamber and recessed pellet receptacle is located on the opposite wall. Small feed pellets (45 mg) are dispensed from a magazine into this receptacle for consumption by the rat. A speaker and house light are mounted on walls near the top of the operant chamber, and a small fan is used to ventilate both chambers. A videocamera within the acoustical chamber permitted visual monitoring of the activity of the rat during both behavioral training and subsequent recording sessions.

Operant conditioning protocols are controlled and monitored automatically by a computer-generated protocol delivered through a MED-SYST-8 interface (Med Associates). For the auditory detection task, a 1 kHz tone (500 ms duration) is delivered randomly (interstimulus interval 28-38s) through the chamber speaker via a programmable audio generator (Med Associates). Responses (nosepoke breaking photocell in response receptacle) are rewarded by immediate dispensation of a food pellet only if they occurred within 5 s of tone onset. Animals reached a stable criterion level of performance (>90% correct; <3 total responses/rewarded response within ˜10 daily 1 hour training sessions once the initial instrumental association between tone and response is established. All animals are required to be performing at criteria prior to the initiation of recordings.

Recording Protocol: For each recording session, animals are connected to either a unity gain HS-27 micro headstage pre-amplifier (Neuralynx Corp., Tuscon, Ariz.) or a 20× gin HST/16V-G20 headstage (Plexon Corp., Dallas, Tex.) aligned so that the leads from the implant connector are routed to appropriate channels (recording and reference leads to either OP-AMP of FET buffered amplifiers, animal ground to unbuffered connector) and attached to a multi-wire tether. The opposite end of the tether is connected to a freely rotating commutator attached to the top of the behavioral chamber. Leads from the commutator are routed to a second stage of programmable amplifiers/filters, and the A/D interface of a Neuralynx 24 channel Cheetah data acquisition system (Neuralynx). Continuous EEG data are filtered at 1 Hz low-pass, 325 Hz high-pass, digitized at 32 kHz and stored on a desktop computer. Simultaneously, the Cheetah system captures and stores digital TTL pulses corresponding to relevant event flags (i.e. tone, nosepoke) generated by the operant conditioning chamber for the subsequent alignment of neural activity and behavior. After each recording session, data are uploaded to a server for analysis.

During compound testing sessions, animals are initially allowed to re-acclimate to the operant chamber for 10-20′ minutes prior to a 30-minute baseline session in which performance and EEG are continuously monitored. Following this baseline session, animals are briefly disconnected at the commutator, removed from the chamber, dosed with the test dose of the appropriate compound (or equivalent volume of vehicle), and reintroduced to the chamber. The entire dosing procedure is completed within 2 min with little disruption to the animal. Following reintroduction to the chamber, electrophysiological recordings are reestablished and maintained for another 30 minutes. In a typical experiment, animals receive 3-4 ascending doses of either compound or vehicle resulting in a total recording time of 2-2.5 hr. Following a recording session, animals are disconnected and returned to their home cages. Animals are subjected to a washout period>one week before subsequent recordings, but trained in the operant paradigm for at least one hour every two days during this interval to sustain criterion performance. Drug and vehicle treatments are randomized in all animals and each animal typically contributes 1-2 replicates to the total data set for a given treatment and/or vehicle condition, although technical difficulties occasionally necessitate the removal of a recording session from analysis.

### Data Analysis

Behavioral Analysis: Behavioral performance data acquired by Med Associates software (% correct response, ratio of correct/non-rewarded responses), and Neuralynx (response latency) are compiled for each treatment condition within an experimental session and normalized to pre-dosing (baseline) values on a session-by-session basis. Main effects are determined both by a 1-way ANOVA and individual paired comparisons using a significance level p<0.05. Behavioral data are analyzed and displayed using Origin Ver. 7.5 graphical software (Micorcal Corp., Northhampton, Mass.).

Spontaneous EEG: Continuous EEG data acquired by Neuralynx are imported to the NeuroExplorer Ver. 3.183 software suite (Plexon Corp.) for analysis. Consecutive 10-s epochs of EEG data from each channel are subjected to a fast Fourier transform (FFT) from which EEG power density is then computed from 1-50 Hz with a resolution of 0.068 Hz. Successive power density spectra are plotted in a spectrogram format as a time-frequency series over each 30-minute treatment (control and drug/compound treatment) within a given experiment.

For analysis of drug/compound effects, power spectrum density data are evaluated for the 20 minute block just prior to vehicle or compound dosing (i.e., −20-0 min), and also for the 20 minute period encompassing +10-+30 min post-dosing interval. The 10-minute delay following dosing is taken as sufficient to allow adequate exposure for pharmacodynamic effect, based on pharmacokinetic measures made in other AZ studies. For each 20-minute block, power density data from consecutive FFTs are parsed into component EEG bands in accordance with the International Pharmacological EG Group (IPEG) Guidelines as follows: delta (1-5 Hz), theta (6-8 Hz), alpha (9-12 Hz), beta (13-30 Hz), and gamma (31-50 Hz). For comparison within and across treatments and dose levels, the averaged EEG power density in each band within each 20-minute post-dosing block is represented as a fraction of the pre-dosing control block.

Task-associated EEG: Task associated changes in EEG are analyzed in both time and frequency domains to look at drug effects on both tone-event related potentials (ERPS) and induced/evoked oscillations respectively. For both types of analysis raw voltage data is exported from NeuroExplorer software environment into MATLAB v.R2006a (MathWorks, Natick, Mass.), and data from each condition is initially separated into trials be parceling out voltages ±10 seconds around each tone presentation within an epoch. For ERP analysis, raw voltage traces are averaged across all trials excluding both the first and last tone presentation within each condition. Average waveforms are smoothed to remove high frequency voltage fluctuations, and the amplitudes and latencies of peaks and troughs at various time points (typically: 0-20 ms; 25-55 ms; 55-150 ms; 150-500 ms) following tone presentation are extracted and compared across drug and baseline conditions. Comparisons are done using both average peak values as well as average area under the curve within the different time windows. To the extent that various components of tone-evoked ERPs are shown to be disrupted in both schizophrenic patients and in various animal models, these measurements are likely to serve as sensitive markers of early information processing within cortex and can be used to evaluate the potential therapeutic value of various GABAergic compounds.

In addition to evaluating task-associated EEG in the time domain, we have developed protocols for evaluating drug-dependent changes in both induced and evoked oscillations across a range of frequencies. For induced oscillations, tone-locked voltage fluctuations are converted into time-frequency spectrograms using both custom and commercially available MATLAB software including the Chronux toolbox (Partha Mitra, Cold Spring Harbor Laboratory). Spectrograms are created for frequencies from 15-80 (or 160) Hz using the mtspecgramc command (Chronux) with a window size 125 ms and a step size of 10 ms. Trial-specific spectrograms are averaged together, specific frequency ranges of interest are determined (i.e. 25-55 Hz), average power within distinct frequency ranges is determined as a function of time and converted into a z-scores using the mean and standard deviation of power fluctuations during the pre-tone period. Area under the associated curves at different time points relative to tone presentation are used to profile the effects of drugs relative to the pre-injection baseline. For evoked oscillations, similar analytical techniques are used except for the fact that raw voltage fluctuations around tone presentation are averaged prior to the creation of a single spectrogram. Statistical analysis is done using paired-comparisons, non-parametric tests and multivariate ANOVAs depending on the comparison being made. For all statistical comparisons, a p-value of p<0.05 is used as evidence for a statistically significant difference between populations.

1. A compound of Formula I: 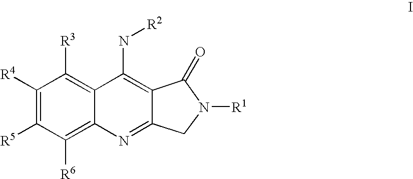
or a pharmaceutically acceptable salt, tautomer, atropisomer, or in vivo-hydrolysable precursor thereof, wherein:
R¹ is C₁₋₆ alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl, wherein each of the C₁₋₆ alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl is optionally substituted by 1, 2, 3, 4 or 5 R⁷;
R² is H, —C(═O)R^{b}, C(═O)NR^{c}R^{d}, —C(═O)OR^{a}, —S(═O)₂R^{b}, C₁₋₆ alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl, wherein each of the C₁₋₆ alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl is optionally substituted by 1, 2, 3, 4 or 5 R⁸;
R³, R⁴ and R⁵ are each, independently, H, halo, —Si(C₁₋₁₀ alkyl)₃, —CN, —NO₂, —OR^{a}, —SR^{a}, —OC(═O)R^{a}, —OC(═O)OR^{b}, —OC(═O)NR^{c}R^{d}, —C(═O)R^{a}, —C(═O)OR^{b}, —C(═O)NR^{c}R^{d}, —NR^{c}R^{d}, —NR^{c}C(═O)R^{a}, —NR^{c}C(═O)OR^{b}, —NR^{c}S(═O)₂R^{b},—S(═O)R^{a}, —S(═O)NR^{c}R^{d}, —S(═O)₂R^{a}, —S(═O)₂NR^{c}R^{d}, C₁₋₆ alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl, wherein each of the C₁₋₆ alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl is optionally substituted by 1, 2 or 3 R⁹;
R⁵ is C₆₋₁₀aryl, C₆₋₁₀aryloxy, C₂₋₅heteroaryloxy, or C₂₋₅heteroaryl, each optionally substituted by 1, 2, 3, 4 or 5 A¹;
R⁷, R⁸ and R⁹ are each, independently, halo, C₁₋₄alkyl, C₁₋₄haloalkyl, C₆₋₁₀aryl, C₃₋₇cycloalkyl, C₂₋₅heteroaryl, C₂₋₅heterocycloalkyl, —CN, —NO₂, —OR^{a′}, —SR^{a′}, —C(═O)R^{b′}, —C(═O)NR^{c′}R^{d′}, —C(═O)OR^{a′}, —OC(═O)R^{b′}, —OC(═O)NR^{c′}R^{d′}, —NR^{c′}R^{d′}, —NR^{c′}C(═O)R^{b′}, —NR^{c′}C(═O)OR^{a′}, —NR^{c′}S(═O)₂R^{b′}, —S(═O)R^{b′}, —S(═O)NR^{c′}R^{d′}, —S(═O)₂R^{b′}, or —S(═O)₂NR^{c′}R^{d′};
A¹ is halo, —CN, —NO₂, —OR^{a}, —SR^{a}, —C(═O)R^{b}, —C(═O)NR^{c}R^{d}, —C(═O)OR^{a}, —OC(═O)R^{b}, —OC(═O)NR^{c}R^{d}, —NR^{c}R^{d}, —NR^{c}C(═O)R^{d}, —NR^{c}C(═O)OR^{a}, —NR^{c}S(═O)R^{b}, —NR^{c}S(═O)₂R^{b}, —S(═O)R^{b}, —S(═O)NR^{c}R^{d}, —S(═O)₂R^{b}, —S(═O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, C₁₋₆ alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl, wherein each of the C₁₋₆ alkyl, C₂₋₆ alkenyl, C₂₋₆ alkynyl, C₁₋₆ alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅hetercycloalkyl-C₁₋₄alkyl is optionally substituted by 1, 2, 3, 4 or 5 substituents independently selected from halo, C₁₋₆ alkyl, C₂₋₆ alkenyl, C₂₋₆ alkynyl, C₁₋₄ haloalkyl, C₆₋₁₀aryl, C₃₋₇cycloalkyl, C₂₋₅heteroaryl, C₂₋₅heterocycloalkyl, —CN, —NO₂, —OR^{a′}, —SR^{a′}, —C(═O)R^{b}, —C(═O)NR^{c′}R^{d′}, —C(═O)OR^{1′}, —OC(═O)R^{b′}, —OC(═O)NR^{c′}R^{d′}, —NR^{c′}R^{d′}, —NR^{c′}C(═O)R^{b′}, —NR^{c′}C(═O)OR^{a′}, —NR^{c′}S(═O)R^{b′}, —NR^{c′}S(═O)₂R^{b′}, —S(═O)R^{b′}, —S(═O)NR^{c′}R^{d′}, —S(═O)₂R^{b′}, or —S(═O)₂NR^{c′}R^{d′};
R^{a} and R^{a′} are each, independently, H, C₁₋₆ alkyl, C₁₋₆ haloalkyl, C₂₋₆ alkenyl, C₂₋₆ alkynyl, C₁₋₆ alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl;
R^{b} and R^{b′} are each, independently, H, C₁₋₆ alkyl, C₁₋₆ haloalkyl, C₂₋₆ alkenyl, C₂₋₆ alkynyl, C₁₋₆ alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl;
R^{c} and R^{d} are each, independently, H, C₁₋₆ alkyl, C₁₋₆ haloalkyl, C₂₋₆ alkenyl, C₂₋₆ alkynyl, C₁₋₆ alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl;
or R^{c} and R^{d} together with the N atom to which they are attached form a 4-, 5-, 6- or 7-membered heterocycloalkyl group; and
R^{c′} and R^{d′} are each, independently, H, C₁₋₆ alkyl, C₁₋₆ haloalkyl, C₂₋₆ alkenyl, C₂₋₆ alkynyl, C₁₋₆ alkyl, C₆₋₁₀aryl, C₂₋₅heteroaryl, C₃₋₇cycloalkyl, C₂₋₅heterocycloalkyl, C₆₋₁₀aryl-C₁₋₄alkyl, C₂₋₅heteroaryl-C₁₋₄alkyl, C₃₋₇cycloalkyl-C₁₋₄alkyl or C₂₋₅heterocycloalkyl-C₁₋₄alkyl;
or R^{c′} and R^{d′} together with the N atom to which they are attached form a 4-, 5-, 6- or 7-membered heterocycloalkyl group;
with the proviso that when R², R³, R⁴ and R⁵ are each H, then R⁶ is not selected from unsubstituted phenyl, 4-fluorophenyl, 4-chlorophenyl, 4-methoxyphenyl, 4-methylphenyl, 3-methoxyphenyl, 2-methoxyphenyl, and 4-N,N-dimethylaminophenyl

2. A compound of claim 1 wherein R¹ is selected from C₁₋₆ alkyl, C₃₋₆cycloalkyl, C₃₋₆cycloalkyl-C₁₋₃alkyl, C₆₋₁₀aryl-C₁₋₃alkyl, and C₂₋₅heteroaryl-C₁₋₃alkyl, each optionally substituted by 1, 2, 3, 4 or 5 substituents independently selected from halo, C₁₋₄ alkyl, C₁₋₄ haloalkyl, —CN, —NO₂, —OH, C₁₋₄ alkoxy, —O—(CH₂)ₙ—O—, C₁₋₄ haloalkoxy, amino, C₁₋₄alkylamino, and C₂₋₈ dialkylamino, wherein n is 1, 2, or 3.

3. A compound of claim 1, wherein R¹ is selected from C₁₋₆ alkyl, C₃₋₆ cycloalkyl and benzyl optionally substituted with one or more substituents selected from halogen, methoxy, and —O—CH₂—O—.

4. A compound of claim 1 wherein R¹ is selected from 4-methoxybenzyl, 3,4-dimethoxybenzyl, 2,5-dimethoxybenzyl, benzo[1,3]dioxol-5-ylmethyl, cyclopropyl, ethyl, cyclobutyl, methyl, 1-butyl, and 1-propyl.

5. A compound of claim 1, wherein R² is H, —C(═O)—(C₁₋₄ alkyl), —C(═O)-(aryl-C₁₋₃alkyl), —C(═O)O—(C₁₋₄ alkyl), —C(═O)O-(aryl-C₁₋₃alkyl), —C(═O)NH₂, —C(═O)NH(C₁₋₄ alkyl), —C(═O)N(C₁₋₄ alkyl)₂, or C₁₋₃ alkyl.

6. A compound of claim 1, wherein R² is H.

7. A compound of claim 1, wherein R³, R⁴ and R⁵ are each, independently, —H, halo, C₁₋₃alkyl, C₁₋₃alkoxy, —CN, —NO₂, —OH, halogenated C₁₋₃alkyl, or halogenated C₁₋₃alkoxy.

8. A compound of claim 1, wherein R³, R⁴ and R⁵ are each, independently, —H or halo.

9. A compound of claim 1, wherein R³ and R⁴ are each —H and R⁵ is fluoro.

10. A compound of claim 1, wherein R⁶ is phenyl or heteroaryl, each optionally substituted by 1, 2, 3, 4 or 5 substituents independently selected from halo, C₁₋₄alkoxy, C₁₋₄alkyl, halogenate C₁₋₄alkyl, —OH, amino, C₁₋₄alkylamino, C₂₋₈dialkylamino and —CN.

11. A compound of claim 1, wherein R⁶ is phenyl, naphthyl, pyridyl, pyrimidinyl, pyrazinyl, pyrazolyl, quinolyl or indolyl, each optionally substituted by 1, 2, 3, 4 or 5 substituents independently selected from halo, C₁₋₄alkoxy, C₁₋₄alkyl, halogenate C₁₋₄alkyl, —OH, amino, C₁₋₄alkylamino, C₂₋₈dialkylamino and —CN.

12. A compound of claim 1, wherein:
R⁶ is phenyl or phenoxy, each optionally substituted by 2 substituents independently selected from halo, —CN, —OH, C₁₋₄ alkoxy, C₁₋₄ haloalkoxy, amino, C₁₋₄ alkylamino, C₂₋₈ dialkylamino, C₁₋₆ alkyl, and C₁₋₆ haloalkyl.

13. A compound of claim 1, wherein
R⁶ is phenyl substituted by 2 substituents independently selected from fluoro, chloro, —CN, methyl and methoxy.

14. A compound of claim 1, wherein
R⁶ is selected from pyridyl and pyrimidinyl, wherein said pyridyl and pyrimidinyl are optionally substituted by 1 or 2 substituents independently selected from fluoro, chloro, —CN, methyl and methoxy.

15. A compound selected from:
9-amino-5-(2-fluoro-6-methoxyphenyl)-2-(4-methoxybenzyl)-2,3-dihydropyrrolo[3,4-b]quinolin-1-one;
9-amino-5-(2,5-difluorophenyl)-2-(4-methoxybenzyl)-2,3-dihydropyrrolo[3,4-b]quinolin-1-one;
9-amino-2-(4-methoxybenzyl)-5-(2-methoxypyridin-3-yl)-2,3-dihydropyrrolo[3,4-b]quinolin-1-one;
9-amino-2-(2,5-dimethoxybenzyl)-5-(2-methoxypyridin-3-yl)-2,3-dihydropyrrolo[3,4-b]quinolin-1-one;
9-amino-5-(2-fluoro-6-methoxyphenyl)-2-propyl-2,3-dihydropyrrolo[3,4-b]quinolin-1-one;
9-amino-5-(2,3-dimethylphenyl)-2-propyl-2,3-dihydropyrrolo[3,4-b]quinolin-1-one;
9-amino-5-(3,5-dimethylphenyl)-2-propyl-2,3-dihydropyrrolo[3,4-b]quinolin-1-one;
9-amino-5-(6-chloropyridin-3-yl)-2-(3,4-dimethoxybenzyl)-2,3-dihydropyrrolo[3,4-b]quinolin-1-one;
9-amino-5-(2,6-dimethoxypyridin-3-yl)-2-propyl-2,3-dihydropyrrolo[3,4-b]quinolin-1-one;
9-amino-5-(6-methylpyridin-3-yl)-2-propyl-2,3-dihydropyrrolo[3,4-b]quinolin-1-one;
9-amino-2-(3,4-dimethoxybenzyl)-5-(2,5-dimethoxyphenyl)-2,3-dihydropyrrolo[3,4-b]quinolin-1-one;
9-amino-5-(6-methoxy-4-methylpyridin-3-yl)-2-propyl-2,3-dihydropyrrolo[3,4b]quinolin-1-one;
9-amino-5-(2-fluoropyridin-3-yl)-2-propyl-2,3-dihydropyrrolo[3,4-b]quinolin-1-one;
9-amino-2-benzo[1,3]dioxol-5-ylmethyl-5-(2-methoxy-5-methylphenyl)-2,3-dihydropyrrolo[3,4-b]quinolin-1-one;
9-amino-5-(2-chloro-6-methylpyridin-3-yl)-2-propyl-2,3-dihydropyrrolo[3,4-b]quinolin-1-one;
9-amino-2-cyclopropyl-5-(2-fluoro-6-methoxyphenyl)-2,3-dihydropyrrolo[3,4-b]quinolin-1-one;
9-amino-2-ethyl-5-(2-fluoro-6-methoxyphenyl)-2,3-dihydropyrrolo[3,4-b]quinolin-1-one;
9-amino-2-cyclobutyl-5-(2-fluoro-6-methoxyphenyl)-2,3-dihydropyrrolo[3,4-b]quinolin-1-one;
9-amino-2-ethyl-5-(2-methoxypyridin-3-yl)-2,3-dihydropyrrolo[3,4-b]quinolin-1-one;
9-amino-5-(2-fluoro-6-methoxyphenyl)-2-methyl-2,3-dihydropyrrolo[3,4-b]quinolin-1-one;
9-amino-2-cyclopropyl-5-(2,5-dimethoxyphenyl)-2,3-dihydropyrrolo[3,4-b]quinolin-1-one;
9-amino-2-cyclopropyl-5-(2-fluoro-3-methoxyphenyl)-2,3-dihydropyrrolo[3,4-b]quinolin-1-one;
9-amino-5-(2-chloro-6-methylpyridin-3-yl)-2-(3,4-dimethoxybenzyl)-2,3-dihydropyrrolo[3,4-b]quinolin-1-one;
9-amino-5-(2,6-dimethoxypyridin-3-yl)-6-fluoro-2-propyl-2,3-dihydropyrrolo[3,4-b]quinolin-1-one;
2-(9-amino-2-ethyl-1-oxo-2,3-dihydro-1h-pyrrolo[3,4-b]quinolin-5-yl)-benzonitrile;
9-amino-5-(2,6-dimethoxypyridin-3-yl)-2-ethyl-6-fluoro-2,3-dihydropyrrolo[3,4b]quinolin-1-one;
9-amino-5-(2,6-dimethoxypyridin-3-yl)-2-ethyl-2,3-dihydropyrrolo[3,4-b]quinolin-1-one;
9-amino-2-cyclopropyl-5-(2,4-dimethoxyphenyl)-6-fluoro-2,3-dihydropyrrolo[3,4-b]quinolin-1-one;
9-amino-2-ethyl-5-(3,4-dimethoxyphenyl)-2,3-dihydropyrrolo[3,4-b]quinolin-1-one;
9-amino-5-(2,5-dimethoxyphenyl)-2-ethyl-6-fluoro-2,3-dihydro-1h-pyrrolo[3,4-b]quinolin-1-one;
9-amino-5-(2,6-dimethoxypyridin-3-yl)-2-methyl-2,3-dihydropyrrolo[3,4-b]quinolin-1-one;
9-amino-2-ethyl-5-(4-fluoro-2-methoxyphenyl)-2,3-dihydropyrrolo[3,4-b]quinolin-1-one;
9-amino-2-ethyl-5-(2-fluoro-3-methoxyphenyl)-2,3-dihydropyrrolo[3,4-b]quinolin-1-one;
9-amino-5-(2,4-dimethoxypyrimidin-5-yl)-2-ethyl-6-fluoro-2,3-dihydropyrrolo[3,4-b]quinolin-1-one;
9-amino-2-cyclopropyl-6-fluoro-5-(2-fluoro-6-methoxyphenyl)-2,3-dihydropyrrolo[3,4-b]quinolin-1-one;
9-amino-2-ethyl-6-fluoro-5-(4-methoxy-pyridin-3-yl)-2,3-dihydropyrrolo[3,4-b]quinolin-1-one;
9-amino-2-cyclopropyl-5-(2,5-dimethoxy-phenyl)-6-fluoro-2,3-dihydropyrrolo[3,4-b]quinolin-1-one;
9-amino-2-cyclopropyl-6-fluoro-5-(2-methoxypyridin-3-yl)-2,3-dihydropyrrolo[3,4-b]quinolin-1-one;
9-amino-2-(2,5-dimethoxybenzyl)-5-(4-methoxypyridin-3-yl)-2,3-dihydropyrrolo[3,4-b]quinolin-1-one;
9-amino-2-propyl-5-pyridin-3-yl-2,3-dihydropyrrolo[3,4-b]quinolin-1-one;
9-amino-2-cyclopropyl-6-fluoro-5-(4-methoxypyridin-3-yl)-2,3-dihydropyrrolo[3,4-b]quinolin-1-one;
9-amino-2-cyclobutyl-5-(2,5-dimethoxyphenyl)-2,3-dihydropyrrolo[3,4-b]quinolin-1-one;
9-amino-2-butyl-5-(2,6-dimethoxypyridin-3-yl)-2,3-dihydropyrrolo[3,4-b]quinolin-1-one;
9-amino-2-cyclopropyl-5-(2,4-dimethoxypyrimidin-5-yl)-6-fluoro-2,3-dihydropyrrolo[3,4-b]quinolin-1-one;
9-amino-2-ethyl-6-fluoro-5-(2-methoxyphenyl)-2,3-dihydropyrrolo[3,4-b]quinolin-1-one;
9-amino-2-ethyl-6-fluoro-5-(5-fluoro-2-methoxyphenyl)-2,3-dihydropyrrolo[3,4-b]quinolin-1-one;
9-amino-5-(3,4-dimethoxyphenyl)-2-ethyl-6-fluoro-2,3-dihydropyrrolo[3,4-b]quinolin-1-one;
9-amino-2-cyclobutyl-5-(2-methoxypyridin-3-yl)-2,3-dihydropyrrolo[3,4-b]quinolin-1-one;
9-amino-5-(5-chloro-2-methoxyphenyl)-2-cyclobutyl-2,3-dihydropyrrolo[3,4-b]quinolin-1-one;
9-amino-2-cyclopropyl-5-(3,4-dimethoxyphenoxy)-2,3-dihydro-1h-pyrrolo[3,4-b]quinolin-1-one;
9-amino-2-cyclobutyl-5-(2,6-dimethoxypyridin-3-yl)-6-fluoro-2,3-dihydropyrrolo[3,4-b]quinolin-1-one;
9-amino-2-cyclobutyl-5-(2,4-dimethoxyphenyl)-2,3-dihydro-pyrrolo[3,4-b]quinolin-1-one;
9-amino-2-cyclopropyl-5-(2,6-dimethoxypyridin-3-yl)-6-fluoro-2,3-dihydropyrrolo[3,4-b]quinolin-1-one;
9-amino-6-fluoro-5-(2-fluoro-6-methoxyphenyl)-2-propyl-2,3-dihydropyrrolo[3,4-b]quinolin-1-one;
9-amino-5-(2,6-difluoro-phenyl)-2-propyl-2,3-dihydro-pyrrolo[3,4-b]quinolin-1-one;
9-amino-2-ethyl-5-(4-methoxypyridin-3-yl)-2,3-dihydropyrrolo[3,4-b]quinolin-1-one;
9-amino-2-ethyl-6-fluoro-5-(2-methoxypyridin-3-yl)-2,3-dihydropyrrolo[3,4-b]quinolin-1-one;
9-amino-2-cyclopropyl-5-(2,5-dichlorophenyl)-2,3-dihydropyrrolo[3,4-b]quinolin-1-one;
9-amino-2-cyclopropyl-5-(2-fluoro-5-methoxyphenyl)-2,3-dihydropyrrolo[3,4-b]quinolin-1-one;
9-amino-2-cyclopropyl-6-fluoro-5-(5fluoro-2-methoxyphenyl)-2,3-dihydropyrrolo[3,4-b]quinolin-1-one;
9-amino-2-cyclobutyl-5-(4-methoxy-pyridin-3-yl)-2,3-dihydropyrrolo[3,4-b]quinolin-1-one;
9-amino-2-cyclobutyl-5-(2-methoxyphenyl)-2,3-dihydropyrrolo[3,4-b]quinolin-1-one;
9-amino-2-cyclobutyl-5-(2,6-dimethoxypyridin-3-yl)-2,3-dihydropyrrolo[3,4-b]quinolin-1-one;
9-amino-2-(3,4-dimethoxybenzyl)-5-(2-fluoro-6-methoxyphenyl)-2,3-dihydro-pyrrolo[3,4-b]quinolin-1-one;
9-amino-2-cyclopropyl-5-(2-methoxypyridin-3-yl)-2,3-dihydropyrrolo[3,4-b]quinolin-1-one;
9-Amino-2-ethyl-6-fluoro-5-(2-fluoro-6-methoxyphenyl)-2,3-dihydropyrrolo[3,4-b]quinolin-1-one;
9-Amino-5-(2,4-dimethoxy-phenyl)-2-ethyl-6-fluoro-2,3-dihydropyrrolo[3,4-b]quinolin-1-one;
9-Amino-5-(2-fluoro-6-methoxyphenyl)-2-isopropyl-2,3-dihydropyrrolo[3,4-b]quinolin-1-one;
9-Amino-6-fluoro-5-(2-fluoro-6-methoxyphenyl)-2-methyl-2,3-dihydropyrrolo[3,4-b]quinolin-1-one;
9-Amino-5-(2-fluoro-3-methoxyphenyl)-2-methyl-2,3-dihydropyrrolo[3,4-b]quinolin-1-one;
9-Amino-2-ethyl-5-(2-fluoro-5-methoxyphenyl)-2,3-dihydropyrrolo[3,4b]quinolin-1-one;
9-Amino-2-ethyl-5-(5-fluoro-2-methoxyphenyl)-2,3-dihydropyrrolo[3,4-b]quinolin-1-one;
9-Amino-2-ethyl-5-(4-fluoro-3-methoxyphenyl)-2,3-dihydropyrrolo[3,4-b]quinolin-1-one;
9-Amino-2-ethyl-5-(4-methylpyridin-3-yl)-2,3-dihydropyrrolo[3,4-b]quinolin-1-one;
2-(9-Amino-2-cyclobutyl-1-oxo-2,3-dihydro-1H-pyrrolo[3,4-b]quinolin-5-yl)benzonitrile;
9-Amino-2-cyclobutyl-5-(4-fluoro-2-methoxyphenyl)-2,3-dihydropyrrolo[3,4-b]quinolin-1-one;
9-Amino-2-cyclobutyl-5-(2-fluoro-3-methoxyphenyl)-2,3-dihydropyrrolo[3,4-b]quinolin-1-one;
9-Amino-2-cyclobutyl-5-(2-fluoro-5-methoxyphenyl)-2,3-dihydropyrrolo[3,4-b]quinolin-1-one;
9-Amino-2-cyclobutyl-5-pyrazin-2-yl-2,3-dihydropyrrolo[3,4-b]quinolin-1-one;
9-Amino-2-cyclobutyl-5-(3-methoxypyridin-4-yl)-2,3-dihydropyrrolo[3,4-b]quinolin-1-one;
9-Amino-2-cyclobutyl-5-pyridin-4-yl-2,3-dihydropyrrolo[3,4-b]quinolin-1-one;
9-Amino-2-cyclobutyl-5-pyridin-2-yl-2,3-dihydropyrrolo[3,4-b]quinolin-1-one;
9-Amino-2-cyclobutyl-5-(3,6-dimethoxypyridazin-4-yl)-2,3-dihydropyrrolo[3,4-b]quinolin-1-one;
9-Amino-2-cyclobutyl-5-(6-methoxypyridin-2-yl)-2,3-dihydropyrrolo[3,4-b]quinolin-1-one;
9-Amino-2-cyclobutyl-3-hydroxy-5-(6-methylpyridin-2-yl)-2,3-dihydropyrrolo[3,4-b]quinolin-1-one;
9-Amino-2-cyclobutyl-5-(5-methylpyridin-2-yl)-2,3-dihydropyrrolo[3,4-b]quinolin-1-one;
9-Amino-2-cyclobutyl-5-pyrimidin-2-yl-2,3-dihydropyrrolo[3,4-b]quinolin-1-one;
6-(9-Amino-2-cyclobutyl-1-oxo-2,3-dihydro-1H-pyrrolo[3,4-b]quinolin-5-yl)-nicotinonitrile;
5-(9-Amino-2-cyclobutyl-1-oxo-2,3-dihydro-1H-pyrrolo[3,4-b]quinolin-5-yl)-nicotinonitrile;
9-Amino-2-cyclobutyl-5-(3-methoxypyridazin-4-yl)-2,3-dihydropyrrolo[3,4-b]quinolin-1-one;
9-Amino-2-cyclobutyl-5-(4-methoxy-pyrimidin-5-yl)-2,3-dihydropyrrolo[3,4-b]quinolin-1-one;
9-Amino-2-cyclobutyl-5-(3-fluoropyridin-2-yl)-2,3-dihydropyrrolo[3,4-b]quinolin-1-one;
2-(9-Amino-2-cyclobutyl-1-oxo-2,3-dihydro-1H-pyrrolo[3,4-b]quinolin-5-yl)-5-fluorobenzonitrile;
2-(9-Amino-2-cyclobutyl-1-oxo-2,3-dihydro-1H-pyrrolo[3,4-b]quinolin-5-yl)-4-fluorobenzonitrile;
4-(9-Amino-2-cyclobutyl-1-oxo-2,3-dihydro-1H-pyrrolo[3,4-b]quinolin-5-yl)-6-methoxynicotinonitrile;
9-Amino-5-(1,3-dimethyl-1H-pyrazol-4-yl)-6-fluoro-2-(R)-tetrahydro-furan-3-yl-2,3-dihydropyrrolo[3,4-b]quinolin-1-one;
9-Amino-2-cyclobutyl-5-(5-fluoro-2-methoxypyridin-4-yl)-2,3-dihydropyrrolo[3,4-b]quinolin-1-one;
9-Amino-2-cyclobutyl-5-(5-fluoro-2-methoxyphenyl)-2,3-dihydropyrrolo[3,4-b]quinolin-1-one;
9-Amino-2-cyclobutyl-5-(2,4-dimethoxy-phenyl)-6-fluoro-2,3-dihydropyrrolo[3,4-b]quinolin-1-one;
9-Amino-2-cyclobutyl-6-fluoro-5-(6-methylpyridin-3-yl)-2,3-dihydropyrrolo[3,4-b]quinolin-1-one;
9-Amino-2-cyclobutyl-6-fluoro-5-(2-fluoropyridin-3-yl)-2,3-dihydropyrrolo[3,4-b]quinolin-1-one;
9-Amino-2-cyclobutyl-6-fluoro-5-(4-fluoro-2-methoxyphenyl)-2,3-dihydropyrrolo[3,4-b]quinolin-1-one;
9-Amino-2-cyclobutyl-6-fluoro-5-pyrimidin-5-yl-2,3-dihydropyrrolo[3,4-b]quinolin-1-one;
9-Amino-2-cyclobutyl-6-fluoro-5-(3-methoxypyridin-4-yl)-2,3-dihydropyrrolo[3,4-b]quinolin-1-one;
9-Amino-2-cyclobutyl-6-fluoro-5-(2-fluoro-3-methoxyphenyl)-2,3-dihydropyrrolo[3,4-b]quinolin-1-one;
9-Amino-2-cyclobutyl-6-fluoro-5-(6-methoxypyridin-3-yl)-2,3-dihydropyrrolo[3,4-b]quinolin-1-one;
9-Amino-2-cyclobutyl-6-fluoro-5-(2-vinylphenyl)-2,3-dihydropyrrolo[3,4-b]quinolin-1-one;
9-Amino-2-(3-chloro-4-methoxybenzyl)-5-(2-fluoro-6-methoxyphenyl)-2,3-dihydropyrrolo[3,4-b]quinolin-1-one;
2-(9-Amino-2-cyclopropyl-1-oxo-2,3-dihydro-1H-pyrrolo[3,4-b]quinolin-5-yl)-benzonitrile;
9-Amino-2-cyclopropyl-5-(6-methyl-pyridin-3-yl)-2,3-dihydropyrrolo[3,4-b]quinolin-1-one;
9-Amino-2-cyclopropyl-5-(2,5-difluoro-phenyl)-2,3-dihydropyrrolo[3,4-b]quinolin-1-one;
9-Amino-2-cyclopropyl-5-(2-fluorophenyl)-2,3-dihydropyrrolo[3,4-b]quinolin-1-one;
9-Amino-2-cyclopropyl-5-(2,6-difluoro-phenyl)-2,3-dihydropyrrolo[3,4-b]quinolin-1-one;
9-Amino-2-cyclopropyl-5-(2-fluoro-4-methoxy-phenyl)-2,3-dihydropyrrolo[3,4-b]quinolin-1-one;
9-Amino-5-(2-chloro-5-methoxyphenyl)-2-cyclopropyl-2,3-dihydropyrrolo[3,4-b]quinolin-1-one;
9-Amino-2-cyclopropyl-5-(2,6-difluoro-4-methoxyphenyl)-2,3-dihydropyrrolo[3,4-b]quinolin-1-one;
9-Amino-2-cyclopropyl-5-(2,3-difluorophenyl)-2,3-dihydropyrrolo[3,4-b]quinolin-1-one;
9-Amino-2-cyclopropyl-5-(2,4-difluorophenyl)-2,3-dihydropyrrolo[3,4-b]quinolin-1-one;
9-Amino-2-cyclopropyl-5-(2-fluoro-6-methylpyridin-3-yl)-2,3-dihydropyrrolo[3,4-b]quinolin-1-one;
9-Amino-2-cyclobutyl-5-(6-methylpyridin-3-yl)-2,3-dihydropyrrolo[3,4-b]quinolin-1-one;
9-Amino-2-cyclobutyl-5-(2,6-difluoro-4-methoxyphenyl)-2,3-dihydropyrrolo[3,4-b]quinolin-1-one;
9-Amino-2-cyclobutyl-5-(2,4-dimethoxy-pyrimidin-5-yl)-2,3-dihydropyrrolo[3,4-b]quinolin-1-one;
9-Amino-2-cyclobutyl-5-(2-fluorophenyl)-2,3-dihydro-pyrrolo[3,4-b]quinolin-1-one;
5-(9-Amino-2-cyclobutyl-1-oxo-2,3-dihydro-1H-pyrrolo[3,4-b]quinolin-5-yl)-pyridine-2-carbonitrile;
9-Amino-2-cyclobutyl-5-(6-fluoro-2-methylpyridin-3-yl)-2,3-dihydropyrrolo[3,4-b]quinolin-1-one;
9-Amino-2-cyclobutyl-5-(2-fluoropyridin-3-yl)-2,3-dihydropyrrolo[3,4-b]quinolin-1-one;
9-Amino-2-cyclobutyl-5-(6-methoxy-5-methylpyridin-3-yl)-2,3-dihydropyrrolo[3,4-b]quinolin-1-one;
9-Amino-2-cyclobutyl-5-((P)-2-fluoro-6-methoxyphenyl)-2,3-dihydro-pyrrolo[3,4-b]quinolin-1-one;
9-Amino-2-cyclobutyl-5-((M)-2-fluoro-6-methoxyphenyl)-2,3-dihydro-pyrrolo[3,4-b]quinolin-1-one;
2-(9-Amino-2-cyclobutyl-1-oxo-2,3-dihydro-1H-pyrrolo[3,4-b]quinolin-5-yl)-6-methoxybenzonitrile;
2-(9-Amino-2-cyclobutyl-1-oxo-2,3-dihydro-1H-pyrrolo[3,4-b]quinolin-5-yl)-3-methoxybenzonitrile;
9-Amino-2-cyclobutyl-5-(2,6-difluoropyridin-3-yl)-2,3-dihydropyrrolo[3,4-b]quinolin-1-one;
9-Amino-5-(2-fluoro-6-methoxyphenyl)-2-(3-methylcyclobutyl)-2,3-dihydropyrrolo[3,4-b]quinolin-1-one;
9-Amino-2-cyclobutyl-5-(1-methyl-1H-pyrazol-4-yl)-2,3-dihydropyrrolo[3,4-b]quinolin-1-one;
9-Amino-5-(6-methoxy-2-methylpyridin-3-yl)-2-((1s,3s)-3-methylcyclobutyl)-2,3-dihydro-1H-pyrrolo[3,4-b]quinolin-1-one;
9-Amino-5-(2-fluoro-6-methoxyphenyl)-2-((1s,3s)-3-methylcyclobutyl)-2,3-dihydro-1H-pyrrolo[3,4-b]quinolin-1-one;
9-Amino-5-(2-methoxypyridin-3-yl)-2-((1s,3s)-3-methylcyclobutyl)-2,3-dihydro-1H-pyrrolo[3,4-b]quinolin-1-one;
2-(9-Amino-2-cyclopropyl-1-oxo-2,3-dihydro-1H-pyrrolo[3,4-b]quinolin-5-yl)-3-methoxybenzonitrile;
9-Amino-2-cyclopropyl-5-((P)2-fluoro-6-methoxyphenyl)-2,3-dihydropyrrolo[3,4-b]quinolin-1-one;
9-Amino-2-cyclobutyl-5-(2-fluoro-6-methylpyridin-3-yl)-2,3-dihydropyrrolo[3,4-b]quinolin-1-one;
9-Amino-2-cyclobutyl-5-(6-methoxy-2-methylpyridin-3-yl)-2,3-dihydropyrrolo[3,4-b]quinolin-1-one;
9-Amino-2-cyclobutyl-5-(1,3-dimethyl-1H-pyrazol-4-yl)-2,3-dihydropyrrolo[3,4-b]quinolin-1-one;
9-Amino-2-cyclobutyl-5-(6-fluoro-5-methylpyridin-3-yl)-2,3-dihydropyrrolo[3,4-b]quinolin-1-one;
9-Amino-2-cyclopentyl-5-(2-fluoro-6-methoxyphenyl)-2,3-dihydropyrrolo[3,4-b]quinolin-1-one;
2-(9-Amino-2-cyclopentyl-1-oxo-2,3-dihydro-1H-pyrrolo[3,4-b]quinolin-5-yl)-benzonitrile;
9-Amino-2-cyclopentyl-5-(6-methoxy-pyridin-3-yl)-2,3-dihydro-pyrrolo[3,4-b]quinolin-1-one;
9-Amino-2-cyclobutyl-5-(6-morpholin-4-yl-pyridin-3-yl)-2,3-dihydropyrrolo[3,4-b]quinolin-1-one;
9-Amino-2-cyclobutyl-5-(6-methoxypyridin-3-yl)-2,3-dihydropyrrolo[3,4-b]quinolin-1-one;
9-Amino-2-cyclobutyl-5-(4-methylpyridin-3-yl)-2,3-dihydropyrrolo[3,4-b]quinolin-1-one;
9-Amino-2-cyclobutyl-5-(3-fluoropyrazin-2-yl)-2,3-dihydropyrrolo[3,4-b]quinolin-1-one;
9-Amino-2-cyclobutyl-5-(5-methoxy-pyridin-3-yl)-2,3-dihydropyrrolo[3,4-b]quinolin-1-one;
9-Amino-2-cyclopropyl-6-fluoro-5-(2-fluoro-3-methoxyphenyl)-2,3-dihydropyrrolo[3,4-b]quinolin-1-one;
9-Amino-2-cyclopropyl-5-(2,6-difluoro-3-methoxyphenyl)-6-fluoro-2,3-dihydropyrrolo[3,4-b]quinolin-1-one;
9-Amino-2-cyclopropyl-6-fluoro-5-(2-fluoro-5-methoxyphenyl)-2,3-dihydropyrrolo[3,4-b]quinolin-1-one;
9-Amino-2-ethyl-6-fluoro-5-(4-methylpyridin-3-yl)-2,3-dihydropyrrolo[3,4-b]quinolin-1-one;
9-Amino-2-ethyl-6-fluoro-5-(2-fluoro-5-methoxyphenyl)-2,3-dihydropyrrolo[3,4-b]quinolin-1-one;
9-Amino-2-cyclobutyl-5-(2,4-dimethoxypyrimidin-5-yl)-6-fluoro-2,3-dihydropyrrolo[3,4-b]quinolin-1-one;
9-Amino-2-cyclobutyl-5-(2,5-dimethoxyphenyl)-6-fluoro-2,3-dihydropyrrolo[3,4-b]quinolin-1-one;
9-Amino-2-cyclobutyl-6-fluoro-5-(2-methoxypyridin-3-yl)-2,3-dihydropyrrolo[3,4-b]quinolin-1-one;
9-Amino-2-cyclopropyl-6-fluoro-5-(2-methoxyphenyl)-2,3-dihydropyrrolo[3,4-b]quinolin-1-one;
9-Amino-2-cyclobutyl-6-fluoro-5-(2-methoxyphenyl)-2,3-dihydropyrrolo[3,4-b]quinolin-1-one;
9-Amino-5-(5-chloro-2-methoxyphenyl)-2-ethyl-6-fluoro-2,3-dihydropyrrolo[3,4-b]quinolin-1-one;
9-Amino-2-cyclobutyl-6-fluoro-5-(2-fluoro-5-methoxyphenyl)-2,3-dihydropyrrolo[3,4-b]quinolin-1-one;
9-Amino-2-cyclobutyl-6-fluoro-5-(5-fluoro-2-methoxyphenyl)-2,3-dihydropyrrolo[3,4-b]quinolin-1-one;
9-Amino-2-cyclobutyl-6-fluoro-5-(6-methoxy-4-methylpyridin-3-yl)-2,3-dihydropyrrolo[3,4-b]quinolin-1-one;
9-Amino-2-cyclobutyl-6-fluoro-5-(6-methoxy-2-methylpyridin-3-yl)-2,3-dihydropyrrolo[3,4-b]quinolin-1-one;
9-Amino-2-cyclobutyl-5-(2,5-dimethoxypyridin-3-yl)-2,3-dihydropyrrolo[3,4-b]quinolin-1-one;
9-Amino-6-fluoro-5-(2-methoxypyridin-3-yl)-2-(R)-tetrahydrofuran-3-yl-2,3-dihydropyrrolo[3,4-b]quinolin-1-one;
9-Amino-6-fluoro-5-(2-methoxypyridin-3-yl)-2-(S)-tetrahydrofuran-3-yl-2,3-dihydro-pyrrolo[3,4-b]quinolin-1-one;
9-Amino-2-cyclobutyl-5-(3,4-dimethoxyphenyl)-6-fluoro-2,3-dihydropyrrolo[3,4-b]quinolin-1-one;
and pharmaceutically acceptable salts thereof.

16. A compound selected from:
9-amino-2-cyclopropyl-5-(2-fluoro-6-methoxyphenyl)-2,3-dihydro-1H-pyrrolo[3,4-b]quinolin-1-one;
9-amino-2-cyclobutyl-5-(2,5-dimethoxyphenyl)-6-fluoro-2,3-dihydro-1H-pyrrolo[3,4-b]quinolin-1-one;
9-amino-2-cyclobutyl-6-fluoro-5-(2-methoxypyridin-3-yl)-2,3-dihydro-1H-pyrrolo[3,4-b]quinolin-1-one;
9-amino-2-cyclobutyl-5-(2-fluorophenyl)-2,3-dihydro-1H-pyrrolo[3,4-b]quinolin-1-one;
9-amino-2-cyclobutyl-5-(2,4-dimethoxypyrimidin-5-yl)-6-fluoro-2,3-dihydro-1H-pyrrolo[3,4-b]quinolin-1-one;
2-(9-amino-2-cyclobutyl-1-oxo-2,3-dihydro-1H-pyrrolo[3,4-b]quinolin-5-yl)benzonitrile;
9-amino-2-ethyl-6-fluoro-5-(2-fluoro-5-methoxyphenyl)-2,3-dihydro-1H-pyrrolo[3,4-b]quinolin-1-one;
9-amino-2-cyclobutyl-5-(2,6-difluoro-4-methoxyphenyl)-2,3-dihydro-1H-pyrrolo[3,4-b]quinolin-1-one;
9-amino-2-ethyl-6-fluoro-5-(2-fluoro-4-methoxyphenyl)-2,3-dihydro-1H-pyrrolo[3,4-b]quinolin-1-one;
9-amino-2-(3,4-dimethoxybenzyl)-5-(4-methoxypyridin-3-yl)-2,3-dihydro-1H-pyrrolo[3,4-b]quinolin-1-one;
9-amino-2-ethyl-6-fluoro-5-(2-fluorophenyl)-2,3-dihydro-1H-pyrrolo[3,4-b]quinolin-1-one;
9-amino-2-ethyl-6-fluoro-5-(4-methylpyridin-3-yl)-2,3-dihydro-1H-pyrrolo[3,4-b]quinolin-1-one;
9-amino-2-(benzo[d][1,3]dioxol-5-ylmethyl)-5-(4-methoxypyridin-3-yl)-2,3-dihydro-1H-pyrrolo[3,4-b]quinolin-1-one;
9-amino-5-(2-chloro-6-methoxyphenyl)-2-cyclobutyl-2,3-dihydro-1H-pyrrolo[3,4-b]quinolin-1-one;
9-amino-2-(3,4-dimethoxybenzyl)-5-(2-methoxypyridin-3-yl)-2,3-dihydro-1H-pyrrolo[3,4-b]quinolin-1-one;
9-amino-5-(2,6-dimethylpyridin-3-yl)-2-propyl-2,3-dihydro-1H-pyrrolo[3,4-b]quinolin-1one;
9-amino-2-(3,4-dimethoxybenzyl)-5-(2-fluoro-6-methoxyphenyl)-2,3-dihydro-1H-pyrrolo[3,4-b]quinolin-1-one;
9-amino-2-cyclopropyl-6-fluoro-5-(2-fluoro-3-methoxyphenyl)-2,3-dihydro-1H-pyrrolo[3,4-b]quinolin-1-one;
9-amino-2-(3,4-dimethoxybenzyl)-5-(2,6-dimethylpyridin-3-yl)-2,3-dihydro-1H-pyrrolo[3,4-b]quinolin-1-one;
9-amino-2-cyclopentyl-5-(2-fluoro-6-methoxyphenyl)-2,3-dihydro-1H-pyrrolo[3,4-b]quinolin-1-one;
9-amino-2-cyclobutyl-5-(2,5-dichlorophenyl)-2,3-dihydro-1H-pyrrolo[3,4-b]quinolin-1-one;
9-amino-2-(4-methoxybenzyl)-5-(4-methoxypyridin-3-yl)-2,3-dihydro-1H-pyrrolo[3,4-b]quinolin-1-one;
9-amino-2-ethyl-6-fluoro-5-(2-fluoro-6-methoxyphenyl)-2,3-dihydro-1H-pyrrolo[3,4-b]quinolin-1-one;
9-amino-2-cyclopropyl-5-(2,6-difluorophenyl)-2,3-dihydro-1H-pyrrolo[3,4-b]quinolin-1-one;
9-amino-5-(2-methoxy-5-methylpyridin-3-yl)-2-propyl-2,3-dihydro-1H-pyrrolo[3,4-b]quinolin-1-one;
9-amino-5-(2,4-difluorophenyl)-2-ethyl-6-fluoro-2,3-dihydro-1H-pyrrolo[3,4-b]quinolin-1-one;
9-amino-2-cyclobutyl-5-(2-methoxy-5-methylphenyl)-2,3-dihydro-1H-pyrrolo[3,4-b]quinolin-1-one;
9-amino-2-cyclopropyl-5-(2,6-difluoro-3-methoxyphenyl)-6-fluoro-2,3-dihydro-1H-pyrrolo[3,4-b]quinolin-1-one;
9-amino-2-cyclobutyl-5-(2,4-dimethoxypyrimidin-5-yl)-2,3-dihydro-1H-pyrrolo[3,4-b]quinolin-1-one;
9-amino-2-cyclobutyl-5-(6-methylpyridin-3-yl)-2,3-dihydro-1H-pyrrolo[3,4-b]quinolin-1-one;
9-amino-5-(2,6-difluoro-4-methoxyphenyl)-2-ethyl-6-fluoro-2,3-dihydro-1H-pyrrolo[3,4-b]quinolin-1-one;
9-amino-2-butyl-5-(2-fluoro-6-methoxyphenyl)-2,3-dihydro-1H-pyrrolo[3,4-b]quinolin-1-one;
9-amino-2-(3,4-dimethoxybenzyl)-5-(6-methoxy-2-methylpyridin-3-yl)-2,3-dihydro-1H-pyrrolo[3,4-b]quinolin-1-one;
9-amino-2-cyclopropyl-5-(2-methoxy-5-methylphenyl)-2,3-dihydro-1H-pyrrolo[3,4-b]quinolin-1-one;
9-amino-5-(2-chlorophenyl)-2-propyl-2,3-dihydro-1H-pyrrolo[3,4-b]quinolin-1-one;
9-amino-5-(2,4-dimethoxyphenyl)-2-ethyl-6-fluoro-2,3-dihydro-1H-pyrrolo[3,4-b]quinolin-1-one;
9-amino-5-(2-chloro-6-methoxyphenyl)-2-cyclopropyl-6-fluoro-2,3-dihydro-1H-pyrrolo[3,4-b]quinolin-1-one;
9-amino-2-ethyl-6-fluoro-5-(2-fluoro-3-methoxyphenyl)-2,3-dihydro-1H-pyrrolo[3,4-b]quinolin-1-one;
9-amino-2-propyl-5-(2-methylphenyl)-2,3-dihydro-1H-pyrrolo[3,4-b]quinolin-1-one;
9-amino-2-(benzo[d][1,3]dioxol-5-ylmethyl)-5-(2-methoxypyridin-3-yl)-2,3-dihydro-1H-pyrrolo[3,4-b]quinolin-1-one;
9-amino-2-(4-methoxybenzyl)-5-(5-methoxypyridin-3-yl)-2,3-dihydro-1H-pyrrolo[3,4-b]quinolin-1-one;
9-amino-5-(5-fluoro-2-methylphenyl)-2-propyl-2,3-dihydro-1H-pyrrolo[3,4-b]quinolin-1-one;
9-amino-5-(2-chloro-5-methylpyridin-3-yl)-2-propyl-2,3-dihydro-1H-pyrrolo[3,4-b]quinolin-1-one;
9-amino-5-(2-fluoro-5-methylphenyl)-2-propyl-2,3-dihydro-1H-pyrrolo[3,4-b]quinolin-1-one;
9-amino-5-(2-fluoropyridin-3-yl)-2-(4-methoxybenzyl)-2,3-dihydro-1H-pyrrolo[3,4-b]quinolin-1-one;
9-amino-2-cyclopropyl-5-(2,5-difluorophenyl)-2,3-dihydro-1H-pyrrolo[3,4-b]quinolin-1-one;
9-amino-2-ethyl-5-(2-fluoro-4-methoxyphenyl)-2,3-dihydro-1H-pyrrolo[3,4-b]quinolin-1-one;
9-amino-2-cyclopropyl-6-fluoro-5-(2-fluoro-4-methoxyphenyl)-2,3-dihydro-1H-pyrrolo[3,4-b]quinolin-1-one;
9-amino-2-cyclopropyl-6-fluoro-5-(2-fluoro-5-methoxyphenyl)-2,3-dihydro-1H-pyrrolo[3,4-b]quinolin-1-one;
9-amino-5-(2-fluoro-5-methylpyridin-3-yl)-2-propyl-2,3-dihydro-1H-pyrrolo[3,4-b]quinolin-1-one;
9-amino-2-cyclopropyl-6-fluoro-5-(2-methoxy-5-methylphenyl)-2,3-dihydro-1H-pyrrolo[3,4-b]quinolin-1-one;
9-amino-5-(2-chloro-6-methylpyridin-3-yl)-2-cyclopropyl-2,3-dihydro-1H-pyrrolo[3,4-b]quinolin-1-one;
9-amino-5-(2-fluoro-3-methoxyphenyl)-2-(4-methoxybenzyl)-2,3-dihydro-1H-pyrrolo[3,4-b]quinolin-1-one;
9-amino-5-(2-fluoro-5-methoxypyridin-4-yl)-2-propyl-2,3-dihydro-1H-pyrrolo[3,4-b]quinolin-1-one;
9-amino-2-cyclopropyl-5-(4-methoxypyridin-3-yl)-2,3-dihydro-1H-pyrrolo[3,4-b]quinolin-1-one;
9-amino-6-fluoro-5-(2-fluoro-6-methoxyphenyl)-2-methyl-2,3-dihydro-1H-pyrrolo[3,4-b]quinolin-1-one;
9-amino-2-cyclopropyl-5-(2,6-difluoro-4-methoxyphenyl)-2,3-dihydro-1H-pyrrolo[3,4-b]quinolin-1-one;
9-amino-5-(2,4-dimethoxypyrimidin-5-yl)-6-fluoro-2-propyl-2,3-dihydro-1H-pyrrolo[3,4-b]quinolin-1-one;
9-amino-5-(5-chloro-2-methoxyphenyl)-2-cyclopropyl-2,3-dihydro-1H-pyrrolo[3,4-b]quinolin-1-one;
9-amino-2-(3,4-dimethoxybenzyl)-5-(2-methoxy-5-methylphenyl)-2,3-dihydro-1H-pyrrolo[3,4-b]quinolin-1-one;
9-amino-5-(2-chloro-5-methoxypyridin-3-yl)-2-propyl-2,3-dihydro-1H-pyrrolo[3,4-b]quinolin-1-one;
9-amino-2-(3-chloro-4-methoxybenzyl)-5-(2-methoxypyridin-3-yl)-2,3-dihydro-1H-pyrrolo[3,4-b]quinolin-1-one;
9-amino-5-(1,3-dimethyl-1H-pyrazol-5-yl)-2-(4-methoxybenzyl)-2,3-dihydro-1H-pyrrolo[3,4-b]quinolin-1-one;
9-amino-2-(3-chloro-4-methoxybenzyl)-5-(2-fluoro-6-methoxyphenyl)-2,3-dihydro-1H-pyrrolo[3,4-b]quinolin-1-one;
9-amino-5-(4-methoxy-2-(trifluoromethyl)phenyl)-2-propyl-2,3-dihydro-1H-pyrrolo[3,4-b]quinolin-1-one;
9-amino-5-(5-bromo-2-methoxypyridin-3-yl)-2-propyl-2,3-dihydro-1H-pyrrolo[3,4-b]quinolin-1-one;
9-amino-5-(2-methylpyridin-3-yl)-2-propyl-2,3-dihydro-1H-pyrrolo[3,4-b]quinolin-1-one;
9-amino-5-(2-fluoro-3-methoxyphenyl)-2-propyl-2,3-dihydro-1H-pyrrolo[3,4-b]quinolin-1-one;
9-amino-5-(2,5-dichlorophenyl)-2-propyl-2,3-dihydro-1H-pyrrolo[3,4-b]quinolin-1-one;
9-amino-5-(2,6-difluoro-4-methoxyphenyl)-2-propyl-2,3-dihydro-1H-pyrrolo[3,4-b]quinolin-1-one;
9-amino-5-(3,4-dimethoxyphenyl)-2-propyl-2,3-dihydro-1H-pyrrolo[3,4-b]quinolin-1-one;
9-amino-5-(2-fluoro-5-methoxyphenyl)-2-propyl-2,3-dihydro-1H-pyrrolo[3,4-b]quinolin-1-one;
9-amino-5-(2-fluoro-4-methoxyphenyl)-2-propyl-2,3-dihydro-1H-pyrrolo[3,4-b]quinolin-1-one;
9-amino-2-cyclopentyl-5-(2,5-dimethoxyphenyl)-2,3-dihydro-1H-pyrrolo[3,4-b]quinolin-1-one;
9-amino-2-ethyl-5-(2-fluoro-5-methoxyphenyl)-2,3-dihydro-1H-pyrrolo[3,4-b]quinolin-1-one;
9-amino-2-(3,4-dimethoxybenzyl)-5-(2,4-dimethoxypyrimidin-5-yl)-2,3-dihydro-1H-pyrrolo[3,4-b]quinolin-1-one;
9-amino-5-(2,5-dimethoxyphenyl)-2-ethyl-6-fluoro-2,3-dihydro-1H-pyrrolo[3,4-b]quinolin-1-one;
9-amino-5-(2,4-dimethoxyphenyl)-6-fluoro-2-propyl-2,3-dihydro-1H-pyrrolo[3,4-b]quinolin-1-one;
9-amino-5-(3,5-dimethylphenyl)-2-ethyl-2,3-dihydro-1H-pyrrolo[3,4-b]quinolin-1-one;
9-amino-2-cyclopropyl-5-(2-fluoro-4-methoxyphenyl)-2,3-dihydro-1H-pyrrolo[3,4-b]quinolin-1-one;
9-amino-5-(2,4-difluorophenyl)-2-propyl-2,3-dihydro-1H-pyrrolo[3,4-b]quinolin-1-one;
9-amino-5-(2-chloro-6-methoxyphenyl)-2-cyclopropyl-2,3-dihydro-1H-pyrrolo[3,4-b]quinolin-1-one;
9-amino-5-(2-ethoxypyridin-3-yl)-2-propyl-2,3-dihydro-1H-pyrrolo[3,4-b]quinolin-1-one;
9-amino-5-(3,5-difluorophenyl)-2-(3,4-dimethoxybenzyl)-2,3-dihydro-1H-pyrrolo[3,4-b]quinolin-1-one;
2-(9-amino-2-cyclopropyl-1-oxo-2,3-dihydro-1H-pyrrolo[3,4-b]quinolin-5-yl)-3-methoxybenzonitrile;
9-amino-5-(2-chloro-5-fluoropyridin-3-yl)-2-propyl-2,3-dihydro-1H-pyrrolo[3,4-b]quinolin-1-one;
2-(9-amino-2-cyclopropyl-1-oxo-2,3-dihydro-1H-pyrrolo[3,4-b]quinolin-5-yl)benzonitrile;
9-amino-5-(6-methoxy-2-methylpyridin-3-yl)-2-propyl-2,3-dihydro-1H-pyrrolo[3,4-b]quinolin-1-one;
9-amino-2-propyl-5-(2-(trifluoromethyl)phenyl)-2,3-dihydro-1H-pyrrolo[3,4-b]quinolin-1-one;
9-amino-2-cyclopropyl-5-(2,3-difluorophenyl)-2,3-dihydro-1H-pyrrolo[3,4-b]quinolin-1-one;
9-amino-2-cyclopropyl-5-(2-fluorophenyl)-2,3-dihydro-1H-pyrrolo[3,4-b]quinolin-1-one;
9-amino-2-propyl-5-(quinolin-6-yl)-2,3-dihydro-1H-pyrrolo[3,4-b]quinolin-1-one;
9-amino-5-(2-fluoro-6-methylpyridin-3-yl)-2-propyl-2,3-dihydro-1H-pyrrolo[3,4-b]quinolin-1-one;
9-amino-2-butyl-5-(2-methoxypyridin-3-yl)-2,3-dihydro-1H-pyrrolo[3,4-b]quinolin-1-one;
9-amino-2-cyclopropyl-5-(2,4-difluorophenyl)-2,3-dihydro-1H-pyrrolo[3,4-b]quinolin-1-one;
9-amino-5-(6′-chloro-2,3′-bipyridin-5-yl)-2-(3,4-dimethoxybenzyl)-2,3-dihydro-1H-pyrrolo[3,4-b]quinolin-1-one;
9-amino-5-(2-fluoro-6-methoxyphenyl)-2-((R)-1-(4-methoxyphenyl)ethyl)-2,3-dihydro-1H-pyrrolo[3,4-b]quinolin-1-one;
3-(9-amino-1-oxo-2-propyl-2,3-dihydro-1H-pyrrolo[3,4-b]quinolin-5-yl)benzonitrile;
9-amino-2-butyl-5-(2-methoxy-5-methylphenyl)-2,3-dihydro-1H-pyrrolo[3,4-b]quinolin-1-one;
9-amino-5-(1,3-dimethyl-1H-pyrazol-5-yl)-2-propyl-2,3-dihydro-1H-pyrrolo[3,4-b]quinolin-1-one;
9-amino-5-(5-chloro-2-methoxypyridin-3-yl)-2-propyl-2,3-dihydro-1H-pyrrolo[3,4-b]quinolin-1-one;
9-amino-5-(2,6-dichloropyridin-3-yl)-2-propyl-2,3-dihydro-1H-pyrrolo[3,4-b]quinolin-1-one;
9-amino-5-(6-fluoro-2-methylpyridin-3-yl)-2-propyl-2,3-dihydro-1H-pyrrolo[3,4-b]quinolin-1-one;
9-amino-2-ethyl-5-(3-fluoro-2-methoxyphenyl)-2,3-dihydro-1H-pyrrolo[3,4-b]quinolin-1-one;
9-amino-2-cyclopropyl-5-(2-(trifluoromethyl)phenyl)-2,3-dihydro-1H-pyrrolo[3,4-b]quinolin-1-one;
9-amino-5-(6-chloro-2-methylpyridin-3-yl)-2-propyl-2,3-dihydro-1H-pyrrolo[3,4-b]quinolin-1-one;
9-amino-2-ethyl-5-(4-methylpyridin-3-yl)-2,3-dihydro-1H-pyrrolo[3,4-b]quinolin-1-one;
9-amino-2-cyclopropyl-5-(2,6-dimethylphenyl)-2,3-dihydro-1H-pyrrolo[3,4-b]quinolin-1-one;
9-amino-5-(2-methoxy-6-methylpyridin-3-yl)-2-propyl-2,3-dihydro-1H-pyrrolo[3,4-b]quinolin-1-one;
9-amino-5-(2-fluoro-5-(trifluoromethyl)phenyl)-2-propyl-2,3-dihydro-1H-pyrrolo[3,4-b]quinolin-1-one;
9-amino-5-(2,3-dimethoxyphenyl)-2-propyl-2,3-dihydro-1H-pyrrolo[3,4-b]quinolin-1-one;
9-amino-2-propyl-5-(pyridin-4-yl)-2,3-dihydro-1H-pyrrolo[3,4-b]quinolin-1-one;
9-amino-2-ethyl-5-(4-fluoro-3-methoxyphenyl)-2,3-dihydro-1H-pyrrolo[3,4-b]quinolin-1-one;
9-amino-2-ethyl-5-(2-methoxy-5-methylphenyl)-2,3-dihydro-1H-pyrrolo[3,4-b]quinolin-1-one;
9-amino-5-(5-fluoro-2-methylphenyl)-2-(4-methoxybenzyl)-2,3-dihydro-1H-pyrrolo[3,4-b]quinolin-1-one;
9-amino-5-(1H-indol-5-yl)-2-propyl-2,3-dihydro-1H-pyrrolo[3,4-b]quinolin-1-one;
9-amino-5-(2-fluoro-6-methoxyphenyl)-2-isopropyl-2,3-dihydro-1H-pyrrolo[3,4-b]quinolin-1-one;
9-amino-5-(2,5-dimethoxyphenyl)-2-(3-methoxybenzyl)-2,3-dihydro-1H-pyrrolo[3,4-b]quinolin-1-one;
9-amino-5-(3,5-dimethoxyphenyl)-2-ethyl-2,3-dihydro-1H-pyrrolo[3,4-b]quinolin-1-one;
9-amino-2-ethyl-5-(5-fluoro-2-methoxyphenyl)-2,3-dihydro-1H-pyrrolo[3,4-b]quinolin-1-one;
9-amino-2-(3-chlorobenzyl)-5-(2-fluoro-6-methoxyphenyl)-2,3-dihydro-1H-pyrrolo[3,4-b]quinolin-1-one;
9-amino-5-(6-chloro-2-methylpyridin-3-yl)-2-cyclopropyl-2,3-dihydro-1H-pyrrolo[3,4-b]quinolin-1-one;
9-amino-2-cyclopropyl-5-(6-methylpyridin-3-yl)-2,3-dihydro-1H-pyrrolo[3,4-b]quinolin-1-one;
9-amino-2-(benzo[d][1,3]dioxol-5-ylmethyl)-5-(2,4-dimethoxypyrimidin-5-yl)-2,3-dihydro-1H-pyrrolo[3,4-b]quinolin-1-one;
9-amino-5-(6-fluoro-5-methylpyridin-3-yl)-2-propyl-2,3-dihydro-1H-pyrrolo[3,4-b]quinolin-1-one;
2-(9-amino-1-oxo-2-propyl-2,3-dihydro-1H-pyrrolo[3,4-b]quinolin-5-yl)benzonitrile;
9-amino-2-propyl-5-(thiophen-3-yl)-2,3-dihydro-1H-pyrrolo[3,4-b]quinolin-1-one;
9-amino-5-(4-fluoro-2-methoxyphenyl)-2-(4-methoxybenzyl)-2,3-dihydro-1H-pyrrolo[3,4-b]quinolin-1-one;
9-amino-2-cyclopropyl-5-(4-methoxy-2-(trifluoromethyl)phenyl)-2,3-dihydro-1H-pyrrolo[3,4-b]quinolin-1-one;
9-amino-5-(2,4-dimethoxyphenyl)-2-(4-methoxybenzyl)-2,3-dihydro-1H-pyrrolo[3,4-b]quinolin-1-one;
9-amino-5-(2-fluoro-3-methoxyphenyl)-2-methyl-2,3-dihydro-1H-pyrrolo[3,4-b]quinolin-1-one;
9-amino-5-(2-chloro-5-methoxyphenyl)-2-cyclopropyl-2,3-dihydro-1H-pyrrolo[3,4-b]quinolin-1-one;
9-amino-2-(4-methoxybenzyl)-5-(2-(trifluoromethyl)phenyl)-2,3-dihydro-1H-pyrrolo[3,4-b]quinolin-1-one;
9-amino-2-ethyl-5-(3-fluoro-5-methoxyphenyl)-2,3-dihydro-1H-pyrrolo[3,4-b]quinolin-1-one;
9-amino-5-(2,5-difluorophenyl)-2-propyl-2,3-dihydro-1H-pyrrolo[3,4-b]quinolin-1-one;
9-amino-2-cyclopropyl-5-(2-methoxypyridin-3-yl)-2,3-dihydro-1H-pyrrolo[3,4-b]quinolin-1-one;
9-amino-5-(6-methoxy-5-methylpyridin-3-yl)-2-propyl-2,3-dihydro-1H-pyrrolo[3,4-b]quinolin-1-one;
9-amino-2-cyclopropyl-5-(2,5-dimethoxyphenyl)-6-(methylthio)-2,3-dihydro-1H-pyrrolo[3,4-b]quinolin-1-one;
9-amino-2-cyclopropyl-5-(2,5-difluoro-4-methoxyphenyl)-2,3-dihydro-1H-pyrrolo[3,4-b]quinolin-1-one;
9-amino-5-(3-(dimethylamino)phenyl)-2-propyl-2,3-dihydro-1H-pyrrolo[3,4-b]quinolin-1-one;
9-amino-5-(furan-3-yl)-2-propyl-2,3-dihydro-1H-pyrrolo[3,4-b]quinolin-1-one;
9-amino-5-(4-methoxypyridin-3-yl)-2-propyl-2,3-dihydro-1H-pyrrolo[3,4-b]quinolin-1-one;
9-amino-2-cyclobutyl-5-(3,5-difluorophenyl)-2,3-dihydro-1H-pyrrolo[3,4-b]quinolin-1-one;
9-amino-5-(5-chloro-2-methoxyphenyl)-2-propyl-2,3-dihydro-1H-pyrrolo[3,4-b]quinolin-1-one;
9-amino-5-(2-methoxypyridin-3-yl)-2-propyl-2,3-dihydro-1H-pyrrolo[3,4-b]quinolin-1-one;
9-amino-2-ethyl-5-(3-fluoro-4-methoxyphenyl)-2,3-dihydro-1H-pyrrolo[3,4-b]quinolin-1-one;
9-amino-2-cyclopropyl-5-(2-fluoro-6-methylpyridin-3-yl)-2,3-dihydro-1H-pyrrolo[3,4-b]quinolin-1-one;
9-amino-5-phenyl-2-propyl-2,3-dihydro-1H-pyrrolo[3,4-b]quinolin-1-one;
9-amino-2-propyl-5-(3-methylphenyl)-2,3-dihydro-1H-pyrrolo[3,4-b]quinolin-1-one;
9-amino-2-(2-butyl)-5-(2-fluoro-6-methoxyphenyl)-2,3-dihydro-1H-pyrrolo[3,4-b]quinolin-1-one;
9-amino-5-(5-fluoro-2-methoxyphenyl)-2-(4-methoxybenzyl)-2,3-dihydro-1H-pyrrolo[3,4-b]quinolin-1-one;
9-amino-5-(3,5-dimethoxyphenyl)-2-propyl-2,3-dihydro-1H-pyrrolo[3,4-b]quinolin-1-one;
9-amino-5-(2-methoxy-5-methylphenyl)-2-propyl-2,3-dihydro-1H-pyrrolo[3,4-b]quinolin-1-one;
9-amino-5-(3,4-dimethoxyphenyl)-2-(4-methoxybenzyl)-2,3-dihydro-1H-pyrrolo[3,4-b]quinolin-1-one;
9-amino-5-(2,5-difluoro-4-methoxyphenyl)-2-(4-methoxybenzyl)-2,3-dihydro-1H-pyrrolo[3,4-b]quinolin-1-one;
9-amino-5-(2,4-dimethoxyphenyl)-2-propyl-2,3-dihydro-1H-pyrrolo[3,4-b]quinolin-1-one;
9-amino-2-propyl-5-(2,3,4-trimethoxyphenyl)-2,3-dihydro-1H-pyrrolo[3,4-b]quinolin-1-one;
9-amino-5-(2-methoxy-5-(pyridin-4-yl)phenyl)-2-propyl-2,3-dihydro-1H-pyrrolo[3,4-b]quinolin-1-one;
4-(9-amino-1-oxo-2-propyl-2,3-dihydro-1H-pyrrolo[3,4-b]quinolin-5-yl)benzonitrile;
9-amino-2-cyclopropyl-5-(2-methoxyphenyl)-2,3-dihydro-1H-pyrrolo[3,4-b]quinolin-1-one;
9-amino-5-(2-methoxy-5-methylphenyl)-2-(4-methoxybenzyl)-2,3-dihydro-1H-pyrrolo[3,4-b]quinolin-1-one;
9-amino-2-cyclopropyl-5-(6-methoxy-5-methylpyridin-3-yl)-2,3-dihydro-1H-pyrrolo[3,4-b]quinolin-1-one;
9-amino-5-(4-methylpyridin-3-yl)-2-propyl-2,3-dihydro-1H-pyrrolo[3,4-b]quinolin-1-one;
9-amino-5-(2-fluoro-5-methoxyphenyl)-2-methyl-2,3-dihydro-1H-pyrrolo[3,4-b]quinolin-1-one;
9-amino-5-(2,5-dimethylphenyl)-2-propyl-2,3-dihydro-1H-pyrrolo[3,4-b]quinolin-1-one;
9-amino-5-(3-fluoro-2-methoxyphenyl)-2-propyl-2,3-dihydro-1H-pyrrolo[3,4-b]quinolin-1-one;
9-amino-2-cyclopropyl-5-(2,4-dimethoxyphenyl)-2,3-dihydro-1H-pyrrolo[3,4-b]quinolin-1-one;
9-amino-5-(2,5-dimethoxyphenyl)-2-ethyl-2,3-dihydro-1H-pyrrolo[3,4-b]quinolin-1-one;
9-amino-2-(4-methoxybenzyl)-5-(6-methylpyridin-3-yl)-2,3-dihydro-1H-pyrrolo[3,4-b]quinolin-1-one;
9-amino-2-(benzo[d][1,3]dioxol-5-ylmethyl)-5-(2,5-dimethoxyphenyl)-2,3-dihydro-1-pyrrolo[3,4-b]quinolin-1-one;
9-amino-5-(4-fluoro-2-methoxyphenyl)-2-propyl-2,3-dihydro-1H-pyrrolo[3,4-b]quinolin-1-one;
9-amino-5-(3-methoxyphenyl)-2-propyl-2,3-dihydro-1H-pyrrolo[3,4-b]quinolin-1-one;
9-amino-5-(5-methoxypyridin-3-yl)-2-propyl-2,3-dihydro-1H-pyrrolo[3,4-b]quinolin-1-one;
2-(9-amino-2-methyl-1-oxo-2,3-dihydro-1H-pyrrolo[3,4-b]quinolin-5-yl)benzonitrile;
9-amino-2-cyclopropyl-5-(pyridin-3-yl)-2,3-dihydro-1H-pyrrolo[3,4-b]quinolin-1-one;
5-(9-amino-1-oxo-2-propyl-2,3-dihydro-1H-pyrrolo[3,4-b]quinolin-5-yl)nicotinonitrile;
9-amino-2-methyl-5-(4-methylpyridin-3-yl)-2,3-dihydro-1H-pyrrolo[3,4-b]quinolin-1-one;
9-amino-5-(3,5-dimethylphenyl)-2-methyl-2,3-dihydro-1H-pyrrolo[3,4-b]quinolin-1-one;
9-amino-5-(2,5-dimethoxyphenyl)-2-(4-methoxybenzyl)-2,3-dihydro-1H-pyrrolo[3,4-b]quinolin-1-one;
9-amino-5-(2-methoxy-5-methylphenyl)-2-methyl-2,3-dihydro-1H-pyrrolo[3,4-b]quinolin-1-one;
9-amino-2-(4-methoxybenzyl)-5-(1-methyl-1H-pyrazol-4-yl)-2,3-dihydro-1H-pyrrolo[3,4-b]quinolin-1-one;
9-amino-2-cyclopropyl-5-(5-fluoro-2-methoxyphenyl)-2,3-dihydro-1H-pyrrolo[3,4-b]quinolin-1-one;
9-amino-5-(3,5-dimethoxyphenyl)-2-methyl-2,3-dihydro-1H-pyrrolo[3,4-b]quinolin-1-one;
5-(9-amino-1-oxo-2-propyl-2,3-dihydro-1H-pyrrolo[3,4-b]quinolin-5-yl)-2-fluorobenzonitrile;
9-amino-5-(2,6-difluoropyridin-3-yl)-2-propyl-2,3-dihydro-1H-pyrrolo[3,4-b]quinolin-1-one;
2-(9-amino-1-oxo-2-propyl-2,3-dihydro-1H-pyrrolo[3,4-b]quinolin-5-yl)-3-methoxybenzonitrile;
9-amino-5-(2-methoxypyrimidin-5-yl)-2-propyl-2,3-dihydro-1H-pyrrolo[3,4-b]quinolin-1-one;
9-amino-5-(3-chlorophenyl)-2-propyl-2,3-dihydro-1H-pyrrolo[3,4-b]quinolin-1-one;
9-amino-2-ethyl-5-(6-methylpyridin-3-yl)-2,3-dihydro-1H-pyrrolo[3,4-b]quinolin-1-one;
9-amino-5-(2-fluoro-4-methoxyphenyl)-2-methyl-2,3-dihydro-1H-pyrrolo[3,4-b]quinolin-1-one;
9-amino-2-cyclopropyl-5-(2,4-dimethoxypyrimidin-5-yl)-2,3-dihydro-1H-pyrrolo[3,4-b]quinolin-1-one;
9-amino-5-(4-fluorophenyl)-2-propyl-2,3-dihydro-1H-pyrrolo[3,4-b]quinolin-1-one;
9-amino-5-(2,4-dimethoxyphenyl)-2-methyl-2,3-dihydro-1H-pyrrolo[3,4-b]quinolin-1-one;
9-amino-5-(5-fluoro-2-methoxyphenyl)-2-propyl-2,3-dihydro-1H-pyrrolo[3,4-b]quinolin-1-one;
9-amino-5-(3-fluoro-5-methoxyphenyl)-2-methyl-2,3-dihydro-1H-pyrrolo[3,4-b]quinolin-1-one;
9-amino-2-propyl-5-(4-methylphenyl)-2,3-dihydro-1H-pyrrolo[3,4-b]quinolin-1-one;
9-amino-5-(5-fluoro-6-methoxypyridin-3-yl)-2-propyl-2,3-dihydro-1H-pyrrolo[3,4-b]quinolin-1-one;
9-amino-5-(2,3-dimethoxyphenyl)-2-ethyl-2,3-dihydro-1H-pyrrolo[3,4-b]quinolin-1-one;
9-amino-5-(2,6-dimethoxyphenyl)-2-ethyl-2,3-dihydro-1H-pyrrolo[3,4-b]quinolin-1-one;
9-amino-2-cyclopropyl-5-(4-fluoro-2-methoxyphenyl)-2,3-dihydro-1H-pyrrolo[3,4-b]quinolin-1-one;
9-amino-5-(6-chloro-5-methylpyridin-3-yl)-2-propyl-2,3-dihydro-1H-pyrrolo[3,4-b]quinolin-1-one;
9-amino-5-(3,4-dimethoxyphenyl)-2-methyl-2,3-dihydro-1H-pyrrolo[3,4-b]quinolin-1-one;
9-amino-2-propyl-5-(3,4,5-trimethoxyphenyl)-2,3-dihydro-1H-pyrrolo[3,4-b]quinolin-1-one;
9-amino-2-butyl-5-(6-methylpyridin-3-yl)-2,3-dihydro-1H-pyrrolo[3,4-b]quinolin-1-one;
9-amino-5-(2,3-difluorophenyl)-2-(4-methoxybenzyl)-2,3-dihydro-1H-pyrrolo[3,4-b]quinolin-1-one;
9-amino-5-(2,4-dimethoxyphenyl)-2-ethyl-2,3-dihydro-1H-pyrrolo[3,4-b]quinolin-1-one;
9-amino-5-(1-methyl-1H-pyrazol-4-yl)-2-propyl-2,3-dihydro-1H-pyrrolo[3,4-b]quinolin-1-one;
9-amino-2-(3-methoxybenzyl)-5-(4-methoxypyridin-3-yl)-2,3-dihydro-1H-pyrrolo[3,4-b]quinolin-1-one;
9-amino-5-(2,5-difluoro-4-methoxyphenyl)-2-propyl-2,3-dihydro-1H-pyrrolo[3,4-b]quinolin-1-one;
9-amino-2-cyclopropyl-5-(3,4-dimethoxyphenyl)-6-fluoro-2,3-dihydro-1H-pyrrolo[3,4-b]quinolin-1-one;
9-amino-2-cyclobutyl-5-(2-fluoro-6-methylpyridin-3-yl)-2,3-dihydro-1H-pyrrolo[3,4-b]quinolin-1-one;
9-amino-5-(3,4-dimethoxyphenyl)-6-fluoro-2-propyl-2,3-dihydro-1H-pyrrolo[3,4-b]quinolin-1-one;
9-amino-2-cyclobutyl-5-(3,4-dimethoxyphenyl)-6-fluoro-2,3-dihydro-1H-pyrrolo[3,4-b]quinolin-1-one;
9-amino-2-cyclopentyl-5-(2,4-dimethoxypyrimidin-5-yl)-2,3-dihydro-1H-pyrrolo[3,4-b]quinolin-1-one;
9-amino-2-cyclopentyl-5-(2-methoxypyridin-3-yl)-2,3-dihydro-1H-pyrrolo[3,4-b]quinolin-1-one;
9-amino-5-(2-chloro-6-methylpyridin-3-yl)-2-cyclobutyl-2,3-dihydro-1H-pyrrolo[3,4-b]quinolin-1-one;
5-(9-amino-2-cyclobutyl-1-oxo-2,3-dihydro-1H-pyrrolo[3,4-b]quinolin-5-yl)picolinonitrile;
9-amino-5-(3,4-dimethoxyphenyl)-2-ethyl-6-(methylthio)-2,3-dihydro-1H-pyrrolo[3,4-b]quinolin-1-one;
9-amino-2-cyclobutyl-5-(pyridazin-4-yl)-2,3-dihydro-1H-pyrrolo[3,4-b]quinolin-1-one;
2-(9-amino-2-cyclopentyl-1-oxo-2,3-dihydro-1H-pyrrolo[3,4-b]quinolin-5-yl)benzonitrile;
9-amino-2-cyclopentyl-5-(2,5-difluoro-4-methoxyphenyl)-2,3-dihydro-1H-pyrrolo[3,4-b]quinolin-1-one;
9-amino-2-cyclopentyl-5-(5-methoxypyridin-3-yl)-2,3-dihydro-1H-pyrrolo[3,4-b]quinolin-1-one;
9-amino-2-cyclopentyl-5-(6-methoxypyridin-3-yl)-2,3-dihydro-1H-pyrrolo[3,4-b]quinolin-1-one;
9-amino-2-cyclopentyl-5-(2,6-dimethoxypyridin-3-yl)-2,3-dihydro-1H-pyrrolo[3,4-b]quinolin-1-one;
9-amino-2-cyclopentyl-5-(2-ethoxypyridin-3-yl)-2,3-dihydro-1H-pyrrolo[3,4-b]quinolin-1-one;
and pharmaceutically acceptable salts thereof.

17. A pharmaceutical composition comprising a compound of claim 1 and a pharmaceutically acceptable carrier, diluent or excipient.

18. (canceled)

19. A method for the treatment of schizophrenia comprising administrating an effective amount of a compound according to claim 1 to a patient in need thereof.

20. A method for the treatment of cognitive disorder associated with schizophrenia comprising administrating an effective amount of a compound according to claim 1 to a patient in need thereof.

21. A method for the treatment of anxiety disorder comprising administrating an effective amount of a compound according to claim 1 to a patient in need thereof.

22. A method for the treatment of cognitive disorder comprising administrating an effective amount of a compound according to claim 1 to a patient in need thereof.

23. The method according to claim 22 wherein the cognitive disorder is Alzheimer's disease, dementia, dementia due to Alzheimer's disease, or dementia due to Parkinson's disease.

24. A method for the treatment of mood disorder comprising administrating an effective amount of a compound according to claim 1 to a patient in need thereof.

25. The method according to claim 24 wherein the mood disorder is a depressive disorder.

26. A method of modulating activity of a GABAA receptor comprising contacting said GABAA receptor with a compound of claim 1.

27. The method of claim 26, wherein said GABAA receptor is a GABAA1 receptor, GABAA2 receptor, GABAA3 receptor or GABAA5 receptor.

28. The method of claim 26, wherein said GABAA receptor is a GABAA2 receptor.
